(19) United States
(12) Patent Application Publication
(10) Pub. No.: US 2009/0183269 A1
(43) Pub. Date: Jul. 16, 2009

(54) GENE EXPRESSION SYSTEM USING ALTERNATIVE SPLICING IN INSECTS

(75) Inventor: Luke Alphey, Abingdon (GB)

Correspondence Address: GREENLEE WINNER AND SULLIVAN P C, 4875 PEARL EAST CIRCLE, SUITE 200, BOULDER, CO, 80301, US

(73) Assignee: Oxitec Limited, Abingdon (GB)

(21) Appl. No.: 12/278,849

(22) PCT Filed: Feb. 12, 2007
(86) PCT No.: PCT/GB07/00488

§ 371 Date: Mar. 6, 2009

Related U.S. Application Data
(63) Continuation-in-part of application No. 11/352,117, filed on Feb. 9, 2006, now Pat. No. 7,530,898.

(30) Foreign Application Priority Data
Oct. 25, 2006 (GB) 0621234.4

(51) Int. Cl. A01K 67/027; C12N 15/00

(52) U.S. Cl. 800/21; 435/320.1

(57) ABSTRACT

A polynucleotide expression system is provided that is capable of alternative splicing of RNA transcripts of a polynucleotide sequence to be expressed in an organism.

### INTRODUCTION

The present invention relates to a gene expression system, in combination with splice control sequences, said control sequences providing a mechanism for alternative splicing.

Alternative splicing involves the removal of one or more introns and ligation of the flanking exons. This reaction is catalyzed by the spliceosome, a macromolecular machine composed of five RNAs and hundreds of proteins (Jurica, M. S. & Moore, M. J. (2003) Mol. Cell 12, 5-14). Alternative splicing generates multiple mRNAs from a single gene, thus increasing proteome diversity (Graveley, B. R. (2001) Trends Genet. 17, 100-107).

Alternative splicing also plays a key role in the regulation of gene expression in many developmental processes ranging from sex determination to apoptosis (Black, D. L. (2003) Annu. Rev. Biochem. 72, 291-336), and defects in alternative splicing have been linked to many human disorders (Caceres, J. F. & Kornblihtt, A. R. (2002) Trends Genet. 18, 186-193). In general, alternative splicing is regulated by proteins that associate with the pre-mRNA and function to either enhance or repress the ability of the spliceosome to recognize the splice site(s) flanking the regulated exon (Smith, C. W. & Valcarcel, J. (2000) Trends Biochem. Sci. 25, 381-388).

Whether a particular alternative exon will be included or excluded from a mature RNA in each cell is thought to be determined by the relative concentration of a number of positive and negative splicing regulators and the interactions of these factors with the pre-mRNA and components of the spliceosome (Smith, C. W. & Valcarcel, J. (2000) Trends Biochem. Sci. 25, 381-388).

Spliceosomes are large complexes of small nuclear RNA and protein particles (snRNPs) which assemble with pre-mRNA to achieve RNA splicing, by removing introns from eukaryotic nuclear RNAs, thereby producing mRNA which is then translated to protein in ribosomes.

Although at least 74% of human genes encode alternatively spliced mRNAs (Johnson, J. M., Castle, J., Garrett-Engele, P., Kan, Z., Loerch, P. M., Armour C. D., Santos, R., Schadt, E. E., Stoughton, R. & Shoemaker, D. D. (2003) Science 302, 2141-2144), relatively few splicing regulators have been identified.

### SUMMARY OF THE INVENTION

Thus, in a first aspect, the present invention provides a polynucleotide expression system comprising:
at least one heterologous polynucleotide sequence encoding a functional protein, defined between a start codon and a stop codon, and/or polynucleotides for interference RNA (RNAi), to be expressed in an organism;
at least one promoter operably linked thereto; and
at least one splice control sequence which, in cooperation with a spliceosome, is capable of (i) mediating splicing of an RNA transcript of the coding sequence to yield a first spliced messenger RNA (mRNA) product, and (ii) mediating at least one alternative splicing of said RNA transcript to yield an alternative spliced mRNA product;
wherein, when the at least one heterologous polynucleotide sequence encodes a functional protein, at least one of the mature mRNA products comprising a continuous Open Reading Frame (ORF) extending from said start codon to said stop codon, thereby defining a protein, which is said functional protein, or is related to said functional protein by at least one amino acid deletion, and which is functional when translated and, optionally, has undergone post-translational modification;
the mediation being selected from the group consisting of: sex-specific mediation, stage-specific mediation, germline-specific mediation, tissue-specific mediation, and combinations thereof.

The expression system may be DNA or RNA or a hybrid or combination of both. It is envisaged that the system comprises both ribo- and deoxy-ribonucleotides, i.e. portions of DNA and portions of RNA. These could correspond to different genetic elements, such that the system is a DNA/RNA hybrid, with some functional elements provided by DNA and others by RNA.

Preferably, the mediation is in a sex-specific, stage-specific, germline-specific or tissue-specific manner. In particular, sex-specific mediation is particularly preferred. However, it is also preferred that a combination of these four manners of mediation can be utilised. It is particularly preferred that, when a combination of these modes is used, that this includes sex-specific mediation. A particularly preferred example of such a combination is a combination of sex-specific, tissue-specific and stage-specific mediation of alternative splicing.

The system may be adapted for expression of a gene. Preferably, the polynucleotide sequence to be expressed comprises a coding sequence for a protein or polypeptide, i.e. at least one exon, and preferably 2 or more exons, capable of encoding a polypeptide, such as a protein or fragment thereof.

It will be understood that an exon is any region of DNA within a gene, that is present in a mature RNA molecule derived from that gene, rather than being spliced out from the transcribed RNA molecule. For protein coding genes, mature RNA molecules correspond to mature mRNA molecules, which may encode one or more proteins or polypeptides. Exons of many eukaryotic genes interleave with segments of non-coding DNA.

The at least one heterologous polynucleotide sequence may encode a functional protein, defined between a start codon and a stop codon to be expressed in an organism. Alternatively, or in addition, the at least one heterologous polynucleotide sequence encodes or comprises polynucleotides for interference RNA (RNAi), to be expressed in an organism.

These sequences, to be expressed in the organism, may also be referred to as sequences, the expression of which is to be regulated in said organism.

Preferably, the polynucleotide sequence to be expressed comprises two or more coding exons, being segments or sequences of polynucleotides that encode amino acids when translated from mRNA. Preferably, the different exons are differentially spliced together to provide alternative mRNAs. Preferably, said alternative spliced mRNAs have different coding potential, i.e. encode different proteins or polypeptide sequences. Thus, the expression of the coding sequence is regulated by alternative splicing in the above-mentioned manners of mediation.

The polynucleotide sequence to be expressed may comprise polynucleotides for interference RNA (RNAi). Such sequences are capable of providing, for instance, one or more stretches of double-stranded RNA (dsRNA), preferably in the form of a primary transcript, which in turn is capable of processing by the RNA Pol III-like enzyme “Dicer.” Such stretches include, for instance, stretches of single-stranded RNA that can form loops, such as those found in short-hairpin RNA (shRNA), or with Longer regions that are substantially self-complementary.

Thus, where the system is DNA, the polynucleotides for interference RNA are deoxyribonucleotides that, when transcribed into pre-RNA ribonucleotides, provide a stretch of dsRNA, as discussed above.

Polynucleotides for interference RNA are particularly preferred when said polynucleotides are positioned to minimise interference with alternative splicing. This may be achieved by distal positioning of these polynucleotides from the alternative splicing control sequences, preferably 3′ to the control sequences. In another preferred embodiment, substantially self-complementary regions may be separated from each other by one or more splice control sequences, such as an intron, that mediate alternative splicing. Preferably, the self-complementary regions are arranged as a series of two or more inverted repeats, each inverted repeat separated by splice control sequence, preferably an intron, as defined elsewhere.

In this configuration, different alternatively spliced transcripts may have their substantially self-complementary regions separated by different lengths of non-self-complementary sequence in the mature (post-alternative-splicing) transcript. It will be appreciated that regions that are substantially self-complementary are those that are capable of forming hairpins, for instance, as portions of the sequence are capable of base-pairing with other portions of the sequence. These two portions do not have to be exactly complementary to each other, as there can be some mismatching or toleration of stretches in each portion that do not base-pair with each other. Such stretches may not have an equivalent in the other portion, such that symmetry is lost and “bulges” form, as is known with base-pair complementation in general.

In another preferred embodiment, one or more segment of sequence substantially complementary to another section of the primary transcript is positioned, relative to the at least one splice control sequence, so that it is not included in all of the transcripts produced by alternative splicing of the primary transcript. By this method, some transcripts are produced that tend to produce dsRNA while others do not; by mediation of the alternative splicing, e.g. sex-specific mediation, stage-specific mediation, germline-specific mediation, tissue-specific mediation, and combinations thereof, dsRNA may be produced in a sex-specific, stage-specific, germline-specific or tissue-specific manner, or combinations thereof.

The system is preferably capable of expressing at least one protein of interest, i.e. said functional protein to be expressed in an organism. Said at least one protein of interest may have a therapeutic effect or may, preferably, be a marker, for instance DsRed, Green Fluorescent Protein (GFP) or one or more of their mutants or variants, or other markers that are well known in the art.

Most preferably, the functional protein to be expressed in an organism has a lethal, deleterious or sterilizing effect. Where reference is made herein to a lethal effect, it will be appreciated that this extends to a deleterious or sterilizing effect, such as an effect capable of killing the organism per se or its offspring, or capable of reducing or destroying the function of certain tissues thereof, of which the reproductive tissues are particularly preferred, so that the organism or its offspring are sterile. Therefore, some lethal effects, such as poisons, will kill the organism or tissue in a short time-frame relative to their life-span, whilst others may simply reduce the organism's ability to function, for instance reproductively.

A lethal effect resulting in sterilization is particularly preferred, as this allows the organism to compete in the natural environment (“in the wild”) with wild-type organisms, but the sterile insect cannot then produce viable offspring. In this way, the present invention achieve a similar result to techniques such as the Sterile Insect Technique (SIT) in insects, without the problems associated with SIT, such as the cost, danger to the user, and reduced competitiveness of the irradiated organism.

Preferably, the system comprises at least one positive feedback mechanism, namely at least functional protein to be differentially expressed, via alternative splicing, and at least one promoter therefor, wherein a product of a gene to be expressed serves as a positive transcriptional control factor for the at least one promoter, and whereby the product, or the expression of the product, is controllable. Preferably, an enhancer is associated with the promoter, the gene product serving to enhance activity of the promoter via the enhancer. Preferably, the control factor is the tTA gene product or an analogue thereof, and wherein one or more tetO operator units is operably linked with the promoter and is the enhancer, tTA or its analogue serving to enhance activity of the promoter via tetO. It is preferred that functional protein encodes the tTAV or tTAF product and preferably, the promoter is substantially inactive in the absence of the positive transcriptional control factor. Suitable, preferably minimal, promoters for this system can be selected from: hsp70, a P minimal promoter, a CMV minimal promoter, an Act5C-based minimal promoter, a BmA3 promoter fragment, a promoter fragment from hunchback, an Adh core promoter, and an Act5C minimal promoter, or combinations thereof.

In one embodiment, the functional protein is preferably an apoptosis-inducing factor, such as the AIF protein described for instance in Candé et al (Journal of Cell Science 115, 4727-4734 (2002)) or homologues thereof. AIF homologues are found in mammals and even in invertebrates, including insects, nematodes, fungi, and plants, meaning that the AIF gene has been conserved throughout the eukaryotic kingdom. Also preferred is Hid, the protein product of the head involution defective gene of Drosophila melanogaster, or Reaper (Rpr), the product of the reaper gene of Drosophila, or mutants thereof. Use of Hid was described by Heinrich and Scott (Proc. Natl Acad. Sci USA 97, 8229-8232 (2000). Use of a mutant derivative, Hid^{Ala5} was described by Horn and Wimmer (Nature Biotechnology 21, 64-70 (2003)). Use of a mutant derivative of Rpr, Rpr^{KR}, is described herein (see also White et al 1996, Wing et al., 2001, and Olson et al., 2003). Both Rpr and Hid are pro-apoptotic proteins, thought to bind to IAP1. IAP1 is a well-conserved anti-apoptotic protein. Hid and Rpr are therefore expected to work across a wide phylogenetic range (Huang et al., 2002, Vernooy et al., 2000) even though their own sequence is not well conserved.

Also preferred is Nipp1Dm, the Drosophila homologue of mammalian Nipp1 (Parker et al Biochemical Journal 368, 789-797 (2002); Bennett et al., Genetics 164, 235-245 (2003)). Nipp1Dm is another example of a protein with lethal effect if expressed at a suitable level, as would be understood by the skilled person. Indeed, many other examples of proteins with a lethal effect will be known to the person skilled in the art.

It is also preferred that the functional protein itself a transcriptional transactivator, such as the tTAV system described above.

It is preferred that the promoter can be activated by environmental conditions, for instance the presence or absence of a particular factor such as tetracycline in the tet system described herein, such that the expression of the gene of interest can be easily manipulated by the skilled person. Alternatively, a preferred example of a suitable promoter is the hsp70 heat shock promoter, allowing the user to control expression by variation of the environmental temperature to which the hosts are exposed in a lab or in the field, for instance. Another preferred example of temperature control is described in Fryxell and Miller (Journal of Economic Entomology 88, 1221-1232 (1995)).

Also preferred as a promoter is the srya embryo-specific promoter (Horn & Wimmer (2003) from Drosophila melanogaster, or its homologues, or promoters from other embryo-specific or embryo-active genes, such as that of the Drosophila gene slow as molasses (slam), or its homologues from other species.

It is also preferred that the system comprises other upstream, 5′ factors and/or downstream 3′ factors for controlling expression. Examples include enhancers such as the fat-body enhancers from the Drosophila yolk protein genes, and the homology region (hr) enhancers from baculoviruses, for example AcMNPV. It will also be appreciated that the RNA products will include suitable 5′ and 3′ UTRs, for instance.

The splice control sequence allows an additional level of control of protein expression, in addition to the promoter and/or enhancer of the gene. For instance, tissue or sex-specific expression in insect embryos only would be extremely difficult by conventional methods. Promoters with this specificity are unknown, even in Drosophila. However, using combinatorial control according to the present invention, an embryo-specific promoter, for example srya, can be combined with a suitable alternative splicing system.

It is preferred that any combination of promoter and alternative splicing mechanism is envisaged. The promoter is preferably specific to a particular protein having a short temporal or confined spatial effect, for example a cell-autonomous effect.

Alternatively, it is preferred that the promoter may be specific for a broader class of proteins or a specific protein that has a long-term and/or wide system effect, such as a hormone, positive or negative growth factor, morphogen or other secreted or cell-surface signalling molecule. This would allow, for instance, a broader expression pattern so that a combination of a morphogen promoter with a stage-specific alternative splicing mechanism could result in the morphogen being expressed only once a certain life-cycle stage was reached, but the effect of the morphogen would still be felt (i.e. the morphogen can still act and have an effect) beyond that life-cycle stage. Preferred examples would be the morphogen/signaling molecules Hedgehog, Wingless/WNTs, TGFβ/BMPs, EGF and their homologues, which are well-known evolutionarily-conserved signalling molecules.

It is also envisaged that a promoter that is activated by a range of protein factors, for instance transactivators, or which has a broad systemic effect, such as a hormone or morphogen, could be used in combination with an alternative splicing mechanism to achieve a tissue and sex-specific control or sex and stage-specific control, or other combinations of stage-, tissue, germ-line- and sex-specific control.

It is also envisaged that more than one promoter, and optionally an enhancer therefor, can be used in the present system, either as alternative means for initiating transcription of the same protein or by virtue of the fact that the genetic system comprises more than one gene expression system (i.e. more than one gene and its accompanying promoter).

In a further aspect, the present invention provides a method of transformation, comprising expressing two or more RNA molecules, derived from a single primary transcript, or substantially similar primary transcripts, by alternative splicing, said two or more RNA molecules preferably encoding different proteins or polypeptides, in an organism by contacting the organism with the expression system and preferably inducing expression of the expression system. Methods of introduction or transformation of the gene system and induction of expression are well known in the art with respect to the relevant organism.

Also provided are organisms (i.e. transformants) transformed by the present system.

Where reference to a particular nucleotide or protein sequence is made, it will be understood that this includes reference to any mutant or variant thereof, having substantially equivalent biological activity thereto. Preferably, the mutant or variant has at least 85%, preferably at least 90%, preferably at least 95%, preferably at least 99%, preferably at least 99.9%, and most preferably at least 99.99% sequence identity with the reference sequences.

The sequences provided can tolerate some sequence variation and still splice correctly. There are a few nucleotides known to be important. These are the ones required for all splicing, e.g. as shown in FIG. 34 below. The initial GU and the final AG of the intron are particularly important and therefore preferred, as discussed elsewhere, though 5% of introns start GC instead. This consensus sequence is preferred, although it applies to all splicing, not specifically to alternative splicing. In FIG. 34, Pu=A or G; Py=C or U

Preferably, the system is or comprises a plasmid. As mentioned above, this can be either DNA, RNA or a mixture of both. If the system comprises RNA, then it may be preferable to reverse-translate the RNA into DNA by means of a Reverse Transcriptase. If reverse transcription is required, then the system may also comprise a coding sequence for the RT protein and a suitable promoter therefor. Alternatively, the RTase and promoter therefore may be provided on a separate system, such as a virus. In this case, the system would only be activated following infection with that virus. The need to include suitable cis-acting sequences for the reverse transcriptase or RNA-dependent RNA polymerase would be apparent to the person skilled in the art.

However, it is particularly preferred that the system is predominantly DNA and more preferably consists only of DNA, at least with respect to the sequences to be expressed in the organism.

Whilst in some embodiments the at least one heterologous polynucleotide sequence to be expressed in an organism is a polynucleotide sequence for interference RNA (RNAi), it is particularly preferred that it is a polynucleotide sequence capable off encoding a functional protein. The description will predominantly focus on polynucleotide sequences encoding a functional protein, but it will be understood that this also refers to polynucleotides for interference RNA (RNAi), unless otherwise apparent.

It will be understood that reference is made to start and stop codons between which the polynucleotide sequence to be expressed in an organism is defined, but that this does not exclude positioning of the at least one splice control sequence, elements thereof, or other sequences, such as introns, in this region. In fact, it will be apparent form the present description that the splice control sequence, can, in some embodiments, be positioned in this region.

Furthermore, the splice control sequence, for instance, can overlap with the start codon at least, in the sense that the G of the ATG can be, in some embodiments, be the initial 5′ G of the splice control sequence. Thus, the term “between” can be thought of as referring to from the beginning (3′ to the initial nucleotide, i.e. A) of the start codon, preferably 3′ to the second nucleotide of the start codon (i.e. T), up to the 5′ side of the first nucleotide of the stop codon. Alternatively, as will be apparent by a simple reading of a polynucleotide sequence, the stop codon may also be included.

The at least one heterologous polynucleotide sequence to be expressed in an organism is a heterologous sequence. By “heterologous”, it would be understood that this refers to a sequence that would not, in the wild type, be normally found in association with, or linked to, at least one element or component of the at least one splice control sequence. For example, where the splice control sequence is derived from a particular organism, and the heterologous polynucleotide is a coding sequence for a protein or polypeptide, i.e. is a polynucleotide sequence encoding a functional protein, then the coding sequence could be derived, in part or in whole, from a gene from the same organism, provided that that the origin of at least some part of the transcribed polynucleotide sequence was not the same as the origin of the at least one splice control sequence. Alternatively, the coding sequence could be from a different organism and, in this context, could be thought of as “exogenous”. The heterologous polynucleotide could also be thought of as “recombinant”, in that the coding sequence for a protein or polypeptide are derived from different locations, either within the same genome (i.e. the genome of a single species or sub-species) or from different genomes (i.e. genomes from different species or subspecies).

Heterologous can refer to a sequence other than the splice control sequence and can, therefore, relate to the fact the promoter, and other sequences such as 5′ UTR and/or 3′UTR can be heterologous to the polynucleotide sequence to be expressed in the organism, provided that said polynucleotide sequence is not found in association or operably linked to the promoter, 5′ UTR and/or 3′UTR, in the wildtype, i.e. the natural context of said polynucleotide sequence, if any.

It will be understood that heterologous also applies to “designer” or hybrid sequences that are not derived from a particular organism but are based on a number of components from different organisms, as this would also satisfy the requirement that the sequence and at least one component of the splice control sequence are not linked or found in association in the wildtype, even if one part or element of the hybrid sequence is so found, as long as at least one part or element is not. Preferably, a portion of at least 50 nucleotides of the hybrid sequence is not found in association with the at least one component of the splice control sequence, more preferably 200 nucleotides and most preferably 500 nucleotides.

It will also be understood that synthetic versions of naturally occurring sequences are envisioned. Such synthetic sequences are also considered as heterologous, unless they are of identical sequence to a sequence which would, in the wild type or natural context, be normally found in association with, or linked to, at least one element or component of the at least one splice control sequence.

This applies equally to where the heterologous polynucleotide is a polynucleotide for interference RNA.

In one embodiment, where the polynucleotide sequence to be expressed comprises a coding sequence for a protein or polypeptide, it will be understood that reference to expression in an organism refers to the provision of one or more transcribed RNA sequences, preferably mature mRNAs, but this may, preferably, also refer to translated polypeptides in said organism.

RT-PCR, which demonstrates the presence of a transcript, not of a protein, may be used to identify transcribed RNA sequences. This is also particularly useful when the protein itself is not translated or is not functional or not identifiable by antibodies raised against the naturally-occurring or wildtype protein, due to RNAi, post-translational modification or distorted folding.

In another embodiment, where the polynucleotide sequence to be expressed comprises polynucleotides for interference RNA, it will also be understood that reference to expression in an organism refers to the interaction of the polynucleotides for interference RNA, or transcripts thereof, in the RNAi pathway, for instance by binding of Dicer or formation of small interfering RNA (siRNA). Indeed, it is particularly preferred that the polynucleotides for interference RNA comprise siRNA sequences and are, therefore, preferably 20-25 nucleotides long, especially where the organism is mammalian.

In insects and nematodes especially, it is preferred to provide portion of dsRNA, for instance by hairpin formation, which can then be processed by the Dicer system. Mammalian cells generally produce an interferon response against long dsRNA sequences, so for mammalian cells it is more common to provide shorter sequences, such as siRNAs. Antisense sequences or sequences having homology to microRNAs that are naturally occurring RNA molecules targeting protein 3′ UTRs are also envisaged as sequences for RNAi according to an embodiment of the present invention.

Each splice control sequence in the system comprises at least one splice acceptor site and at least one splice donor site. The number of donor and acceptor sites may vary, depending on the number of segments of sequence that are to be spliced together. Preferably, branch sites are included in each splice control sequence. A branch site is the sequence to which the splice donor is initially joined, see FIG. 32, which shows that splicing occurs in two stages, in which the 5′ exon is separated and then is joined to the 3′ exon.

Referring to said figure, the A is the only essential nucleotide, and is, therefore, preferably included. Without being bound by theory, it is believed that pre-mRNA splicing proceeds via a lariat intermediate, just as it does in group II self-splicing. First, cleavage occurs at the 5′ junction—sometimes called the splice donor site. The phosphate at the 5′end of the intron then becomes linked to the 2′ OH of an adenine approximately 25 nucleotides upstream of the 3′ end of the intron, which is sometimes called the acceptor site. This A residue is called the branch point. The next step is that cleavage occurs at the 3′ splice junction and the 5′ phosphate of the downstream exon is joined to the 3′ OH of the upstream exon.

It is particularly preferred that the manner or mechanism of alternative splicing is sex-specific. Preferably, the splice control sequence is derived from a tra intron. However, it is particularly preferred that the alternative splicing mechanism is derived from the Medfly transformer gene Cctra, or from another ortholog or homolog of the Drosophila transformer gene, preferably from C. rosa, or B. zonata especially one derived from a tephritid fruit fly.

It is also preferred that the splice control sequence is derived from the alternative splicing mechanism of the Actin-4 gene, in particular that from Aedes spp. and most preferably from AaActin-4, which is a gene from Aedes/Stegomyia aegypti which shows tissue, stage and sex-specific splicing.

Preferably, alternative splicing, particularly that mediated by Actin-4, may add sequences that affect RNA translation or stability, for instance.

It is also preferred that the splicing mechanism comprises at least a fragment of the doublesex (dsx) gene, preferably that derived from Drosophila, B. mori. Pink Boll Worm, Codling Moth, or a mosquito, in particular A. gambiae or especially A. aegypti.

It is preferred that the splice control sequence and the heterologous polynucleotide sequence encoding a functional protein, defined between a start codon and a stop codon, and/or polynucleotides for interference RNA (RNAi), to be expressed in an organism, are provided in the form of a minigene construct or a cassette exon.

This is particularly preferred when the splice control sequence is derived from dsx (preferably minigene 1 as described in the Examples and represented in SEQ ID NO. 149 (exons are present at positions 1-135, 1311-2446 and 3900-4389 of SEQ ID NO. 149) which was included in construct LA3491) or Actin-4.

Particularly preferred examples of the present invention are provided in the Examples, and can be selected from the group consisting of the plasmids or constructs, in particular any of those according to any one of FIGS. 19-31, especially any of the plasmids shown in FIGS. 16-18, 22-24, 26-32, 49, 52-55, and 61-69, and/or SEQ ID NOs 46-48, 50-56, 143-145 and 151-162.

Preferably, the functional protein to be expressed in an organism is tTAV, tTAV2 or tTAV3.

Further proteins to be expressed in the organism are, or course envisaged, in combination with said functional protein, preferably a lethal gene as discussed elsewhere.

A continuous ORF may be also be thought of as an uninterrupted ORF, i.e. a polynucleotide sequence in mature mRNA, which does not include non-coding nucleotides, for instance those having the potential to be translated into amino acids. In this definition, it is preferred that the stop codon is not included.

In some embodiments, the at least one splice control sequence regulates the alternative splicing by means of both intronic and exonic nucleotides. However, in one embodiment, it is particularly preferred that the at least one splice control sequence is an intronic splice control sequence. In other words, it is preferred that the at least one splice control sequence is substantially derived from polynucleotides that form part of an intron and are thus excised from the primary transcript by splicing, such that these nucleotides are not retained in the mature mRNA sequence.

Therefore, intronic sequences can be thought of as distinct from “exonic” sequences, which are retained in the processed (post-splicing) RNA molecule. Where the processed RNA molecule encodes a protein or polypeptide sequence, and is capable of being translated, i.e. has the correct structure and modifications such as a cap, and a polyadenylation signal, for instance, it is known as mature or processed mRNA and some of the exonic sequences then code for amino acids, when translated.

It will be understood that in alternative splicing, sequences may be intronic under some circumstances (i.e. in some alternative splicing variants), but exonic under other circumstances (i.e. in other variants). Thus, the at least one splice control sequence of the present invention is preferably substantially derived from polynucleotides that form part of an intron in at least one alternative splicing variant, i.e. in either the first spliced mRNA product or the at least one alternatively spliced mRNA product. Thus, introns or intronic sequences can be viewed as spliced out in at least one transcript or transcript type.

For example, consider the tra intron from C. capitata (Cctra intron), which is a particularly preferred example of an at least one splice control sequence according to the present invention. According to FIG. 2A of Pane et al, reproduced as FIG. 33, all 8 of the putative Tra/Tra2 binding sites highlighted are in intronic sequence in the sense that they are in portions of sequence spliced out in transcript F1, but on the other hand 6 out of the 8 are exonic in the sense that they are in exons that are included or retained in either transcript M1 or M2, or both. Thus, these Tra/Tra2 binding sites are intronic in the present sense as they are capable of controlling alternative splicing, but are spliced out, i.e. not present, in at least one alternative splicing variant, i.e. at least one mRNA that has been spliced in an alternative manner from pre-RNA.

In “normal” (non-alternative) splicing and in alternative splicing, introns are generally removed from the pre-RNA to form a spliced mRNA, which may then be translated into a polypeptide, such as a protein or protein fragment, having an amino acid sequence. Thus, it will be readily apparent to the skilled person how to determine those sequences of the present system that are to be considered intronic, rather than exonic.

It will, of course be appreciated that only part of an mRNA is actually translated, i.e. typically the part between the start codon and the stop codon, although it will be understood that sometimes multiple starts and stops are present. Thus, when reference is made herein to translation of an mRNA sequence, it will be appreciated that this is referring to translation of the portion starting at the first nucleotide of the start codon and ending after the last nucleotide before the start of the stop codon, which may be considered as the coding portion.

As mentioned above, exonic sequences may be involved in the mediation of the control of alternative splicing, but it is preferred that at least some intronic control sequences are involved in the mediation of the alternative splicing. In other words, the gene expression system of the present invention may also include splice control sequences present in exons, as long as there is some intronic involvement of control. Particularly preferred examples of these are splice control sequences derived from or containing elements of the dsx gene, where, without being bound by theory, it is thought that exonic sequences assist in the mechanism of alternative splicing.

Thus, in some embodiments, the at least one splice control sequence does comprise exonic sequence and it will be understood that this is envisaged by definitions used to describe the present invention. Thus, as will be apparent, it is possible for some nucleotides to be encompassed within the definition of the at least one splice control sequence and also within the definition of a polynucleotide sequence encoding a functional protein. In other words, the definition of these elements can overlap, such that certain nucleotides can be covered by the definition of more than one element.

However, the skilled person will recognise that this is not unusual in molecular biology, as nucleotides can often perform more than one role. For instance, in the present invention, a nucleotide can form part of a coding sequence for a functional protein, but could also form part of a sequence recognised and bound by a splicing factor, an example of which the TRA protein or TRA/TRA complex, as discussed elsewhere. This is not unusual as, for instance, some viruses have highly concentrated genome where the same stretch of polynucleotides can code for two or even three different proteins, each read in a different frame.

Of course, it may also be that the splice control sequence or sequences are solely intronic, i.e. with no exonic influence. Indeed, this is particularly preferred.

In some embodiments, it is preferred that the at least one splice control sequence is capable of being removed from the pre-RNA, by splicing. Preferably, the at least one splice control sequence does not result in a frameshift in at least one splice variant. Preferably this is a splice variant encoding a full-length functional protein. In other words, at least the one splice control sequence preferably does not mediate the removal of nucleotides that form part, or were intended to form part of, the polynucleotide sequence encoding a functional protein, defined between a start codon and a stop codon, and/or polynucleotides for interference RNA (RNAi), to be expressed in an organism. By this it is meant that nucleotides that are excised by splicing, in at least one splice variant, are not nucleotides that encode amino acids in the wild type form of the protein or gene. One or more splice variants may have said nucleotides excised, but at least one variant must retain these nucleotides, so that a frameshift is not induced in the at least one variant. These removed nucleotides are those that are removed in addition to the sequences that are normally spliced out such as the intron.

However, in view of the above, it is also envisaged that different splice variants may result in the same sequence being read in different frames.

Interaction of the at least one splice control sequence with cellular splicing machinery, e.g. the spliceosome, leads to or mediates the removal of a series of, preferably, at least 50 consecutive nucleotides from the primary transcript and ligation (splicing) together of nucleotide sequences that were not consecutive in the primary transcript (because they, or their complement if the antisense sequence is considered, were not consecutive in the original template sequence from which the primary transcript was transcribed). Said series of at least 50 consecutive nucleotides comprises an intron. This mediation acts preferably in a sex-specific, stage-specific, germline-specific or tissue-specific manner, or combination thereof, such that equivalent primary transcripts in different sexes, stages, tissue types, etc, tend to remove introns of different size or sequence, or in some cases may remove an intron in one case but not another. This phenomenon, the removal of introns of different size or sequence in different circumstances, or the differential removal of introns of a given size or sequence, in different circumstances, is known as alternative splicing. Alternative splicing is a well-known phenomenon in nature, and many instances are known, see above.

In some preferred embodiments, the at least one splice control sequence is associated with a heterologous open reading frame such that, in at least one splice variant, the heterologous open reading frame is disrupted, e.g. by a stop codon or frameshift, while in at least one alternative splice variant the heterologous open reading frame is not disrupted. Transcripts of the second type encode or potentially encode a functional protein, whereas those of the first type encode a protein with altered, disrupted or even no function, activity or stability relative to those of the second type.

In general, it will be apparent to the person skilled in the art that the heterologous open reading frame may itself be a composite or fusion of sequences from various sources. Splicing to produce a functional protein may still produce an altered protein relative to the prototype heterologous open reading frame, for example if the inserted alternatively spliced intron includes sequence that is exonic in all alternative splicing forms, and therefore retained in mature mRNAs of the second type. However, it is particularly preferred that at least one transcript removes all, or substantially all, of the inserted alternatively spliced sequence, such that the heterologous open reading frame is restored, or substantially restored, to intact form, with little or no sequence endogenously associated with the intron remaining in the mature mRNA. Endogenous is used here in contrast to heterologous, so it will be understood that this refers to a sequence that would, in the wild type, be normally found in association with, or linked to, at least one element or component of the at least one splice control sequence.

Alternatively, one or more transcripts may remove additional nucleotides, so that the heterologous open reading frame is disrupted, not by the insertion of extra nucleotides (for example stop codon or frame shift, but also potentially coding sequence that disrupts the function), but rather by deletion of nucleotides from the heterologous open reading frame, for example in such a way as to induce a frameshift. One or more splice variants may have said nucleotides excised, but at least one variant must retain these nucleotides, so that a frameshift is not induced in the at least one variant. These removed nucleotides are those that are removed in addition to the sequences that are normally spliced out such as the intron, where an intronic sequence may be considered as one that forms part of an intron in at least one alternative splicing variant of the natural analogue.

When exonic nucleotides are to be removed, then these must be removed in multiples of three, if it is desired to avoid to avoid a frameshift, but as a single nucleotide or multiples of two (that are not also multiples of three) if it is desired to induce a frameshift. It will be appreciated that if only one or certain multiples of two nucleotides are removed, then this could lead to a completely different protein sequence being encoded at or around the splice junction of the mRNA.

This is particularly the case in an embodiment of the system where cassette exons are used to interrupt an open reading frame in some splice variants but not others, such as in, for example, tra, especially Cctra.

In another preferred embodiment of the present invention, all or part of an open reading frame is on a cassette exon, for example some Dsx embodiments derived from Aedes, are provided with, for instance, a tTAV coding region on a cassette exon that is only present in female-specific splice variants.

Where mediation of alternative splicing is sex-specific, it is preferred that the splice variant encoding a functional protein to be expressed in an organism is the F1 splice variant, i.e. a splice variant found only or predominantly in females, and preferably is the most abundant variant found in females, although this is not essential. Correspondingly for configurations where all or part of a functional open reading frame is on a cassette exon, it is preferred that this cassette exon is included in transcripts found only or predominantly in females, and preferably such transcripts are, individually or in combination, the most abundant variants found in females, although this is not essential.

In one preferred embodiment, sequences are included in a hybrid or recombinant sequence or construct which are derived from naturally occurring intronic sequences which are themselves subject to alternative splicing, in their native or original context. Therefore, an intronic sequence may be considered as one that forms part of an intron in at least one alternative splicing variant of the natural analogue. Thus, sequences corresponding to single contiguous stretches of naturally occurring intronic sequence are envisioned, but also hybrids of such sequences, including hybrids from two different naturally occurring intronic sequences, and also sequences with deletions or insertions relative to single contiguous stretches of naturally occurring intronic sequence, and hybrids thereof. Said sequences derived from naturally occurring intronic sequences may themselves be associated, in the invention, with sequences not themselves part of any naturally occurring intron. If such sequences are transcribed, and preferably retained in the mature RNA in at least one splice variant, they may then be considered exonic.

It will also be appreciated that reference to a “frame shift” could also refer to the direct coding of a stop codon, which is also likely to lead to a non-functioning protein as would a disruption of the spliced mRNA sequence caused by insertion or deletion of nucleotides. Production from different splice variants of two or more different proteins or polypeptide sequences of differential function is also envisioned, in addition to the production of two or more different proteins or polypeptide sequences of which one or more has no predicted or discernable function. Also envisioned is the production from different splice variants of two or more different proteins or polypeptide sequences of similar function, but differing subcellular location, stability or capacity to bind to or associate with other proteins or nucleic acids.

Preferably, the at least one splice control sequence is intronic and comprises on its 5′ end a guanine (G) nucleotide. In other words, the 5′ nucleotide of the splice control sequence, 3′ to the splice donor site, and preferably at the interface or junction of the exon with the splice control sequence, is Guanine (G), in the pre-RNA, or C in an antisense DNA sequence corresponding thereto.

Furthermore, the adjacent nucleotide (3′ to said G) is preferably Cytosine (C) in the pre-RNA, or a corresponding G in a DNA sequence, but is most preferably Uracil (U) in the pre-RNA, or a corresponding A in a DNA antisense sequence. Thus, the two 5′ nucleotides of the splice control sequence are preferably 5′GT with respect to the DNA sense strand, 5′-GU in the primary transcript.

Preferably, at least one intronic splice control sequence also comprises on its 3′ end a 3′ Guanine nucleotide and preferably AG-3′ at the junction of the splice acceptor site with the exon, for instance, see FIG. 34.

Preferably, the flanking sequence 5′ to the splice donor site in the system comprises 5′-TG, so that the sequence can be represented 5′-TG-*-splice control sequence-**-3′, where * represents the splice donor site and ** represents the splice acceptor site.

Preferably, the splice control sequence is also flanked on its 3′ side by a G nucleotide, and most preferably by GT nucleotides, such that the sequence could be represented as: 5′-TG-*-splice control sequence-**-GT-3′. It will be appreciated that this is the sense strand DNA sequence (TG). Thus, the transcribed pre-RNA will read UG for instance, where U replaces T.

Derivatives of Guanine or Thymine having the same function are also envisaged.

It is particularly preferred that the splicing is sex-specific and further mediated or controlled by binding of the TRA protein or TRA/TRA2 protein complex, or homologues thereof. In insects, for instance, the TRA protein is differentially expressed in different sexes. In particular, the TRA protein is known to be present largely in females and, therefore, mediates alternative splicing in such a way that a coding sequence is expressed in a sex-specific manner, i.e. that in some cases a protein is expressed only in females or at a much higher level in females than in males or, alternatively, in other cases a protein is expressed only in males, or at a much higher level in males than in females. Whilst it is preferred that the protein is expressed only in males, it is particularly preferred that the protein is expressed only in females, however. The mechanism for achieving this sex-specific alternative splicing mediated by the TRA protein or the TRA/TRA-2 complex is known and is discussed, for instance, in Pane et al (Development 129, 3715-3725 (2002)).

Preferably, the at least one splice control sequence comprises, and more preferably consists of, the tra intron derived from the tra gene of Ceratitis capitata (Cctra), which has one alternatively spliced region. In the F1 transcript, as illustrated by FIG. 33 (FIG. 2A of Pane et al (2002) supra), this is the first intron. Homologues of the tra gene in other species, such as Bactrocera oleae, Ceratitis rosa, Bactrocera zonata and Drosophila melanogaster also have alternatively spliced regions in a similar location within the tra coding sequence. tra introns derived from these insects are also particularly preferred.

The splicing pattern in Cctra in particular is well conserved, with those transcripts found in males containing additional exonic material relative to the F1 transcript, such that these transcripts do not encode fill-length, functional Tra protein. By contrast, the F1 transcript does encode full-length, functional Tra protein; this transcript is substantially female-specific at most life-cycle stages, though it is speculated that very early embryos of both sexes may contain a small amount of this transcript. We describe the sequence spliced out of the F1 transcript, but not the male-specific or non-sex-specific transcripts, as the tra intron, or even the tra F1 intron. Thus the version of this sequence found in the Cctra gene is the Cctra intron.

Thus the tra gene is regulated in part by sex-specific alternative splicing, while its key product, the Tra protein, is itself involved in alternative splicing. In insects, sex-specific alternative splicing mediated by the TRA protein, or a complex comprising the TRA and TRA2 proteins, include Dipteran splice control sequences derived from the doublesex (dsx) gene and also the tra intron itself, although this would exclude the tra intron from Drosophila (Dmtra), which is principally mediated by the Sxl gene product in Drosophila, rather than TRA or the TRA/TRA2 complex.

Outside of Drosophila, the Sxl gene product is not differentially expressed in the different sexes. Sxl is not thought to act in the mediation of sex-specific alternative splicing in non-Drosophilid insects.

Examples of the TRA protein that binds to the binding protein sites (the nucleotide sequences specifically recognised by the TRA protein) in the tra intron are preferably from Diptera, preferably from the family Tephritidae, more preferably from the genera Ceratitis, Anastrepha or Bactrocera. However, it is also envisaged that other Dipterans, such as Drosophilids or mosquitoes of the various forms discussed below, are also capable of providing the TRA protein or homologues thereof that are capable of binding to the appropriate sites on the splice control sequences derived from dsx gene, the tra gene or the tra intron, i.e. the alternatively spliced tra intron completely removed in the F1 transcript, even in those cases, such as Drosophila, where the natural tra gene (Dmtra) is not itself regulated by TRA protein. In some embodiments, the “tra intron” may be defined as a splice control sequence wherein alternative splicing of the RNA transcript is regulated by TRA, for instance binding thereof, alone or in combination (i.e. when complexed) with TRA2. This excludes the tra intron from Drosophila.

It is particularly preferred that the splice control sequences are derived from the tra intron. Said tra intron may be derived, as discussed elsewhere, from Ceratitis, Anastrepha or Bactrocera The Ceratitis capitata tra intron from the transformer gene was initially characterised by Pane et al (2002), supra. However, it will be appreciated that homologues exist in other species, and can be easily identified in said species and also in their various genera. Thus, when reference is made to tra it will be appreciated that this also relates to tra homologues in other species, especially in Ceratitis, Anastrapha or Bactrocera species.

By “derived” it will be understood that, using reference to the tra intron, this refers to sequences that approximate to or replicate exactly the tra intron, as described in the art, in this case by Pane et al (2002), supra. However, it will be appreciated that, as these are intronic sequences, that some nucleotides can be added or deleted or substituted without a substantial loss in function.

Preferred examples of this include the dsx intron, preferably provided in the form of a minigene. In this instance, it may be preferable to delete, as we have done in the Examples, sizable amounts from alternatively spliced introns, e.g. 90% or more of an intron in some cases, whilst still retaining the alternative splicing function. Thus, whilst large deletions are envisioned, it is also envisaged that smaller, e.g. even single nucleotide insertions, substitutions or deletions are also preferred.

The exact length of the splice control sequence derived from the tra intron is not essential, provided that it is capable of mediating alternative splicing. In this regard, it is thought that around 55 to 60 nucleotides is the minimum length for a modified tra intron, although the wild type tra intron (F1 splice variant) from C. capitata is in the region of 1345 nucleotides long.

It is particularly preferred that the fill length 1345 ntd sequence of Cctra is used.

As with all nucleotide sequences discussed herein, it is preferred that a certain degree of sequence homology is envisaged, unless otherwise apparent. Thus, it is preferred that the splice control sequence has at least 80% sequence homology with the reference SEQ ID NO., preferably at least 80% sequence homology with the reference SEQ ID NO., preferably at least 80% sequence homology with the reference SEQ ID NO., more preferably at least 90% sequence homology with the reference SEQ ID NO., more preferably at least 95% sequence homology with the reference SEQ ID NO., even more preferably at least 99% sequence homology with the reference SEQ ID NO., and most preferably at least 99.9% sequence homology with the reference SEQ ID NO. A suitable algorithm such as BLAST may be used to ascertain sequence homology. If large amounts of sequence are deleted of the wildtype, then the sequence comparison may be over the full length of the wildtype or over aligned sequences of similar homology.

However, it will be understood that despite the above sequence homology, certain elements, in particular the flanking nucleotides and splice branch site must be retained, for efficient functioning of the system. In other words, whilst portions may be deleted or otherwise altered, alternative splicing functionality or activity, to at least 30%, preferably 50%, preferably 70%, more preferably 90%, and most preferably 95% compared to the wildtype should be retained. This could be increased cf the wildtype, as well, by suitably engineering the sites that bind alternative splicing factors or interact with the spliceosome, for instance.

In particular, it is preferred that where the splice control sequence comprises a modified TRA intron, this comprises at least 20 to 40 base pairs from the 5′ and, preferably, so the 3′ end of said intron. Furthermore, it is preferred that at least 3 or 4 and most preferably, at least 5, preferably 6, more preferably 7 and most preferably all 8 of the 8 putative TRA binding domains of the C. capitata tra intron, as taught by Pane et al (2002), or homologues thereof, are provided. Of course, if further such sites are discovered in due course, then it is envisaged that the splice control sequence could include more than 8 sites. In fact, it is envisaged that the more than 8 sites may be engineered in to the splice control sequence and that alternative splicing may be regulated in this way, especially if some sites are bound with differing affinities leading to different alternative splicing outcomes.

A consensus sequence for the putative TRA binding domains of the C. capitata tra intron is given below as SEQ ID NO 1, a DNA sequence, although the corresponding RNA equivalent is also preferred.

The preferred consensus sequences is 1: TCWWCRATCAACA (SEQ ID NO. 1), where W=A or T and R=A or G.

Similar considerations apply to doublesex, where the consensus sequence for the TRA protein is also that given in SEQ ID NO. 1, as a protein complex comprising the Tra and TRA2 proteins is a key regulator of alternative splicing of doublesex, as it is for tra homologues (though not the tra homologues found in Drosophilids).

As mentioned above, the splice control sequences are preferably derived from the tra intron, preferably from the family Tephritidae. It is particularly preferred that the tra intron is derived from B. zonata or, preferably, from other non-Drosophilid fruit flies. However, it is particularly preferred that the tra intron is derived from the Ceratitis genus, in particular C. rosa and, most preferably, C. capitata. These are more widely known as the Natal and Mediterranean fruit flies, respectively.

With regard to the tra intron derived from B. zonata, we have shown that this can lead to sex-specific alternative splicing in transgenic Mexfly (Anastrapha ludens) and in transgenic Medfly (C. capitata). We have also shown that a variety of proteins can be expressed in a sex-specific manner via alternative splicing, including tTAV 3 and Rpr.

In relation to the tra intron derived from C. rosa, we have successfully provided alternative splicing in a sex-specific manner of a transgene in Medfly.

With regard to the tra intron derived from C. capitata (Medfly), we have shown that this can mediate sex-specific splicing in transgenic Medfly, and other Tephritids, and other Tephritids such as A. ludens (Mexfly). Not only that, we have shown that this intron can work successfully across a whole range of insects and, in particular, Dipterans. Indeed, we have shown that the TRA intron from C. capitata (referred to as Cctra) can provide sex-specific alternative splicing in transgenic Drosophila, which is not a Tephritid, and also in the mosquito Aedes aegypti. Although mosquitoes are Diptera, they diverged from Drosophila and the Tephritids about 250 million years ago and, therefore, are much more distantly related than Drosophilids are to Tephritids, for which the divergence time has been estimated as 120-150 million years. Thus, this shows the broad applicability of the present invention across a wide range of insects.

With regard to splice control sequences derived from the dsx intron, we have also shown that this can be used to alternatively splice, in a sex-specific manner, in a broad range of insects. Accordingly, it is particularly preferred that the dsx is derived from Bombyx mori (silk moth), Pectinophora gossypiella (Pink Bollworm) Pectinophora gossypiella, Cydia pomonella (codling moth), Drosophila, and mosquitoes such as Anopheles sp., for instance A. gambiae. Particularly preferred mosquitoes include Stegomyia spp., particularly S. aegypti (also known as Aedes aegypti).

Indeed, in A. aegypti, we have shown a considerable number of DNA constructs, which are capable of providing sex-specific alternative splicing.

It will be appreciated that the system or construct is preferably administered as a plasmid, but generally tested after integrating into the genome. Administration can be by known methods in the art, such as parenterally, intravenous intramuscularly, orally, transdermally, delivered across a mucous membrane, and so forth. Injection into embryos is particularly preferred. The plasmid may be linearised before or during administration, and not all of the plasmid may be integrated into the genome. Where only part of the plasmid is integrated into the genome, it is preferred that this part include the at least one splice control sequence capable of mediating alternative splicing.

Preferably, the polynucleotide expression system is a recombinant dominant lethal genetic system, the lethal effect of which is conditional. Suitable conditions include temperature, so that the system is expressed at one temperature but not, or to a lesser degree, at another temperature, for example. The lethal genetic system may act on specific cells or tissues or impose its effect on the whole organism. Systems that are not strictly lethal but impose a substantial fitness cost are also envisioned, for example leading to blindness, flightlessness (for organisms that could normally fly), or sterility. Systems that interfere with sex determination are also envisioned, for example transforming or tending to transform all or part of an organism from one sexual type to another. It will be understood that all such systems and consequences are encompassed by the term lethal as used herein. Similarly, “killing”, and similar terms refer to the effective expression of the lethal system and thereby the imposition of a deleterious or sex-distorting phenotype, for example death.

More preferably, the polynucleotide expression system is a recombinant dominant lethal genetic system, the lethal effect of which is conditional and is not expressed under permissive conditions requiring the presence of a substance which is absent from the natural environment of the organism, such that the lethal effect of the lethal system occurs in the natural environment of the organism

In other words, the coding sequences encode a lethal linked to a system such as the tet system described in WO 01/39599 and/or WO2005/012534.

Indeed it is preferred that the expression of said lethal gene is under the control of a repressible transactivator protein. It is also preferred that the gene whose expression is regulated by alternative splicing encode a transactivator protein such as tTA. This is not incompatible with the regulated protein being a lethal. Indeed, it is particularly preferred that it is both. In this regard, we particularly prefer that the system includes a positive feedback system as taught in WO2005/012534.

Preferably, the lethal effect of the dominant lethal system is conditionally suppressible.

Suitable organisms under which the present system can be used include mammals such as mice, rats and farm animals. Also preferred are fish, such as salmon and trout. Plants are also preferred, but it is particularly preferred that the host organism is an insect, preferably a Dipteran or tephritid. Preferably, the organism is not a human, preferably non-mammalian, preferably not a bird, preferably an invertebrate, preferably an arthropod.

In particular, it is preferred that the insect is from the Order Diptera, especially higher Diptera and particularly that it is a tephritid fruit fly, preferably Medfly (Ceratitis capitata), preferably Mexfly (Anastrepha ludens), preferably Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata) Caribbean fruit fly (Anastrepha suspensa) or West Indian fruit fly (Anastrepha obliqua). It is also particularly preferred that the host organism is a mosquito, preferably from the genera Stegomyia, Aedes, Anapheles or Culex. Particularly preferred are Stegomyia aegyptae, also known as Aedes aegypti, Stegomyia albopicta (also known as Aedes albopictus), Anopheles stephensi, Anopheles albimanus and Anopheles gambiae.

Within Diptera, another preferred group is Calliphoridae, particularly the New world screwworm (Cochliomyia hominivorax), Old world screwworm (Chrysomya bezziana) and Australian sheep blowfly (Lucilia cuprina). Lepidoptera and Coleoptera are also preferred, especially moths, including codling moth (Cydia pomonella), and the silk worm (Bombyx mori), the pink bollworm (Pectinophora gossypiella), the diamondback moth (Plutella xylostella), the Gypsy moth (Lymantria dispar), the Navel Orange Worm (Amyelois transitella), the Peach Twig Borer (Anarsia lineatella) and the rice stem borer (Tryporyza incertulas), also the noctuid moths, especially Heliothinae. Among Coleoptera, Japanese beetle (Popilla japonica), White-fringed beetle (Graphognatus spp.), Boll weevil (Anthonomous grandis), corn root worm (Diabrotica spp) and Colorado potato beetle (Leptinotarsa decemlineata) are particularly preferred.

Preferably, the insect is not a Drosphilid, especially Dm. Thus, in some embodiments, expression in Drosophilids, especially Dm is excluded. In other embodiments, the splice control sequence is not derived from the tra intron of a Drosphilid, especially Dm.

It is preferred that the expression of the heterologous polynucleotide sequence leads to a phenotypic consequence in the organism. It is particularly preferred that the functional protein is not beta-galactosidase, but can be associated with visible markers (including fluorescence), viability, fertility, fecundity, fitness, flight ability, vision, and behavioural differences. It will be appreciated, of course, that, in some embodiments, the expression systems are typically conditional, with the phenotype being expressed only under some, for instance restrictive, conditions.

In a further aspect, there is also provided a method of population control of an organism in a natural environment therefor, comprising:

i) breeding a stock of the organism,
the organism carrying a gene expression system comprising a system according to the present invention which is a dominant lethal genetic system,

ii) distributing the said stock animals into the environment at a locus for population control; and

iii) achieving population control through early stage lethality by expression of the lethal system in offspring that result from interbreeding of the said stock individuals with individuals of the opposite sex of the wild population.

Preferably, the early stage lethality is embryonic or before sexual maturity, preferably early in development, most preferably in the early larval or embryonic life stages.

Preferably, the lethal effect of the lethal system is conditional and occurs in the said natural environment via the expression of a lethal gene,
the expression of said lethal gene being under the control of a repressible transactivator protein,

the said breeding being under permissive conditions in the presence of a substance, the substance being absent from the said natural environment and able to repress said transactivator.

Preferably, the lethal effect is expressed in the embryos of said offspring. Preferably, the organism is an invertebrate multicellular animal or is as discussed elsewhere.

Also provided is a method of biological control, comprising:
i) breeding a stock of males and female organisms transformed with the expression system according to the present invention under permissive conditions, allowing the survival of males and females, to give a dual sex biological control agent;
ii) optionally before the next step imposing or permitting restrictive conditions to cause death of individuals of one sex and thereby providing a single sex biological control agent comprising individuals of the other sex carrying the conditional lethal genetic system;
iii) releasing the dual sex or single sex biological control agent into the environment at a locus for biological control; and
iv) achieving biological control through expression of the genetic system in offspring resulting from interbreeding of the individuals of the biological control agent with individuals of the opposite sex of the wild population:

Preferably, there is sex-separation prior to organism distribution by expression of a sex specific lethal genetic system.

Preferably, the lethal effect results in killing of greater than 90% of the target class of the progeny of matings between released organisms and the wild population.

Also provided is a method of sex separation comprising:
i) breeding a stock of male and female organisms transformed with the gene expression system under permissive or restrictive conditions, allowing the survival of males and females; and
ii) removing the permissive or restrictive conditions to induce the lethal effect of the lethal gene in one sex and not the other by sex-specific alternative splicing of the lethal gene.

Preferably, the lethal effect results in killing of greater than 90% of the target class of the progeny of matings between released organisms and the wild population.

Also provided is a method or biological or population control comprising;
i) breeding a stock of male and female organisms transformed with the gene expression system under permissive or restrictive conditions, allowing the survival of males and females;
ii) removing the permissive or restrictive conditions to induce the lethal effect of the lethal gene in one sex and not the other by sex-specific alternative splicing of the lethal gene to achieve sex separation;
iii) sterilising or partially sterilising the separated individuals and
iv) achieving said control through release of the separated sterile or partially sterile individuals in to the natural environment of the organism.

Preferably, the sterilising is achieved through the use of ionising radiation. In general, however, methods avoiding irradiation, as used in the Sterile Insect Technique (SIT) are especially preferred and have many cost and health advantages over methods associated with or followed by the use of radiation.

Also provided is a method to selectively eliminate females from a population. The equivalent for males is also envisaged.

Methods of sex separation are hugely important commercially in, for example silk worms, where males produce more and better silk than females. Thus, methods of sex separation that eliminate females and, in particular female silk worms are particularly preferred.

It is also envisaged that the functional protein may be a expressed differentially, but detectably in more than one splice variant and preferably, therefore, in both sexes, for instance. Such examples include a fluorescent protein, such as eGFP, CopGFP and DsRed2. This may be used in a method of non-lethal sex separation or sorting, so that one can separate the two types without killing either of them

We have also surprisingly discovered that the positioning of the splice control sequence can be altered and better results obtained. Preferably, the splice control sequence is the “first” splice control sequence, when read from the promoter, in 5′ to 3′ direction We have found that in certain constructs with an intron in the 5′ UTR of the system that this leads to reduced levels or alternatively spliced protein expression mediated by the splice control sequence of the present invention.

Preferably, the splice control sequence is 3′ to the start codon. Preferably, the splice control sequence is inserted within the first exon, i.e. the stretch of sequence immediately 3′ to the transcription start site. It will be understood that such terms may refer to the DNA sequence which encodes the transcript, or to the RNA transcript itself.

Where the splice control sequence is 3′ to the start codon, it is preferred that it is also 5′ to the first in-frame stop codon (that is 3′ to and in frame with the start codon), so that alternative splicing yields transcripts that encode different protein or polypeptide sequences. Thus in a preferred embodiment, the construct or polynucleotide sequence comprises the following elements in 5′ to 3′ order, with respect to the sense strand or primary transcript: transcription start, translation start, intron capable of alternative splicing, coding sequence for all or part of a protein, stop codon.

The splice control sequence may be defined as preferably up to and including the 5′ G (GT/C) and its 3′ G equivalent, especially in tra, but as mentioned above, this can include some exonic sequence and therefore, could include the 3′ most (last) nucleotide of the exon (i.e. G).

It is particularly preferred that the splice control sequence is immediately adjacent, in the 3′ direction, the start codon, so that the G of the ATG is 5′ to the start (5′ end) of the splice control sequence. This is particularly advantageous as it allows the G of the ATG start codon to be the 5′G flanking sequence to the splice control sequence.

Alternatively, the splice control sequence is 3′ to the start codon but within 1000 exonic bp, preferably 500 exonic bp, preferably 300 exonic bp, preferably 200 exonic bp, preferably 150 exonic bp, preferably 100 exonic bp, more preferably 75 exonic bp, more preferably 50 exonic bp, more preferably 30 exonic bp, more preferably 20 exonic bp, and most preferably 10 or even 5, 4, 3, 2, or 1 exonic bp.

The present invention is an improvement on the system defined as LA1188 in WO2005/012534. This plasmid had a number of defects, principal of which is that exonic nucleotides were excised with the Cctra intron used therein, thereby resulting in an induced frameshift in the transcript.

Specifically, in addition to the sequence derived from Cctra (the Cctra intron), 4 nucleotides of tTAV sequence were removed in the female-specific transcript. Therefore, though several alternatively spliced transcripts were produced, including one female-specific transcript, none were capable of encoding functional tTAV protein. Therefore, this construct was not capable of providing sex-specific expression of functional tTAV protein.

Since splicing was not directed to the splice donor sequence (5′-GT . . . ) normally used in the Cctra intron, clearly this construct did not contain all of the regulatory sequences necessary to direct splicing in the form of the Cctra intron in “its native context.” However, this highlights another issue. Probably the only thing missing was the flanking TG . . . GT, of which it is possible that only the 5′ G mattered.

A key benefit of the present invention is, in particular in relation to tra, that the requirements for exonic sequence are so minimal (e.g. 2 nucleotides at each end) that they can easily be designed into most coding sequences, using the redundancy in the genetic code. So the “extra” exonic nucleotides can both be part of the heterologous protein sequence, and the flanking sequence of the intron in its native context at the same time.

Furthermore, the Cctra intron in LA1188 was +132 bp 3′ to the G of the ATG start codon (to the last exonic nucleotide). Indeed, although the Cctra intron in LA1188 is the first intron read in the 5′ to 3; direction from the ATG start codon, it is not the “first” intron when read in the 5′ to 3′ direction from promoter. In fact, it is the 2^{nd} intron, as there is a further intron (derived from the Drosophila melanogaster Adh gene) upstream of the ATG start codon. This information is included in the Table 3.

It will be understood that where reference is made to ATG start codons or flanking G, or 5′-TG . . . GT-3′ sequences, that this is in relation to a DNA sequence, but this is also covers the corresponding DNA antisense sequence and, equally, the corresponding RNA sequence.

### DESCRIPTION OF THE SEQUENCES OF THE PRESENT INVENTION

SEQ ID NO. 1 tra consensus sequence

SEQ ID NO. 2 LA3097 5′ flanking sequence

SEQ ID NO. 3 LA3097 3′ flanking sequence

SEQ ID NO. 4 primer 688-ie1-transcr

SEQ ID NO. 5 primer 790-Aedsx-m-r2

SEQ ID NO. 6 primer 761-Aedsx-fem-r

SEQ ID NO. 7 primer AedsxR1

SEQ ID NO. 8 Pane et al consensus sequence

SEQ ID NO. 9 Scali et al 2005 consensus sequence

SEQ ID NOS. 10-33 and 107-138 consensus sequences of putative Tra/Tra2 binding sites deduced for Drosophila (see Table 2).

SEQ ID NO. 34: Open reading frame of tTAV

SEQ ID NO. 35: Protein sequence of tTAV

SEQ ID NO. 36: Open reading frame of tTAV2

SEQ ID NO. 37: Protein sequence of tTAV2

SEQ ID NO. 38: Open reading frame of tTAV3

SEQ ID NO. 39: Protein sequence of tTAV3

SEQ ID NO. 40: Pink Bollworm dsx female specific sequence fragment 1

SEQ ID NO. 41: Pink Bollworm (PBW, Pectinophora gossypiella) dsx female specific sequence fragment 2

SEQ ID NO. 42: Pink Bollworm (PBW, Pectinophora gossypiella) dsx male specific sequence

SEQ ID NO. 43: Partial gene sequence of Aedes aegypti dsx. All exonic sequence is included, but only partial intronic sequence—see FIGS. 47 and 48 for annotation.

SEQ ID NO. 44: Codling moth (Cydia pomonella) dsx female gene sequence: includes a stretch of unknown nucleotides, preferably than then 100, preferably less than 50, more preferably less than 20, more preferably less than 10, and most preferably less than 5.

SEQ ID NO. 45: Codling moth (Cydia pomonella) dsx-male sequence.

SEQ ID NO. 46: Sequence of pLA3435-Bombyx mori-dsx construct/plasmid.

SEQ ID NO. 47: Sequence of pLA3359-Anopheles gambiae dsx construct.

SEQ ID NO. 48: Sequence of pLA3433-Agdsx (Anopheles gambiae) construct with exon 2 included.

SEQ ID NO. 49: Sequence of pLA1188-cctra intron construct

SEQ ID NO. 50: Sequence of pLA3077-a Cctra intron-tTAV construct.

SEQ ID NO. 51: Sequence of pLA3097-a Cctra intron-tTAV construct.

SEQ ID NO. 52: Sequence of pLA3233-Cctra-intron-tTAV2 construct.

SEQ ID NO 53: Sequence of pLA3014-Cctra-intron-Ubiquitin-reaperKR construct.

SEQ ID NO 54: Sequence of pLA3166-Cctra intron-Ubiquitin-reaperKR construct.

SEQ ID NO. 55: Sequence of pLA3376-Bztra intron-reaperKR and Bztra-intron-tTAV3.

SEQ ID NO. 56: Sequence of pLA3242-Crtra intron-reaperKR construct.

SEQ ID NO. 57: Partial sequence of a male transcript generated in Drosophila melanogaster from LA3077 transformants that differs to the sequence generated in Medfly LA3077 lines. This sequence corresponds to the M3 transcript depicted in FIG. 36.

SEQ ID NO. 58: Partial sequence of Bactrocera zonata tra homologue. Sequence of intron predicted to be spliced out in a female-specific transcript of B. zonata tra (+3 to +970 bp in sequence). Exonic flanking nucleotides are at positions 1-2 and 971-972, i.e. at the 5′ and 3′ ends of the intronic sequence. In fact, it is worth noting that the intronic sequence is flanked on its 5′ end by a Guanine nucleotide, which is thought critical for a clean exit of the intron.

SEQ ID NO 59: Partial sequence of Ceratitis rosa tra homologue. Sequence of intron predicted to be spliced out in a female-specific transcript of C. rosa tra (+3 to 1311 bp in sequence). Exonic flanking nucleotides are present at positions 1-2 and 1312-3. Again, it is noteworthy that the intronic sequence is flanked on its 5′ end by a Guanine nucleotide, which is thought critical for a clean exit of the intron.

SEQ ID NOS. 60-70: Primers as referred to in FIGS. 44-
46 and 50-51.
SEQ ID NO. 71: Pink Bollworm (PBW, Pectinophora
gossypiella) dsx female specific fragment 3.
SEQ ID NO. 72: Open reading frame of Drosophila
melanogaster ubiquitin.
SEQ ID NO. 73: Protein sequence of Drosophila
melanogaster Ubiquitin.
SEQ ID NOS. 74-105 are primers as discussed above in
the Examples.
SEQ ID NO. 106 is the LA1172 nucleotide sequence,
including plasmid backbone.
SEQ ID NOs 107-138 are described above.
SEQ ID NO. 139 HSP primer
SEQ ID NO. 140 VP16 primer
SEQ ID NO. 141 primer Agexon1F
SEQ ID NO. 142 primer TETRR1
SEQ ID NO. 143 LA3576 plasmid sequence
SEQ ID NO. 144 LA3582 plasmid sequence
SEQ ID NO. 145 LA3596 plasmid sequence
SEQ ID NO. 146 PBW-dsx (FIG. 6)
SEQ ID NO. 147 bombyx-dsx (FIG. 6)
SEQ ID NO. 148 codling-dsx (FIG. 6)
SEQ ID NO. 149 DSX Minigenel from construct LA3491
SEQ ID NO. 150 DSX Minigene2 from construct LA3534
SEQ ID NO. 151 LA3619 whole plasmid sequence
SEQ ID NO. 152 LA3612 whole plasmid sequence
SEQ ID NO. 153 LA3491 plasmid sequence
SEQ ID NO. 154 LA3515 plasmid sequence
SEQ ID NO. 155 LA3545 plasmid sequence
SEQ ID NO. 156 LA3604 plasmid sequence
SEQ ID NO. 157 LA3646 plasmid sequence
SEQ ID NO. 158 LA3054 plasmid sequence
SEQ ID NO. 159 LA3056 plasmid sequence
SEQ ID NO. 160 LA3488 plasmid sequence
SEQ ID NO. 161 LA3641 plasmid sequence
SEQ ID NO. 162 LA3570 plasmid sequence

The invention will now be described by reference to the following, non-limiting Examples.

### EXAMPLES

### Transformer

### Example 1

### Ceratitis capitata tra Intron

We have prepared an insertion of a Cctra intron cassette into a synthetic open reading frame (ORF). Two versions of this splice correctly in Medfly, in other words the splicing of the Cctra intron cassette faithfully recapitulates what it would normally do in the context of the endogenous Cctra gene. This is to produce 3 (major or only) splice variants in females, one of which is female-specific (called F1), while the other two are found in both males and females (called M1 and M2). Since each of the non-sex-specific transcripts contain additional exonic material with stop codons, we have also arranged this so that only the female splice variant produces functional protein.

Each of these constructs (LA3077 and LA3097) has the Cctra intron flanked by TG and GT (to give 5′ . . . TG|intron|GT . . . 3′. An older construct, which does not work perfectly, is LA1188. LA1188 is quite well characterized—splicing is exactly as above except that an additional 4 nucleotides are removed. The intron is in the context 5′ . . . TGGCAC|intron|GT . . . 3′; splicing removes an additional 4 bases, i.e. 5′ . . . TG|GCACintron|GT . . . 3′ (FIG. 33).

In all cases the intron is invariant, and is simply the complete Cctra intron sequence. As is normal for introns, it begins GT and ends AG. Almost all introns start with GT, so the use of the rare alternative GC in LA1188 is surprising [GC-AG introns are a known alternative—in one large-scale survey, 0.5% of all introns were reported to use GC-AG (Burset et al., 2001), though this may be an underestimate, particularly for alternatively spliced introns, of which perhaps 5% might use GC-AG (Thanaraj and Clark, 2001)].

RT-PCR analysis was performed on LA3077, (a positive feedback construct with the CcTRA intron in the tTAV open reading frame). Transformed adult flies of both sexes were reared on diet substantially free of tetracycline (“off tetracycline”) for 7 days. Flies were then collected for RNA extraction and RT PCR using primers (HSP-SEQ ID NO. 104 and VP16 SEQ ID NO. 105) were used to analyse the splicing pattern of the CcTRA intron (FIG. 34). In two female samples we found the correct splice pattern of the Cctra (776 bp, corresponding to precise removal of the Cctra intron) and saw no such band in males.

We found that LA3077 and LA3097 correspondingly gave repressible female-specific lethality. LA3077 was tested phenotypically through crossing flies heterozygous for LA3077 to wild type, on and off tetracycline. Female lethality ranged from 50 to 70%. LA3097 (a modified version of LA3077 whereby the Cctra intron immediately follows the start codon in the tTAV ORF), demonstrated a much higher level of female specific lethality, peaking at 100% (FIG. 35). The Cctra intron was also inserted in tTAV2 at the same position as LA3097, in construct LA3233, and this gave a similar phenotypic result as LA3097 (FIG. 35).

We have also prepared transformants of LA3077 in Drosophila. Phenotypically, the construct works perfectly, which is to say it is a highly effective female-specific lethal. However, sequencing of the splice variants of one of these insertions has shown that the splicing of this construct in Drosophila is not quite the same as it is in Medfly (SEQ ID NO. 57). The critical transcript, the female-specific one, is the same in both, but at least one of the non-sex-specific transcripts is different. It still incorporates extra exonic sequence, with stop codons, but the splice junctions are not quite the same (FIG. 36). This observation is extremely important in that it shows that this method (regulation of gene expression by use of alternatively spliced introns) can be used across quite a wide phylogenetic range.

A simple test to determine whether an as yet uncharacterized exonic splice regulator (such as enhancers and suppressors) may be modifying the function of the alternatively spliced intron, could include making the construct and introducing it into a target tissue, then examining its splice pattern. In many cases this will not require germline transformation, so the test can be quite rapid, for instance by transient expression in suitable tissue culture cells or in vivo. For instance, in vivo testing in insects could be achieved by delivering the DNA by microinjection. However, as the skilled person will appreciate, microinjection coupled with electroporation, or electroporation, chemical transformation, ballistic methods, for instance, have all been used in a number of various contexts and such methods of plasmid introduction and protein expression therefrom are will known in the art.

We have also recently made, and have obtained transgenics with, the Cctra intron in a different gene (LA3014) (all the above examples are in tTAV). LA3014 contains a ubiquitin-reaper^{KR} fusion downstream of a Cctra intron. Phenotypic data (FIG. 35) shows that LA3014 transgenic Medfly gave repressible female-specific lethality. RT-PCR analysis on RNA extracted from adult males and females raised off tetracycline, using primers (HSP, SEQ ID NO 74) and ReaperKR (SEQ ID NO. 75), demonstrate that correct splicing was occurring in females (508 bp band) and no such band was found in males (FIG. 37). LA3166 is another construct with the Cctra intron placed inside the ubiquitin coding region fused to reaper^{KR}, but placed in a different position in ubiquitin. LA3166 also produces a dominant repressible female-specific lethal effect in Medfly (FIG. 35).

We have also recently made, and have obtained transgenics with, ‘intron-only’ Cctra-based constructs with the intron in a different gene (all the above examples are in tTAV or one of its variants, i.e. tTAV2 or tTAV3). These constructs work as predicted. This is an important result, thus showing that there are not essential exonic sequences in Cctra that we have simply duplicated (in function, if not necessarily in sequence) by chance, in tTAV. We also have ubi-rpr^{KR} constructs of this type (LA3014 and LA3166), which also validates the ubiquitin fusion method described above.

In order to demonstrate the phylogenetic range of the Cctra intron we generated transgenic LA3097 and LA3233 Anastrepha ludens. LA3097 and LA3233 were selected for injection into Anastrepha ludens as they demonstrated the best female specific lethality in Ceratitis capitata (see Example 13). Phenotypic data was generated for 4 independent LA3097 lines and 1 LA3233 line (see FIG. 38). Female specific lethality was generally somewhat lower in Anastrepha ludens when compared to C. capitata but reached 100% in one line.

Anastrepha ludens transformed with LA3097 and raised on tetracycline until eclosion were isolated and maintained off tetracycline for 7 days. RNA was then extracted and RT-PCR analysis was performed using primers HSP (SEQ ID NO. 76) and TETRR1 (SEQ ID NO. 77). The correct female specific (F1-like) splice pattern was observed RNA isolated from in females (348 bp) but not from males demonstrating the function of the Cctra intron in a different species (FIG. 39)

The brightest male band and the female specific band were purified and precipitated for sequencing. The female specific transcript was found to be correctly spliced in Mexfly females as expected for LA3097:

LA3097: AGCCACCATG|GT . . . intron . . . AG|GTCAGCCGCC

The two flanking sequences above are SEQ ID NOS. 2 and 3, respectively.

### Example 2

### Bactocera zonata tra Intron

We isolated the tra intron from Bactocera zonata (B. zonata) (SEQ ID NO. 58) using primers ROSA1 (SEQ ID NO. 78), ROSA2 (SEQ ID NO. 79), and ROSA3 (SEQ ID NO. 80).

These primer sequences were designed based on conserved coding sequence of Ceratitis capitata and Bactrocera oleae tra homologs. Using ROSA2 and ROSA3 or ROSA1 and ROSA3 as primers, the tra intron and its flanking coding region were amplified from Bactrocera zonata genomic DNA. Then we used these PCR products as a template and amplified the tra intron fragment to make the construct-LA3376 (FIG. 31 and SEQ ID NO. 55). The primers (BZNHE-SEQ ID NO. 81 and BZR-SEQ ID NO. 82) were used for making the constructs: these primers contain additional sequences for cloning purposes. The Bztra intron in LA3376 is cloned into the ORF of tTAV3 and also of reaper^{KR}. Medfly transformants were generated and RNA extracted from male and female flies.

RT-PCR was then performed on both the reaper^{KR} (HB-SEQ ID NO. 83) and Reaper KR-SEQ ID NO. 84) and tTAV3 (SRY-SEQ ID NO. 85) and AV3F-SEQ ID NO. 86) splice. The expected fragments of 200 bp for reaper^{KR} and 670 bp for tTAV3, corresponding to splicing in a pattern equivalent to the F1 transcript of Cctra (Pane et al., 2002), were generated in females (FIG. 40).

### Example 3

### Isolation and Splicing of the Ceratitis rosa (C. rosa, Natal Fruit Fly) tra Intron

Primers ROSA2 (SEQ ID NO. 87) and ROSA3 (SEQ ID NO. 88) were designed based on conserved coding sequence of Ceratitis capitata and Bactrocera oleae. Using ROSA2 and ROSA3 as primers, the tra intron and its flanking coding region were amplified from Ceratitis rosa genomic DNA (SEQ ID NO. 59). We then used the PCR products as a template and amplified the tra intron fragment to make constructs. The primers (CRNHE-SEQ ID NO 89 and CRR SEQ ID NO 90) were used during the construction of LA3242 (SEQ ID NO. 56 and FIG. 32. LA3242 contains the C. rosa intron at the 5′ end of the reaper^{KR} ORF. Ceratitis capitata embryos were injected with DNA of LA3242, injected embryos were raised to adulthood on a diet substantially free of tetracycline. RNA was extracted from adult males and females; this was used as a template for RT PCR using primers HB (SEQ ID NO. 91) and ReaperKR (SEQ ID NO. 92). The expected female-specific splice band (200 bp), corresponding to splicing in the equivalent pattern to that of transcript F1 of Cctra, was observed in females and not males (FIG. 41).

### Double-Sex

### Example 4

### Bombyx mori dsx in PBW

The sequence of a Bombyx mori (silk moth) homolog of Drosophila Dsx (Bmdsx) has been previously described and a male- and a female-specific splice product have been identified (Suzuki et al, 2001). Both males and females use the same 3′ polyA, and there are two female specific exons. One paper has suggested that the sex-specific splicing is not dependent on tra/tra2, in other words even though the pattern looks the same, the underlying mechanism may be different (Suzuki et al., 2001), though their data, principally the lack of recognisable tra-tra2 binding sites, however, is not compelling. In addition, a B. mori dsx mini-gene construct (containing exonic sequence and truncated intronic sequence) has been transformed into B. mori and the germline transformants show sex-specific splicing (Funaguma et al., 2005).

We have generated a Bmdsx minigene based on the sequence used in the Funaguma et al paper, with some significant changes, and injected this into the moth Pink Bollworm to ascertain if one can obtain sex-specific splicing in a divergent species. The mini-gene construct we generated does not included exon 1, which is present in both males and females. In addition, we removed the intron between exon 3 and 4 (the two female specific exons), included a heterologous sequence (containing multiple cloning sites, MCS), used the Hr5-IE1 enhancer/promoter sequence from the baculovirus AcNPV and used a 3′ transcriptional termination sequence derived from SV40 (see FIG. 42 for a schematic). The individual exon/flanking intron fragments used were amplified and recombined together by PCR and ligated into a construct carrying a Hr5/IE1 enhancer promoter fragment and SV40 3′UTR (FIG. 22 and SEQ ID NO. 22).

LA3435 was injected into pink bollworm (Pectinophora gossypielia) embryos. First instar larvae were collected after 5-7 days and analysed individually by RT-PCR (using primers IE 1 transcr-SEQ ID NO. 93 and SV40-RT-P2-SEQ ID NO. 94) to determine if BMdsx can undergo male and female specific splicing (FIG. 43). Our analysis detected the male specific band (predicted to be 442 bp) in 4 samples (Lanes 1, 2, 3 and 4) and the female specific band (predicted to be 612 bp) in 1 sample (Lane 5).

The correct splicing of B. mori dsx in PBW demonstrates that we can achieve (have achieved) sex-specific expression of a heterologous sequence (here, the MCS) in a Lepidopteran by utilizing an alternative splicing system. Furthermore, since this splicing system was derived from a heterologous species, this suggests that such constructs might work over a wide phylogenetic range. However, the identification of alternative splicing systems in the species of interest is also envisioned, and methods for identifying such alternative splicing systems are provided herein or will be known to the person skilled in the art. By providing a MCS in our Example (see FIG. 42), the expression of a sequence of interest, for example a coding region for a protein of interest could readily be achieved by inserting said sequence. If said sequence encoded a suitable protein, a sex-specific phenotype, for example conditional sex-specific lethality, could thereby be introduced, for example into pink bollworm.

### Example 5

### Isolation of Codling Moth dsx

The dsx gene from Codling moth (Cydia pomonella) was isolated by performing 3′ RACE using primers which were based on sequence alignments from B. oleae, B. tyroni, C. capitata, D. melanogaster, B. mori, and A. gambiae. RNA was isolated from a male and female codling moth and 3′ RACE, to generate cDNA, was performed using the TT7T25 primer (SEQ ID NO. 95).

PCR was performed using the primers ds1c (SEQ ID NO. 96) and TT7 (SEQ ID NO. 97). Two rounds of nested PCR were then performed on the product of the first PCR using the primers codling2a (SEQ ID NO. 98) and TT7 (SEQ ID NO. 99) and the product of the second round of PCR using Codling2b (SEQ ID NO. 100) and TT7. The isolated male and female specific sequences share sequence similarity to previously isolated dsx homologues (Male-SEQ ID NO. 43 and Female-SEQ ID NO. 42).

### Example 6

### Isolation of PBW dsx

The dsx gene from pink bollworm was isolated by performing 3′ RACE using primers which were based on sequence alignments from B. oleae, B. tyroni, C. capitata, D. melanogaster, B. mori, and A. gambiae. RNA was isolated from a male and female codling moth and 3′ RACE, to generate cDNA, was performed using TT7T25 (sequence defined herein). PCR was performed using the primers Pbwdsx2 (SEQ ID NO. 101) and TT7 (SEQ ID NO. 102). Nested PCR was then performed on the product of the first PCR using the primers Pbwdsx3 (SEQ ID NO. 103) and TT7. Three female specific sequences were isolated: PBWdsx-F1 (SEQ ID NO. 40), PBWdsx-F2 (FIG. 10), and PBWdsx-F3 (SEQ ID NO. 71) and one male specific sequence (SEQ ID NO. 42). The isolated male and female specific sequences share sequence similarity to previously isolated dsx homologues.

### Example 7

### dsx in Anopheles gambiae

The sequence of the dsx gene of Anopheles gambiae has previously been described (Scali et al 2005). However, when we have tried to repeat the work described in the paper we find that there are some differences in the splicing that occurs. When we tried to repeat the amplification of the female specific transcript using primers designed from the mRNA sequence (Accession; AY903308 for female coding sequence and AY903307 for male coding sequence), the amplification failed. However, when Scali and colleagues showed that there was a shared exon, which had previously not been described, we designed primers to amplify the entire dsx transcript and gene. Using these primers and primers designed from genomic DNA sequence (Accession; GI:19611767) we find that the splicing of the female transcript is different from that described by Scali et al 2005 (FIG. 44). The transcript showed that the female exon was in a different position. There are several explanations for these differences, but the most likely are either some sort of strain difference in the Anopheles that we used to get the data from, or the published sequence is not from Anopheles gambiae, or there is more than one female isoform as shown for Stegomyia aegypti in Example 20.

We have also successfully used primers, designed around our version of the Anopheles gambiae dsx splicing, that are able to distinguish between males and females of Anopheles gambiae (FIG. 45). This provides good evidence that the system will be functional as a sex-specific splicing mechanism when fused to a protein of interest, such as tTAV or a killer.

The Anopheles gambiae dsx gene that we have isolated from genomic DNA, which has several changes in nucleotide sequence compared to the reported genomic sequence, was cloned into LA3359 (SEQ ID NO. 47) and LA3433 (SEQ ID NO. 48), schematics can be found in FIG. 23 and FIG. 24, respectively.

### Example 8

### dsx in Siegomyia aegypti

The splicing of the gene appears to be similar to Anopheles gambiae dsx (Scali et al 2005). The Stegomyia aegypti dsx gene is illustrated diagrammatically in FIG. 47 or 48. A male-specific transcript (M1) is produced which does not include exons 5a or 5b. Two female specific splice variants (F1 and F2) have the following structure; F1 comprises exons 1-4, 5a, 6 and 7 but not 5b, F2 comprises exons 1-4 and 5b (FIG. 46). In addition, a further transcript (C1) is present in both males and females; this comprises exons 1-4 and 7, but not exons 5a, 5b or 6.

The splicing of the gene appears to be similar to Anopheles gambiae dsx (Scali et al 2005). The Stegomyia aegypti dsx gene is illustrated diagrammatically in FIG. 47 or 48.

### Actin 4

### Example 9

### Stegomyia aegypti Actin-4 Gene

One way to get sex-, tissue- and stage-specific expression of a gene of interest is to link it with the Stegomyia aegypti Actin-4 (AeAct-4) gene. This gene is only expressed in the developing flight muscles of female Stegomyia aegypti (Munoz et al 2004). They used in-situ hybridisation to an RNA to detect the expression profile of AeAct-4. We have taken a fragment of the Stegomyia aegypti Actin-4 gene, comprising a putative promoter region, an alternatively spliced intron, and a section of 5′ untranslated region (UTR) and placed it in front of sequence coding for tTAV (FIG. 49) to test the function of the sex specific splicing when fused to tTAV.

We integrated LA1172 into the Stegomyia aegypti genome using piggyBac. Two independent lines were generated (lines 2 and 8). Both of these lines show the correct splicing of the Actin-4-tTAV gene (FIGS. 50 and 51). The Actin-4 promoter and alternatively spliced intron can therefore be used successfully to provide sex-, tissue- and stage-specific splicing of a gene of interest in Stegomyia aegypti.

### DESCRIPTION OD THE FIGURES AND SEQUENCE LISTINGS OF EXAMPLES 1-9

FIG. 19: One use of the P element in generating germline-specific expression of a gene of interest (Gene E).

Insertion of the P element IVS3 and flanking exonic sequences upstream of an ubiquitin-Gene E fusion with allow germline-specific expression of Gene E under a germline active promoter. A—Germline active promoter; B—P-element open reading frame; C—P intron ‘IVS3’; D—Ubiquitin; E—Coding region for protein of Interest e.g. tTAV.

FIG. 20: Sex-specific expression using dsx.

A: Intron used as Cctra intron above, but giving male-specific expression. A fragment of dsx (here the Anopheles version) is inserted into a heterologous coding region (shaded boxes). The intron is completely removed in males, but in females the coding region is prematurely terminated.

B: An alternative approach to male-specific expression, in which a heterologous coding region is fused to a fragment of dsx.

C: Female-specific expression: the heterologous coding region is inserted into the female-specific exon, either as an in-frame fusion to a fragment of Dsx, or with its own start and stop codons.

D: Differential expression: designs B and C can be combined to give expression of gene a in females and b in males.

FIG. 21: Sex-specific alternative splicing of Cctra

A: Cctra is spliced in females to produce three transcripts: F1, which encodes functional Tra protein, and M1 and M2, which do not, because they include additional exons with stop codons (redrawn from Pane et al. 2002). Males produce only transcripts M1 and M2 and therefore do not produce functional Tra protein at all.

B If this intron were to function similarly in a heterologous coding region, this would similarly allow females, but not males, to produce functional protein X.

FIG. 22: Diagrammatic representation of pLA3435 construct/plasmid (SEQ ID NO. 46).

FIG. 23: Plasmid map of pLA3359 Anopheles gambiae dsx gene placed under the control of a Hr5-IE1 promoter for assessing splicing via transient expression.

FIG. 24: pLA3433-Anopheles gambiae dsx gene placed under the control of a Hr5-IE1 promoter, with the addition of exon 2, for assessing splicing via transient expression.

FIG. 25: Schematic representation of pLA1188 construct.

FIG. 26: Schematic diagram of pLA3077 construct.

FIG. 27: Schematic diagram of pLA3097 construct.

FIG. 28: Schematic diagram of pLA3233 construct.

FIG. 29: Schematic diagram of pLA3014 construct.

FIG. 30: Schematic diagram of pLA3166 construct.

FIG. 31: Schematic diagram of pLA3376 construct.

FIG. 32: Schematic diagram of pLA3242 construct.

FIG. 33: Flanking sequence of Cctra

Splicing of the Cctra intron in LA3077 and LA3097 is exactly as you would see in the native Cctra intron. Splicing in LA1188 results in the removal of 4 additional nucleotides. In all cases the introns are flanked by 5′ exonic TG and 3′ GT.

FIG. 34: Gel showing correct sex-specific splicing of intron(s) derived from CcTra (776 bp band in females) in Ceratitis capitata transformed with LA3077. Lane 1: Marker (SmartLadder™ from Eurogentec, bands of approx 0.8, 1.0 and 1.5 kb are indicated); Lanes 2 and 3: Ceratitis capitata LA3077/+males; Lanes 4 and 5: Ceratitis capitata LA3077/+females.

FIG. 35: Phenotypic data for transformed female specific constructs in Ceratitis capitata.

Column 1: Construct designation LA#, e.g. LA3077, LA3097, LA3233, etc, is indicated by number, with independent insertion lines referred to by letter; Columns 2 and 3: Non-tetracycline (NT) results for each transformed line given in total males (2) and total females (3). Columns 4 and 5: Tetracycline (TET) results for each transformed line given in total males (4) and total females (5).

FIG. 36: Transcripts of Cctra intron constructs in Drosophila and Ceratitis capitata.

The top line represents the construct DNA containing tra intron flanked by desired gene (the open box). The red box represents the male specific exons. Introns are represented by solid lines. Arrow above the first line represents the positions of the oligonucleotides used in the RT-PCR experiments. The bar indicates the scale of the figure.

FIG. 37: Gel showing correct female specific splicing of CcTRA-derived sequence (508 bp band) in female Ceratitis capitata transformed with LA3014. Lane 1: Marker (SmartLadder™ from Eurogentec, bands of approx 0.4 and 1.0 kb are indicated); Lane 2 Ceratitis capitata LA3014/+male; Lane 4: Ceratitis capitata LA3014/+female; Lanes 3 and 5: no reverse transcriptase negative controls (background bands, probably from genomic DNA, can be seen in lanes 2 and 4).

FIG. 38: Phenotypic data for transgenic Anastrepha ludens transformed with LA3097 or LA3233. Column 1: Construct LA# (LA3097 or LA3233) indicated, with independent insertion lines referred to by letter; Columns 2 and 3: Non-tetracycline (NT) results for each transformed line given in total males (2) and total females (3). Columns 4 and 5: Tetracycline (TET) results for each transformed line given in total males (4) and total females (5).

FIG. 39: Gel showing correct sex-specific splicing of CcTRA splicing (348 bp band in females) in Anastrepha ludens transformed with LA3097. Lane 1: Marker (SmartLadder™ from Eurogentec, bands of approx 0.4 and 1.0 kb are indicated); Lanes 2, 3 and 4: A. ludens LA3097/+males; Lanes 5, 6 and 7: A. ludens LA3097/+females.

FIG. 40: Gel showing correct sex-specific splicing of BzTRA in reaperKR (200 bp band in females) and tTAV3 (670 bp band in females) regions of LA3376, in Ceratitis capitata transformed with LA3376. Lane 1: Marker (SmartLadder™ from Eurogentec, bands of approx 0.2, 0.6 and 1.0 kb are indicated); Lanes 2 and 3: C. capitata LA3376/+males tested for splicing in reaperKR; Lanes 4 and 5: C. capitata LA3376/+females tested for splicing in reaperKR; Lane 6: SmartLadder™; Lanes 7 and 8: C. capitata LA3376/+males tested for splicing in tTAV; Lanes 9 and 10: C. capitata LA33761+females tested for splicing in tTAV; Lane 11: SmartLadder™.

FIG. 41: Gel showing correct sex-specific CrTRA splicing in CrTRA-reaperKR (200 bp band in females) in Ceratitis capitata injected with LA3242. Lane 1: Marker (SmartLadder™ from Eurogentec, bands of approx 0.2, 0.6 and 1.0 kb are indicated); Lanes 2-7: C. capitata wild type males injected with LA3242; Lane 8: SmartLadder™; Lanes 9-14: C. capitata wild type females injected with LA3242; Lane 15: SmartLadder™.

FIG. 42: Schematic representation of Bmdsx minigene constructs.

Two minigene constructs derived from the Bombyx mori dsx gene are illustrated diagrammatically, together with the predicted alternative splicing of these constructs (female pattern shown above the construct, male pattern below). (A) is the Bombyx mori dsx mini-gene construct used in Funaguma et al., 2005) (B) is pLA3435. A and B differ from each other in several ways: (i) Exon 1 is excluded from pLA3435, (ii) the intron between female specific exons 3 and 4 has been removed and a short heterologous sequence has been inserted in pLA3435 (iii) Funaguma et al., use the ie1 promoter from the baculovirus BmNPV and a BmA3 3′UTR compared with pLA3435 which uses the hr5-IE1 enhancer/promoter from the baculovirus AcNPV and a 3′SV40 3′UTR. (iv) pLA3435 uses slightly longer intron sequences when compared with (A) (see FIG. 15 for sequence). Two minigene constructs derived from the Bombyx mori dsx gene are illustrated diagrammatically, together with the predicted alternative splicing of these constructs (female pattern shown above the construct, male pattern below).

FIG. 43: Sex-specific splicing of BMdsx mini-gene construct in PBW.

Analysis of transient expression from pLA3435 using RT-PCR show the presence of a 442 bp fragment (Lanes 1, 2, 3 and 4) in males and a 612 bp fragment in females (Lane 5), showing that the BMdsx mini-gene with a heterologous fragment inserted between exon 3 and 4 is able to splice correctly in the divergent moth, PBW. Markers are SmartLadder™ from Eurogentec; bands of approx 0.2, 0.4 and 0.6 kb are indicated

FIG. 44: Sex-specific splicing of Anopheles gambiae dsx.

Anopheles (A) shows the splicing that was reported by Scali et al 2005. However, when RT-PCR was performed using our primers (spl-agdsx-e3 (SEQ ID NO. 60) and spl-agdsx-m (SEQ ID NO. 61)) a different splicing pattern for females was revealed, represented by Anopheles (B).

FIG. 45: Identification of male and female Anopheles gambiae using dsx primers.

RNA was extracted from male and female Anopheles gambiae and the dsx transcripts were amplified by RT-PCR using the primers spl-agdsx-e3 (SEQ ID NO. 62) and spl-agdsx-m (SEQ ID NO. 63); the resulting banding pattern is shown in the gel above. The expected bands for the male and female transcripts are indicated by the white arrows, the bands have been cloned and sequenced and are identical to the predicted sequence of our version of the dsx transcript (see SEQ ID NO. 47 (LA3359) and SEQ ID NO. 48 (LA3433)). The molecular weight markers are shown in kb (SmartLadder™ from Eurogentec; sizes are approximate).

FIG. 46; Identification of male and female Stegomyia aegypti using dsx primers.

The primers for the Stegomyia aegypti RT-PCR for A and B were aedesxF1 (SEQ ID NO. 64) and aedesxR5 (SEQ ID NO. 65) were tested initially on pupae, a life stage of Stegomyia aegypti that can be sexed conveniently and accurately; the resulting RT-PCR amplification is shown on gel image (A). The male and female pupae show a distinctive sex specific band. Then the primers were tested on RNA extractions from larvae, which can not be readily sexed by their morphology and the resulting RT-PCR amplification shown on gel image (B). The larvae show a clear banding pattern which distinguishes males from females unambiguously. Gel image (C) shows an approximately 600 bp band from RT-PCR using the primers aedessxF1 and aedesxR2 (SEQ ID NO. 66) from individual male and female pupa. Sequencing of this band showed a female specific splice variant which does not appear to possess the male shared exon to which aedesxR5 is predicted to anneal (exon 7, see FIG. 56). The molecular weight markers are shown in kb (SmartLadder™ from Eurogentec; sizes are approximate).

FIG. 47: Diagrammatic representation of part of the Stegomyia aegypti dsx gene (not to scale).

A fragment of the Stegomyia aegypti dsx gene is represented above. Exons 5a and 5b are female specific and exon 6 is a male specific exon. Two female-specific splice variants have been found (F1 and F2) which comprise exons 1-4, 5b, 6 and 7 (F1) or 1-4, 5a (F2); transcripts in males (M1) comprise exons 1-4, 6 and 7 but not exon 5a or 5b and a transcript (C1) of 1-4 and 7 but not exons 5a, 5b or 6 is shown in males and females. The numbers for each of the exons after # relates to contig 1.370 (http://www.broad.mit.edu/annotation/disease_vector/aedes_aegypti/), which reads in the opposite orientation, and after * relate to the nucleotide sequence shown in SEQ ID NO. 43.

FIG. 48: Diagrammatic representation of the Stegomyia aegypti dsx gene.

The entire Stegomyia aegypti dsx gene is represented above Exon 5 is the female specific exon and exon 6 is a putative male specific exon. In principle, transcripts in females comprise exons 1, 2, 3, 4, 5 and 7, and males comprise exons 1, 2, 3, 4, 6 and 7. The numbers for each of the exons after # relates to contig 1.370 (http://www.broad.mit.edu/annotation/disease_vector/aedes_aegypti/) reading in the opposite orientation, and after * relate to FIG. 12.

FIG. 49: Plasmid map of pLA 1172.

A coding region for tTAV has been placed under the control of a fragment from the Stegomyia aegypti actin-4 gene (Munoz et al 2005) which includes the 5′ UTR, first intron, and upstream sequences (putative promoter). The construct also contains a tetO₇ Nipper sequence. The construct has piggyBac ends and a DsRed2 marker for stable integration into a genome.

FIG. 50: Sex-specific splicing of tTAV in LA1172 transformants.

Gel image of RT-PCR of RNA extracted from LA1172 line 2 male and female pupa. The primers used were Agexon1 (SEQ ID NO. 67) and Tra (tTAV) seq+(SEQ ID NO. 68). Sequencing of the RT-PCR bands showed the expected splicing occurring in males and females. The data shown in the above diagram is for LA1172 line 2, line 8 showed exactly the same results (data not shown). Markers are SmartLadder™ from Eurogentec; approximate sizes are indicated, in kb).

FIG. 51: RT-PCR of wild type samples, showing sex-specific splice variants of the Stegomyia aegypti Actin-4 gene.

Gel image of RT-PCR of RNA extracted from different developmental stages, and dissections of adults, of LA1172 line 8. The primers used were Agexon1 (SEQ ID NO. 69) and Exon 3 (SEQ ID NO. 70). The gel image shows that strong expression from the Actin-4 gene only occurs at the pupal stage, and that adult expression is generally limited to the female thorax where the flight muscles are found. Table 17, below show the contents of each lane.

TABLE 1

| | |
|---|---|
| E = pool of ~100 embryos | MH = head from male adult |
| L4 = 4^{th} instar larva | MT = thorax from male adult |
| ME = early male pupa | MA = abdomen from male adult |
| (<4 hours old) | FH = head from female adult |
| FE = early female pupa | FT = thorax from female adult |
| (<4 hours old) | FA = abdomen from female adult |
| MP = male pupa | -ve = water control |
| FP = female pupae | |

### FURTHER EXAMPLES

### Example 10

### Moths

We have newly made constructs based on our transient expression data using a recombinant minigene construct derived from Bombyx mori. This is discussed further below in the section entitled “Moth dsx sequence alignment and conserved motifs”

### Example 11

### Use of Bztra

We have newly made two Bztra-based constructs, expressed in Mexfly (LA3376). LA3376 gives repressible female-specific lethality. LA3376 we have previously shown to function and splice correctly in Medfly. Transformants in Mexfly (Anastrepha ludens) were also generated with LA3376. These were analysed for correct splicing of the Bztra intron in order to demonstrate the phylogenetic range of the Bztra intron by RT-PCR using primers SRY and AV3F (FIG. 15 and “Medfly RT-PCR gels” section above). This shows correct splicing of the Bztra intron in Mexfly.

### Example 12

### Dmdsx in Medfly (DmDsx in Transgenic Medfly Example: Nipper Fusion in #797)

We also have newly made data on a Dmdsx construct in Medfly. The construct used a fragment of the Drosophila melanogaster gene doublesex to give sex-specific expression of a fragment of the Drosophila melanogaster gene Nipp1Dm (we call this fragment “nipper”). We didn't see clear sex-specific splicing. However, the phenotypic data shows some sex-specificity; we saw increased lethality of females, to about 75% penetration. Of course this incomplete penetrance could be due to expression level, lack of toxicity of nipper in Medfly, etc. We also had a significant reduction in the number of males, but the tTA source, LA670, used in this experiment could itself be killing some of the males.

We have tested three independent Medfly transgenic lines that carry a fusion of nipper to DmDsx sequence that was intended to be expressed specifically in females. This construct may not have worked perfectly possibly due to essential sequence for correct alternative splicing and/or the Sxl binding sites required by DmDsx, and since Medfly do not use Sxl in the sex-determining pathway, DmDsx may be unable to completely splice this fusion in the correct way in Medfly. However, we were successful in reproducibly causing increased lethality in females compared to males across all three lines at a very similar efficiency (approximately 75% more lethality observed in females than in males). This demonstrates the dsx system can work across quite distantly related species (evolutionary separation is around 120-150 Million years), and if the Ccdsx sequence were used it may have well worked due to the Sxl requirement of Dmdsx.

The 797 results are shown below, using a Tet014 dsx splice nipper (Pub EGFP) system. They show that this system is lethal at the larval stage (˜50%), and is likely to be acting more successfully in females (˜75%). 797 is marked with green (G), 670 with red (R). 670 is a tTAV source, so one expects to see a phenotype in the R+G flies; G (and R) only are controls. NF—non-fluorescent (i.e. wild type) is also a control where included. All progeny reared on tet-free media.

All three independent Lines seem to act in similar way.

| | | |
|---|---|---|
| Pupae | Adults | Males:Females |

797A/797A M2 × 670A/+:

| | | | |
|---|---|---|---|
| G | 184 | 176 | 85:91 |
| R + G | 74 | 57 | 44:13 |

797C/797C M1 × 670A/+:

| | | | |
|---|---|---|---|
| G | 169 | 157 | 89:68 |
| R + G | 94 | 67 | 54:13 |

797C/797C M2 × 670A/+:

| | | | |
|---|---|---|---|
| G | 406 | 377 | 179:198 |
| R + G | 171 | 147 | 121:26 |

670A/+ × 797C/+M2:

| | | | |
|---|---|---|---|
| NF | 198 | 192 | 92:100 |
| G | 162 | 147 | 67:80 |
| R | 149 | 72 | 43:29 |
| R + G | 45 | 22 | 20:2 |

Average of all 3 lines: number of R+G females 21% of the number of R+G males, therefore substantial excess mortality in R+G females relative to males. This effect is not seen in R only or G only control females, nor in wild type.

### Examples 13-15

We have newly demonstrated:

(5) sex-specific splicing in recombinant Aadsx-based minigene constructs;

(6) sex-specific phenotype from a Cctra-based construct; and

(7) sex-specific splicing in Aedes-Actin4-based constructs.

At least some of each of these examples not only shows minigenes, but actually shows splicing to generate tTAV/tTAV2 or ubi-tTAV2

### Example 13

### Aedes Doublesex (dsx) Minigenes

See also section entitled Aedes dsx Tra2 binding sites. We have isolated the Aedes aegypti dsx gene (Aadsx) and identified 6 transcripts from this region (FIG. 1). These are: 2 male-specific transcripts (M1 and M2), 3 female-specific transcripts (F1, F2 and F3) and a transcript found in both males and females (MF). We made two minigene constructs. In these constructs, the large majority of the intronic sequence was deleted. For example, DSX minigene1 is approximately 4.4 kb in length, whereas its terminal sequences are separated by approximately 26 kb in its natural context, i.e. in the genomic DNA of Aedes aegypti.

The splicing in minigene2 of FIG. 1 is illustrative as splicing occurs in the “female” form in both males and females. This may mean that this system depends on alternative splice acceptor use. In this model, there is competition between alternative splice acceptors, with some sex-specific factor biasing this, the sex-specific factor probably being Tra. But deleting the M1 and M2 3′ splice acceptors forces splicing in the F forms, by removing the alternative.

Therefore, it is preferred that one or more of the female-specific (F1 and/or F2) 3′ splice acceptors are provided together with an additional 3′ splice acceptor. Most preferably, said additional splice acceptor is the 3′ splice acceptor of M1 or M2 splice variant (or both), although it is envisaged that this is not essential as other known 3′ splice acceptors are likely to function.

FIG. 1 illustrates the various transcripts produced by alternative splicing of the Aedes aegypti doublesex gene (Aadsx). It will be appreciated that Aedes aegypti is also known as Stegomyia aegypti. The figure shows the Aadsx gene from the fourth exon, which is not alternatively spliced, i.e. is present in all transcripts discussed here. Numbering is from the first nucleotide of the fourth exon (acgacgaact . . . ). Note that the diagram is not to scale—the introns are much longer than the exons. The total alternatively spliced region comprises over 43 kb.

This minigene fragment was included in an expression construct (LA3515). Transgenic Aedes aegypti were generated by site-specific recombination into an attP site, using the method of Nimmo et al (2006: Nimmo, D. D., Aiphey, L. Meredith, J. M. and Eggleston, P (2006). High efficiency site-specific genetic engineering of the mosquito genome. Insect Molecular Biology, 15: 129-136)

A second, smaller minigene was constructed similarly (DSX minigene2) and an expression construct for this was inserted into the same attP site as DSX minigene1, to allow direct comparison (LA3534). DSX minigene2 did not show sex-specific splicing. This indicates that sequences present in DSX minigene1 but not in DSX minigene2 (approx 2029 bp, see FIG. 1 and SEQ ID NO. 150, where exons are found at positions 29-163 and 1535-2572) are essential for correct alternative splicing, even though the first alternatively spliced intron, and the exonic sequence immediately flanking it, is present in both constructs.

We have produced two transgenic lines (LA3491 and LA3534) using minigene constructs of Aedes aegypti dsx gene. LA3491 is a fusion of shared exon4, the female-specific cassette exons, and part of the first shared 3′ exon (exon 5 in transcript M1).

Transcripts from the minigene region of LA3491 were analysed by reverse transcriptase PCR (RT-PCR) and sequencing. Transcripts corresponding to alternative splicing in the F2 form were found in females but not in males (FIGS. 2 and 3) and in the F1 form there was some male expression but it was very low (FIG. 4). While transcripts corresponding to the M1 form were detected in males but not in females (FIG. 2). Since the minigene did not contain the 3′ splice acceptor of the M2 variant, this transcript was not possible from this construct. This minigene does not contain any exogenous sequence, though it clearly demonstrates sex-specific splicing of an Aadsx fragment, indeed a highly deleted “minigene” fragment.

It will be apparent that certain sequences are important for controlling splicing and should therefor be retained, as discussed elsewhere. This can be easily established by deletion of certain portions and testing for alternative splicing by RT-PCR for instance.

FIG. 2 shows RT-PCR of males and females from LA3491 Aedes aegypti transgenic line using the primers 688-ie1-transcr (SEQ ID NO. 4) and 790-Aedsx-m-r2 (SEQ ID NO. 5). Using these primers, splicing in the F2 pattern would give a band of approximately 985 bp while splicing in the M1 pattern would give a band of approximately 516 bp. A band of approx 985 bp (F2) appeared only in lanes representing females and a band of approx 516 bp male specific transcript 1 (M1) appeared only in males. These bands have been sequenced and show that correct splicing had occurred, i.e. F2-type and M1-type respectively. The absence of bands in the no RT controls (-RT CON) shows that there was no genomic DNA contamination in the samples. Lanes 1 and 11 are Marker (SmartLadder™ from Eurogentec, bands from 1.5 kb to 0.2 kb are indicated). Lanes 2 and 3 are negative controls (no reverse transcriptase) and lanes 2-9 represent reactions performed on extracts from males or females as marked.

FIG. 3 shows RT-PCR of males and females from LA3491 Aedes aegypti transgenic lines using the primers 688-ie1-transcr (SEQ ID NO. 4) and 761-Aedsx-fem-r (SEQ ID NO. 6). Using these primers, splicing in the F2 pattern would give a band of approximately 525 bp. A band of approximately 525 bp was present in reactions on extracts from females, but not from corresponding reactions on extracts from males. Sequencing of this 525 bp band confirmed that correct, i.e. F2-type splicing had occurred. Marker (SmartLadder™ from Eurogentec, bands from 1.5 kb to 0.2 kb are indicated).

FIG. 4 shows RT-PCR of males and females from LA3491 Aedes aegypti transgenic lines using the primers 688-ie1-transer (SEQ ID NO. 4) and AedsxR1 (SEQ ID NO. 4). Using these primers splicing in the F1 pattern would give a band of 283 bp. A band of approximately 283 bp is present predominantly in females, although there is evidence of a small amount of splicing in males. Sequencing confirmed that this band did indeed correspond to splicing in the F1 pattern. Marker (SmartLadder™ from Eurogentec, bands from 1.5 kb to 0.2 kb are indicated).

LA3534 is identical to LA3491 except for a 3′ deletion of approx 2 kb. This construct showed no differential splicing between male and females (FIG. 1, minigene 2). RT-PCR gels have not been shown for this case. Based on these results several constructs have been designed to incorporate the sex-specific splicing of LA3491 (FIG. 1, minigene 1) into a positive-feedback system. LA3612 (FIG. 5), which incorporates a fusion of ubiquitin and tTAV2 into the dsx coding region, is designed so that when the F2 female transcript is produced, the ubiquitin is cleaved and the tTAV2 is released to initiate and sustain the positive feedback system. LA3619 (FIG. 5) has tTAV2 without ubiquitin and using its own translation start codon. LA3646 (FIG. 5) is identical to LA3619 except the start codons for the dsx gene have been mutated; this should improve the quantity of tTAV2 produced by removing non-specific translation.

FIG. 5 is a diagrammatic representation of plasmids based around the splicing in Aedes aegypti dsx minigene. For clarity it will be understood that the first female intron represents any of F1, F2 or F3 splicing, and tTAV in the diagram refers to tTAV2 (it will be appreciated that other proteins or other versions of tTA or tTAV could alternatively be used). In each of these plasmids, apart from LA3491, heterologous sequence has been added to the F2 exon. “Putative ATG” represents any ATG triplet sequence in exonic sequence located 5′ relative to the heterologous DNA. In LA3646 these putative translation start codons (“putative ATG”) were removed or modified. In the case of construct LA3612, translation from an upstream (5′) ATG that is in frame with the ubi-tTAV coding region will still (assuming no intervening stop codon) produce functional tTAV, following separation of the ubiquitin and tTAV moieties by protease action. The various alternative splicing cassettes are operably linked to a suitable promoter, transcriptional terminator and other regulatory sequences.

This example shows sex-specific splicing of a highly compressed “minigene” fragment in a heterologous context (i.e. heterologous promoter, 5′ UTR and 3′UTR). Although it does not show differential expression of a non-Aedes sequence, as the alternatively spliced exons are derived from the Aadsx gene and do not contain additional material, it does clearly illustrate the feasibility of this approach. In any case, the promoter, 5′ UTR and 3′UTR are heterologous. We have additional constructs which illustrate several different methods for obtaining differential (sex-specific) expression of a heterologous protein by this dsx.

TRA Sequence Alignment

Pane et al. (2002) suggested that certain sequences related to the known binding sites of the Tra/Tra-2 complex in Drosophila might be important in regulating the splicing of Cctra, and this also known for Drosophila dsx and has also been suggested for Anopheles gambiae dsx (Scali et al 2005). The consensus sequence is variously described as

SEQ ID NO. 8

UC(U/A)(U/A)C(A/G)AUCAACA (Pane et al),,
or

SEQ ID NO. 9

UC(U/A)(U/A)CAAUCAACA (Scali et al 2005),.

It is noteworthy that these definitions are extremely similar. Pane et al identify 8 partial matches to this consensus in the Cctra sequence (7 or more nucleotides matching the 13 nucleotide consensus sequence. Scali et al identify 6 matches in Agdsx (9/13 or better). Such sequences are also known to regulate the alternative splicing of the Drosophila gene fruitless; Scali et al review 3 matches in that sequence (12/13 or better). Correct splicing of dsx may also require a purine-rich region, as discussed by Scali et al.

As can be seen from the Table 2 and FIG. 7, we have identified what are thought to be significant clusters of binding sites for Tra/Tra2 in our Aedes aegypti dsx minigene1.

Moth dsx Sequence Alignment and Conserved Motifs

FIG. 6 shows an alignment of the second female-specific exons and flanking sequences of dsx genes from pink bollworm (Pectinophora gossypiella, PBW-dsx, SEQ ID NO. 146), silk worm (Bombyx mori, bombyx-dsx, SEQ ID NO. 147) and codling moth (Cydia pomonella, codling-dsx, SEQ ID NO. 148). The second female-specific exon is shown in bold. We identified multiple copies of a short, repeated nucleotide sequence, conserved in sequence and approximate location between these relatively distantly related moths; these are located just 5′ to the female-specific exon. The conserved repeats AGTGAC/T are underlined. Asterisks (*) represent identical nucleotides, dashes (−) represent gaps for best alignment. The exons are represented in the SEQ ID NOS. by the following nucleotide numbering: SEQ ID NO. 146 289-439; SEQ ID NO. 147 339-492; and SEQ ID NO. 148 285-439.

Aedes dsx Tra2 Binding Sites.

In females of Drosophila melanogaster, Tra and a product from the constitutively active gene tra2, act as splicing regulators by binding to splice enhancer sites on the pre-mRNA of dsx, which activates the weak 3′ acceptor site of the female-specific exon (Scali et al). In males there is no expression of TRA and the weak 3′ acceptor site is not recognised and splicing occurs at the male exon. To look for putative Tra/Tra2 binding sites we used the consensus sequence of these binding sites deduced for Drosophila Tra/Tra2 and looked for the distribution of these in the Aedes aegypti dsx gene sequence. This is shown in Table 2, below.

TABLE 2

| | | | | | | |
|---|---|---|---|---|---|---|
| | Sequence | | | Identity | Identity | SEQ |
| | w = T or A | Present in | | with | with | ID |
| Name | r = A or G | Minigenel | Position | consensus | wwcrat | NO. |

| | | | | | | |
|---|---|---|---|---|---|---|
| Consensus | tcwwcratcaaca | / | / | /13 | /6 | 138 |
| 1 | tcaacaagcaaca | Y | 14917 | 12 | 5 | 10 |
| 2 | ttatcaaacaaca | Y | 364 | 11 | 5 | 11 |
| 3 | tcatcaattaaaa | | 1015 | 11 | 6 | 12 |
| 4 | tcatcaatcaaac | | 6502 | 11 | 6 | 13 |
| 5 | tcttcaaccaacc | Y | 14958 | 11 | 5 | 14 |
| 6 | cctacaatctaca | Y | 14973 | 11 | 6 | 15 |
| 7 | tcttagatcaaaa | | 16553 | 11 | 5 | 16 |
| 8 | tcttcgatcatta | | 17386 | 11 | 6 | 17 |
| 9 | ccaacaatctaca | | 28802 | 11 | 6 | 18 |
| 10 | tcaaagatcacca | | 142096 | 11 | 5 | 19 |
| 11 | tcttcggtcgacg | Y | 256 | 11 | 5 | 20 |
| 12 | tcgacaaacaaaa | | 1277 | 11 | <5 | 21 |
| 13 | tattcaaacaacg | | 4061 | 11 | 5 | 22 |
| 14 | ttttcgataaaaa | | 4380 | 10 | 6 | 23 |
| 15 | tcttcagtctgca | | 5399 | 10 | 5 | 24 |
| 16 | gattcaatcatca | | 7723 | 10 | 6 | 25 |
| 17 | ttatcgagcaaaa | | 8137 | 10 | 5 | 26 |
| 18 | tcataactcaaga | | 9062 | 10 | <5 | 27 |
| 19 | tcagaaatcaaaa | | 9126 | 10 | <5 | 28 |
| 20 | tctttaatttaca | | 10639 | 10 | 5 | 29 |
| 21 | tttacaatcctca | | 10646 | 10 | 6 | 30 |
| 22 | tcatagatcagga | | 11214 | 10 | 5 | 31 |
| 23 | acctcaaacaaca | | 11989 | 10 | <5 | 32 |
| 24 | tcatcgaacaccc | | 12020 | 10 | 5 | 33 |
| 25 | tcaataatcgtca | | 12199 | 10 | 5 | 107 |
| 26 | tcatcaaacgtca | | 13287 | 10 | 5 | 108 |
| 27 | ttatcgttaaaca | Y | 13439 | 10 | 5 | 109 |
| 28 | taaacagtcaata | Y | 13446 | 10 | 5 | 110 |
| 29 | tacacgatcagca | Y | 14096 | 10 | s | 111 |
| 30 | aatacaaacaaca | Y | 14637 | 10 | 5 | 112 |
| 31 | tcatcaacaagca | Y | 14914 | 10 | 5 | 113 |
| 32 | tctacaaaccaga | Y | 14980 | 10 | 5 | 114 |
| 33 | acatcgattcaca | | 16085 | 10 | 6 | 115 |
| 34 | cgctcaatcaaca | | 16175 | 10 | 5 | 116 |
| 35 | tctaccataaaaa | | 16511 | 10 | 5 | 117 |
| 36 | aaatgaatcaaca | | 20044 | 10 | 5 | 118 |
| 37 | acatcgttcaacg | | 21374 | 10 | 5 | 119 |
| 38 | tcttgattcacca | | 21580 | 10 | <5 | 120 |
| 39 | tctgcagacaaca | | 22408 | 10 | <5 | 121 |
| 40 | tcttcggtaatca | | 23285 | 10 | 5 | 122 |
| 41 | tctataaacaata | Y | 25436 | 10 | <5 | 123 |
| 42 | taaacaataaata | Y | 25440 | 10 | 6 | 124 |
| 43 | taaacaagcaaaa | | 28242 | 10 | 5 | 125 |
| 44 | tcaacgatcggcg | | 30309 | 10 | 6 | 126 |
| 45 | tgatccatcatca | | 30910 | 10 | 5 | 127 |
| 46 | tcaacatgcaaga | | 32295 | 10 | <5 | 128 |
| 47 | tcttaaataaaga | | 32862 | 10 | 5 | 129 |
| 48 | tcaaagatctata | | 40551 | 10 | 5 | 130 |
| 49 | taatgaattaaca | | 40847 | 10 | 5 | 131 |
| 50 | tttaccatcaact | | 41712 | 10 | 5 | 132 |
| 51 | taatgaaacaaca | | 43380 | 10 | <5 | 133 |
| 52* | gtttcaattaaaa | Y | 13500 | 9 | 6 | 134 |
| 53* | tattcaattataa | Y | 13602 | 9 | 6 | 135 |
| 54* | tcttcaatcgttt | Y | 15002 | 9 | 6 | 136 |
| 55* | tcaacgatccttt | Y | 15533 | 9 | 6 | 137 |

*= in 3491, only 9/13 but 6/6 in core. This table does not include 9/13 identities apart from the ones that are in 3491 with 6/6 identity with core sequence of wwcrat. This consensus core sequence (WWCRAT) is particularly preferred.

FIG. 7 is a diagrammatic representation of putative Tra/Tra2 binding sites within the dsx coding region of plasmid LA3491. This diagram is approximately to scale and represents a sequence of approximately 4 kb. We can calculate the chance of a random match to the Tra/Tra2 consensus sequence. Assuming all 4 nucleotides occur at equal frequency, the chances of any given nucleotide in a random sequence being the first nucleotide of a 10/13 or better match to the consensus is approx 7×10⁻⁴. Therefore, one would expect slightly less than one such match per 1000 nucleotides of such random sequence. The calculation for this is below:

Sex-Specific Splicing: Probabilities

Questions

A binding site consensus sequence consists of 13 bases. Ten of those (fixed) positions (call this set X) must each be one specific base. The other three (call this set Y) can each be one of two specific bases. Assuming that each possible base A, G, C and T is equally likely and that the base at each position is independent of the bases at the other positions, what is the probability of a 13-base sequence selected at random exactly matching this sequence? What are the probabilities of such a sequence being a near mismatch (allowing for up to one, two, three or four differences)? The answers are provided in Table 2 below and the workings are shown thereafter.

Answers

TABLE 3

| No. of positions | Probability | Probability |
|---|---|---|
| mismatched | (fraction) | (to 3 d.p.) |
| none, i.e. exact match | $\frac{1}{{2}^{23}}$ | 1.192 × 10⁻⁷ |
| up to 1, i.e. at least 12 positions match | $\frac{17}{{2}^{22}}$ | 4.053 × 10⁻⁶ |
| up to 2, i.e. at least 11 positions match | $\frac{133}{{2}^{21}}$ | 6.342 × 10⁻⁵ |
| up to 3, i.e. at least 10 positions match | $\frac{23}{{2}^{15}}$ | 7.019 × 10⁻⁴ |
| up to 4, i.e. at least 9 positions match | $\frac{33863}{{2}^{23}}$ | 4.037 × 10⁻³ |

Workings

$$\begin{matrix} P ( exact match ) = {P}_{0} \\ = {( \frac{1}{4} )}^{10} {( \frac{1}{2} )}^{3} \\ = \frac{1}{{4}^{10} \times {2}^{3}} \\ = \frac{1}{{2}^{23}} \\ = 1.192 \times {10}^{- 7} to 3 d . p . ( 3 d . p . all below )\end{matrix} \\ P ( mismatch in exactly 1 position ) = P ( mismatch at one of the 10 X positions or mismatch at one of the 3 Y positions ) \begin{matrix} = {P}_{1} \\ = 10 {( \frac{1}{4} )}^{9} ( \frac{3}{4} ) {( \frac{1}{2} )}^{3} + 3 {( \frac{1}{4} )}^{10} {( \frac{1}{2} )}^{3} \\ = \frac{( 10 \times 3 ) + 3}{{4}^{10} \times {2}^{3}} \\ = \frac{33}{{2}^{23}} \\ = 3.934 \times {10}^{- 6}\end{matrix} \\ P ( mismatch in exactly 2 positions ) = P ( mismatches at 2 of the 10 X or mismatch at 1 of the 10 X and 1 of the 3 Y or mismatches at 2 of the 3 Y ) \\ \begin{matrix} = {P}_{2} \\ = \frac{10 !}{2 ! 8 !} {( \frac{1}{4} )}^{8} {( \frac{3}{4} )}^{2} {( \frac{1}{2} )}^{3} + 10 \times 3 {( \frac{1}{4} )}^{9} ( \frac{3}{4} ) {( \frac{1}{2} )}^{3} + 3 {( \frac{1}{4} )}^{10} {( \frac{1}{2} )}^{3} \\ = \frac{( ( 45 \times {3}^{2} ) + ( 30 \times 3 ) + 3 )}{{2}^{23}} = \frac{498}{{2}^{23}} = \frac{249}{{2}^{22}} = 5.937 \times {10}^{- 5}\end{matrix} \\ P ( mismatch in exactly 3 positions ) = P ( mismatches at 3 of the 10 X or mismatches at 2 of the 10 X and 1 of the 3 Y or mismatches at 1 of the 10 X and 2 of the 3 Y or mismatches at 3 of the 3 Y ) \\ \begin{matrix}= {P}_{3} \\ = \frac{10 !}{3 ! 7 !} {( \frac{1}{4} )}^{7} {( \frac{3}{4} )}^{3} {( \frac{1}{2} )}^{3} + \frac{10 !}{2 ! 8 !} 3 {( \frac{1}{4} )}^{8} {( \frac{3}{4} )}^{2} {( \frac{1}{2} )}^{3} + \\ 10 \times 3 {( \frac{1}{4} )}^{9} ( \frac{3}{4} ) {( \frac{1}{2} )}^{3} + {( \frac{1}{4} )}^{10} {( \frac{1}{2} )}^{3} \\ = \frac{( \begin{matrix}( 120 \times {3}^{3} ) + ( 45 \times {3}^{3} ) + \\ ( 30 \times 3 ) + 1\end{matrix} )}{{2}^{23}} = \frac{5356}{{2}^{23}} = \frac{1339}{{2}^{21}} = 6.385 \times {10}^{- 4}\end{matrix} \\ P ( mismatch in exactly 4 positions ) = P ( mismatches at 4 of the 10 X or mismatches at 3 of the 10 X and 1 of the 3 Y or mismatches at 2 of the 10 X and 2 of the 3 Y or mismatches at 1 of the 10 X and 3 of the 3 Y ) \\ \begin{matrix}= {P}_{4} \\ = \frac{10 !}{4 ! 6 !} {( \frac{1}{4} )}^{6} {( \frac{3}{4} )}^{4} {( \frac{1}{2} )}^{3} + \frac{10 !}{3 ! 7 !} 3 {( \frac{1}{4} )}^{7} {( \frac{3}{4} )}^{3} {( \frac{1}{2} )}^{3} + \\ \frac{10 !}{2 ! 8 !} 3 {( \frac{1}{4} )}^{8} {( \frac{3}{4} )}^{2} {( \frac{1}{2} )}^{3} + 10 {( \frac{1}{4} )}^{9} ( \frac{3}{4} ) {( \frac{1}{2} )}^{3} \\ = \frac{( \begin{matrix}( 210 \times {3}^{4} ) + ( 120 \times {3}^{4} ) + \\ ( 45 \times {3}^{3} ) + ( 10 \times 3 )\end{matrix} )}{{2}^{23}} = \frac{27975}{{2}^{23}} = 3.335 \times {10}^{- 3}\end{matrix} \\ P ( mismatch in up to 1 position ) = {P}_{0} + {P}_{1} = \frac{1 + 33}{{2}^{23}} = \frac{17}{{2}^{22}} = 4.053 \times {10}^{- 6} \\ P ( mismatch in up to 2 positions ) \\ \begin{matrix}= {P}_{0} + {P}_{1} + {P}_{2} \\ = \frac{1 + 33 + 498}{{2}^{23}} \\ = \frac{532}{{2}^{23}} \\ = \frac{133}{{2}^{21}} \\ = 6.342 \times {10}^{- 5}\end{matrix} \\ P ( mismatch in up to 3 positions ) \\ \begin{matrix}= {P}_{0} + {P}_{1} + {P}_{2} + {P}_{3} \\ = \frac{1 + 33 + 498 + 5356}{{2}^{23}} \\ = \frac{5888}{{2}^{23}} \\ = \frac{23}{{2}^{15}} = 7.019 \times {10}^{- 4}\end{matrix} \\ P ( mismatch in up to 4 positions ) \\ \begin{matrix}= {P}_{0} + {P}_{1} + {P}_{2} + {P}_{3} + {P}_{4} \\ = \frac{1 + 33 + 498 + 5356 + 27975}{{2}^{23}} \\ = \frac{33863}{{2}^{23}} \\ = 4.037 \times {10}^{- 3}\end{matrix}$$

Experiment 14: Cctra

We have one line of LA3097 (LA3097A) which shows very good expression of its fluorescent marker; it is unknown if this line is a single integration event. This line does show evidence of sex-specific splicing, when reared off tetracycline all the females die as embryos, and when it is on 3 μg/ml of tetracycline both males and females survive.

This example is important. It shows that Cctra provides sex-specific alternative splicing in Aedes, and that this can be used to give sex-specific lethality. This, therefore, provides evidence of the phylogenetic range for Cctra splicing. Thus, it is entirely plausible that the present invention can be applied to all Diptera, as we have shown that Cctra works in Drosophila, tephritids and mosquitoes, which essentially spans the whole Dipteran Order.

It is surprising that Cctra works in Aedes, given the rapid sequence evolution of tra.

We transformed Aedes aegypti with construct LA3097. Heterozygous males from the resultant transgenic line were crossed to wild type and the progeny reared in aqueous medium supplemented with tetracycline to a final concentration of 30 μg/ml. Adults were recovered as follows: 14 males and one female, thus showing significant female-specific lethality.

This species and strain normally has a sex ratio of approximately 1:1, therefore this construct gave female-specific lethality in Aedes aegypti. Equivalent constructs which did not contain the Cctra intronic sequence gave non-sex-specific lethality. Therefore, the Cctra intron can be used to provide differential (i.e. sex-specific) regulation of gene expression in mosquitoes, and this can further be used to provide sex-specific lethality and a method for the selective elimination of females from a population.

In more detail: on 0 μg/ml tetracycline, males survive only to pupae, i.e. don't make it to adult. Females die so early that we don't see them, probably as embryos, so there is still a differential effect between the sexes. However, the pupal lethality in males suggests that the system is not completely switched off in males. The single insertion line that we recovered is unusual, in that it shows extremely strong expression of the marker; other insertions with more typical expression levels might well not show male lethality.

Splicing in LA3097A

Analysis of splicing of LA3097 from LA3097A transgenic mosquitoes by RT-PCR showed that males and females shared two transcripts, an approximately 950 bp band and a fainter band of approximately 800 bp (FIG. 59). Sequencing of these bands showed that the ˜900 bp band corresponds to a non-sex-specific splice variant (AcM2, ˜920 bp), and the fainter band was a mixture of a non-sex-specific splice variant (AeM1, ˜804 bp) and the female form (AeF1, ˜765 bp), see FIG. 60. The splicing of the AeF1 transcript was identical to that shown for this construct in Medfly (FIG. 33). The splicing of the M transcripts differs somewhat from that seen in the native context (Cctra splicing in Medfly, either the native gene or as we observed from LA3097 in transgenic Medfly); in AeM1 the second alternatively spliced exon (ME1b) is not included in the mature AeM1 transcript and in AeM2 the second alternatively spliced exon (ME2b) is similarly not included in the mature AeM2 transcript. In other words, for each of these transcripts the first but not the second cassette exon is present, relative to the Medfly prototype. Note that, as a consequence of the absence of the second cassette exon in AeM1, and the reading frame of tTAV2 relative to the first cassette exon in this construct, splicing in the AeM1 pattern does not lead to interruption of the tTAV2 open reading frame, but rather to the addition of 39 nucleotides (corresponding to 13 amino acids) between the ATG and the rest of the tTAV2 open reading frame. It is likely that this variant of tTAV2 may retain some activity, relative to normal or prototypic tTAV2 (as encoded by the F1 splice variant). In the absence of tetracycline, a phenotypic effect was observed in males as well as in females, though weaker in males than females. Production of a partially active variant of tTAV2 from the AeM1 transcript in males (and females) may explain this.

FIG. 59 shows RT-PCR of males and females from LA3097A Aedes aegypti transgenic line using the primers HSP (SEQ ID NO. 139) and VP16 (SEQ ID NO. 140). Using these primers, splicing in the CcF1 pattern (i.e. corresponding to the F1 variant of Ceratitis capitata) would give a band of approximately 765 bp and splicing in the CcM1 1005 bp and CcM2 1094 bp. In both males and females, a strong band of approximately 950 bp (1) was observed along with a fainter band of approximately 800 bp (2). Marker (SmartLadder™ from Eurogentec, bands from 1.5 kb to 0.4 kb are indicated).

Sequence analysis of several clones from band 2 (i.e. AeM1/AeF1 splice variants) from males and females showed that one of five clones from females showed AeM2 splicing (20%), whereas in males three of the four clones showed AeM2 splicing (75%); all the other clones showed AeF1 splicing. This indicates that there is more AeF1 transcript present in females than in males and this would explain the differential killing effect seen between them.

FIG. 60 Illustrates the various transcripts produced by alternative splicing of Cctra from LA3097A Aedes aegypti transgenic line. 3097 represents the DNA sequence of Cctra and the numbers relate to figure described elsewhere. Shading and boxes also relate to FIG. 33. Note that the diagram is not to scale.

### Example 15

### Aedes Actin-4;

We have eleven lines of LA3545, which uses the Aedes actin-4 gene (AeAct-4 or AaAct4) to drive expression of tTAV2. In construct LA3545, a sequence encoding tTAV2 has been inserted into the second exon of AaAct4 (FIG. 10). For transcripts spliced in the pattern characteristic of AaAct4 splicing in females, the ATG of the tTAV2 coding region will be the first (5′-most) ATG of the transcript. Splicing in the pattern characteristic of AaAct4 splicing in males introduces an array of start and stop codons before the tTAV2 sequence which tends to inhibit or interfere with translation from the ATG of the tTAV2 coding region. These lines should only express tTAV2 in female pupae. The splicing is shown in FIG. 8, below.

FIG. 8 shows RT-PCR of male and female adults from LA3545AeC Aedes aegypti transgenic line using the primers Agexon1F (SEQ ID NO. 141) and TETRR1 (SEQ ID NO. 142). Using these primers, splicing in a pattern equivalent to that of the native AaAct4 gene would give bands of approx 347 bp for the female-type splice variant and of approx 595 bp for the male-type splice variant. A band of approx 347 bp band (F) was found only in reactions on extracts from females; a band of approx 595 bp (M) was found in both males and females. Sequencing has confirmed that the correct splicing occurred in males and females. Marker (SmartLadder™ from Eurogentec, bands from 1.5 kb to 0.2 kb are indicated).

We also have transgenic Aedes aegypti carrying construct LA3604, which is similar to LA3545 except it has an engineered start codon in the portion of exon 1 that is present in both male-type and female-type transcripts (FIG. 10). This is arranged to be the first ATG in either transcript type. LA3604 encodes tTAV2 fused to ubiquitin (LA3545 codes tTAV, while LA3604 codes ubi-tTAV2). This construct should produce a fully functional tTAV2 protein in females only, even if the male form is expressed in females the extra male exon contains several start and stop codons that would prevent translation of the Ubi-tTAV2 fusion protein.

The alternative splicing of AaAct4 occurs in the 5′ UTR (of the native gene). It may or may not have a regulatory role in the native gene. One possibility is as follows: in the female-specific splice variant, the start codon of the AaAct4 coding region is the first ATG of the transcript. However, in the male-specific splice variant there are several additional ATG sequences 5′ to the start codon of the AaAct4 coding region; most of these have in-frame stop codons a short distance 3′. This sequence arrangement may interfere with the efficient translation of the AaAct4 protein and thereby reduce expression of the protein in males as compared with females. This is the arrangement in LA3545.

However, a greater differential effect between males and females would be expected if the intron was included in coding region (rather than 5′ UTR), i.e. inserted between the start and stop codons of the polynucleotide for expression in the organism. In this case, the male-specific cassette exon would change the coding potential of the transcript, rather than simply interfering with translation.

This is achieved in construct LA3604. We modified the shared first exon to include an ATG sequence in a suitable sequence context for translational initiation. In this modified sequence, this is the first ATG in either the male-type (M) or female-type (F) splice variants. Following splicing in the F form, this (engineered) 5′ ATG is in frame with the ubi-tTAV coding region. F-type transcripts would therefore encode a fusion protein, comprising sections encoded by (i) part of what is normally Act4 5′ UTR (but here obviously translated, and so not UTR at all), (ii) ubiquitin coding region and (iii) tTAV2 coding region.

Activity of cellular ubiquitin proteases will release the tTAV2 protein. Translation from the engineered 5′ ATG would be terminated by in-frame stop codons in the additional sequence (cassette exon) present in transcripts spliced in the M form. This would therefore prevent expression of functional tTAV2 in males, thereby giving sex-specific expression of tTAV2. Obviously, this gives a general method for sex-specific expression of a protein, by replacing the tTAV2 segment with another protein or sequence of interest. Using this strategy we have provided transgenics and shown sex-specific splicing (FIG. 9).

FIG. 9 shows RT-PCR of males and females from LA3604AeA Aedes aegypti transgenic line using the primers Agexon1F (SEQ ID NO. 141) and TETRR1 (SEQ ID NO. 142). Using these primers, splicing in the female form would give a band of approximately 575 bp, while inclusion of the male-specific cassette exon would increase this to approximately 823 bp. A band of approx 575 bp was seen from each female analyzed, while a band of approx 823 bp was seen from each male analyzed. These bands appear to be substantially specific to the respective sexes. Sequencing of these bands showed the correct splicing had occurred in males and females. Marker: SmartLadder™ from Eurogentec, bands from 1.5 kb to 0.2 kb are indicated.

FIG. 10, below, is a diagrammatic representation of plasmids LA3545 and LA3604. S1: shared exon 1; M1: additional sequence included in male-specific exon 1; S2: shared exon 2 (5′ end only); ubi: sequence encoding ubiquitin; tTAV2: sequence encoding tTAV2.

In several of the LA3545 transgenic lines a sex- and tissue-specific effect was observed: females are flightless. Two of the lines show a 90-100% female flightless phenotype one line shows 70% flightless and another 50%. This phenotype is presumably due to female-specific expression of tTAV2 in the developing flight muscles. The difference in the phenotypes between the lines is due to positional effects on the expression of the AaAct4 promoter. Due to a genes position in the genome expression can be influenced by a number of factors (heterochromatin or euchromatin regions, enhancer and suppressor elements, proximity to other genes) which can be seen readily in the fluorescent markers used to identify transgenics. All eleven lines of LA3545 were identified because they have different fluorescent profiles, even though they have the same promoters and marker. This variation is due to positional effects. This would then mean that we would expect some lines of LA3545 to express more tTAV2 than other because of positional effects, and those lines that do express more would give a female-specific flightless phenotype.

To test this hypothesis we developed a separate Aedes aegypti line with a tetO-DsRed2 reporter gene (LA3576 see FIG. 17 and SEQ ID NO. 143), when crossed with the different LA3545 lines this would allow the visualisation of where and when the Actin4-tTAV2 was expressing. Out of 8 LA3545 lines crossed to LA3576 all showed female-specific indirect flight muscle fluorescence in late L4 larvae, pupae and adults. In four of the lines DsRed2 expression appeared to be specific (i.e. exclusive) to the female indirect flight muscles; in the other four additional tissues showed expression of DsRed2. This phenomenon, where expression of a transgene depends in part on the region or point in the genome into which it has inserted, is called position effect, and will be well known and understood by the person skilled in the art.

Using LA3576 proved that the expression of tTAV2 in LA3604 was female-specific, occurs mainly in the indirect flight muscles and is stage-specific. Several different tetO-effector constructs were then constructed to analyse their effects. The tetO-MichelobX transgenics (LA3582, see FIG. 15 and SEQ ID NO. 144) when crossed to LA3545 all showed female-specific flightless phenotypes that could be repressed by tetracycline. This proves that Actin4 can be used to drive an effector gene in a stage, tissue and sex-specific manner.

Because some lines of LA3545 had a female-specific flightless phenotype without the presence of an induced effector gene, this showed that tTAV2 could act as an effector molecule. tTAV2 is composed of a tTA, a tetO binding domain and VP16, a herpes simplex virus protein. VP16 activates transcription of immediate early viral genes by using its amino-terminal sequences to attach to one or more host-encoded proteins that recognise DNA sequences in their promoters. In LA3604 a tetO-VP16 effector gene has been added to enhance the effect of tTAV2. In three transgenic lines of LA3604 this has caused a 100% female-specific flightless phenotype when reared without tetracycline, showing that VP16 is an effective effector molecule. Note that LA3604 has a potential start codon (ATG) engineered 5′ to the alternatively spliced intron. Therefore, in this construct, the male-specific exon is expected to interrupt the open reading frame encoding tTAV (ubi-tTAV); since the male-specific sequence contains several stop codons, this will tend to reduce or eliminate production of functional tTAV in males. By way of comparison, the male-specific exon is 5′ to the start codon of tTAV in LA3545. However, by inserting a number of start codons 5′ to the start codon of tTAV (which is the first ATG of the female transcript but not of the male transcript), none of these additional start codons being suitable for efficient production of functional tTAV due to being out of frame or having intervening stop codons, this arrangement will also tend to reduce or eliminate production of functional tTAV in males, consistent with the phenotypic data above.

### Example 16

### Use of Ubiquitin and Intron Positioning

We have newly made Cctra-based constructs with the Cctra intron cassette in a variety of different contexts, i.e. flanked by different sequences. Various lines of transgenic Medfly carrying these have been constructed. This shows that the system is general and robust, i.e. that it will work for a wide range of heterologous sequences of interest.

We also have at least one newly made example of a Cctra-ubi-tTAV fusion giving correct splicing (DsRed-cctra-ubi-tTAV).

Preferred examples of the functional protein place the coding sequence for either ubiquitin or tTA, or their functional mutants and or variants such as tTAV, tTAV2 or tTAV3, 3′ to the intron. These are arranged so that these elements are substantially adjacent to the 3′ end of the intron, more preferably such that the coding region starts within 20 nucleotides or less of the 3′ intron boundary), and most preferably, immediately adjacent the 3′ end of the intron, although this is less relevant if the Ubiquitin system is used.

Preferred examples of constructs according to the present invention are listed in Table 4, below. It will be appreciated that LA1188 is not within the scope of the present invention, as it does not encode a functional protein, i.e. it doesn't work properly. This is thought to be because of the unexpected use of a splice donor 4 bp 5′ to the junction with Cctra intron sequence, leading to a frameshift that is induced in all splices. It is, therefore, included for the sake of information only.

TABLE 4

| | Species tra | | |
|---|---|---|---|
| Construct NO. | intron | position from | tra intron |
| (FIGS. #.) | is from | ATG (bp) | is fused to- |
| LA1188 (80) | Medfly | +132 | tTAV |
| LA3014 (29) | Medfly | +22 | ubiquitin |
| LA3166 (30) | Medfly | +136 | ubiquitin |
| LA3097 (27) | Medfly | +0 | tTAV |
| LA3077 (26) | Medfly | +61 | tTAV |
| LA3233 (28) | Medfly | +0 | tTAV2 |
| LA3376 (31) | Medfly | +0 | tTAV2 |
| LA3376 (31) | B. zonata | +3 | reaper KR |
| LA3376 (31) | B. zonata | +0 | tTAV3 |
| LA3242 (32) | C. rosa | +3 | reaperKR |
| LA1038 (14) | Medfly | +21 | Nipp1 (nipper) |
| LA3054 (61) | Medfly | +811 | DsRed-ubiquitin |
| LA3056 (62) | Medfly | +811 | DsRed-ubiquitin |
| LA3488 (63) | Medfly | +949 | Ubiquitin |
| LA3596 (67) | Medfly | +949 | Ubiquitin |

Table 4 shows constructs which contain a splice control sequence which is derived from a tra intron. The introns were derived from C. capitata (Medfly), B. zonata or C. rosa (see column 2). Said intron was inserted within the coding region such that the distance between the putative initiator ATG and the last nucleotide of the exon immediately preceding the tra intron was as should be indicated in column 3. Intron is inserted into or adjacent to coding region for either ubiquitin, tTAV, reaper^{KR}, nipper or ubiquitin-DsRed as shown in column 4. These were generated and shown to successfully splice, by RT-PCR or phenotypically in Medfly and, in some cases, also either in Drosophila melanogaster (LA3077) or Anastrepha ludens (LA3097, LA3233, LA3376). In addition, the distance between the ATG and the end of the exon immediately preceding the tra intron (assuming splicing in F1-like form) can range from 0 bp to at least +949 bp without adverse consequences to splicing (see Table 4, column 3). Thus, it is reasonable to assume that this distance can be up to at least 900 and preferably up to at least 949 bp.

Further information on these examples is summarized in Table 5. The preferred option is to use no endogenous sequence to achieve correct alternative splicing control of expression (+0 bp in table 4). We prefer to insert the tra intron between the flanking dinucleotides TG . . . GT in the coding region of the protein of interest to be alternatively spliced to ensure correct splicing as this may be important, however we will not restrict ourselves to this if necessary as other flanking nucleotides may function correctly as well. Examples LA1038, LA3054 and LA3056 include some endogenous flanking exonic sequence from the natural Cctra gene. In Table 5, if 6 nucleotides or less (including the ATG start codon) are included of particular fusions to the 3′ or 5′ of the splice junction, for the summary purposes of this table these will not be considered to be part of the fusion. Table 4 can be correlated with table 3 to find which tra intron (Cctra, Bztra or Crtra) is used in each example. Again, LA1188 is included only for the purposes of information and falls outside the present invention.

TABLE 5

| | | | exonic tra | exonic tra |
|---|---|---|---|---|
| | tra intron | tra intron | sequence | sequence |
| Construct NO. | is fused | is fused | fused to | fused to |
| (FIGS. #.) | to 5′ | to 3′ | 5′ (bp) | 3′ (bp) |
| LA1188 (80) | Hsp70-tTAV | tTAV | +0 bp | +0 bp |
| LA3014 (29) | Hsp70- | ubiquitin- | +0 bp | +0 bp |
| | ubiquitin | reaperKR- | | |
| | | sv40 | | |
| LA3166 (30) | Hsp70- | ubiquitin- | +0 bp | +0 bp |
| | ubiquitin | reaperKR- | | |
| | | sv40 | | |
| LA3097 (27) | Hsp70 | tTAV-K10 | +0 bp | +0 bp |
| LA3077 (26) | Hsp70-tTAV | tTAV-K10 | +0 bp | +0 bp |
| LA3233 (28) | Hsp70 | tTAV2-K10 | +0 bp | +0 bp |
| LA3376 (31) | Hsp70 | tTAV2-K10 | +0 bp | +0 bp |
| LA3376 (31) | Sry-a | tTAV3-sv40 | +0 bp | +0 bp |
| LA3376 (31) | HB | reaperKR- | +0 bp | +0 bp |
| | | sv40 | | |
| LA3242 (32) | HB | reaperKR- | +0 bp | +0 bp |
| | | sv40 | | |
| LA1038 (14) | Hsp70-tra | Tra-Nipp1 | +22 bp | +20 bp |
| | | (nipper)- | | |
| | | sv40 | | |
| LA3054 (61) | Opie2-nls- | tra- | +22 bp | +20 bp |
| | DsRed-tra | ubiquitin- | | |
| | | tTAV-sv40 | | |
| LA3056 (62) | Opie2-nls- | tra- | +22 bp | +242 bp |
| | DsRed-tra | ubiquitin- | | |
| | | tTAV-sv40 | | |
| LA3488 (63) | Ie1-nls- | ubiquitin- | +0 bp | +0 bp |
| | TurboGreen- | nls- | | |
| | nls- | DsRed-nls- | | |
| | ubiquitin | sv40 | | |
| LA3596 (67) | Ie1-nls- | ubiquitin- | +0 bp | +0 bp |
| | TurboGreen- | nls- | | |
| | nls- | DsRed-nls- | | |
| | ubiquitin | sv40 | | |

As mentioned above when an intron is placed 5′ to a protein coding region (ORF-X), it is preferred to position or use ubiquitin 3′ to the intron, 5′ to ORF-X, thus and providing female-specific regulation of ORF-X, whilst introducing physical separation between that sequence and the tra intron, thereby reducing the chance that sequences within ORF-X will interfere with the splicing of the tra intron.

Composite constructs and sequences are also envisaged, for example of the form:

X-ubi-Y

with the alternatively spliced intron inserted between coding region X and the region encoding ubiquitin (ubi), or within the ubiquitin coding region, or between the region encoding ubiquitin and coding region Y. Thus X will be expressed irrespective of the splicing of the intron, while Y will only be expressed when the intron is spliced in a suitable form. Further configurations and arrangements of this general type will be apparent to the person skilled in the art. Some examples of this are LA3014, LA3054, LA3056, LA3166, LA3488 and LA3596 which all use ubiquitin fusions in this way demonstrating the ability of this idea to be successfully applied in transgenic Medfly. Alternative examples in transgenic mosquitoes include LA3604 and LA3612, showing the wide phylogenetic applicability of this system in not only different species (mosquitoes and Medfly), but also in different contexts including AaActin4, Aadsx and Cctra.

LA3596 (see FIG. 67 and SEQ ID NO. 145) is of similar design to LA3488, intended to generate green fluorescence (by expression of nuclear localised TurboGreen fluorescent protein) in both sexes, but red fluorescence only in females (by expression of nuclear localised DsRed2 fluorescent protein). This is accomplished by the fusion of these two proteins, driven by the Hr5-Ie1 enhancer/promoter cassette, linked together with a short 11 amino acid linker (SG4 linker) and a coding region comprising ubiquitin (with one intended point mutation to stabilize the resulting protein by reducing its propensity to ubiquitin-mediated degradation) and the Cctra intron to limit DsRed2 expression to females. Transgenic Medfly were generated with this construct. Red fluorescence was limited to females in this line as expected, while green fluorescence was observed in all males and females. This could be used for sex separation by fluorescence screening for a particular fluorescent protein, in this case red fluorescence representing expression of DsRed2.

### Example 17

### Further Cctra Exemplification

Reference is also made to LA3014 and LA3166 and phenotypic data therefrom in other Examples.

We have previously made, and have obtained transgenics with, the Cctra intron in a functional protein other than tTAV, see LA3014 and LA3166. LA3014 contains a ubiquitin-reaper^{KR} fusion downstream of a Cctra intron. Phenotypic data shows that LA3014 transgenic Medfly gave repressible female-specific lethality. RT-PCR analysis on RNA extracted from adult males and females raised off tetracycline, using primers and ReaperKR, demonstrate that correct splicing was occurring in females (508 bp band) and no such band was found in males (FIG. 37). LA3166 is another construct with the Cctra intron placed inside the ubiquitin coding region fused to reaper^{KR}, but placed in a different position in ubiquitin. LA3166 also produces a dominant repressible female-specific lethal effect in Medfly.

LA1038 is a new example of the use of the Cctra intron in a different sequence context, here placed in a fragment of Nipp1Dm called ‘nipper’ that also splices correctly in transgenic Medfly when analysed by RT-PCR (FIG. 12). LA670 was required as a source of tTAV to drive expression of the alternatively spliced nipper.

We have also newly made, and have obtained transgenics with, ‘intron-only’ Cctra-based constructs with the intron in a different gene (many of the above examples, unless otherwise apparent, are in tTAV or one of its variants, i.e. tTAV2 or tTAV3). These constructs work as predicted. This is an important result, thus showing that there are not essential exonic sequences in Cctra that we have simply duplicated (in function, if not necessarily in sequence) by chance, in tTAV. We also have ubi-rpr^{KR} constructs of this type (LA3014 and LA3166), which also validates the ubiquitin fusion method described above. The ubiquitin fusion method is further exemplified by RT-PCR analysis of LA3054, LA3056 and LA3488. (FIGS. 11, 13, 14), as described in Example 16, above.

### Example 17

### Further Cctra Exemplification

Reference is also made to LA3014 and LA3166 and phenotypic data therefrom in other Examples.

We have previously made, and have obtained transgenics with, the Cctra intron in a functional protein other than tTAV, see LA3014 and LA3166. LA3014 contains a ubiquitin-reaper^{KR} fusion downstream of a Cctra intron. Phenotypic data shows that LA3014 transgenic Medfly gave repressible female-specific lethality. RT-PCR analysis on RNA extracted from adult males and females raised off tetracycline, using primers and ReaperKR, demonstrate that correct splicing was occurring in females (508 bp band) and no such band was found in males (FIG. 37). LA3166 is another construct with the Cctra intron placed inside the ubiquitin coding region fused to reaper^{KR}, but placed in a different position in ubiquitin. LA3166 also produces a dominant repressible female-specific lethal effect in Medfly.

LA1038 is a new example of the use of the Cctra intron in a different sequence context, here placed in a fragment of Nipp1Dm called ‘nipper’ that also splices correctly in transgenic Medfly when analysed by RT-PCR (FIG. 12). LA670 was required as a source of tTAV to drive expression of the alternatively spliced nipper.

We have also newly made, and have obtained transgenics with, ‘intron-only’ Cctra-based constructs with the intron in a different gene (many of the above examples, unless otherwise apparent, are in tTAV or one of its variants, i.e. tTAV2 or tTAV3). These constructs work as predicted. This is an important result, thus showing that there are not essential exonic sequences in Cctra that we have simply duplicated (in function, if not necessarily in sequence) by chance, in tTAV. We also have ubi-rpr^{KR} constructs of this type (LA3014 and LA3166), which also validates the ubiquitin fusion method described above. The ubiquitin fusion method is further exemplified by RT-PCR analysis of LA3054, LA3056 and LA3488 (FIGS. 11, 13, 14), and as described in Example 16, above.

FIG. 11: Gel showing sex-specific splicing of intron(s) derived from Cctra (780 bp band in females) in Ceratitis capitata transformed with LA3488. Splicing in the F1 form would yield a product of approximately 780 bp. A band of this size is clearly visible from females (lane 4), but not from males, nor in the lanes with reactions from which the reverse transcriptase enzyme was omitted (“no RT”). Therefore, the Cctra-derived intron is capable of sex-specific alternative splicing in this novel sequence context. Lane 1: Marker (SmartLadder™ from Eurogentec, bands of approx 0.8, 1.0 and 1.5 kb are indicated); Lanes 2 and 3: Ceratitis capitata LA3488/+males (RT and no RT control, respectively); Lanes 4 and 5: Ceratitis capitata LA3488/+females (RT and no RT control, respectively).

FIG. 12: Gel showing sex-specific splicing of intron(s) derived from Cctra in Ceratitis capitata transformed with LA1038. Splicing in the F1 form would yield a product of approximately 230 bp. A band of this size is clearly visible from females (lanes 1, 2, 7, 8, 9 and 10), but not from males. Therefore, the Cctra-derived intron is capable of sex-specific alternative splicing in this novel sequence context. Lane 15: Marker (SmartLadder™ from Eurogentec, bands of approx 0.2, 0.4 and 0.6 kb are indicated); Lanes 1, 2, 7, 8, 9 and 10: Ceratitis capitata LA670; LA1038 females; Lanes 3, 4, 5, 6, 11, 12, 13 and 14: Ceratitis capitata LA670; LA1038 males.

FIG. 13: Gel showing sex-specific splicing of intron(s) derived from CeTra in Ceratitis capitata transformed with LA3054. Splicing in the F1 form would yield a product of approximately 340 bp. A band of this size is clearly visible in lane 7, but not from males. Therefore, the Cctra-derived intron is capable of sex-specific alternative splicing in this novel sequence context. Lane 1: Marker (SmartLadder™ from Eurogentec, bands of approx 0.4, 0.6, 0.8 and 1.0 kb are indicated); Lanes 2-5: Ceratitis capitata LA3054 males; Lane 7: Ceratitis capitata LA3054 female.

FIG. 14: Gel showing sex-specific splicing of intron(s) derived from Cctra in Ceratitis capitata transformed with LA3056. Splicing in the F1 form would yield a product of approximately 200 bp. A band of this size is clearly visible from a female (lane 6), but not from males (lanes 2-4). Therefore, the Cctra-derived intron is capable of sex-specific alternative splicing in this novel sequence context. Lane 1: Marker (SmartLadder™ from Eurogentec, bands of approx 0.2, 0.4, 0.6 and 0.8 kb are indicated); Lanes 2-5: Ceratitis capitata LA3056/+males; Lanes 6-7: Ceratitis capitata LA3056/+females.

FIG. 15: Gel showing sex-specific splicing of intron(s) derived from BzTra in Anastrepha ludens transformed with LA3376. Splicing in the F1 form would yield a product of approximately 672 bp. A band of this size is clearly visible from females (lane 4), but not from males, nor in the lanes with reactions from which the reverse transcriptase enzyme was omitted (“no RT”), primers used were SRY and AV3F. Therefore, the Bztra-derived intron is capable of sex-specific alternative splicing in this novel sequence context and species. Lane 1: Marker (SmartLadder™ from Eurogentec, bands of approx 0.6, 0.8, and 1.0 kb are indicated); Lanes 2 and 3: Anastrepha ludens LA3376/+males (RT and no RT control, respectively); Lanes 4 and 5: Anastrepha ludens LA3376/+females (RT and no RT control, respectively).

FIG. 18 and SE ID NOs 149 and 150 show DSX minigene1, DSX minigene2 sequences and LA3619 plasmid map.

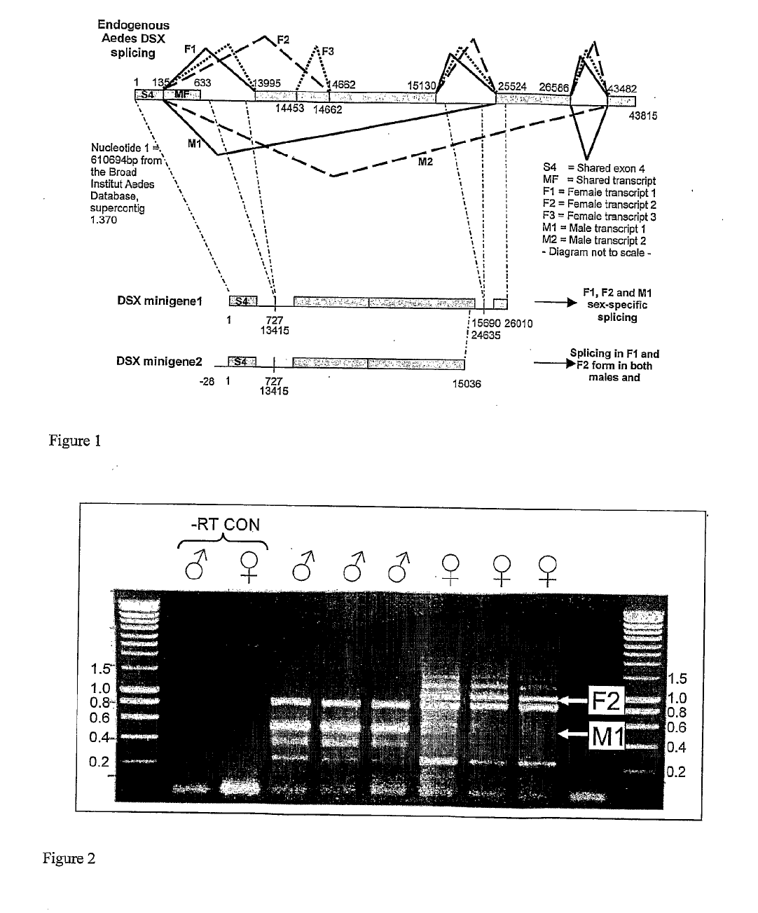

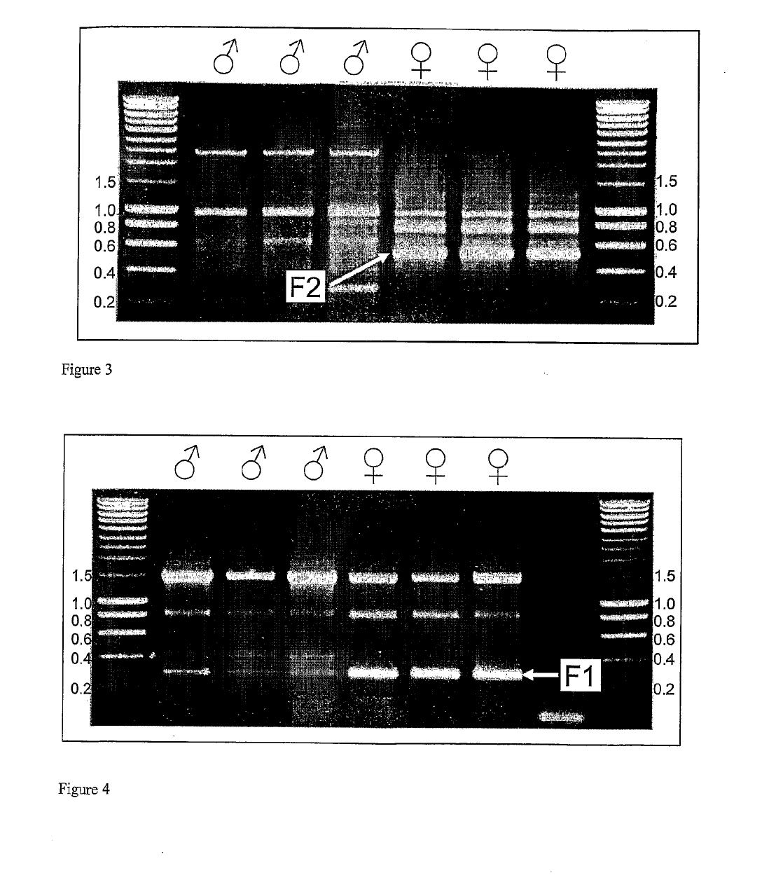

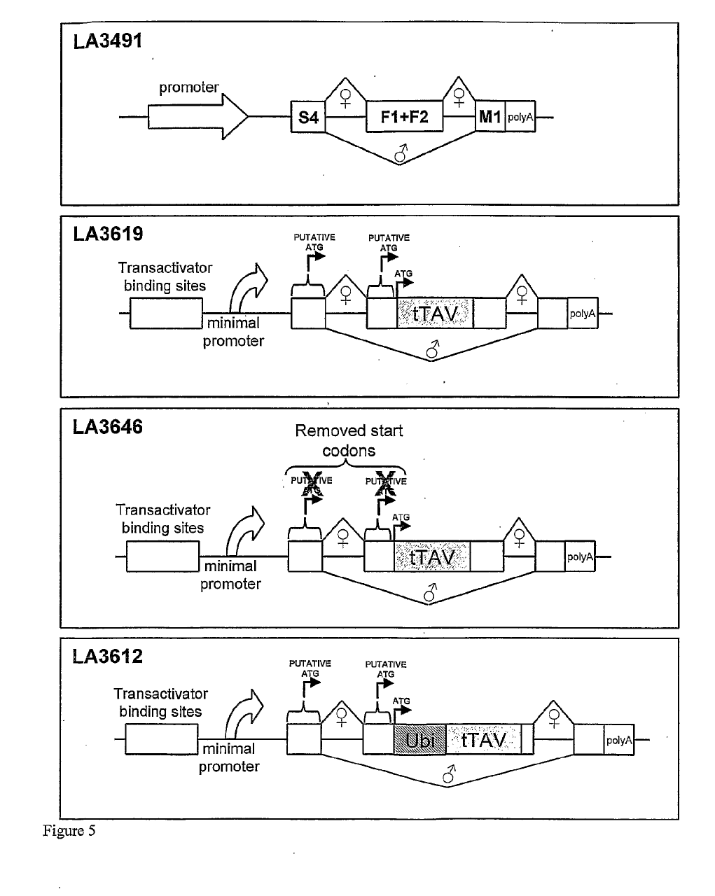

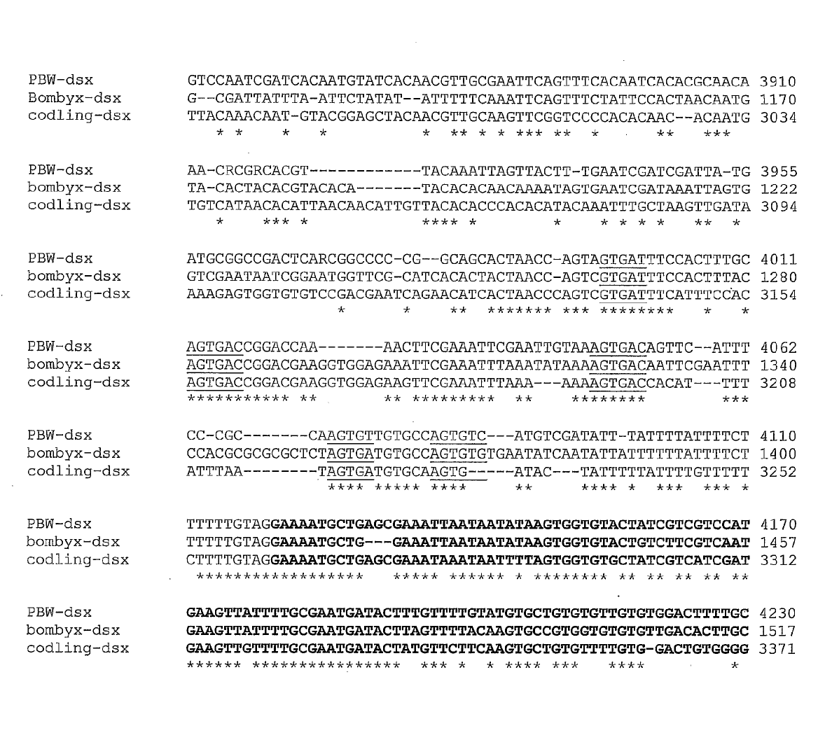

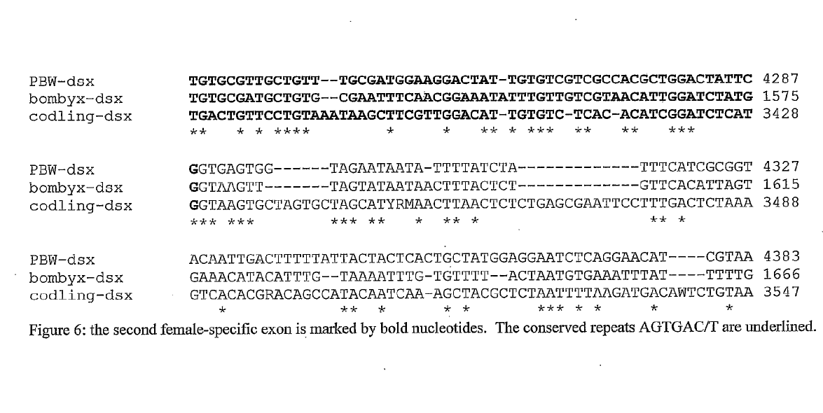

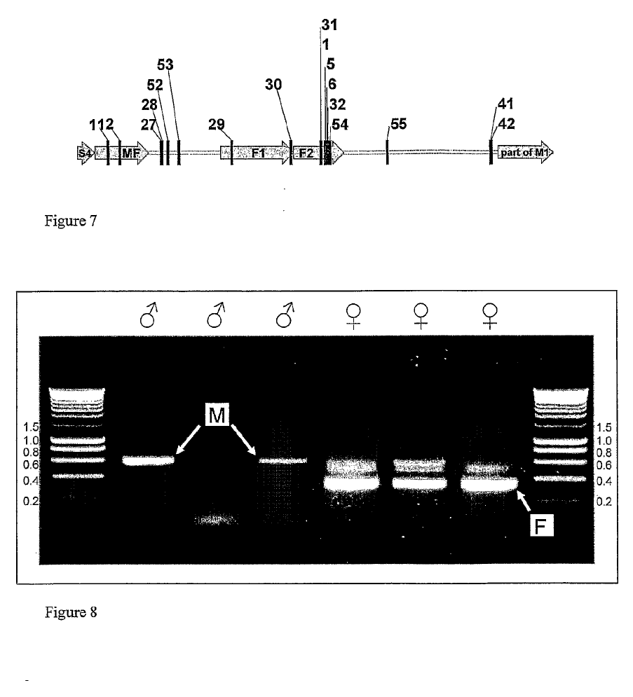

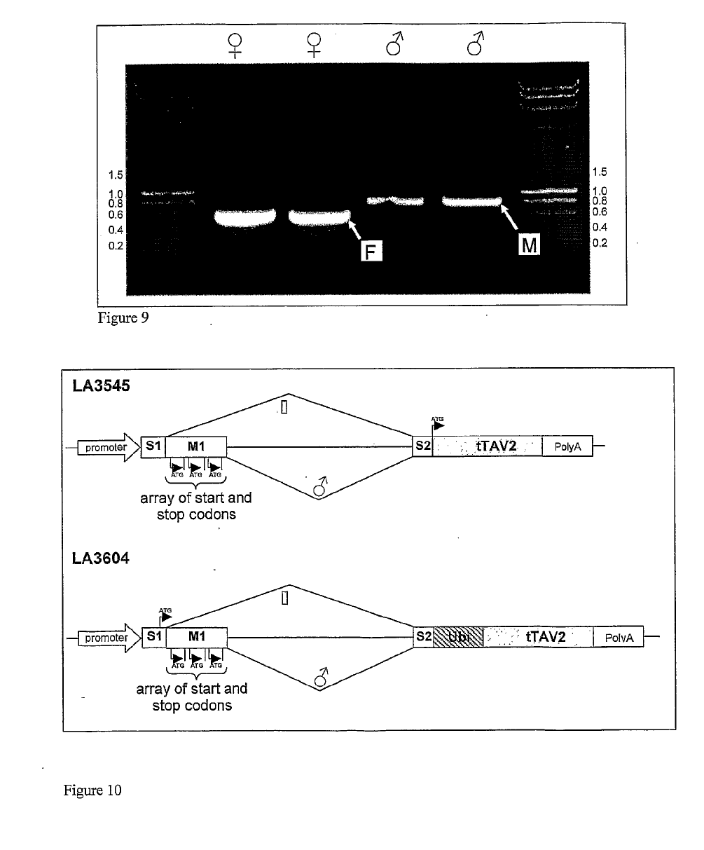

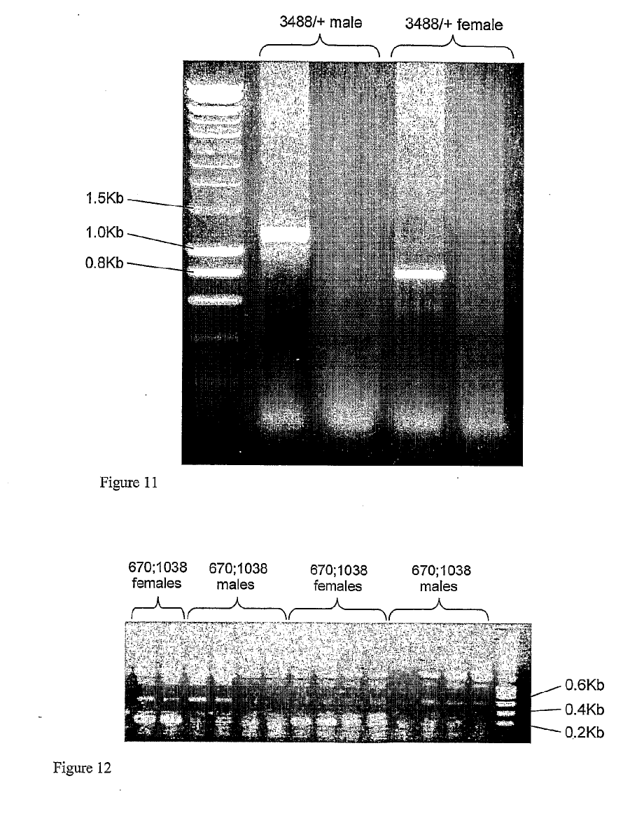

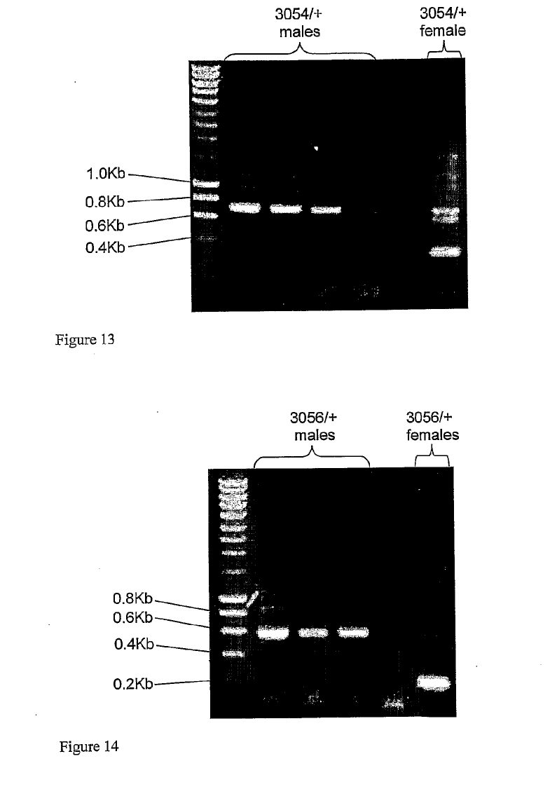

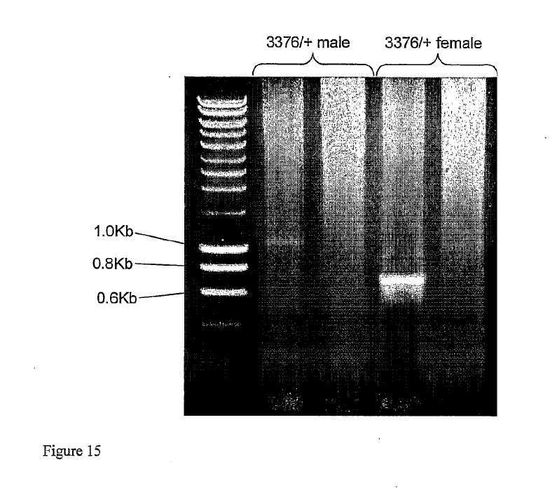

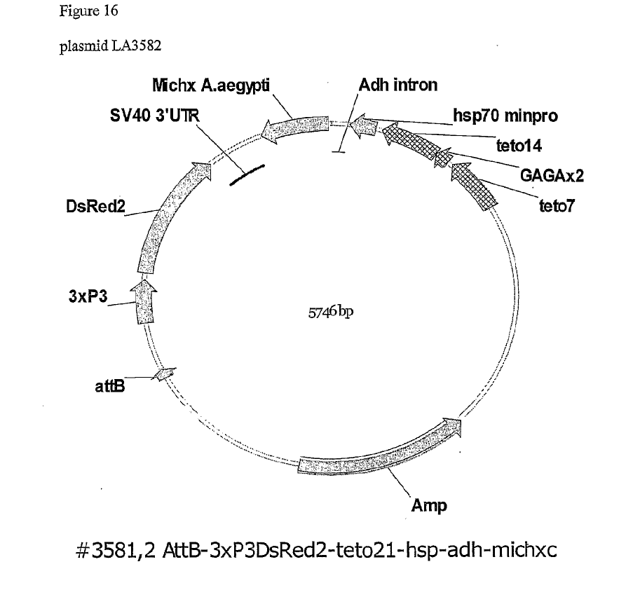

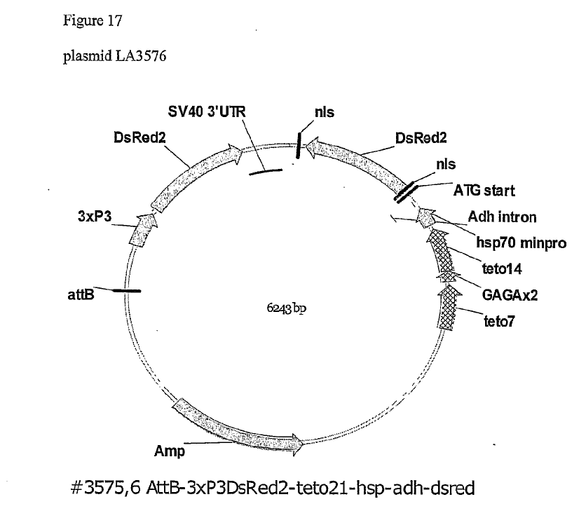

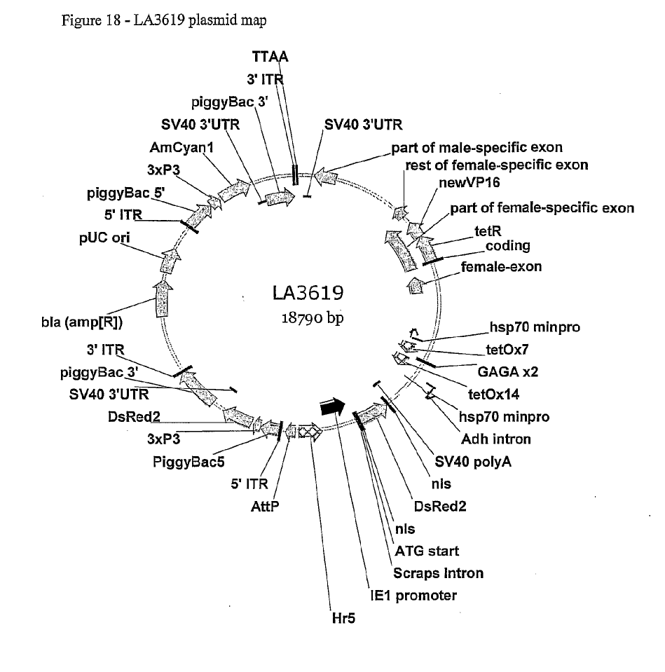

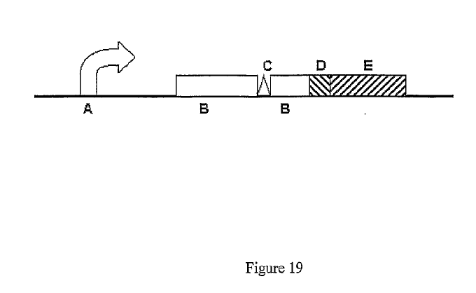

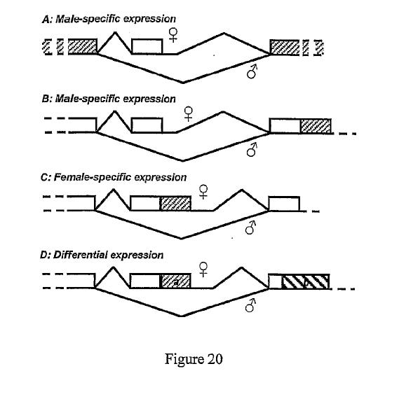

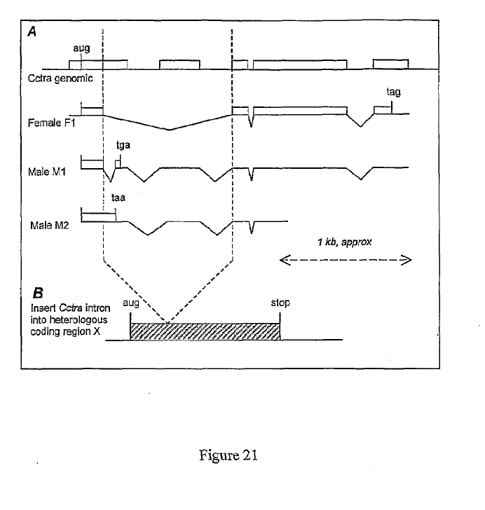

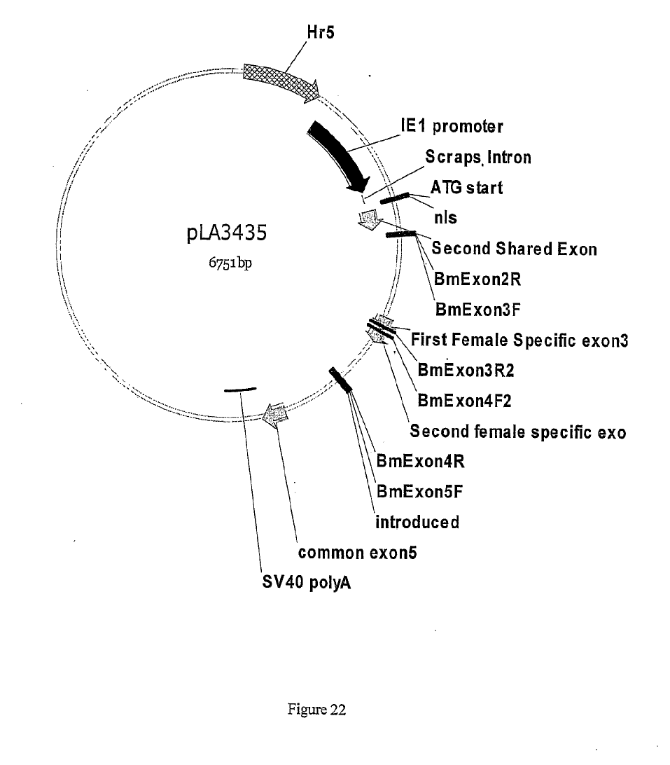

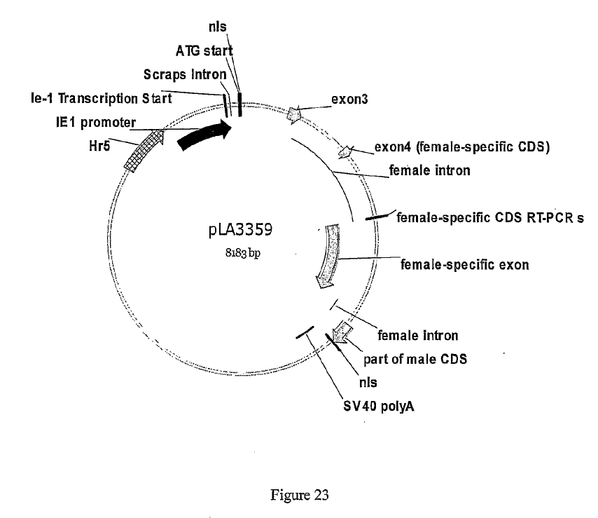

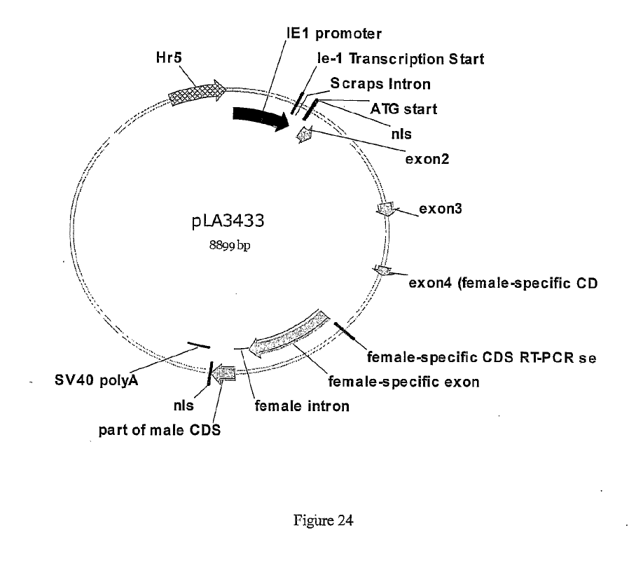

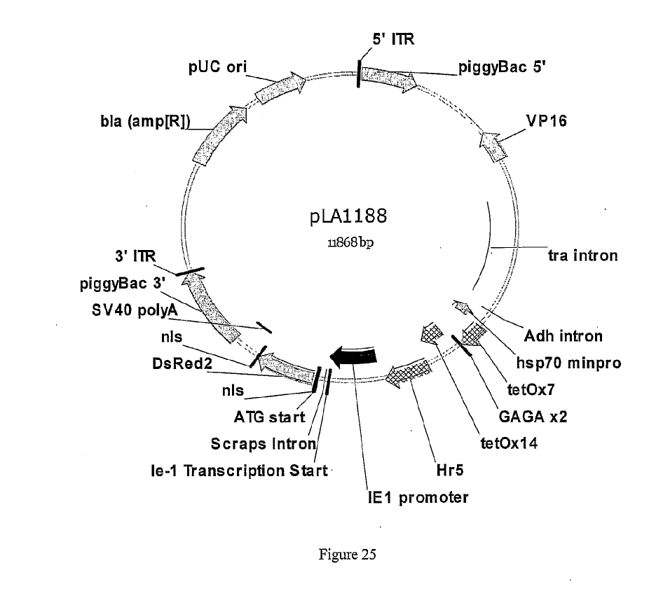

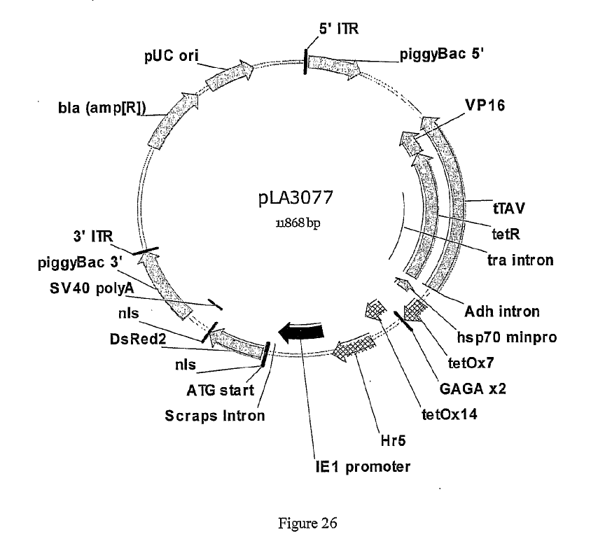

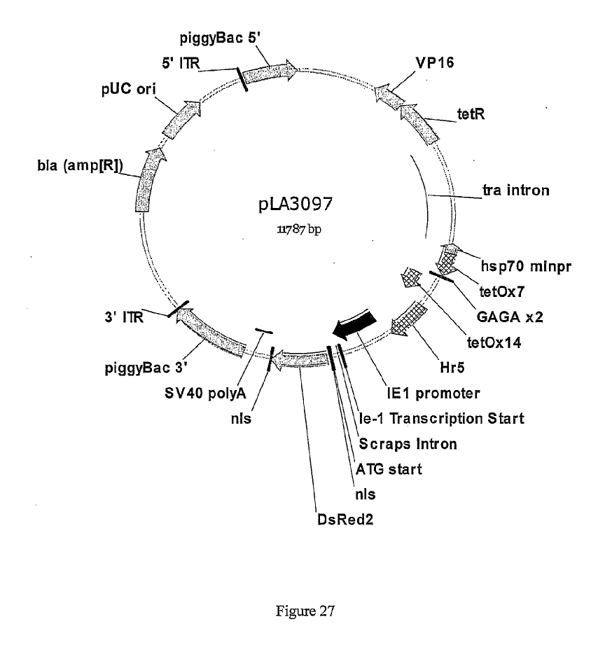

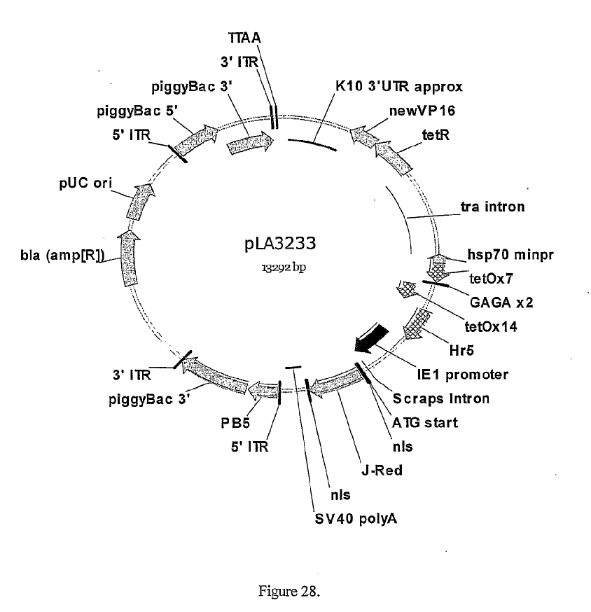

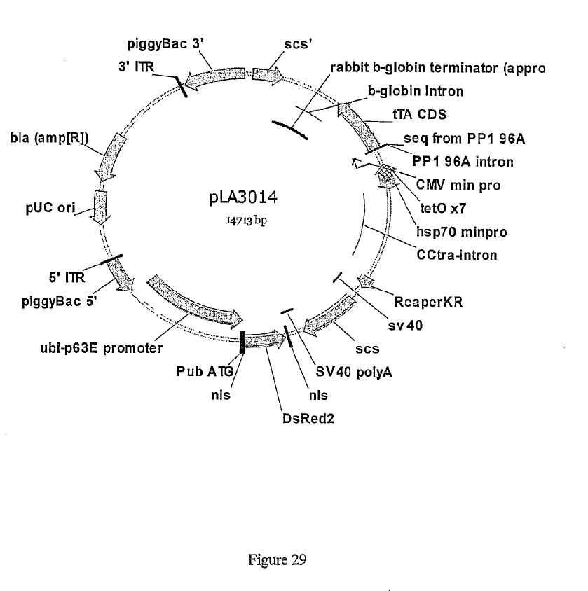

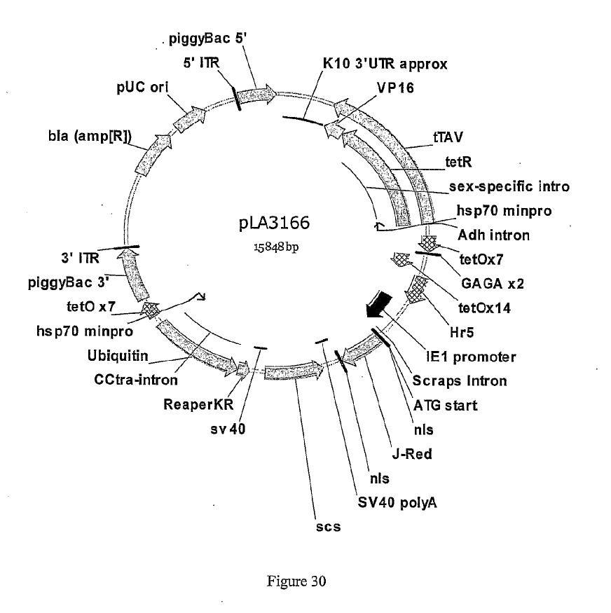

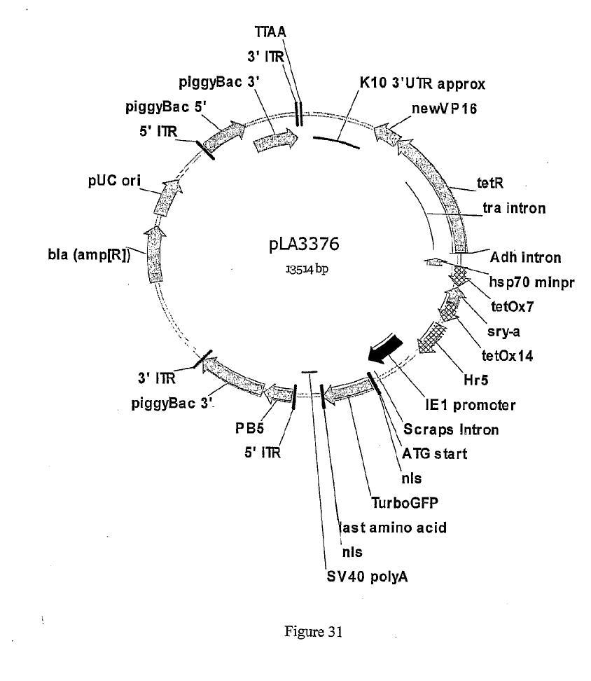

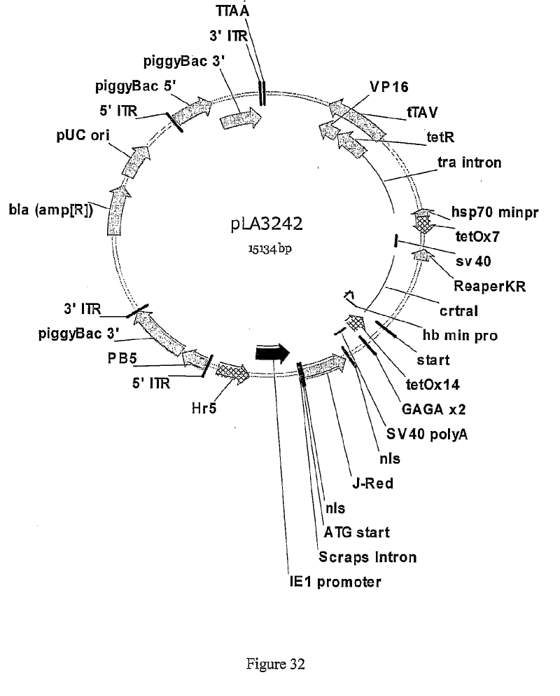

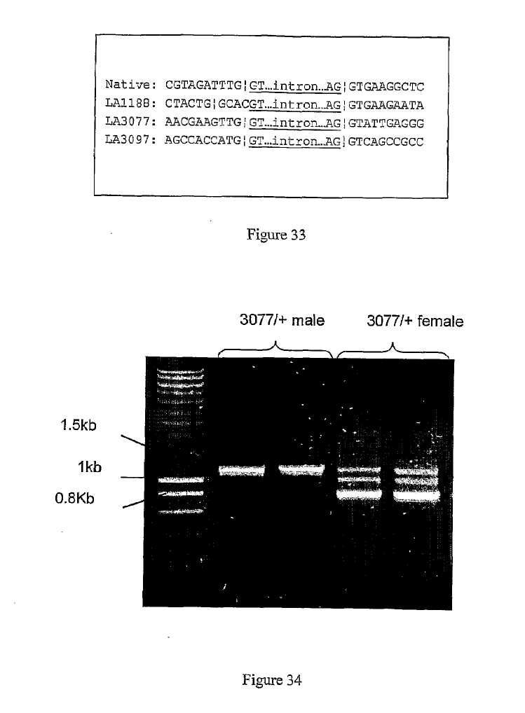

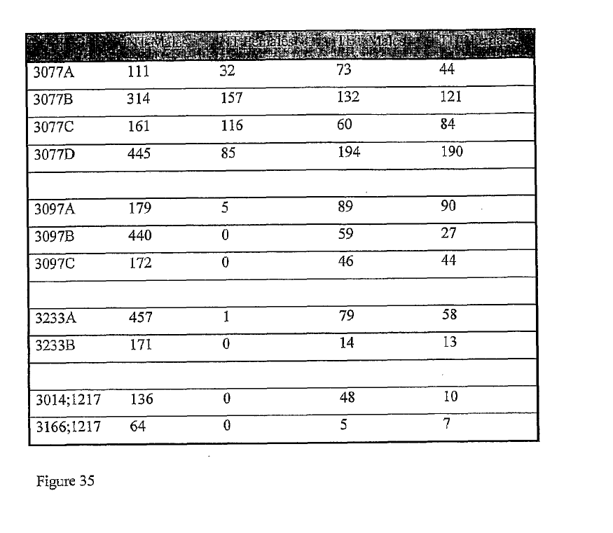

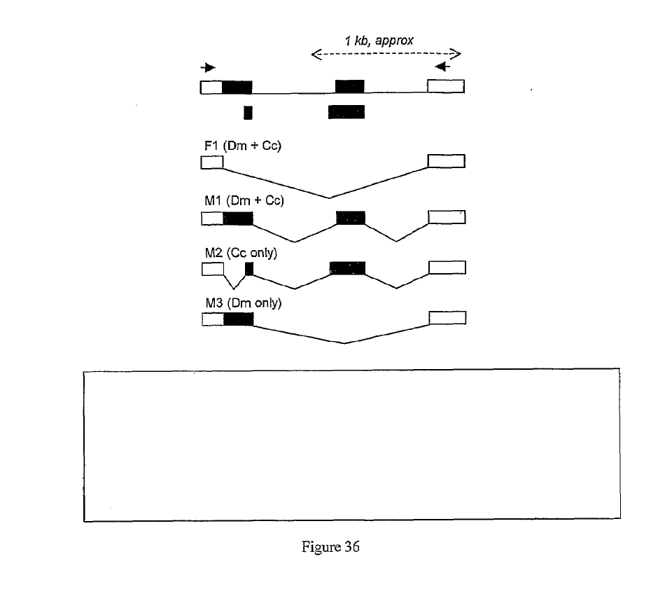

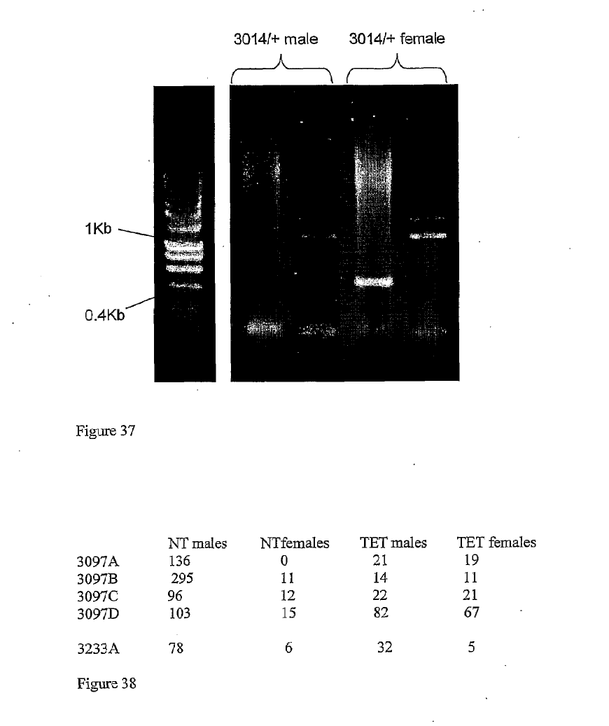

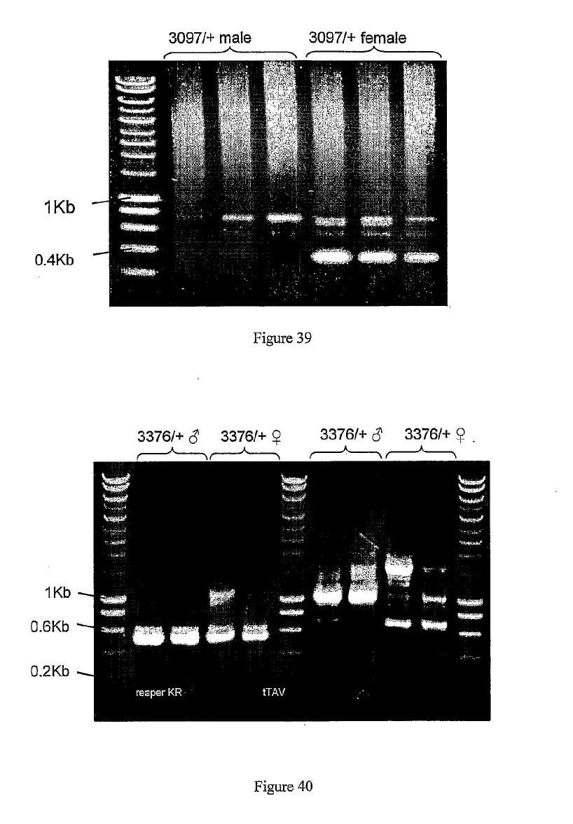

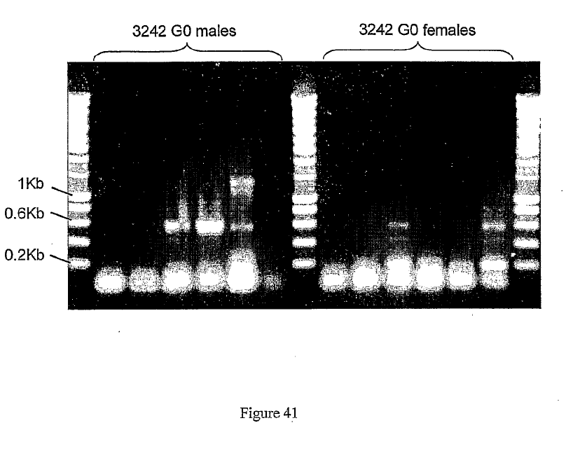

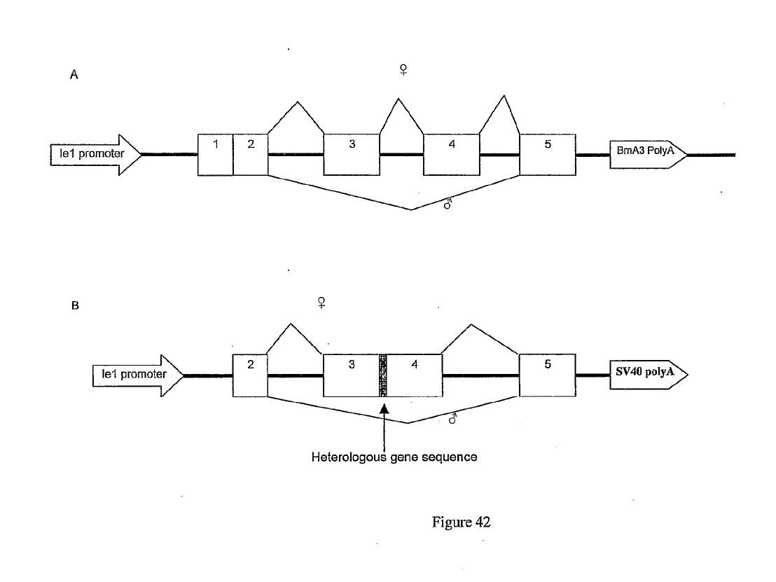

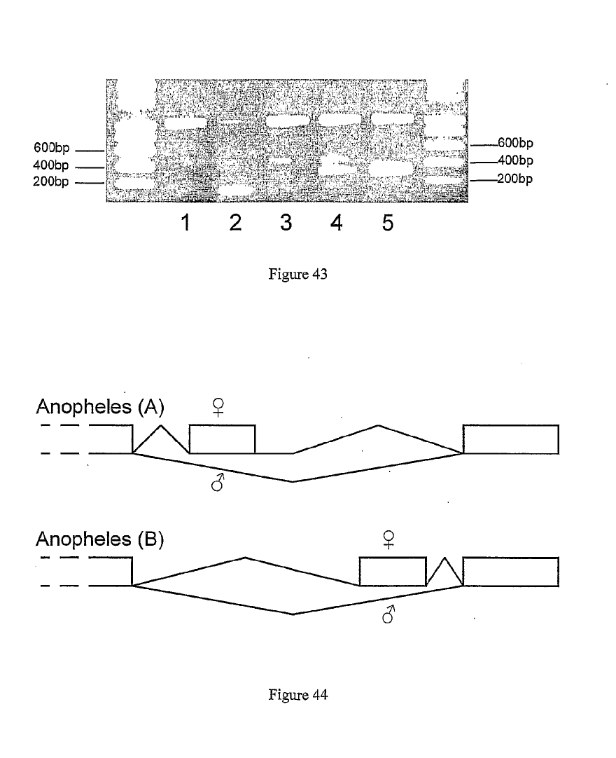

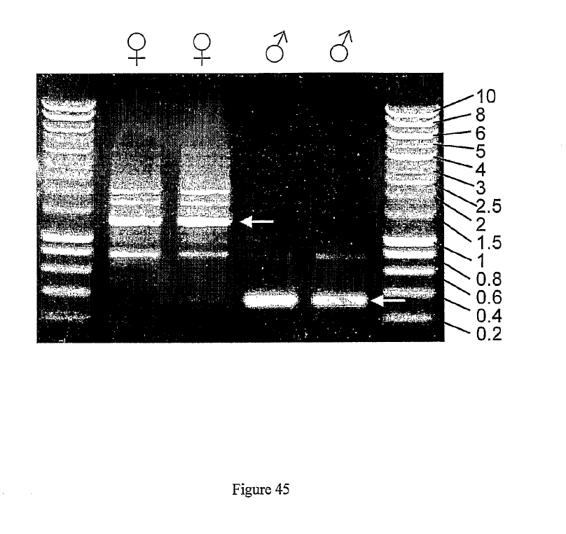

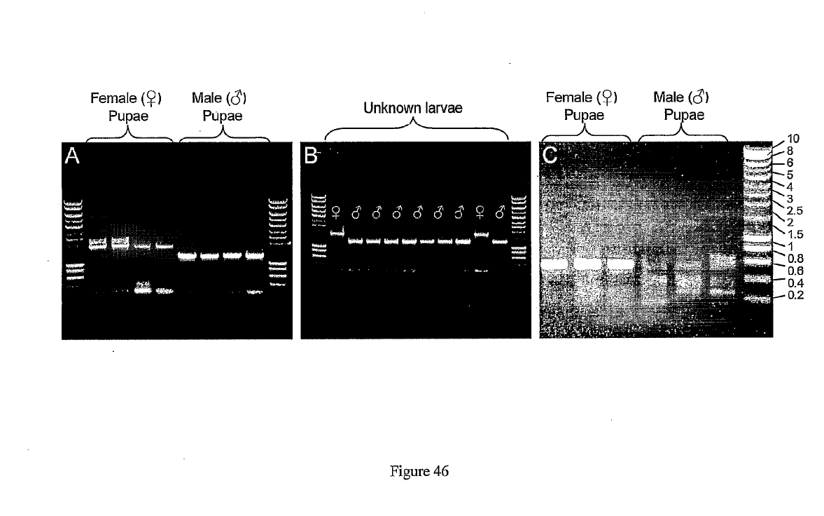

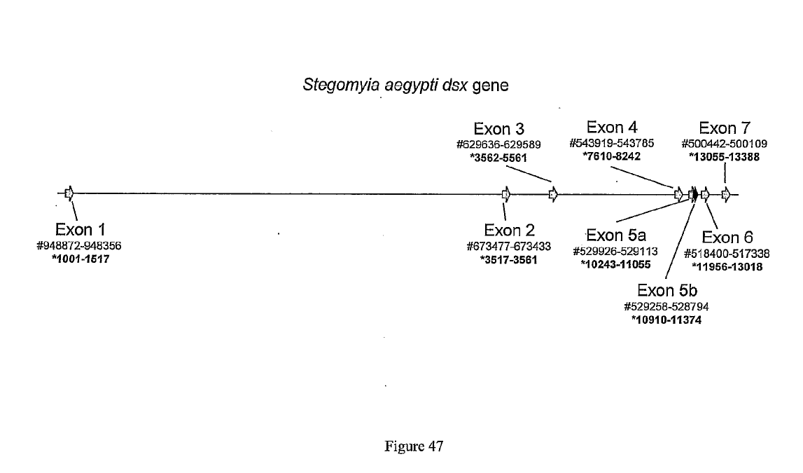

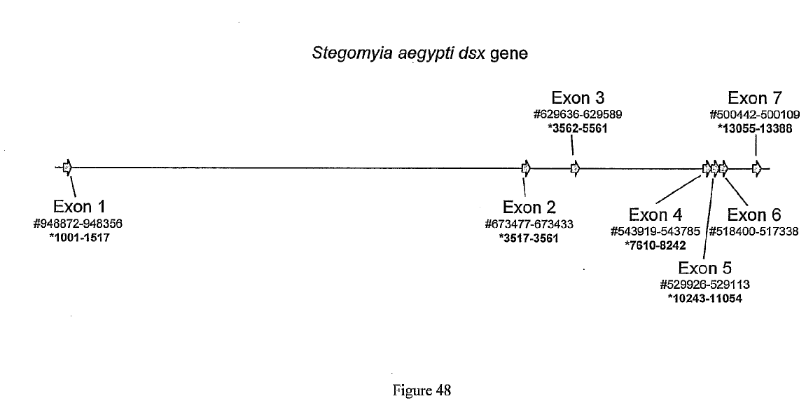

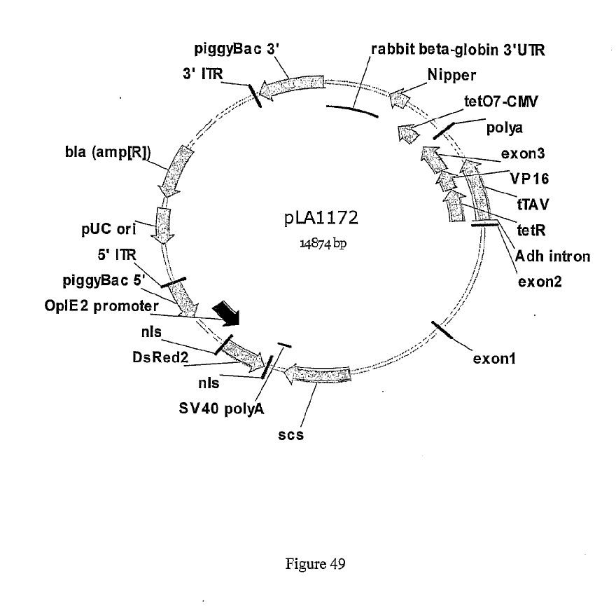

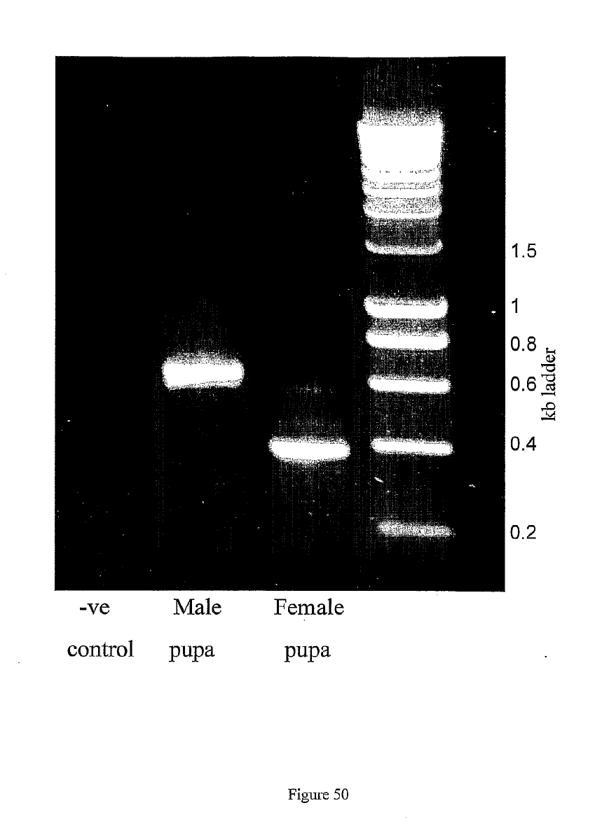

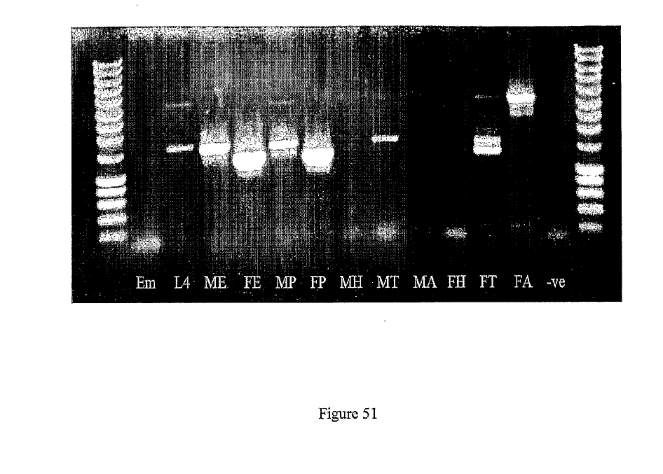

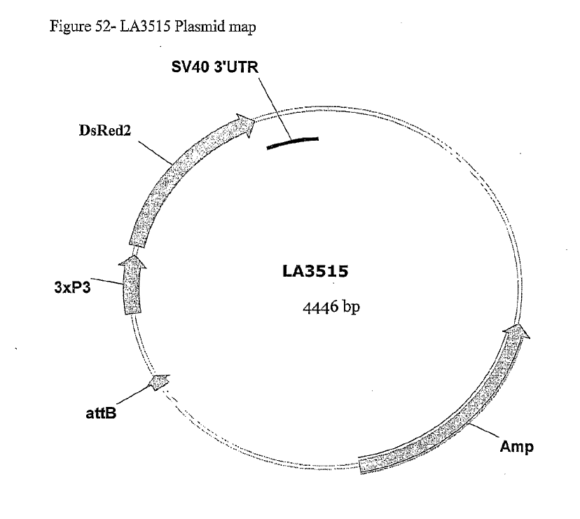

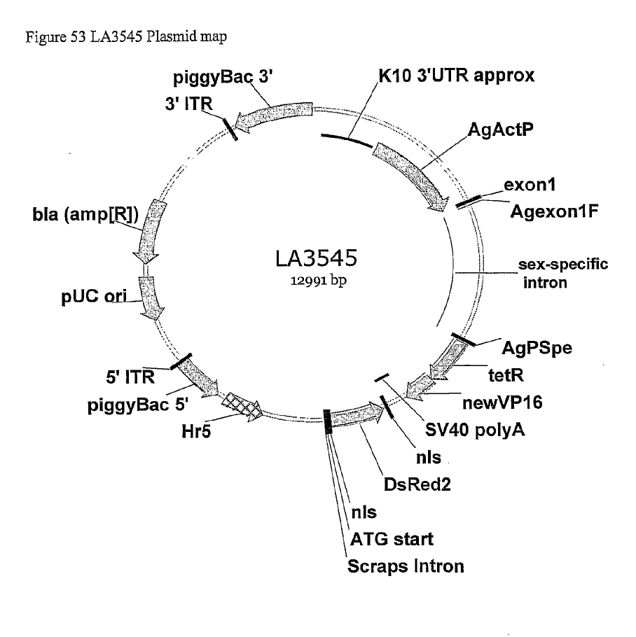

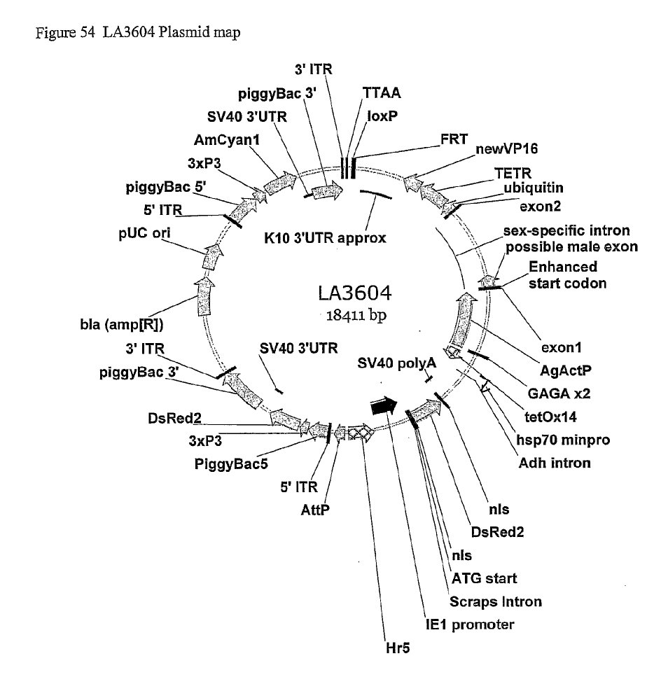

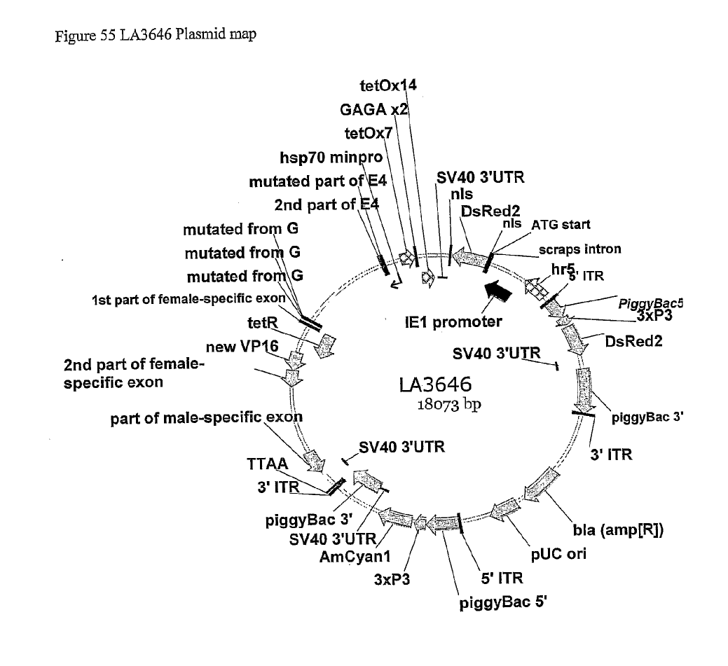

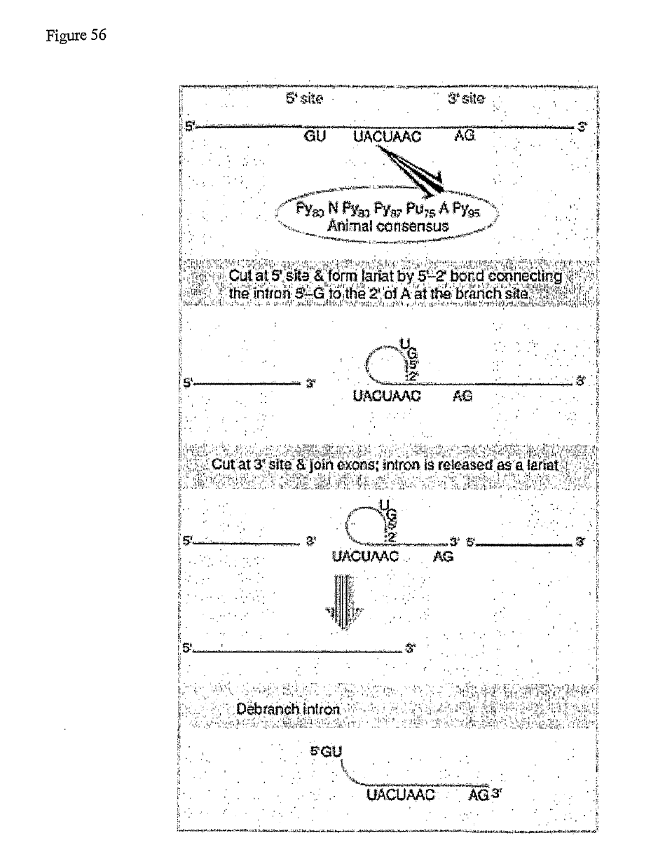

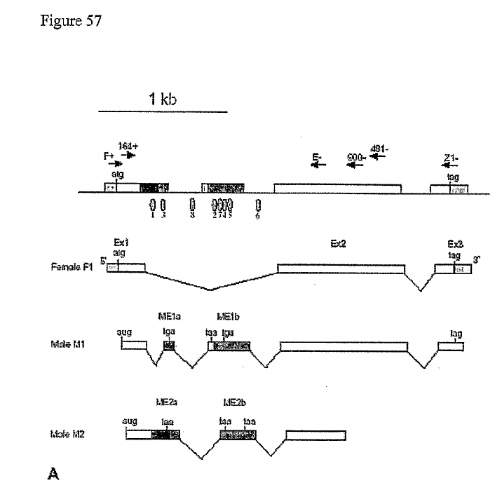

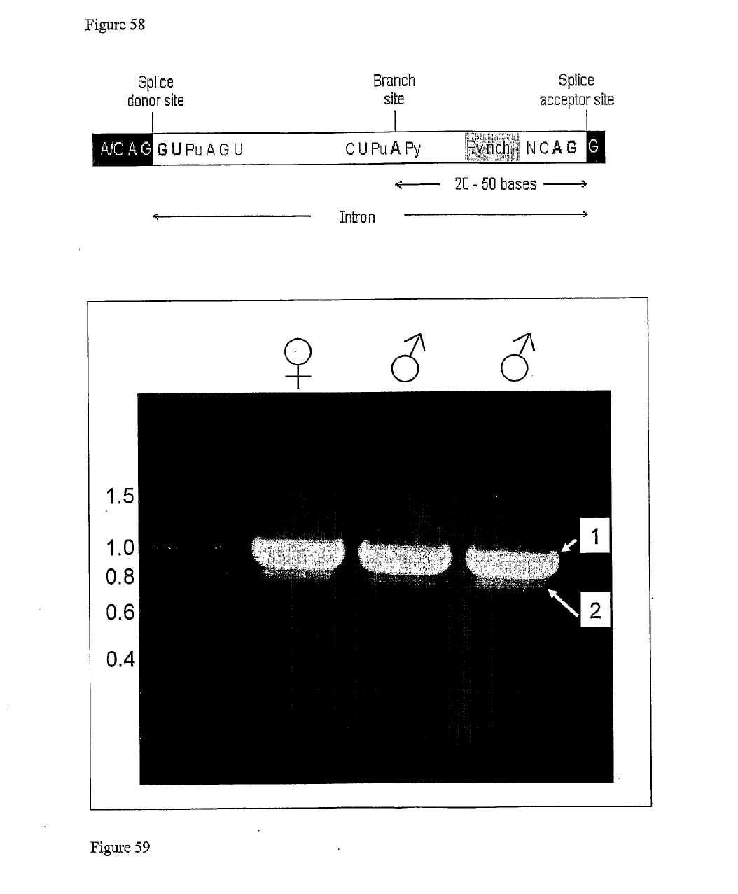

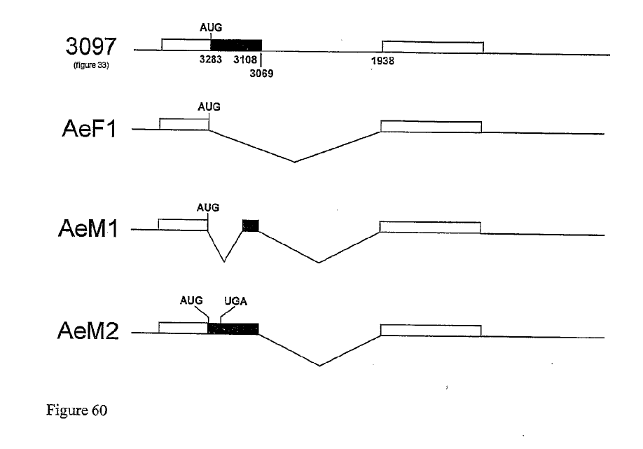

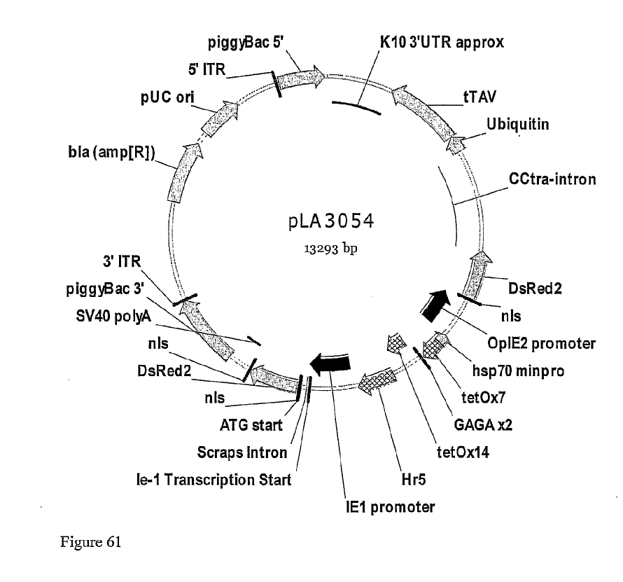

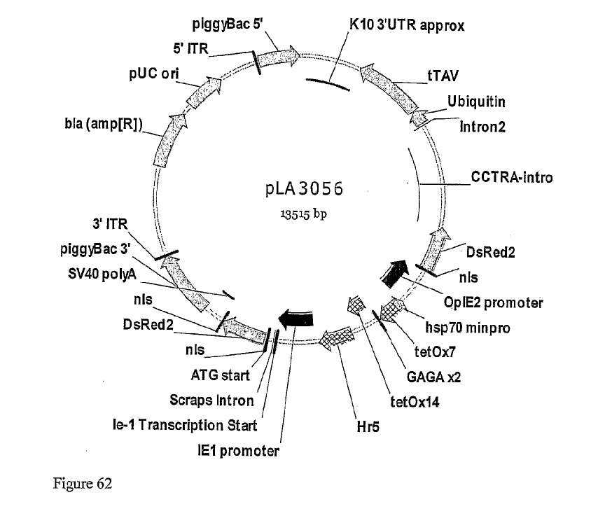

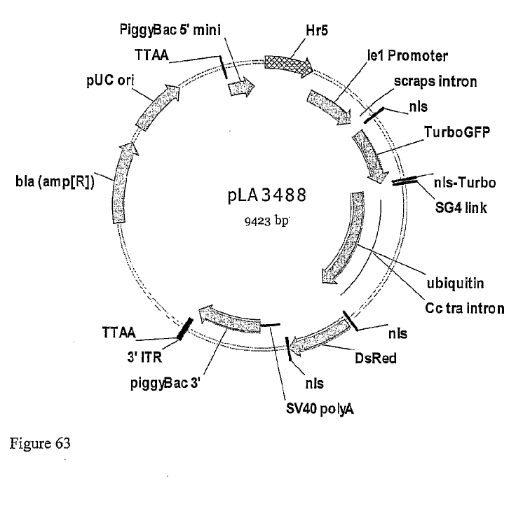

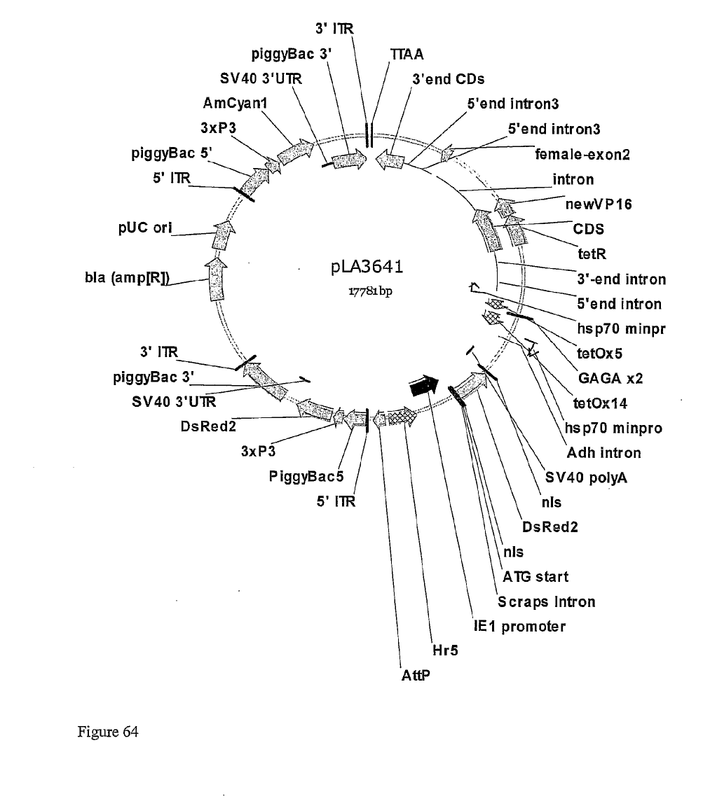

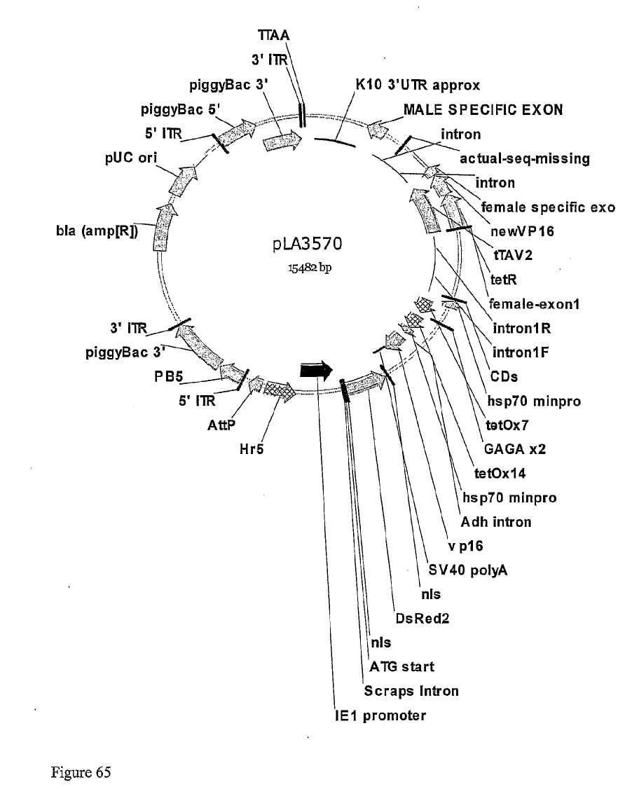

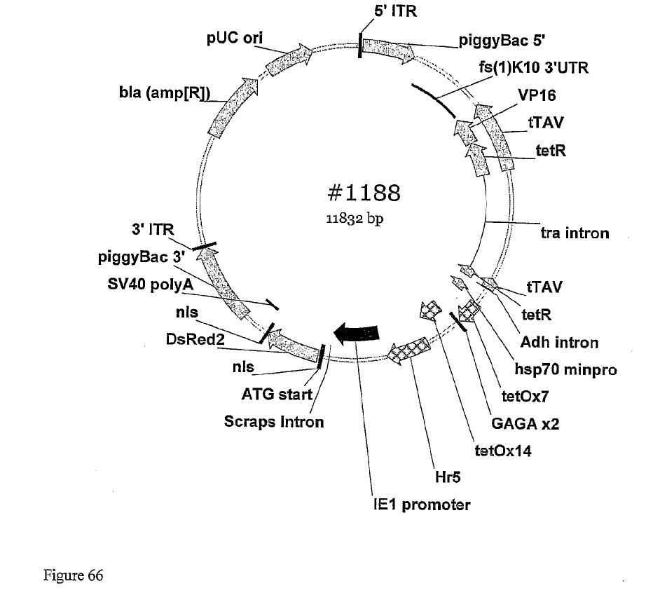

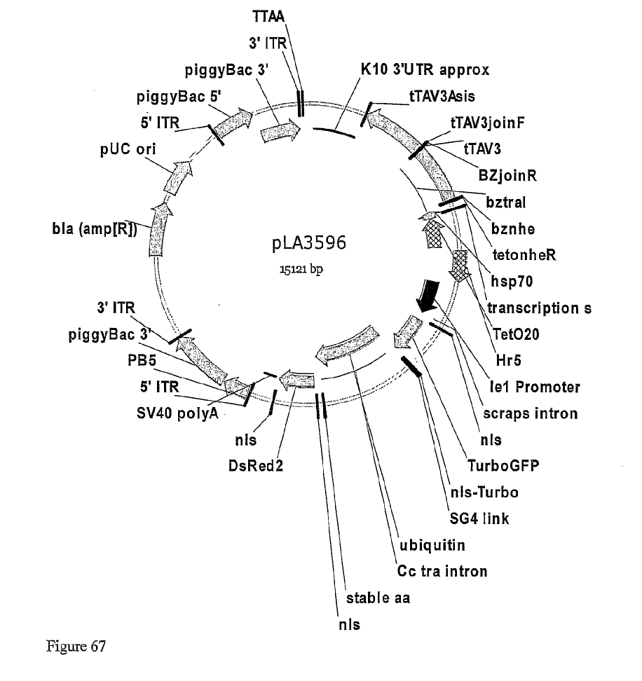

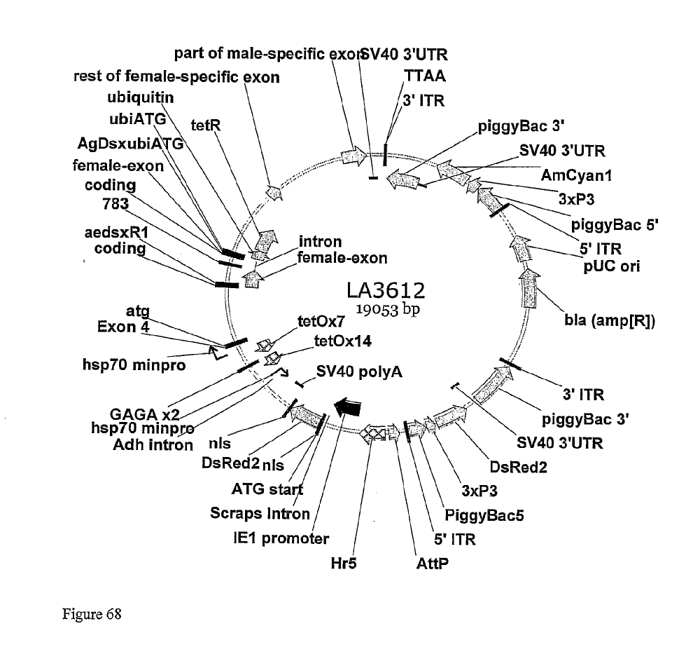

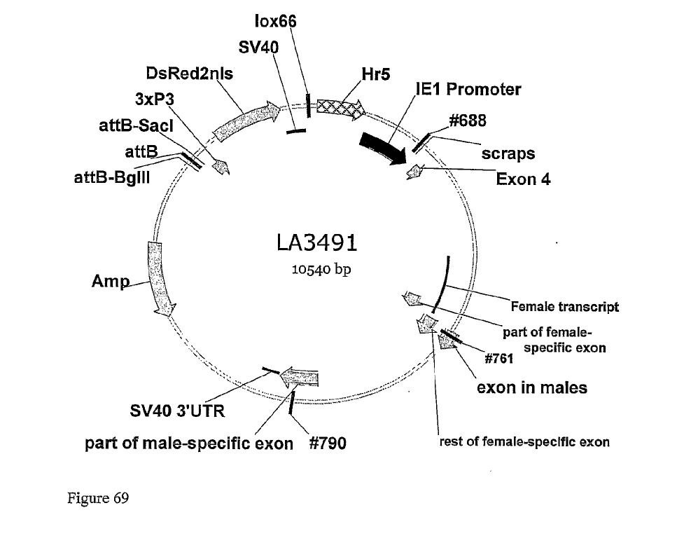

FIGS. 19-51 are as per Examples 1-9 above. FIGS. 52-58, 68 and 69 show various plasmid diagrams and sequences. FIGS. 59-60 are described above and FIGS. 61-66 show various further plasmid diagrams and sequences. FIG. 67 is pLA3596, as discussed elsewhere.

### REFERENCES

Allen M L, Christensen B M. Related 2004 Flight muscle-specific expression of act88F: GFP in transgenic Culex quinquefasciatus Say (Diptera: Culicidae). Parasitol Int. 53(4):307-14.
Bennett D, Szoor B, Gross S, Vereshchagina N, Alphey L. 2003 Ectopic expression of inhibitors of protein phosphatase type 1 (PP1) can be used to analyze roles of PP1 in Drosophila development. Genetics. 164(1):235-45.
Black, D. (2003). Mechanisms of alternative pre-messenger RNA splicing. Annu Rev Biochem 72, 291-336.
Burset, M., Seledtsov, I., and Solovyev, V. (2001). SpliceDB: database of canonical and non-canonical splice sites in mammalian genomes. Nucleic Acids Research 29, 255-259.
Caceres J F, Komblihtt A R. 2002 Alternative splicing: multiple control mechanisms and involvement in human disease. Trends Genet. 18(4):186-93.
Cande C, Cecconi F, Dessen P, Kroemer G. 2002 Apoptosis-inducing factor (AIF): key to the conserved caspase-independent pathways of cell death?
J Cell Sci. 115(24):4727-34.
Cartegni, L., Chew, S., and Krainer, A. (2002). Listening to silence and understanding nonsense: exonic mutations that affect splicing. Nature Reviews Genetics 3, 285-298.
Clark, F., and Thanaraj, T. (2002). Categorization and characterization of transcript-confirmed constitutively and alternatively spliced introns and exons from human. Human Molecular Genetics 11, 451-464.
Funaguma, S., Suzuki, M., Tamura, T., and Shimada, T. (2005). The Bmdsx transgene including trimmed introns is sex-specifically spliced in tissues of the silkworm, Bombyx mori. J Insect Sci 5, 17.
George, E. L., Ober, M. B. and Emerson Jr, C. P. (1989). Functional domains of the Drosophila melanogaster muscle myosin heavy-chain gene are encoded by alternatively spliced exons. Mol. Cell Biol. 9:2957-2974.
Graveley B R. 2001 Alternative splicing: increasing diversity in the proteomic world. Trends Genet. 17(2):100-7.
Hammes, A., Guo, J. K., Lutsch, G., Leheste, J. R., Landrock, D., Zeigler, U., Gubler, M. C. and Schedl, A. (2001). Two splice variants of the Wilms' Tumour 1 gene have distinct functions during sex determination and nephron formation. Cell 106:319-329.
Hastings, G. A. and Emerson Jr, C. P (1991). Myosin functional domains encoded by alternative exons are expressed in specific thoracic muscles of Drosophila. J. Cell Biol. 114: 263-276.
Hedley, M. L. and Maniatis (1991). Sex-specific splicing and polyadenylation of dsx pre-mRNA requires a sequence that binds specifically to a tra-2 protein in vivo. Cell 65:579-586.
Heinrich J. C. and Scott M. J. 2000 A repressible female-specific lethal genetic system for making transgenic insect strains suitable for a sterile-release program PNAS 97 (15): 8229-8232
Horn C, Wimmer E A. 2003 A transgene-based, embryo-specific lethality system for insect pest management. Nat Biotechnol. 21(1):64-70.
Hoshijima, K. K, Inoue, L., Higuchi, I., Sakamoto, H. and Shimura, Y. (1991). Control of doublesex alternative splicing by transformer and transformer-2 in Drosophila. Science 252:833-836.
Huang, Q., Deveraux, Q. L., Maeda, S., Salvesen, G. S., Stennicke, H. R., Hammock, B. D. and Reed, J. C. (2002). Evolutionary conservation of apoptosis mechanisms:
Lepidopteran and baculoviral inhibitor of apoptosis proteins are inhibitor of mammalian caspase-9. Agricultural Sciences 97(4):1427-1432.
Ito, Y., Hirochicka, H. and Kurata, N. (2002). Organ-specific alternative transcripts of KNOX family class 2 homeobox genes of rice. Gene 288:41-47.
Johnson J M, Castle J, Garrett-Engele P, Kan Z, Loereli P M, Armour C D, Santos R, Schadt E E, Stoughton R, Shoemaker D D. 2003 Genome-wide survey of human alternative pre-mRNA splicing with exon junction microarrays. Science. 302(5653):2141-4.
Jurica M S, Moore M J. 2003 Pre-mRNA splicing: awash in a sea of proteins. Mol Cell. 12(1):5-14.
Kazzaz J A, Rozek C E. 1989 Tissue-specific expression of the alternately processed Drosophila myosin heavy-chain messenger RNAs. Dev Biol. 133(2):550-61.
Maniatis, T., and Tasic, B. (2002). Alternative pre-mRNA splicing and proteome expansion in metazoans. Nature 418, 236-243.
Muñoz, D., Jimenez, A., Marinotti, O., and James, A. (2004). The AeAct-4 gene is expressed in the developing flight muscles of females Aedes aegypti. Insect Molecular Biology 13, 563-568.
Nishiyama, R., Mizuno, H., Okada, S., Yamaguchi, T., Takenaka, M., Fukuzawa, H. and Ohyama, K. (1999). Two mRNA species encoding calcium-dependent protein kinases are differentially expressed in sexual organs of Marchantia polymorpha through alternative splicing. Plant Cell Physiol. 40(2):205-212.
Nishiyama, R., Yamato, K. T., Miura, K., Sakida, M., Okada, S., Kono, K., Takahama, M., Sone, T., Takenaka, M., Fukuzawa, H. and Ohyama, K. (2000). Comparison of expressed sequence tags from male and female sexual organs of Marchantia polymorpha. DNA Res. 7:165-174.
Olson, M. R., Holley, C. L., Ji Yoo, S., Huh, J. R, Hay, B. A. and Kombluth, S. (2003). Reaper is regulated by IAP-mediated Ubiquitiation. J. Biol. Chem., 278(6):4028-4034.
Olson, M. R., Holley, C. L., Gan, E. C., Colon-Ramos, D. A., Kaplan, B. and Kombluth, S. (2003). A GH3-like domain in reaper is required for mitochondrial localization and induction of IAP degradation. J. Biol. Chem. 278(45):44758-44768.
Pan, Q., Shai, O., Misquitta, C., Zhang, W., Saltzman, A., Mohammad, N., Babak, T., Siu, H., Huglies, T., Morris, Q., et al. (2004). Revealing global regulatory features of mammalian alternative splicing using a quantitative microarray platform. Mol Cell 16, 929-941.
Pane, A., Salvemini, M., Delli Bovi, P., Polito, C., and Saccone, G. (2002). The transformer gene in Ceratitis capitata provides a genetic basis for selecting and remembering the sexual fate. Development 129, 3715-3725.
Park, J., Parisky, K., Celotto, A., Reenan, R., and Graveley, B. (2004). Identification of alternative splicing regulators by RNA interference in Drosophila. Proc Nat'l Acad Sci (USA) 101, 15974-15979.
Parker L, Gross S, Beullens M, Bollen M, Bennett D, Alphey L. 2002 Functional interaction between nuclear inhibitor of protein phosphatase type 1 (NIPP1) and protein phosphatase type 1 (PP1) in Drosophila: consequences of over-expression of NIPP1 in flies and suppression by co-expression of PP1. Biochem J. 368(3):789-97.
Raphael, K. A., Whyard, S., Shearnian, D., An, X. and Frommer, M. (2004). Bactrocera tyroni and closely related pest-tephritids-molecular analysis and prospects for transgenic control strategies. Insect Biochem. Mol. Biol. 34:167-176.
Ryner, L. and Baker, B. S. (1991). Regulation of doublesex pre-mRNA processing occurs by 3′-splice site activation. Genes Dev. 5:2071-2085.
Saccone, G., Pane, A., and Polito, C. (2002). Sex determination in flies, fruittles and butterflies. Genetica 116, 15-23.
Scali, C., Catteruccia, F., Li, Q., and Crisanti, A. (2005). Identification of sex-specific transcripts of the Anopheles gambiae doublesex gene. J Exp Biol 208, 3701-3709.
Scott, M., Heinrich, J., and Li, X. (2004). Progress towards the development of a transgenic strain of the Australian sheep blowfly (Lucilia cuprina) suitable for a male-only sterile release program. Insect Biochem Mol Biol 34, 185-192.
Seo, S-J., Cheon, H-M., Sun, J., Sappington, T. W. and Raikhel, A. S. (2003). Tissue- and stage-specific expression of two lipophorin receptor variants with seven and eight ligand-binding repeats in the adult mosquito. J. Biol. Chem. 278(43):41954-41962.
Siebel C W, Fresco L D, Rio D C. 1992 The mechanism of somatic inhibition of Drosophila P-element pre-mRNA splicing: multiprotein complexes at an exon pseudo-5′ splice site control U1 snRNP binding. Genes Dev. 6(8):1386-401.
Shivikrupa, Singh., R and Swarup, G. (1999). Identification of a novel splice variant of C3G which shows tissue-specific expression. DNA Cell Biol. 18: 701-708.
Smith, C., and Valcarcel, J. (2000). Alternative pre-mRNA splicing: the logic of combinatorial control. Trends Biochem Sci 25, 381-388.
Stoss, O., Stoilov, P., Hartmann, A. M., Nayler, O., and Stamm, S. (1999). The in vivo miniigene approach to analyze tissue-specific splicing. Brain Research Protocols 4, 383-394.
Stoss, O., Olbrich, M, Hartmann, A. M., Konig, H., Memmott, J., Andreadis, A and Stamm, S. (2001). The STAR/GSG family protein rSLM-2 regulates the selection of alternative splice sites. J. Biol. Chem. 276(12):8665-8673.
Streuli, M. and Saito, H. (1989). Regulation of tissue-specific alternative splicing: exon-specific cis-elements govern the splicing of leukocyte common antigen pre-mRNA. EMBO J. 8(3): 787-796.
Suzuki, M., Ohbayashi, F., Mita, K., and Shimada, T. (2001). The mechanism of sex-specific splicing at the doublesex gene is different between Drosophila melanogaster and Bombyx mori. Insect Biochem Mol Biol 31, 1201-1211.
Thanaraj, T., and Clark, F. (2001). Human GC-AG alternative intron isoforms with weak donor sites show enhanced consensus at acceptor exon positions. Nucleic Acids Research 29, 2581-2593.
Thanaraj, T., Stamm, S., Clark, F., Reithoven, J., Le Texier, V., and Muilu, J. (2004). ASD: the Alternative Splicing Database. Nucleic Acids Research 32, D64-D69.
Varshavsky, A. (2000). Ubiquitin fusion technique and its descendants. Meth Enz 327.
Venables, J. (2002). Alternative splicing in the testes. Curr Opin Genet Dev 12, 615-619.
Venables J P. 2004 Aberrant and alternative splicing in cancer. Cancer Res. 64(21):7647-54.
Vernooy, S. Y., Copeland, J., Ghaboosi, N., Griffin, E. E., Yoo, S. J. and Hay, B. A. (2000). J. Cell Biol. 150(2):F69-F75.
White, K., Tahoaglu, E. and Steller, H. (1996). Cell killing by the Drosophila gene reaper. Science 271 (5250): 805-807.
Wing, J. P., Zhou, L., Schwartz, L. M. and Narnbu, J. R. (2001) Distinct cell killing properties of the Drosophila reaper, head involution defective, and grim genes. Cell Death Diffn 5(11): 930-939
Yali Chiu A., and Pin Ouyang, A. B., (2006). Loss of Pnn expression attenuates expression levels of SR family splicing factors and modulates alternative pre-mRNA splicing in vivo. Bioch. Biophys. Res. Comm.341:663-671.
Yoshirnura, K., Yabuta, Y., Ishikawa, T. and Shigeoka, S. (2002). Identification of a cis element for tissue-specific alternative splicing of chloroplast Ascorbate Peroxidase pre-mRNA in higher plants. J. Biol. Chem 277 (43):40623-40632.

1-44. (canceled)

45. A polynucleotide expression system comprising:
at least one heterologous polynucleotide sequence encoding a functional protein, defined between a start codon and a stop codon, and/or polynucleotides for RNAi, to be expressed in an organism;
at least one promoter operably linked thereto; and
at least one splice control sequence which, in cooperation with a spliceosome, is capable of (i) mediating splicing of an RNA transcript of the coding sequence to yield a first spliced mRNA product, and (ii) mediating at least one alternative splicing of said RNA transcript to yield an alternative spliced mRNA product;
wherein, when the at least one heterologous polynucleotide sequence encodes a functional protein, at least one of the mature mRNA products comprising a continuous Open Reading Frame extending from said start codon to said stop codon, thereby defining a protein, which is said functional protein, or is related to said functional protein by at least one amino acid deletion, and which is functional when translated and, optionally, has undergone post-translational modification;
the mediation being selected from the group consisting of: sex-specific mediation, stage-specific mediation, germline-specific mediation, tissue-specific mediation, and combinations thereof.

46. The polynucleotide expression system of claim 45, wherein said mediation is sex-specific mediation.

47. The polynucleotide expression system of claim 45, wherein said polynucleotide sequence to be expressed comprises two or more coding exons for the functional protein.

48. The polynucleotide expression system of claim 45, wherein said protein is a marker, or has a lethal, deleterious or sterilizing effect.

49. The polynucleotide expression system of claim 48, wherein said protein has a lethal effect resulting in sterilization.

50. The polynucleotide expression system of claim 49, wherein said lethal effect of the protein is conditionally suppressible.

51. The polynucleotide expression system of claim 48, wherein said protein is selected from the group consisting of an apoptosis-inducing factor, Hid, Reaper (Rpr), and Nipp1Dm.

52. The polynucleotide expression system of claim 45, wherein said system comprises at least one positive feedback mechanism, being at least a functional protein to be differentially expressed, via alternative splicing, and at least one promoter therefor, wherein a product of a gene to be expressed serves as a positive transcriptional control factor for the at least one promoter, and whereby the product, or the expression of the product, is controllable.

53. The polynucleotide expression system of claim 52, wherein an enhancer is associated with said promoter, the gene product serving to enhance activity of the promoter via the enhancer.

54. The polynucleotide expression system of claim 53, wherein a control factor is the tTA gene product or an analogue thereof, and wherein one or more tetO operator units is operably linked with the promoter and is the enhancer, tTA or its analogue serving to enhance activity of the promoter via tetO.

55. The polynucleotide expression system of claim 45, wherein said functional protein is itself a transcriptional transactivator, such as the tTAV system, comprising tTAV, tTAV2 or tTAV3.

56. The polynucleotide expression system of claim 45, wherein said promoter is activated by environmental conditions, for instance the presence or absence of a particular factor such as tetracycline in the tet system or by variation of the environmental temperature.

57. The polynucleotide expression system of claim 45, wherein said promoter is selected from the group consisting of the srya embryo-specific promoter, or its homologues, the Drosophila gene slow as molasses (slam), or its homologues.

58. The polynucleotide expression system of claim 45, further comprising an enhancer.

59. The polynucleotide expression system of claim 45, wherein said mediation of alternative splicing is sex-specific mediation and the splice control sequence is derived from a tra intron.

60. The polynucleotide expression system of claim 59, wherein said splice control sequence is derived from the Medfly transformer gene Cctra, or from another ortholog or homolog of the Drosophila transformer gene.

61. The polynucleotide expression system of claim 60, wherein said another ortholog or homolog of the Drosophila transformer gene is from a tephritid fruit fly.

62. The polynucleotide expression system of claim 61, wherein the tephritid fruit fly is C. rosa, or B. zonata.

63. The polynucleotide expression system of claim 45, wherein said splice control sequence is derived from the alternative splicing mechanism of the Actin-4 gene.

64. The polynucleotide expression system of claim 63, wherein said Actin-4 gene is from Aedes spp.

65. The polynucleotide expression system of claim 63, wherein said Actin-4 gene is from Aedes aegypti AeActin-4.

66. The polynucleotide expression system of claim 45, wherein the splicing mechanism comprises at least a fragment of the doublesex (dsx) gene, preferably that derived from Drosophila, B. mori, Pink Boll Worm, Codling Moth, or a mosquito, in particular Aedes gambiae or especially Aedes aegypti.

67. The polynucleotide expression system of claim 63, wherein said splice control sequence and said heterologous polynucleotide sequence encoding a functional protein, defined between a start codon and a stop codon, and/or polynucleotides for interference RNA (RNAi), to be expressed in an organism, are provided in the form of a minigene construct or a cassette exon.

68. The polynucleotide expression system of claim 66, wherein said splice control sequence and said heterologous polynucleotide sequence encoding a functional protein, defined between a start codon and a stop codon, and/or polynucleotides for interference RNA (RNAi), to be expressed in an organism, are provided in the form of a minigene construct or a cassette exon.

69. The polynucleotide expression system of claim 48, wherein said system is a plasmid or construct selected from the group consisting of any one of FIGS. 16-18, 22-24, 26-32, 49, 52-55, and 61-69, and/or SEQ ID NOs 46-48, 50-56, 143-145 and 151-162.

70. The polynucleotide expression system of claim 45, wherein said at least one splice control sequence is intronic and comprises on its 5′ end guanine (G) nucleotide, in RNA.

71. The polynucleotide expression system of claim 45, wherein said at least one splice control sequence is intronic and comprises on its 5′ end UG nucleotides and UT at its 3′ end, in RNA.

72. The polynucleotide expression system of claim 45, wherein said mediation is sex-specific mediation and is further mediated or controlled by binding of the TRA protein or TRA/TRA2 protein complex, or homologues thereof.

73. The polynucleotide expression system of claim 72, wherein said system comprises the consensus sequence: TCWWCRATCAACA, where W=A or T and R=A or G.

74. The polynucleotide expression system of claim 45, wherein said organism is a mammal, a fish an invertebrate, an arthropod, an insect or a plant.

75. The polynucleotide expression system of claim 45, wherein said organism is an insect from the Order Diptera.

76. The polynucleotide expression system of claim 75, wherein said insect is a tephritid fruit fly selected from the group consisting of: Medfly (Ceratitis capitata), Mexfly (Anastrepha ludens), Oriental fruit fly (Bactrocera dorsalis), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata) Caribbean fruit fly (Anastrepha suspensa) and West Indian fruit fly (Anastrepha obliqua).

77. The polynucleotide expression system of claim 75, wherein said insect is a mosquito from the genera Stegomyia, Aedes, Anopheles or Culex.

78. The polynucleotide expression system of claim 77, wherein said mosquito is selected from Aedes aegypti, Aedes albopictus, Anopheles stephensi, Anopheles albimanus and Anopheles gambiae.

79. The polynucleotide expression system of claim 75, wherein said insect is selected from the group consisting of: the New world screwworm (Cochliomyia hominivorax), Old world screwworm (Chrysomya bezziana) and Australian sheep blowfly (Lucilia cuprina), codling moth (Cydia pomonella), the silk worm (Bombyx mori), the pink bollworm (Pectinophora gossypiella), the diamondback moth (Plutella xylostella), the Gypsy moth (Lymantria dispar), the Navel Orange Worm (Amyelois transitella), the Peach Twig Borer (Anarsia lineatella) and the rice stem borer (Tryporyza incertulas), the noctuid moths, especially Heliothinae, the Japanese beetle (Popilla japonica), White-fringed beetle (Graphognatus spp.), Boll weevil (Anthonomous grandis), corn root worm (Diabrotica spp) and Colorado potato beetle (Leptinotarsa decemlineata).

80. The polynucleotide expression system of claim 75, wherein said insect is not a Drosphilid.

81. The polynucleotide expression system of claim 45, wherein the expression of the heterologous polynucleotide sequence leads to a phenotypic consequence in the organism.

82. The polynucleotide expression system of claim 45, wherein said polynucleotide sequence to be expressed comprises a polynucleotides for interference RNA (RNAi).

83. A method of population control of an organism in a natural environment therefor, comprising:
i) breeding a stock of the organism,
the organism carrying a gene expression system comprising the system of claim 45 which is a dominant lethal genetic system,
ii) distributing the said stock animals into the environment at a locus for population control; and
iii) achieving population control through early stage lethality by expression of the lethal system in offspring that result from interbreeding of the said stock individuals with individuals of the opposite sex of the wild population.

84. The method of claim 83, wherein said early stage lethality occurs early in development.

85. The method of claim 84, wherein said early stage lethality is embryonic or before sexual maturity.

86. The method of claim 83, wherein said lethal effect of the lethal system is conditional and occurs in the said natural environment via the expression of a lethal gene, the expression of said lethal gene being under the control of a repressible transactivator protein, the said breeding being under permissive conditions in the presence of a substance, the substance being absent from the said natural environment and able to repress said transactivator.

87. A method of biological control, comprising:
i) breeding a stock of males and female organisms transformed with the system of claim 45 under permissive conditions, allowing the survival of males and females, to give a dual sex biological control agent;
ii) optionally before the next step imposing or permitting restrictive conditions to cause death of individuals of one sex and thereby providing a single sex biological control agent comprising individuals of the other sex carrying the conditional lethal genetic system;
iii) releasing the dual sex or single sex biological control agent into the environment at a locus for biological control; and
iv) achieving biological control through expression of the genetic system in offspring resulting from interbreeding of the individuals of the biological control agent with individuals of the opposite sex of the wild population.

88. A method of sex separation comprising:
i) breeding a stock of male and female organisms transformed with the expression system of claim 45 under permissive or restrictive conditions, allowing the survival of males and females; and
ii) removing the permissive or restrictive conditions to induce the lethal effect of the lethal gene in one sex and not the other by sex-specific alternative splicing of the lethal gene.

89. A method or biological or population control comprising;
i) breeding a stock of male and female organisms transformed with the gene expression system of claim 45 under permissive or restrictive conditions, allowing the survival of males and females;
ii) removing the permissive or restrictive conditions to induce the lethal effect of the lethal gene in one sex and not the other by sex-specific alternative splicing of the lethal gene to achieve sex separation;
iii) sterilising or partially sterilising the separated individuals and
iv) achieving said control through release of the separated sterile or partially sterile individuals in to the natural environment of the organism


SEQUENCE LISTING (162 sequences)

SEQ ID NO: 1 - DNA, 13 bp, artificial
  Ceratitis capitata tra consnesus sequence
tcwwcratcaaca

SEQ ID NO: 2 - DNA, 10 bp, Artificial
  LA3097 flanking sequence
agccaccatg

SEQ ID NO: 3 - DNA, 10 bp, artificial
  LA3097 flanking sequence
gtcagccgcc

SEQ ID NO: 4 - DNA, 21 bp, artificial
  primer 688 - ie1-transcr
gttgcaagttgacactggcgg

SEQ ID NO: 5 - DNA, 21 bp, artificial
  primer 790 - Aedsx-m-r2
ccactgtgtaaggcttcctcc

SEQ ID NO: 6 - DNA, 21 bp, artificial
  primer 761 - Aedsx-fem-r
ggatggttggttgaagatccg

SEQ ID NO: 7 - DNA, 21 bp, artificial
  primer AedsxR1
actgcgcaactctacaccgtc

SEQ ID NO: 8 - RNA, 13 nt, artificial
  Pane et al consensus sequence
ucwwcraucaaca

SEQ ID NO: 9 - RNA, 13 nt, artificial
  Scali et al 2005 consensus sequence
ucwwcaaucaaca

SEQ ID NO: 10 - DNA, 13 bp, Drosophila sp.
tcaacaagcaaca

SEQ ID NO: 11 - DNA, 13 bp, Drosophila sp.
ttatcaaacaaca

SEQ ID NO: 12 - DNA, 13 bp, Drosophila sp.
tcatcaattaaaa

SEQ ID NO: 13 - DNA, 13 bp, Drosophila sp.
tcatcaatcaaac

SEQ ID NO: 14 - DNA, 13 bp, Drosophila sp.
tcttcaaccaacc

SEQ ID NO: 15 - DNA, 13 bp, Drosophila sp.
cctacaatctaca

SEQ ID NO: 16 - DNA, 13 bp, Drosophila sp.
tcttagatcaaaa

SEQ ID NO: 17 - DNA, 13 bp, Drosophila sp.
tcttcgatcatta

SEQ ID NO: 18 - DNA, 13 bp, Drosophila sp.
ccaacaatctaca

SEQ ID NO: 19 - DNA, 13 bp, Drosophila sp.
tcaaagatcacca

SEQ ID NO: 20 - DNA, 13 bp, Drosophila sp.
tcttcggtcgacg

SEQ ID NO: 21 - DNA, 13 bp, Drosophila sp.
tcgacaaacaaaa

SEQ ID NO: 22 - DNA, 13 bp, Drosophila sp.
tattcaaacaacg

SEQ ID NO: 23 - DNA, 13 bp, Drosophila sp.
ttttcgataaaaa

SEQ ID NO: 24 - DNA, 13 bp, Drosophila sp.
tcttcagtctgca

SEQ ID NO: 25 - DNA, 13 bp, Drosophila sp.
gattcaatcatca

SEQ ID NO: 26 - DNA, 13 bp, Drosophila sp.
ttatcgagcaaaa

SEQ ID NO: 27 - DNA, 13 bp, Drosophila sp.
tcataactcaaga

SEQ ID NO: 28 - DNA, 13 bp, Drosophila sp.
tcagaaatcaaaa

SEQ ID NO: 29 - DNA, 13 bp, Drosophila sp.
tctttaatttaca

SEQ ID NO: 30 - DNA, 13 bp, Drosophila sp.
tttacaatcctca

SEQ ID NO: 31 - DNA, 13 bp, Drosophila sp.
tcatagatcagga

SEQ ID NO: 32 - DNA, 13 bp, Drosophila sp.
acctcaaacaaca

SEQ ID NO: 33 - DNA, 13 bp, Drosophila sp.
tcatcgaacaccc

SEQ ID NO: 34 - DNA, 1014 bp, artificial
  Open reading frame of tTAV construct
atgggcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggg

SEQ ID NO: 35 - protein, 338 aa, artificial
  Protein sequence of tTAV
MGSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGPAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 36 - DNA, 1014 bp, artificial
  Open reading frame of tTAV2
atgagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggctaa

SEQ ID NO: 37 - protein, 337 aa, artificial
  Protein sequence of tTAV2
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGPAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 38 - DNA, 1011 bp, artificial
  Open reading frame of tTAV3
atgggcagccgcctggacaagagcaaggtgatcaacagcgccctggagctgctgaacgaagttggtatcgagggcctgaccacccgcaagctggcccagaagctgggcgtggaacagccgaccctgtactggcacgtgaagaacaagcgcgccctgctggacgccctggccatcgaaatgctggatcgccaccacacccacttctgcccgctggagggcgagagctggcaggatttcctgcgcaacaacgccaagagcttccgctgcgccctgctgtcgcaccgcgatggcgccaaggtgcacctgggcacccgcccgaccgagaagcagtacgagaccctggagaaccagctggccttcctgtgccagcagggcttcagcctggagaacgccctgtacgccctgagcgccgtgggccacttcaccctgggctgtgtgctggaggatcaggagcaccaggtggccaaggaggagcgcgagaccccgaccaccgatagcatgccgccgctgctgcgccaggccatcgagctgttcgatcaccagggcgccgagccggccttcctgttcggcctggagctgatcatctgcggcctggaaaagcagctgaagtgcgagagcggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtag

SEQ ID NO: 39 - protein, 336 aa, artificial
  Protein sequence of tTAV3
MGSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 40 - DNA, 568 bp, Pectinophora gossypiella
gctagtggagaactgccacaaactgctggaaaagttccactactcctgggaaatgatgcccctggtgctggtcattctaaactacgccggctccgacctcgacgaggcttctagaaaaattgatgaagggaagatgatcatcaacgagtacgcgaggaagcacaatctgaacatcttcgatggccacgagctaaggaactcgactcgccagtacggactttaatacagtaatattagttttctccaacaacactaaacacgacataacacgctacacgcaaaaaatacacgagtctttaatgttttacacgctcagtaaattattcacttacacgcttaactaaaattttacacaatcggtaaaaaaatacaacaatttattatcgtaaaaattacacaaaataaatgagatttaaatgtcgtttaataaaataaaataaaaatagcatcgggaatatcttttcacctattgccggagaacagtttaaatggatactctcatttgaatcattttaattgtagtagcattttattttattattaatagcaataagtacacaaacataaa

SEQ ID NO: 41 - DNA, 610 bp, Pectinophora gossypiella
gtagtggagaactgccacaaactgctggaaaagttccactactcctgggaaatgatgcccctggtgctggtcattctaaactacgccggctccgacctcgacgaggcttctagaaaaattgatgaagggaagatgatcatcaacgagtacgcgaggaagcacaatctgaacatcttcgatggccacgagctgaggaactcgactcgccagtacggactttaatacagaaaatgctgagcgaaattaataatataagtggtgtactatcgtcgtccatgaagttattttgcgaatgatactttgttttgtatgtgctgtgtgttgtgtggacttttgctgtgcgttgctgtttgcgatggaaggactattgtgtcgtcgccacgctggactattcgcacattgggtggtccaccagtggcggatgtacgagcggtcgctgtgctcgctcctggagctgcaagcgcgcaaagggacgtactcggtgtgctgctcaccccgctacgtcatcgcgcccgagtacgcgtcacacctgttgcctctgccgcttaccacgcagagatcatccccgccgcccgcgcacttgtagcgatgcgaacctgcgccgcgggaa

SEQ ID NO: 42 - DNA, 449 bp, Pectinophora gossypiella
  misc_feature (26)..(26): n is a, c, g, or t
gctagtggagaactgccacaaactgntggaaaagttccactactcctgggaaatgatgcccctggtgctggtcattctaaactacgccggctccgacctcgacgaggcttctagaaaaattgatgaagcacattgggtggtccaccagtggcggatgtacgagcggtcgctgtgctcgctcctggagctgcaagcgcgcaaagggacgtactcggtgtgctgctcaccccgctacgtcatcgcgcccgagtgcgcgtcacacctgttgcctctgccgcttaccacgcagagatcatccccgccgcccgcgcacttgtagcgatgcgaacctgcgccgcgggaagtaagtactatttcatttattattctttttatttttggttttaaggtgctgacagacttgaatttcaagcaaatagtgtctgacaaagagctcaaaatagacatgt

SEQ ID NO: 43 - DNA, 28774 bp, Aedes aegypti
acagtgaaatttgatcgatcactcatcgaaacgagatcactttcgattgatcgtgacaattttttagaatccatttcacagtcgttgggactgttgaccctgtcactttaaactagctagtgagtagctttgctctagtgaaagctaactagcactgttaaaaaatcttaggtaaagtgtcagcaaccctgacaactgggccacctcttgccgaccataagcaaatgaaatcaaatggttcgctacgaaggttaattgggtttcgatctacttcgtcctaagcgctatttttcgtcatacggtggagaacggctggtattcgtttactttagtttaccaagcgatgcttccaattaacccaaagctagatgaagcaggattcgcgataaaaagcagtatgcgaacttaaaatgttctactacattacggcgggtattcaaatttacctgccacataaatttattttccaagtataatttgcgaaagctgcaatggttcatgcttgaattttacaagatgatgtaatgccgcccataagtttaaatggacggtgtatttaaataaaaggttcatattaaacgctttcgacgttaccaagtaccatttgtacacaaacatgtaataaaactattgtatttctataaataacttcagttcaatcatccactttgcacattttcaccgaaatcgcatggacgaaggtaaacatgtgtttgtacattattttgataacataaagatatttattgaagtcaagttagtaggtgaaacgtgtaaaagtggctttagcgtacctgcttgacgtaccgagcgaaatctgattagcggtcgactaagccataaaacttctacaattcacaaaattttgaaaaattccctcgctgccacgatactaatgcactgcatggctcgctttagactaatcgccagctgattcggtattttgaagatgttaagtgttttaaaactttttaagggagcgacggtgctatgattacgtaatcaaatgttctttcttttactttcagaccaattgcagaacaagctttatcctaatccatctcattttgggaacagcactagccgcgaccattagccgtttagtttacaagaaagaaaatgaaagtctggttaacgtcttgttcgaaataggattaggtagagtaaaacccttgtcgtgatcggcgctggtaatcggcatctgcgtagagaacatgttgtacttcctcgaggacgattgctcgcgctcgcacggttcttattgctaccatggtgaaaccactagcgccgaggaagtgctagacgcatctcttgtacaacatgaaggagctcctgctaacgagcgcgagcgtgccaagaataacgatggtaccactttggtgatcgcggctccttcacgataccgttgtgaaggttttctgaattgcgcatcgtctccgaagggtgtgtccaggtgcattgtctcccaactgacctgttcccgacaatatcgagcactaaatggcaacacttccaaaagacttaacgcgtagcagaggcttcccacacaggtccacgtaacagagggttgactggacaagggctgttatagctcgtgattggtttccattagagagcagtatctcgtagtagcgtaggagagtccattagagtgcgatattccgtgagtttgtgtgaccggcgatagagaagccctgacgccaaaggtaatctctcgtcatagagcatcatcgcatcctctcaggtaatctcacgctataaggcactcaaacacactggccgctatctcttcgggactgccgcgcttcaagacgattgtaactcggaaactgacctgattagtacataaaaagagacctattgcgtaagcttataagaaacgagtttgtccacacggttggcgcgaagttctgctaacattgagcctttgactggactaatcatgtatttttctctggataacgcattcgaatattctttgctcaaacaggtgtgccaacatggtttcgcaagatcgctggatggtaaagatgtccgaggcagggtacgataaccgggcggatggcagtggagcttccagcagcagcctgaacccgcgaataccaaagcgttctagcgacctaccatttctacaggctccgtcccatgctattggcccgcctaccgtcacctcgaaggtcgtcgtcggacttgggcgcttcgccgccgaactgtgcccgctgccggaaccacggtcacaagatcggcctgaagggacacaagcgctattgtaagtatcgcaattgtacctgcgaaaagtggcggcggcttgacacgggcgacggccttggtgccagtgttctagccggacttccctgtgttcgcgataacattcatagcgttaacatggacgcttttcacctgcctgacggccgaacggcagcgggtcatggccctgcagacggctctccgaagggcgcaaacccaggacgaacagcggttgctggtagacggagaggtggacggactgccggcttgccgtcgcccagtaccgggacgtctgccgagaggcttcccgcgtttgggtcctgcttgtcgccaacgaccatctgcctctccaccccgccgaaccggtacatagccttcaaataccaaaattgtctgacctaaaagagatgatccataattctcagcagaggtcgttgatcgactgcgactcgtgggcggcttggccatgtatcggaagtttatggttttaacagactggattttctctactaggtattaagagtcgtctccagcaactagctgacgctgagcaccaccggctcgatgaactccaccccgggcagctcgttggtaacgctgtcccagcaccgaagatcaccctgctccgccgcgtcggtccaccccagcgaggcggtggccgagctacttgaggtggggcccgtcgagcaaccattgcgacagggtcgtggcttctagtgggacgaggcggcgcagccaggtggggtcgctccgtcagcaaaacgttgcaggtaggtgtgaggcatatctatttcgttattctctcaatgtttgtggagaaccggccggaattcaacatcgaagtcggtttctgagtcgttttgcaacgtccatccacactccgtatagataaagcaataagagagttacaaacacctcttggccggccttaagttgtagcttcagccaaagacttctattgatttatgataaatttctctcaaatgtttgcgcggagggtggatttttgagagctgagtggtgtagaaacgaaatgggcatcaaacgttatgcaagataactaaatactatttaaagagagtttacaaacgcgcctcccacctaaaaactctcgactcaccacatctttgctttacccgtagtttgcaatacgggcgctgcttgaaacaggtttatgttaggggtttcctgtgtttcatacagtcaccccattgttatgtatagcacacagatatggataaaagttggattaaccgcgacgaactttgtccaaatacaatccccaaaggacacaaagtatgtcagtggggtaacaatacatatcgtgtgtctatacctattttcaacctaattgcagtgaatatcccatcaaatagagttgcaattgagtagaacacattttaccaacgtataaagcatcgtaatcaattataatatacttaagcaaaatacacgtcacttatagggtagtttatctcaacgttaactcatcttgtgtaaaatggttgcatatttcgtagcattagttaatattatatgaattcgttttatgtatggggaaataatttgtcaaccacatttctagaaaagttgattcatacatgtgtgcttttgaaagccatataccacattatgtttgattcatatctcttatacccctttattaaacagttggtgtaaagatcttttcaactaagtatgtacacacgaaaactttcggtatatggtgtaatacaaactaagtatagagaattaatatgagtcgatttatcgcgaaatttttcaaaatgtcctatgtaccaatgaaagatactctcttatctcgctctgttttgaacataacaactgaaactattatactcagctaaatagcgctttaaaaagttttacaggatacatggttactttctatgagagaatagagcgagacaaaacttgtattgttgactttgatttgggaagtttttcactatagataaaaaaatgtccttgactagcgtttcatacaaaaaaaaaaaaaaacgcaaccaaaaatgttaatgtggttcagtgaaaacccttcaaaaagtgatatctatttttttacaggaactgatcgcaaagtatgtttttttttttttttgcgttggtttttacaattacaccaagtcactttgattaaagaggaagtaaactaagatagtgtctcaatgttggataggtcatttagaaaaggtccgcgagattggatccataataatgattctcctctctaactaatttctccttcatttgattctatcacagagttacaacctatccagtaaatcttttccaggcgctctaacctaggtattattactaagaggagagacactgatccgcatctgtgggatggacaacgtttgtaatttctatcggtatcgaaaataatcgcgcattttcgggcgtattccagaaaacaacaatgaaatgtgactaggcgtagacaccctacctgttgcaaacattaaagatagccatagcttttattagcgcgtaaaagcccgcataaggtcttttgttgttactttaatactgaagcaaatgtgcacaattttcattacatgatattattcaatggggtaggtgggcgacaaaatagattcattaatgttggataataggggcgttttatgacttcgtttacacgtgttaaaagtaatgtactataataagttaccccatccacccgctgttttatctaagtaattacaacctattatccccgcaaagtcattatccctaaatgctccacctcagctggtggccccgtcagtcagttgatcgggaaagcagcaatcaatccggagacaggtcgacctccatcgaacacagtaatagggatttacgaggtggagtcgaccaccggggcagtcagtcaactagccctttcgtcgttagttaggcctctgtccagctggaggtagcttgtggaaccgaacaacactagatgttcgatttctaacgaccgactaagaacatcgtcggaagcgtctggttcattcgacgagccggaaggggttcatctttcgccttggcttgttgtgatctacaagctaaagattgctggctgattcttgtagcagccttcgcagaccaagtaagctgctcggccttccccaagtagaaagcctcgtcgtcgaacgaatagctgctgctacacttcgcgtcgttatcgtcgtcgggggattggtgtttgtaactgcgcactcgtttatacattgttgtttgcgagcagcagcttgcttatcgacgacgatgtgaagcgcagcaatagcagcagccccctaaccacaaacattgacgcgtgagcaaatatgtaacaacaaacgcgatcggcgggcgctgtaactgcctgcagtcacgcgttcattcgcagtcgttgtcgtagtcatacacacgccgtcgttcctttgtatcagctgtgtagcagctagccgcccgcgacattgacggacgtcagtgcgcaagtaagcgtcagcaacagcatcagtatgtgtgcggcagcaaggaaacatagtcgacacatcgttttagtggtgttacaacattgagctactttttgcgtttcgctttcgtgctgcggcggcggcggcgggacttcgctgcactgataggaacggaatgcatgcaaatcaccacaatgttgtaactcgatgaaaaacgcaaagcgaaagcacgacgccgccgccgccgccctgaagcgacgtgactatccttgccttacgtacgtgctccggttgaagagagctctgcgccacttgtggcgggtttcactcaaaaggcatcgtcgcgtcgcaacaaagtgcgcacattcgacgcgtaactgtaaacgaggccaacttctctcgagacgcggtgaacaccgcccaaagtgagttttccgtagcagcgcagcgttgtttcacgcgtgtaagctgcgcattgacattgtaaatagaaagactttggtgcgtttagaaaaagggtcacaaagggtggcaagtgagtatgtatgtgagctcatttcattctcgatggcattgagacgtacatttatctttctgaaaccacgcaaatctttttcccagtgtttcccaccgttcactcatacatacactcgagtaaagtaagagctaccgtaactctgcatatctattctgagaacgaaagttcaatggatgcattttatgcaatgccaccggaattttcctatgaactgctttcacacttcttttaagaaaattttgcagtagataagactcttgctttcaagttacctacgtaaaatacgttacggtggccttaaaaggatacttgacgaaagtgtgaagaaaattcttttaaaacgtcatttaatttattcactccatttagttctgacgtaacattccagataacacacttcaaagtcatggtcagttcatgttgaacgaatgtgcaccgcgatccataaattaaataagtgaggtaaatcaagactgcattgtaaggtctattgtgtgaagtttcagtaccagtcaagtacaacttgcttacacgtggcgctaggtcgcagaacgattccatgtcttaatgtcgtcacttatcatataatcacccagtttttgccccacttaaaaaaacgatgtccactttttatctgagtttcttgcgtcttgctaaggtacagaattacagcagtgaatagtatattagtgggtcaaaaacggggtgaatttttttgctacaggtgaaaaatagactcaaagaatctcctctcttttcagccaaccactccagcggaacccctgaacccggaaacatggtaccaggtgagttcgctgttgaaatactaatttgcagaaaacataagaggagagaaaagtcggttggtgaggtcgccttggggacttgggcctttgtaccatggtccactcaagcgacaactttatgattaaacgtcttttgtatagaaattttgctaccgatttaccataactggaatcgaagacaatatgacttcatcacaccagcagtaaacacggcgtaaaaatgattcatcaggacccgctctttaaaacgatggctaaatggtattgaccttagcttctgttatactgaagtagtgtggtcgtcatttgtgccgcatttttactaagtagtcctgggcgtcaatagccctgtttttccacgctcatcttgggtttcacatcggtgaacaccacttggagacgttttcacacaatgttcatgttcttctttgagtaaatgagttatcgggacaaaaaggtgcgagtagaacccaaagtgtagccacttgtggtgaacctctgcaaaagtgtgttacaagtacaagaagaaactcatttacaagttatgcgtggtcccgtgctcatcaagatagtgtgccacacataagaattatcttaattgaggccttctgcgggccgtgagcttgtttgctacgccctttcaatacgcaccagggcacgagtagttctatcacacggtgtgtattcttaatagaattaactccggaagacgcccggcactcgaacaaacgatgcgggatccttggcgttgagttttagtttctttgacagagaaagacttttgataatctactttctgcagctacgacctttctctgaactatttggaaaattataacaggaaccgcaactcaaaatcaaagaaactgtctctttctgaaaactattagatgaaagacgtcgatgctggaaagagacttgataaaccttttaatattgttatgttgacaatatttatcccttcgattaacaaaaaacttcaagccagggaaacatccagtgtgaaaacactaagcggcgcactttggttcatttcattaatacaactgttataaatagggaagctaattgttttttgaagttcggtccctttgtaggtcacacttttgtgattcgccgcgtgaaaccaagtaaagtaacgtatcgatcactcttaattcaagatgacaaagtggttgagtagtagagtacgtggctcacaatcggaaggttcttggctcgaatctcaatgtatgctatgcatagctagtgagaattaagttctactgtttcaccaactcatcatctcatgcaccgagtgttagccttccaagaaccgagcttagagttacatacgatattttaactttttttttattttgtcgatcataaacggatgcgcgactcagcatttttggcatttgaatcatgattccgagtaatcagctacaaaaacctaaaaaattgaaaaaaaaataaaacagctagtatttgcctacgcgctgagtcgtaaaaaccgtaaacttagtactaaggctcattagtcgatgtttttggattcgcgtgtgttgcgttacggcaatctgactcatgatatcatgagtccaaatcatggtgtattttcataagacgaaaacacgctggaatcatgatatcatgagcgcacacaacgcaatgccgttagactgagtactatagtactcaggtttagtaccacataaaagtattctgcttttgtgcgaccttagtactatagtactgtaataatcttgtttttggattctgatttctacccgtgcatttctaaagtttgcaaagaaggaagcttcaaaaaacttccaaaagcttatgttacagaagcattattagaacaaaaacctaagactaaagatgggcacgtaaagatttcaaacgtttcttccttcgaagttttttgaaggttttcgaatacaatgtcttccttggaaagcttaagttacagcagtttccgtaccagaacgttggaaagcttatattacgaaacagtaatagggtttctatgcggtggaagtgctgttatagaacctttcgaattcaatgtcgtcaaaggcatggtcttgcaacctttcgaatataatgctttgtcattatcccaaagatacgccaccttcacgacaatattggcgtgtaagcatttataatacatctgggtatcatcgaaatcattagaaaaaatgcggtataagtttcacttgaattcagatcagtgatcgattgttacaccgcacattcgtaaatattatgtagacccatagtagctttagtaatcttttttacgccatattcaaagtgaacttaagtctagtcactagctaacaatgagttcaaatagatccaaatatatgagggtgaaacgtcattgcgatccactgtgaactgcagttgattggccgcaatttcaaaatatgtacacccgagtgatcaagtttatctaggtttatatactcccactttgcagtaacgctaggtgacacttgacgtcaactaaccggcgttaaagttttatacatgtgggctcacttctgcacggctgttcagctgacatccttcattgtcccagtcgttcatacaaacttgcccgtcaagatcaaggaagttggcgcttgatcaatgttctgtttagacgtgccgacaagtcgactgtaggaagtaacagggtcagcaagtatgtttgaacgggcagttctagttccttcaaccgcgaactagttacaagacaaacatttcttttttcttaagtagtattgggcgctgcggtcacctcatttatctttttgaaattgtttcggaaataatgcacgagatgcaataacggttcttggtaaagaaaaaagaattcatcataacccgcgacgccagtggagtaaatagaaaaactttaacaaagcctttattacgtgctctacgttattgccaagaacaacatagtcatgtagaaccttacaaatgatcagaattgatttgatcaattcatttccagctttcaaactgacgatcgcccaatgctaccgtccatcacgattgtatcagtacatcttggaatgtttactagtcttaactaaactagttaagtaaaggtcgaaagtttgactgctagcgggttacgatggcaggtagtgcttattccacgcactggctgtcatgttccctgccagatttacgtagtgttcttttgtaaaggcaacactgctgcactgctccaagtcactccaagcttcatcataaggtgcgtgaccgacagtacaagggacggtctaaatgcatcacaagaaaacatttccgttgtgacgacgtgacgaggttcagtgaggttcgaagtagtgcgagttgaagcaaactgtgaaggattgatattttgaattaaatcaagctctcgcgttgcaggcagctgtaacttgccaccaagtatgatcggtcttccacgctcaacttcgtttgacacttcctaactataaaacttaatttagttcgagagcgcaacgtccgtcgacattgaacggtggttcatactagccagaagggacttcgttccataaaaagtggaatgctcctcgtccgatttccagaaacagtcggttatgcaataaaacaggatcaggttcgatgactcttggcgatatcctgaagcaaggtatttttcaccttacgaggagcaggctaaaggtctttgtcagccaatacgttattttgtcctagtccaagctactgagaaccgctatagtgaattggagtcgttacctatcccccgataaagatatcctctcgcaattcgagggggattaggattagaaaccgtttgctgatatttgcgagatataaaaacttaacctcagcaatggatagggggctatttctataggagagcgttaagctccccctaatcctaatctttggcaaacgactataaacgctctatattttactaataaaatcttcaattcgctaaaagcacttcaattcttgttttctcttctggtttcagttgacccccatatgcgagtgcagcatcacggaccggacttgattattttagaagttaagcgattttcgtgaagttaagaacaaaagagaagaccaaagtcaactgggggtatacgctcacgtcgtagtgcctggcctgacaggaacaggtgcgtacttccttaacttcactatcaataaaaccgtacctcctccagtccatcgaaacaacaataaaatactgcaccgatcagctggaatgtccttgtccacgcatgaaggaattgaagtgatagttattttggcatggaggaggtcaggtagctttgttgttattttatgacgtggctagtcgaccttattctatcccgggaggtccaatcgctacaatttatgcacatttaattccactggagccatgtgcgttcgggcatcttatcaggcgttcgggaattgaaactaagatagggccctccaggttagcgatgttaaatacgtgtaaattaaggtgacctcggtacacgcaagcccgtagaatagtccgcaagcccttaactttgattacgacctcatttgtcattaacgggatgcattcgtacgcagtcagcgtcttatcggcatatatgcggtagccccccgagtgacaattaaaccatggagcaatgctggagtaaacagtaattgccctacgtaagcatgcgtcagtcgcagaatagccgtatatacgccatcggggggctcactgttaatttggtacctcgcgaaaccaatttcacagcggtccaccaactaccgaatgcgatgcatttttatacgacagtggcgttactaggtgcttaacatatcaaaacttggaagcttgctttggttaaagtgtcgccaggtggttgatggcttacgctacgtaaaaatatgctgtcaccgcaatgatccacgaattgtatagttttgaaccttcgaacctttcaaaagcttgcaaagcttccttccaggagcttggaaagcttccttccaggagcttggaaagcttccttccaggagcttggaaagcttccttccagggaaagttttcgaacgtttcgaaggaaggtcctcgaacctttcgaaggaaggtcctcgaacctttcgaaggaaggtcctcgaacctttcgaaggaaggtcgagcttggaaagcttccttccaggagcttggaaagcttccttccagtagcttggaaagcttccttccaggagcttggaaagcttccttccaggagcttggctcgaacctttcgaaggaaggtcctcgaacctttcgaaggaaggtcatcgaacctttcgaaggaaggtcctcgaacctttcgaaggaaggtcctcgaaccaaagcttccttccaggagcttggaaagcttccttccaggagcttggaaagcttccttccaggagcttggaaagcttccttccaggagcttggaaagcttctttcgaaggaaggtcctcgaacctttcgaaggaaggtcctcgaacctttcgaaggaaggtcctcgaacctttcgaaggaaggtcctcgaacctttcgaagcttccaggagcttggaaagcttccttccaggagcttggaaagcttccttccaggagcttggaaagcttccttccaggagcttggaaagcttccttccagggaaggtcctcgaacctttcgaaggaaggtcctcgaacctttcgaaggaaggtcctcgaacctttcgaaggaaggtcctcgaacctttcgaaggaaggtccagcttggaaagcttccttccaggagcttggaaagcttccttccaggagcttggaaagcttccttccaggagcttggaaagcttccttccaggagcttggatcgaacctttcgaaggaaggtcctcgaacctttcgaaggaaggtcctcgaacctttcgaaggaaggtcctcgaacctttcgaaggaaggtcctcgaacctaagcttccttccaggagcttggaaagcttccttccaggagcttggaaagcttccttccaggagcttggaaagcttccttccaggagcttggaaagcttccttcgaaggaaggtcctcgaacctttcgaaggaaggtcctcgaacctttcgaaggaaggtcctcgaacctttcgaaggaaggtcctcgaacctttcgaaggttccaggagtggaaaagattcctgaaaagtacttggagaaattcctcgagttatttcagtaaagattatactggaggaaccaatggtggaatcacttgagaaggtcctcaccttttctaaggacttttcatgaacctctttaaggagctcaataaagtcatttctaatatgacctccttggttaccaccttagtgaactcgcatttcggcagaaatccctggcaaaatcgctatggaaaaatccctgcaaaaaatcctggaataatccttgccggaatctcatgaggaactcctggtaaacgtaaagccgtctttagggaccgttttagcgatacctttttagggacgttttttaggaccttattaggaacggccttagagtactccttgaggaccatttattctttaacaaatttctgtttattttctctacaaagttacagctcctttaccgtgccgattggccagaaatgaccccaaagactcatggggtacgatcttaagaaattgtttaaagacaaataaaagagatgtttcaatgtcgaggaaatggcacggctaaccggtctttactggggtttctgagtaccccatgctagatatttctgccaaatatactgtatgtttgtttctttctgatatgcttttaagctcaattttctttggaatggtggagatttgttttggcctccaatatactataaagacggtttatatgacatacaaacaaagaaagactatacgaaaattcgagttaaaagaaaccttaccacctctaaacaaaaccggaggttatatgatgctagctcgtagttcgtacctgaagtcaactcctcaattcctaaatgctacaataatatataaaattttaggaaataactgcaaaatattctgaaggccacgatcgagcatcaagcatggacttcagttgaggagttaaggatttacgatgttattatatattttaaaatcctttattgacgttttataagacttccggatgtcttgatctatcttgatgtatctaatatgtaatcccagaagcattctagttttttctgataatctgtgaaataagttgtttttacgaactttgactttacagaactagatagaactacatagattatacattagggtcttcgtaagatcaaaaaagactattagacactttattcaacaaaaatgcttgaaactgaattcgggatttgaggtacaagctttcaaatatattggaggttctgcgatattaacttcaatgaattattggaaattagaaatcgtcttgtgcatacgggttaagccctaaactccatgttcgaaagtttatataacctccaagacgctataattgaagttacttaataacctttaatctttagcagaacacgtatgcccaaaatcgattttagtctctggtagatttcgagagggaatgtctgaagaaattttctgacctacatgtgaagtattgtctgtcaaattcaaaatattttctgtttagctaaaatcagagaccatctaaagctctcccttacagacttctttaaaagactggatgtacacttcataacagacagtttaagttttataaaagacaaggaaattaaaattttttggggaaaactcgaaactccttggatatccaaggaaacaaaaaaaaaagaaatatctgaagaagtgcatcgtcctttttcctttcctttaattttaaaaaaccccttttgagctttgaggaacctataggttcctttgttttttttttctttatagacttcttcacgtagcaggaaaaaggaaaattattgttttaattaactaatagttctgctagaaaggtttttggcagaaccccaaaatgatattcaaagcaactaacagctcgatttcccctcgtttcttaataacaaaattaattgattatcaagacgatctttccaaaaaccgtcttggggttttactataagtttcgttgattgtcgagctaaaggggagcaaagcaatttcagacgacgaacttgtcaaacgatctcaatggctcctggagaagctgcgatacccctgggagatgatgcccctgatgtacgtgatactgaaagggttaaagtctgctgcttgaacagtttgctagagttaccgaggacctcttcgacgctatggggaccctctactacggggactacatgcactatgactttcccgccgacggagacgtcaataaagcgcgccaacggattgacgaaggtatgggggttcttaccggttgggactgtttccgaggtatcgatcgggtgtcactcgcggctgcctctgcagttatttcgcgcggttgcctaactgcttccatacccccaagaatggccaaccctgacaaaggctccatagctagcccacagtgagacttcctgggtgctcccattttgtaactgctaacgcttattattgagtttcaggacatctgggatcttcggtcgacggagtctattcccaacagtgcccttgaaggacccacgagggtaaaacattgacgattgcgaataataactcaaagtcctgtagaccctagaagccagctgcctcagataagggttgtcacgggaggatcaaacactgccatcatgcagtttccgtagcctgttgggctacgctccccgacttgacatcccccattcttatcaaacaacaactcaaggcctgagacctagtttgtgacggtagtacgtcaaaggcatcggacaacccgatgcgaggggctgaactgtagggggtaagaatagtttgttgttgagttccggactctcaacgagtggtggaatttgcgcacgaagtcattggtttgtcctggtaaaagttaaaagggttaactggagggttaattgacacggtttcaactgatggccgttgctcaccaccttaaacgcgtgcttcagtaaccaaacaggaccattttcaattttcccaattgacctcccaattaactgtgccaaagttgactaccggttattgacacacggatgaaagacttgcacgcttgaccttctgtctgtactaataaaagttacgttggctgggttttggggtcataatggccccaaaatcgaataactgtgtgcctactttctgaacgtgcgaactggaagacagacatgattattttcaatgcaaccgacccaaaaccccagtattaccggggttttagcaatcgtcataacttcttgaaatacaactcacgtttaagaccattcaagagtattagatcatcgtctataatagcagatttgaaatttacttcacatttcgttagcagtattgaagaactttatgttgagtgcaaattctggtaagttctcataatctagtagcagatattatcgtctaaactttaaatgaagtgtaaagcgtattgcagtgccccttgcttccacaatggaattagttaaagtttcgagagcattgtcaatatcaagtgttgttagcaaacaaatgctaacatcaagattcataacgtcacggggaacgaaggtgttaccttaatcaatttcaaagctctcgtaacagttatagttcacaacaatcgtttgtttacgattgtagttctaaactatcgatgtttgattcacatgtattccaatcagctcgtaaaaaatggaaagtggagctgatagggttgagaatcgcttcatgggataattggaaacagtgatagctacaaactaagtgtacataaggttagtcgagcattttttacctttcacctcgactatcccaactcttagcgaagtaccctattaacctttgtcggacatgatcagaatgaaaatcagcgtgagtaaccagttgactacaaagatgactagagtcggttaagaaaaattcaagtagggctatcaggttattgaacctgtactagtcttacttttagtcgcactcattggtcaactgatgtttctactgatctcagccaattctttttaagttcatcccgatagtccaataacttttgaaaaatatcccgaagggccctcatcaattaaaattttgcctttggaaatgtttggcattcaagtagcaaattttaacatactgcgattcgatttccgaactttttatagggcttcccgggagtagttaattttaaaacggaaacctttacaaaccgtaagttcatcgtttaaaattgtatgacgctaagctaaaggccaagttagtttgaaacaaattaacttgctacccagtgcattaaaaaggcaagtaggcagctttggaagtataaacttagctgtgttttaacagaagcactgttcaatcaaactttgtttaattgaacgatgggtcacgtaatttttccgttcatccgtcgaaaccttcatatttgaatcgacacaaaattgtcttcgtgacgcaagtttcaaaaattttggtttcgaatgacaaaaaaagttgatgttatatacgcctattgaatgatgattccagttgatcatttcgacaaacaaaaaagcgttcaaagtttttaaaaccaaagcttactgtttttttcaactacaatatatgcggataacttactactaaggtcaactagtaaagctgtttgttttttgaatctcttttgatttcagatccaggattcaaataacattccgttatcagataaagggttaatgccacaatcgtgtggtccattatccccggaaacttcacttagagaaaactaaagtctaggtcctaagtttattgtaaggcaatagtctatttcccaattacggtgttagcacaccaggtaataggggcctttgaagtcaccgtcacactcgatccagatctgatgtgatctctgccgtcgggcgcctcagaagcgaaaaccacattcgcccgcgctctccggaattatgtcgtaaaagtggcagtgtgagctaggtctagactacactagagacggcagcccgcggagtcttcgcttttggtgtaagcgggcgcgagaggccttaatacagcatttttaaaactttacaaccataattattcagaacttcgacgactgcgcgatgacttggccgcggtgtgcctgcttgggatggacctccgagcactgaaagcagtattttgaaatgttggtattaataagtcttgaagctgctgacgcgctactgaaccggcgccacacggacgaaccctacctggaggctcgtgactttcgtcaggtttgtacaaattgaatgggctatttgaaattaattgggctgcgataacttcaaagtgtgacatcaaaatggtgtgagttttttactgcacaaattccaccaaacatgtttaacttacccgataaactttaattaacccgacgctattgaagtttcacactgtagttttaccacactcaaaaaatgacgtgtttaaggtagttatttcctacttcatatcaatcggagctccaggagtgaagatccaaattaccaagcttggccatttcgtatgaaaaacggcaaaatgatcttttttttcaataaaggatgaagtatagttagcctcgaggtcctcacttctaggtttaatggttcgaaccggtaaagcatactttttgccgttttactagaaaaaaacgccagtcactgtatctcatgatccagatgagataaaaaagttcgagtcttcgacaaagttgttttggaagtcatggacattcttaagcaaacaacttaggcggtcagtgacatagagtactaggtctactctattttttcaagctcagaagctgtttcaacaaaaccttcagtacctgtaagaattcgtttgttgaatcttttgccactaggtggcgccagtaagcatattcgtcatcaaacgtcaacatcccaccgcaaaatcgctagtgtttggaggggattttaacctccaaattgaaaacggtgatccaccgcggtcattcgtataagcagtagtttgcagttgtagggtggcgttttagcgatcacaaacctcccctaaaattggaggtttaacccaaataacctccaaatcatcacctccaagttagttctaatacactccgttatatgaaatatggtggtgcgtcgatcgtcgcaagtttatcgttaaacagggtttattggaggtttagtagtggaggttcaatcaagattatgtgaggcaatatactttataccaccacgcagctagcagcgttcaaatagcaatttgtctcaataaaatgagcattttatatcgtgatacatatgagaagatagaggtttcaattaaaacaaatccacatggtgtcgctaataaaattgtgcattttaaagttattttactcgtaaaatatagcactatgtatactcttctatctccaaagttaattttgtttaggtgtaccacagcgattattttaacacgtaaaattgcgagttatatcctctgatcaagataaaatagaaaattcgatttttgaatattcaattataagagcctgaataactacaacatgtagtgaatcgaaactgcgctcaatataggagactagttctattttatcttttaagctaaaaacttataagttaatattctcggacttattgatgttgtacatcacttagctttgacatttatgacggtttgtgaaggttacacgtcctaagcatttggattcaagaaaagcaagagatatgacgaatgtaaactttatcgtatcaatgaagtaacttaaatactgccaaacacttccaatgtgcaggattcgtaaacctaagttcttttcgttctctatactgcttacatttgaaatagcatagttacttcattgaagcgtccagaacagtacaaaccaacatcgtaccgtcgtattccactccggtcgttgcaatatctctaggtccaccgaaaaacactcatgaccaagatcgttcgcaggtcttgtcatgtttggttgtagcatggcagcataaggtgaggccagcaacgttatagagatccaggtggctttttgtgagtactggttctagcagtcgtcgatcttggtccaccgaaacaccgatgtccatatcgtttcgtcgaacttggaccaacgattcatgcaactgatgacaacgcggcccccgggtcgtcagcagctagaaccaggtggctttgtggctacaggtatagcaaagcagcttgaacctggttgctaagtacgttgactactgttgcgccgggggcccagcaaccaatatccgaaaaatccaactgttcttctctgcctcgcaggtcaagccgtggtcaatgaatactcacgattgcacaatctgaacatgttcgacggtgttggttataggctttttaggttgacaagaagagacggagcgtccagttcggcaccagttacttatgagtgctaacgtgttagacttgtacaagctgccacaagagttgcgcagtacgacgcgccagtccggatgatagactttttacacgatcagcacgacccactgcgctgcggcaaaggtcgaaccgaaacaagaataatctcaacgcgtcatgctgcgcggtcaggcctactatctgaaaaatgtgctagtcgtgctgggtgacgcgacgccgtttccagcttggctttgttcttattaccacgaagatcagatcgattcgacggaagaagcaatcgaatgcaaagaagaatcggaacgaagaaaactctaaagcatcgcatatttacaaagcataactggtgcttctagtctagctaagctgccttcttcgttagcttacgtttcttcttagccttgcttcttttgagatttcgtagcgtataaatgtttcgtattgggaaaacccgcaagttcaaactagtgattagtgtaagatgaagcaaagcagaaatgtagtatctagatttttcgacgttagtttacaaagataaaaaatgccttttgggcgttcaagtttgatcactaatcacattctacttcgtttcgtctttacatcatagatctaaaaagctgcaatcaaatgtttctattttttacaggttggacatacaatcgtgggtattcgtctgagttcgtcacaactgcaccggaaactgtgaaacagaatagagccaacctgtgcgcggagaatgttgagtccaacctgtatgttagcacccataagcagactcaagcagtgttgacgtggcctttgacactttgtcttatctcggttggacacgcgcctcttacaactcgtcattataagcttccttagcatccacgggtgaaagtcgatcgacggaagcctgcaagactctgtcgatgggctttcgtcctagaagaataagattaaaccagtaatattcgaaggaatcgtaggtgcccactttcagctagctgccttcggacgttctgagacagctacccgaaagcaggatcttcttattctaatttgctgaaatgtattctcccgtggaatggtttcatttgagtaattctgtatcttctccttcccaattccacgaacgcgacgaactctaatacaaacaacataagactttacataagagggcaccttaccaaagtaaactcattaagacatagaagaggaagggttaaggtgcttgcgctgcttgagattatgtttgttgtatttgaccacagtgcaaatgctgtttaacgataatagcgacatgcagccattctggggctaccacgtgtagctctacttgtgagacagcgttcctaaagagtgactggtgtcacgtttacgacaaattgctattatcgctgtacgtcggtaagaccccgatggtgcacatcgagatgaacactctgtcgcaaggatttctcactgaaagtgcaaacaagtgatgaaaccaatagtgcaaagcaagtttagagggaaaatttaaaaaatgcaaaacagcagtagtacttaacttttaagattgtactttcacgtttgttcactactttggttatcacgtttcgttcaaatctcccttttaaattttttacgttttgtcgtcatcatgaattgaaaattctaacagtttcgaaagccgaagtgtgttccatctgccaccggaaaaaaacgacgacagcagaatcatcaacaagcaacatccatccgaaaaaatccgggaaaccggcaaagctttcggcttcacacaaggtagacggtggcctttttttgctgctgtcgtcttagtagttgttcgttgtaggtaggcttttttaggccctttggccatcttcaaccaaccatcctacaatctacaaaccagagattatatctcttcaatcgtttccgacatcggtcggtttcggtgcccaaaatgatctgataaactagaagttggttggtaggatgttagatgtttggtctctaatatagagaagttagcaaaggctgtagccagccaaagccacgggttttactagactatttgacttatctctctgtagcttgcatgccattgcgagcgtattttggtagctggccgttgccaaacggctccgacaggtactgctattggaggttgtgcacgatgaatagagagacatcgaacgtacggtaacgctcgcataaaaccatcgaccggcaacggtttgccgaggctgtccatgacgataacctccaacacgtgctccacgttgagtttgccttttgagttggagagtgtgtcttttcgtcatatatttggccttttcaagggtgattttcaggctgcgtaaagattgtatagtttggtgcaactcaaacggaaaactcaacctctcacacagaaaagcagtatataaaccggaaaagttcccactaaaagtccgacgcatttctaacatatcaaaaaccagctaaaacatattgatgacaagttctatttcagcaccacaaacaagcctgttaatgtctctcaccgcaaccattgttctgcgcgcgttataatcattggtcgattttgtataactactgttcaagataaagtcgtggtgtttgttcggacaattacagagagtggcgttggtaacaagacgcgcgcaatattagtgcatagaagtttattttctttgggatgattcaaatattacgtgacgcaaagtttgccaattttagaacccctccctcctccacgtaacggcttttgtgtgcgtatcttcaaataaaagaaaccctactaagtttataatgcactgcgtttcaaacggttaaaatcttggggagggaggaggtgcattgccgaaaacacacaaaaatttaaattttgtgtatagaccgtagcatttcggaagaccccctcccttactctgttgagttacgtaaaatttcaacgatccttttgtagttctgatttttaaatttaaaacacatatctggcatcgtaaagccttctgggggagggaatgagacaactcaatgcattttaaagttgctaggaaaacatcaagactattttatatcagcgtgcagtgttatgaagatatccacagtataaaatattattttattttaaattctatgctgattatcaatgtgttactagtggctttttaaaatatagtcgcacgtcacaatacttctataggtgtcatattttataataaaataaaatttaagatacgactaatagttacacaatgatcaccgaaaacatactcatgttgcgagctcgatttggcgcacggggtcatctacacctgatacctttagggtcgttgggggaccacttagcgtgcacgtacggacattcagtatgagtacaacgctcgagctaaaccgcgtgccccagtagatgtggactatggaaatcccagcaaccccctggtgaatcgcacgtgcatgcctgtaagtaaatgttgttcaaatttttttcttaccaagacgagcactttacaatgacaaactctggctctgctctggctctgctctggctctgctctggctctgctcttttacaacaagtttaaaaaaagaatggttctgctcgtgaaatgttactgtttgagaccgagacgagaccgagacgagaccgagacgagaccgagacgagaggctctgctctggctctgctctggctctgctctggctctgctctggctctgctctggctctgctctggctctgctctggctctgctctggctctgctctgccgagacgagaccgagacgagaccgagacgagaccgagacgagaccgagacgagaccgagacgagaccgagacgagaccgagacgagaccgagacgagacgctctgctctggctctgctctggctctgctctggctctgctctggctctgctctggctctgctctggctctgctctggctctgctctggctctgctctggcgagacgagaccgagacgagaccgagacgagaccgagacgagaccgagacgagaccgagacgagaccgagacgagaccgagacgagaccgagacgagaccctctgctctgcaaaatgctctggattaatttattgctcacactcttttgctgttggaccactattcatttcaaatcttcaatatgttcctattacccccagagacgagacgttttacgagacctaattaaataacgagtgtgagaaaacgacaacctggtgataagtaaagtttagaagttatacaaggataatgggggtaacacggtccacacggatcgatttcaactaactccactctcgtatgcatattttgtgtataaattttgaataatcgaaaagggttgctgcaaatgttaatttgtgccaggtgtgcctagctaaagttgattgaggtgagagcatacgtataaaacacatatttaaaacttattagcttttcccaacgacgtttacaattaattttttccctctaccccctcactctgtcgttggcgttggaaaaaaatcaccactgcatacaaaacactcattggttgggtggaaggacggtttagcagataaaaaagggagatgggggagtgagacagcaaccgcaacctttttttagtggtgacgtatgttttgtgagtaaccaacccaccttcctgccaaatcgtctgttgctaaattttccatatcacgctgattgatttgtgattaaaaataaatataaatagaaaatgaataattcccacatgtgtttcggtattaggcaccggcaacgatttaaaaggtatagtgcgactaactaaacactaatttttatttatatttatcttttacttattaagggtgtacacaaagccataatccgtggcccatggggcggcgaagtgcagacggttctagttctcattatttggcatcgattggcggtcaaactacaacctccatggagaaacaggccccatccgtacttgtaccccgccgcttcacgtctgccaagatcaagagtaataaaccgtagctaaccgccagtttgatgttggaggtacctctttgtccggggtaggcatgaaagttattaataaataacaatgatttgaatttgaatcattcatgctgcggcgtggctgatttcggtgaattgttgttctcttagagaaagagggggatttgtcaataattatttattgttactaaacttaaacttagtaagtacgacgccgcaccgactaaagccacttaacaacaagagaatctctttctccccctaaacaatttggacgagtaaataacattgaatattacactttatgactaatcaccagtaatgaaacaacacgggtgatgatttcaaaagcttcattctaaatgcattaaacctgctcatttattgtaacttataatgtgaaatactgattagtggtcattactttgttgtgcccactactaaagttttcgaagtaagatttacgttggttcacttttggtggcagatttaaaactcttatcttcctcttttcttcaacaggtttcacgccatcaaagacgcttggcagccgcttccatttgcgtaaccaagtgaaaaccaccgtctaaattttgagaatagaaggagaaaagaagttgtccaaagtgcggtagtttctgcgaaccgtcggcgaaggtaaacgcatgcaaacgtatgttaaccttaggttttaatgttaaaagtatcaccaaaaatcaagtcccaagacttctgcaagaatggtttatgctgaatttattcgaaatcgtttgcatacaattggaatccaaaattacaattttcatagtggtttttagttcagggttctgaagacgttcttaccaaatacgacttaaataagctttaggttttattttcatcgaaacatgtgtgatgtaggctactattttggtaaaaccgttggcaacgactgtatttaaactcacaaaatttgaaccaaacttatccaaaataaaagtagctttgtacacactacatccgatgataaaaccattttggcaaccgttgctgacataaatttgagtgttttaaacttggtttgaataaattgtaacttttaattgagtaaacataggcgaaagagagtgattcaaatgggattcggaatcgaacggttcttctaagtaagacaaacgaaaaaaacaattaacattgaaaattaactcatttgtatccgctttctctcactaagtttaccctaagccttagcttgccaagaagattcattctgtttgctttttttgttccaaacgagtcaaagctgcaaaaacttcaagtttgaactgtgatatcaatgaaattaaatacgaactatgtatcaagattacagtaaaatttaaagaagaggtttgctcagtttcgacgtttttgaagttcaaacttgacactatagttactttaatttatgcttgatacatagttctaatgtcattttaaatttcttctctttcaacgcatgaaacaggagggtggcaaccgaaaagtgactgaatcaattgcgggttatcattcgagatatccaggggttgaattgtgagaaaacttcgaaagttgcgtactttgtcctcccaccgttggcttttcactgacttagttaacgcccaatagtaagctctataggtccccaacttaacactcttttgaagttcttcttcttattcttggcaatacgtcctcactgggatagagtctgcttcctaacttcatgttcaatgaccacttccacagttattaactgagagctttaagaagaagaataagaaccgttatgcaggagtgaccctatctcagacgaaggattgaagtacaagttactggtgaaggtgtcaataattgactctcgaaactttgccaaagttgccattttcgcattcgtatatcgtgtggcagcagtgttgtgaaaaactcaatttctcataactaacgcttgagatttttcatgcgtggaaacggtttcaacggtaaaagcgtaagcatatagcacaccgtcgtcacaacactttttgagttaaagagtattgattgcgaactctaaaaagtacgcacagttgtcaatcacgcaactcagcagtcaaaattttccacagtatacttacacacggcaataatttcttgctagtctggtaaaattatagtaatcttttcttcaacagttagtgcgttgagtcgtcagttttaaaaggtgtcatatgaatgtgtgccgttattaaagaacgatcagaccattttaatatcattagaaaagaaacgtaaacaacaaaattcgggtttcaagagtttttgacgggagcaagcaaaataggatttagaattttgcatgagacgaagtttgaaaattttattgtcttgcatttgttgttttaagcccaaagttctcaaaaactgccctcgttcgttttatcctaaatcttaaaacgtactctgcttcaaacttttaaaataacagaaatttagtatcggttcaatcgaattttcgaacacaattgtaggctctatataaactacatttattcccttattttgccagatacaatactcgcataacttttaaatcatagccaagttagcttaaaagcttgtgttaacatccgagatatatttgatgtaaataagggaataaaacggtctatgttatgagcgtattgatgagatctcgcctaaaaagccattggtaaccgagtgtgtagctctttgtttctaagccaattaatggacctggatgaaaactatcatcactgggaaatagactctagagcggatttttcggtaaccattggctcacacatcgagaaacaaagattcggttaattacctggacctacttttgatagtagtgaccctttatcaggaggaacttgtctttatcgtagcattgttaaataacgtgtaaacccatttgtttcctcggtagctgcaagctacacactcgattaccaatggcttttatcctccttgaacagaaatagcatcgtaacaatttattgcacatttgggtaaacaaaggagccatcgacgttcgatgtgtgagctaatggttaccgaaaatgggcgagatcacaagttatgcgagaatacttcccgaaatcaccaccttttacccttttaaataacgaaattactacaaacttcgttacccgctctagtgttcaatacgctcttatgaagggctttagtggtggaaaatgggaaaatttattgctttaatgatgtttgaagcaat

SEQ ID NO: 44 - DNA, 3399 bp, Cydia pomonella
  misc_feature (1179)..(1184): n is a, c, g, or t
catcagacgggcccaggctcaggatgaagctagagcgcgggcggcggacgcagggctccaccctcccgggatcgagctagatcggcctgagccgccagtggtgaaagcgccgaggagtcccgtgatcccgccgccgccgccgcgctccatgggatcggcgagctgcgactccgttccgggatcgcccggggtatcgccgtatgcgccgaacccgccgtccgctccgcctccgccgatgccgccgctcccgcctccgcaaccagtggccctggactccctggtagaaaactgccacaagctgctggaaaaattccactacagttgggagatgatgccgctcgtgctggtcatcctcaactacgccggctccgacctggaggaggcctcgcggaagattgacgaaggtaagtttaaatttaagtacataacaatgcttacagacgaattgaaagggaatgtgactcggctaatccaccaggatataattttgtagagtgcgctaaagaattctagcaacggacgctgttattctgccaccgccgttgatgccgccgtcttctgatagtgatactttaagatccgtatactacgctcacttccattcacttatgtcgtacggagtattaatatgggtaaactcgcggacacgaaacgattacgaaaacgcagagtacttagattggagcaaagcccagggattcgccgagacttttttgttacggaagattgatgaaggtaagcaagttgggactgtggcgagttgacacatgaaacaagtcaaggtcacagctggagttccattaaagctggatgctaccgctagtcatcctgaggccggctccgacttcgtgcaatgaggtattaagctgctggaattgaatggaatatagtggtgaaacactactactaggtttaagcgtttagttatatggttgttttcttatttttaatttttaaatgctctgctaagctaaaacggcwaatgtctatttttgattataaagacttatataaaacaacttgtttagcttctttkacgtctttttgttaagctgtgccctggttttaaawkgggcgaacacytcacgaataagacgtaattttaaaaagaaaatagatatcggccctcttggttcgcatttatacatatgtattgctgcccgtgcgaatgttggggannnnnnaaacagtacccctagtgtaartaaattcgatttcgaaacgtgacgtacgcgtttgcgtttagtctcmwtttgtattggatttagaaagagcgcgccaagcgggacgttttggaaactcaaaatcctatacaaaatgagacttaacgcaaasgcgtttcgtcacgttatgatgtcgatcaaatttacactaggggtacagaggtattgcagtaactgtacaaatactaaactaaattaataaattagctaaatctaaaatatacccttcaggcattgtactaaggatgctggcggaattacttgtgcgaggaagccgccagcttttcggtcaccatttacgagtacgtataccaaacgcttcgttgctgcaaaaaagtttcaacgccaaatggtacaaaatgctttatattgttctctatatattatattaacacatcgttattttaacctaggtcttagttatgtacaaggttacataaaatagatgttcctagtccattcctccgtgtatgttgtgtctattataaagcaaggctgcattttgtaatcagtcaatttcaatataaaaaagttgcatcgtttttttttactkttcgacaattaaattcaagtagcaaaaaataacccaccttaatttgtcatggtcataatgaaacaatgacaargttttttttatcgcccgatacatgtacgtgttctccaaaatgcagtctccgcgccgccaagcgaacgttcaaactgtgcgatttccgttgtccccaggcaaaatgatcatcaacgattacgccaggaagcataatctgaacatcttcgacgggctcgagctgaggaactcgacacgccactccatttcggatggcgatgaaaaacgcccaccgcaacctaagcaagtctcaaagtaaggttccatttaaatcatctcaaaaccgttagaaacactcaaaaagaaaccaaaattctgttcggaaaccgacctttgttttttacacacacttagaccgaatttgcaaattttaaccccttattcctaaaactagcaatggtaagctcggctgaatttcacatacaaacggagtttcgttctcattataaaactgcgtgttggattgtaatggaactttgcacatacaatgacatgaggtatgtctagggctgaaattagtttatacttggtatctgaggctacataaactaattacagccttagacttggaggatttaacaactggaaacaccttgtctgtaattctctgtacaacgattttacgggggaggagcaaatatgtcagttaaacgtcagtccaaacaatacatatgactattggccgtggtatttcgacggaggggtaataagctcttaaaggcgactccgatatgcctaatcctattgttagtacaaagtttcagagcaatttagctagtcgttttaaaatgagagcgtaactacgttagcttgctcttcttcctcctgctcttatcccacgttatgtggggtcggcacaacatgttcctctcttctcactcctttctttctcatatcctctttcacacaatccatccatcgtttacttacaaccgagcttgctggggaccgttaaggcgccgcgagttcaggttcttctctcactctcactctcactggtgtgagcggagcgagacagcgttttattttcgccttatcgaggttccactgtattataaataacttacatttataaagacgctgtaatcgataagaagttgagtcacgcttacgtcgcttacgtactacgtatagtaacgtagcctgccgtttacaaacaatgtacggagctacaacgttgcaagttcggtccccacacaacacaatgtgtcataacacattaacaacattgttacacacccacacatacaaatttgctaagttgataaaagagtggtgtgtccgacgaatcagaacatcactaacccagtcgtgatttcatttccacagtgaccggacgaaggtggagaagttcgaaatttaaaaaaagtgaccacattttatttaatagtgatgtgcaagtgatactatttttattttgtttttcttttgtaggaaaatgctgagcgaaataaataattttagtggtgtgctatcgtcatcgatgaagttgttttgcgaatgatactatgttcttcaagtgctgtgttttgtggactgtggggtgactgttcctgtaaataagcttcgttg

SEQ ID NO: 45 - DNA, 996 bp, Cydia pomonella
catcagacgggcccaggctcaggatgaagctagagcgcgggcggcggacgcagggctccaccctcccgggatcgagctagatcggcctgagccgccagtggtgaaagcgccgaggagtcccgtgatcccgccgccgccgccgcgctccatgggatcggcgagctgcgactccgttccgggatcgcccggggtatcgccgtatgcgccgcacccgccgtccgctccgcctccgccgatgccgccgctcccgcctccgcaaccagtggccttggactccctggtagaaaactgccacaagctgctggaaaaattccactacagttgggagatgatgccgctcgtgctggtcatcctcaactacgccggctccgacctggaggaggcctcgcggaagattgacgaagcctcctgggtggtgcaccagtggcggctgtacgagcgctcactgtgctcgctgctggagctgcaagcgcgcaaagagtcgttttgctgctcgccgcgctatgtgctgtcgcgcgagtacgcgccgcacctgcccgtgccgctcatgcgctcgccgccgccagcgcacttgtagccccacaccgcgccgcgacagacggcgcacgagcccactgagccatctacttcggccaaacccgagtaggcccgaggccgacccgagcccgacccgagaggacccgagtgggctattccggactttacctagttttatatgtgctatacgtgttacaacacgcatatttgtatattatcacggacattaagttggagagcggttaccttatcttgttaacccggtccttgaagtaattattcccagatatattaagaaaaccagtgaatactttgcctgatgtataattaacagttgttaagcaaccatgagaattatggtatttcttgtggacatgttgcagctagaaatttcatatcatcggtgataaaatttaaccacactgtggttggcggaaaaccacattgtttgtaatattg

SEQ ID NO: 46 - DNA, 6751 bp, artificial
  Sequence of pLA3435-Bombyx mori-dsx construct/plasmid.
ggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtccgtcccctcggagacgcttgtggagaactgtcacagactcctcgagaagttccattactcgtgggagatgatgccgcttgtgctcgtcatcatgaactacgcccgcagcgacttggatgaggcttcaaggaaaatctacgaaggtaccgaatgtgtaaatacgagtgtagcgttgattagaaaacggacattgttcgtgagtttannnnnnggtctctctggccagcaagacatttgaaacactgtaaaaaaattcattgaaaaaaaagaacactgtaatgaaaatattctgaatgcttaatctggtatttcagggattaaactgattgtgatgaaaagtgattaaactattttctttaagtaccaaattaaccgaacaggtttgggtctttcctttcagtaacaaacaaaatctatcgaaggtaagaaataaacaacaggatattttcttttactaaaaatcaataaggagactgcactatttcaatgttcaacttcctttatcgaatgcatgaaaaatttaattgtctaaaaatctaaattactaattaacgcaaaggaacctttgcctaaaaaaaaaaataagctattaaacgaatgcctaaaatacgtaacagtgttgccagttgtaaaaattgcgaatccgagaagtgcagtttcctgaaatgcccagcgatacgaatttcctatgttagagtcttgtccgcagggaagatgatcgtcgacgagtacgcgaggaagcacaacttgaacgtgttcgacggactagaactaaggaactcgacacgccaggcgcgccggatccggccggccgaaaatgctggaaattaataatataagtggtgtactgtcttcgtcaatgaagttattttgcgaatgatacttagttttacaagtgccgtggtgtgtgttgacacttgctgtgcgatgctgtgcgaatttcaacggaaatatttgttgtcgtaacattggatctatgggtaagtttagtataataactttactctgttcacattagtgaaacatacatttgtaaaatttgtgttttactaatgtgaaatttatttttggaaattcacgttaacactattgaataaaaaaaaatcgataatgtaatttaaaaaaaatacaaaaatatagttttcgcttattgttagaaagaaaattttacatacgccattttgaataattccttccgggtacattgggccctaaaccagcgatcggggaacttttttaattattaccctaaaatatttttatgtaagttgatattaccgatggcgaagaacaacaaaaaaaaaaacgaaatcgcttctttttagcatctttcatattatagaccccacgataattttaaatcacaacgattataaagaagtttcacttcaatatatactttttactcacaaaagtttcatttttaccccatttgggataatttagcccggttccccccccgaccgctggcctaaacgtatcaccgacaatagctaaaataacaaggtacgttcgatttgccgagctgaactaacattacacagctttgcattattcatatgtacattgcgactgaaacgtccggaccgttacaggttattggatgatgcatcaatggcgattgcagcagtattcgttgtgctacggagcgctggagttgtcggcgcgcaaggatgtggccgcgctatgttgcctccgagatacgtgctggcgcccgaggtcccgccgcgtctggtgcccctccagctgatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaagtgcgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatagcgatgc

SEQ ID NO: 47 - DNA, 8183 bp, artificial
  Sequence of pLA3359-Anopheles gambiae dsx construct
ccggtgctgctgttgctgatgctacgatcctcgacagtgattggaaacgcctggagatggtgggaaaaaatcaaacacaaaaacggtcctaatgaacatcgtgtgttctcattcgctgccacgattgacaccttcgataagacgcacataatgagctaaaggagaggggacagggtcttgtctttgccacgagcgataagattgcaatcactcgtgagcgtgtgctgctgggctgaagaagaaacactttccacagcagtaggtgggaagtgggattgtggaacgtggcattgaaaagaacctattttctaaagcccgagagcccgttctcgaactggaaaacgagatgcagaagttttttattgtcccccgccaggaaaacaaatgtatttaatgctttctctgccttttccgccccgtttcagacgacgagctagtgaagcgagcccaatggctgttggagaaactcggctacccgtgggagatgatgcccctgatgtacgtcatactgaagagcgccgatggcgatgtacaaaaagcacaccagcggatcgacgaaggtaagctggcgatgatggtgtcgttcgacatcactttcatcaccgtgtcagacatctactgtgcctagcaccggtccagtggtcacagggtgtagcaaaaacgtgttcttttttgcgagagactctacctcatgatgcagctgttaaggaaaggtttcagatgaagacaatttttcctaggataatatgatcttaagttacctgcgtatgagtgtttaacattgtcgtctcaactccaaggaatgttttaaccgtctagggctagtttatttatactgttctcattgaaatgtcgttaaatccaacatgttaagttagctagctcagacacgagaagttaggagtatctgcatcttgaaggtagcggcatatggtgttatgccacgttcactgacttcaaaattcgatacaaaaaaaaacaaaatcaaaaacaaaattgtgaattccgtcagccagcagcagtgaccttcaaagccttacctttccattcatttatgtttaacacaggtcaagcggtggtcaacgaatactcacgattgcataatctgaacatgtttgatggcgtggagttgcgcaataccacccgtcagagtggatgataaactttccgcaccactgtaactgtccgtatctttgtatgtgggtgtgtgtatgtgtgtttggtgaaacgaattcaattgttctgtgctattttaaatcaagccgcgtgcgcaactgatgccgataagttcaaactagtgtttaaggagtggagagagagagccgcaccacggtacagaagggcagcagaatgggtcggcagcctagctgcactggtgcggtgcgtccggcgtctcggggggagggcggggaaattctagtgttaaatcggagcagcaaaaacaaaacagtggtcgtcccgttcaagaaacggcctgtacacacacagaaaacactgcagcatgtttgtacatagtagatcctagagcaggtggtcgttgctcctcgaacgctctggacgcacggcttcgcgcgtacttgcgtagcgttccaccgatcgtgggtattcgtactgccacaagcccgctttctcccatgcaatctctgcaaccaaaccaacaaacaacaacaaaataccaatcgacacaatgaatcacaccccttttgtatcatctgtatattcttgttctttgcgttcttttccatgtggcccacgccccggcgggtacgtaattgcgtcgaaaaccccgaaaaccccggcacatacagtgtacatacggtttgaggacaactttgacctgcagcccttctggggctgccacgtgtagctatacttgtgagatcgggcgccgacggtgtaaagcgcgaatggccgccacacagtgtgtccactccaacactacccctctggaactaccccgtccagggatgcaccggctcggctcatgcccctgcaaaacagtccgggctccactgtagtagctccggcgttgctctgagagaaggatgcccttcgaagtgtcgaaagcgtgcattgggcgttcaagtgtgtgtctgtgttaggtttagcgagaaacagcagcagttgcgtgtgctgaaaagcgaaggagtaatagagtgcataatgaaaatgaaaatgaaaatgaagcaaaagtagaaggcggaggagagcaacctgtgttccactagtagcgaatagtttagtctagtttcgtcaccaatcaaccttccaaccatcgttcaaccaatacctgagtcaacatcgtcatcgttatcgtgccacaactttattaaaaatgaaccttgtccgcgccaccgtagggtgatctgaggcgacctttcttacgggcgcgactcacatgccatcgtcaccttctccaatcaaaaccaacagcctgtaccgatggtgtgcaattgtgcgtgcgtgtgtgttattagcaaaaaaagagaaagagacggcgagagagagatagatcgagatcgagagtacaaaagagcagtagaaatgttcgttgtttgttttccgtaacacagttgtttagccaaaatgggaatttccaataatcccgggggcggggaaatgcgggaatactgcgtacacacatacatcaatcaaaaagaaaaatccttgcgctacatcactaccgtttgcgcggtgctgatctagagcagaccactttccacgccattctacaatcaatcaatctgtgcagaaggtatggtaagacggcctttgagcgagtcacggtcgccaccataacgccgtccgacgagggctgaatgcgaactttgctaatcgattttccgctttctttttatcccacccccctttctctctctctcttttgcaccgccccttgtaacccccaaaaaggtaaacgacacattaagacctacgaagcgctggtgaagtcatcgctcgatccgaacagcgaccggctgacggaagacgacgacgaggacgagaacatctcggtgacccgcaccaactccaccattcggtcgaggtccagctcgctgtcgcggtcccggtcctgctcgcgccaggccgaaactccccgggccgacgatcgggccctgaaccttgacaccaaatagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaagtgcgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatagcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaa

SEQ ID NO: 48 - DNA, 7342 bp, artificial
  Sequence of pLA3433-Agdsx (Anopheles gambiae) construct with exon 2 included
ctagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgaccgccggcgccttaattaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctgagtccggaatcctcgtcgctaccgatggcgctggtgatgcggggcacgctgtgggcgtaggtcacctcgcgctggcacacgtggtcgcgcttgtcgctggtgtccctcatctgcttggtgatgatggtcacgaagtgggggccggggatcttgatggcgcggctgccgttgaaggtcatcttgctgtcgaagtggcccatcatcaggccgccgtcggcggtggtgaagccgatgaaggccagctggcgcacggcgttggggccgtgggggaacatgtgggtctcgttgggcaggatgtccaccagctggtcgcgcatgatggggccgtcgggctggaagccgtcgcagttcacggtgatgcggctgaccacgcaggtgccgtccagctcgtaggtgtggtggctggtcatggtgccgtcgttctcgaagcgcacggtgcggtcgatgctcaggccctcggggaagcactcctgggcgaagtggctgatgccgttggggtagcgggcgaagaagggctcgccgtactggatcaggtggcagatgggcttccagctcatgggcagcttgccggtctcgcacacggcgtgcacgttgaagtcgccgtgggggaacttgctgctgccgtcggccacgatggtgaacttctggccgttcacctcgccgtcgatgaagattttgaaggtcatgtcgctctggaacagggcggggccgccctctgaaccatcctcgtccatggtggcgaccggtttgcgcttcttcttgggtggggtgggatccaccagagacaggttgcggcggcggttggatggcgtgggcgcgttggcgttgttggaccggctcatgttgtgtcgctgtaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtatggtaccatgcggccgcgtacgcgcccggggagcccaagggcacgccctggcacccgtccggtgcttatctagagcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgctagcgtttaaacgagctctaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcagctacgccgctacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggaagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatggcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgcaagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattctcgcgctcgcgcgactgacggtcgtaagcacccgcgtacgtgtccatggcggatccgtgtcgctgtaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtata

SEQ ID NO: 49 - DNA, 11868 bp, artificial
  49 Sequence of pLA1188-cctra intron construct
gtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccgggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtggtttctaaaggtgttataaatcaaattagttttgttttttcttgaaaactttgcgtttcctttgatcaacttaccgccagggtaccgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaat

SEQ ID NO: 50 - DNA, 11868 bp, artificial
  Sequence of pLA3077-a Cctra intron-tTAV construct.
gtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccgggcgccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccaacttcgttcaacagctccaacgcggagttgatgactttggacttatccaggcggctgcccatggtggtttctaaaggtgttataaatcaaattagttttgttttttcttgaaaactttgcgtttcctttgatcaacttaccgccagggtacctgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaat

SEQ ID NO: 51 - DNA, 11788 bp, artificial
  51 Sequence of pLA3097-a Cctra intron-tTAV construct.
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccatggtggctagcgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggccc

SEQ ID NO: 52 - DNA, 13292 bp, artificial
  Sequence of pLA3233-Cctra-intron-tTAV2 construct.
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagacaccggtgttagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccatggtggctagcgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggacgaggatggttcagagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccaacggcatcagccacttcgcccaggagtgcttccccgagggcctgagcatcgaccgcaccgtgcgcttcgagaacgacggcaccatgaccagccaccacacctacgagctggacggcacctgcgtggtcagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcaagatccccggcccccacttcgtgaccatcatcaccaagcagatgagggacaccagcgacaagcgcgaccacgtgtgccagcgcgaggtgacctacgcccacagcgtgccccgcatcaccagcgccatcggtagcgacgaggattccggactcagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccaggaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgccc

SEQ ID NO: 53 - DNA, 14713 bp, artificial
  Sequence of pLA3014-Cctra-intron-Ubiquitin- reaperKR construct.
cgcgccggacgcggcaagtctgcgagcttatatttacgtggatctccggtgtgtccatgattcggcatcatatcataaacgacgaattccaataaaaactttgcttgttgataacacctgatgttcagagatgcccgataaaatcacagctgttctggttcacagtcaccagaaataaaaaatattggaattgagatgtacacaattaacgatatttataaatatcttccgatagtctatcgtccggttaatcaaaataaagtgcgacgaattaacatattttcaaaattaagacgctttgatagatgtatttgtatagagatagaaattaaggttaaaataacataaatgccaaagtttagagcactattcaataattctcttgatttcaaattgaaataatacacaatataacattttctaacactacaaagtcacgatattcttccaccaaccgatagtatcgcacacttgccattcgcctcatcacgcacacgcccgcttcacaattcaaacgaacggcattttattttcacaggatcccgggagtcgtgaatgttttacccaatatcgactttcattgttaactgaccaaaattgtaatctgttctgttagttgtcgagtgcctgtgccgcgatcgctatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccacaccagccaccaccttctgataggcagcctgcacctgaggagtgaattctttgccaaaatgatgagacagcacaacaaccagcacgttgcccaggagctgtaggaaagagaagaaggcatgaacatggttagcagaggggcccggtttggactcagagtattttatcctcatctcaaacagtgtatatcattgtaaccataaagagaaaggcaggatgatgaccagggtgtagttgtttctaccaataagaatatttccacgccagccagaatttatatgcagaaatattctaccttatcatttaattataacaattgttctctaaaactgtgctgaagtacaatataatataccctgattgccttgaaaaaaaagtgattagagaaagtacttacaatctgacaaataaacaaaagtgaatttaaaaattcgttacaaatgcaagctaaagtttaacgaaaaagttacagaaaatgaaaagaaaataagaggagacaatggttgtcaacagagtagaaagtgaaagaaacaaaattatcatgagggtccatggtgatacaagggacatcttcccattctaaacaacaccctgaaaactttgccccctccatataacatgaattttacaatagcgaaaaagaaagaacaatcaagggtccccaaactcaccctgaagttctcagctctagacgcgtttcactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctcaattccatggtggcaacctgcaaggcgaatgaataaacaagattgtggcgaacagtgtaatgcgaagaacccacctctgctccaattcccaattccctattcagctcgagcggggatccccgggtaccgagctcgaattcggggccgcggaggctggatcggtcccggtgtcttctatggaggtcaaaacagcgtggatggcgtctccaggcgatctgacggttcactaaacgagctctgcttatataggcctcccaccgtacacgcctacctcgacccgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaaatgtcgactatgcggaccgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcgctagccaccatggttgttattaaacgtagatttggtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaagcaaccaactacaatcaacattctatgggataatcgacaagtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataagtatcaaaaaagtaaacctctttttcagtctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaatgtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctataggtgaaggctcaaagcctctgatgcagatctttgtgaagactttgaccggaaagaccatcaccctcgaggtagagccatcggacaccattgagaatgtaaaggccaagattcaggataaggagggaatccccccagatcagcagcgtctgatcttcgctggcaagcaactggaagacggacgcaccctgtccgattacaacatccagaaggagtccacccttcacttggtccttcgtctccgtggtggcgccgtggccttctacatcccggatcaggccaccctgctgcgcgaggccgagcagcgcgagcagcagatcctgcgcctgcgcgagagccagtggcgcttcctggccaccgtggtgctggagaccctgcgccagtacaccagctgccacccgcgcaccggccgccgcagcggccgttaccgccgtccgagccagtaacaccggtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagtttaaacgcgtccgcatacgtccgctcacgttaagttccgcagagagaagttgttgaaaacataaacagaatcacttgttgcactctttgagaaaactggggctattgcggaaaaaaccaactaaaaatattgcaggttaggggtactacgctcgattggcgtacggccaccacttttgcgacttcactgttaaccgctaccttcatagagacttttacccgataaatgttatgtagtttgactttctctgttaatcacaagaaaaaatattgtggaaattaaaattatctcaaactcaataaggaaataataatatatacacctatgttttatagaagtcaacagtaaataagttatttggaaaaccattgtagccgtttaaataaatctccttgagtgtgttttaaataacggtcattaagtatattacttggccctctgaatttcttgaattacaccattttttgaaataaatcaatccaaaagactactttttggtggcaaatgaactgcataaaaagtaacaaaagaaatatgtttttgaaataacagtatagctgaagtgtattaaaaaataccgtcatatgagcgacccgctgttaccgcttcgctgcgaatgacaaaacgggctgagcaagaaaatggcgtagaaggcgacgaaaattcgtttcactcgtgaagaaaacctcgataactgaggaatacagctgggatttaaagagcatattcgaactacaagcagagatgtttcctggtggaaacggaaacgccgatttgggctacaacaagcatgcccacgtccatggacttggacaacatggccatgggcacaaccataatcacaatcagttcctgcgcagcccccaccaccccccacacatttttcactgccctccgggggcggtcagggcatggtgacgcccatggtagccgccggcctgccgctcgccatgcagggtggcgttggcatcgattggcgcagctcgcccagcaatggattaattaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatccccgatctgcattttggattattctgcgggtcaaaatagagatgtggaaaattagtacgaaatcaaatgagtttcgttgaaattacaaaactattgaaactaacttcctggctggggaataaaaatgggaaacttatttatcgacgccaactttgttgagaaacccctattaaccctctacgaatattggaacaaaggaaagcgaagaaacaggaacaaaggtagttgagaaacctgttccgttgctcgtcatcgttttcataatgcgagtgtgtgcatgtatatatacacagctgaaacgcatgcatacacattattttgtgtgtatatggtgacgtcacaactactaagcaataagaaattttccagacgtggctttcgtttcaagcaacctactctatttcagctaaaaataagtggatttcgttggtaaaatacttcaattaagcaaagaactaactaactaataacatgcacacaaatgctcgagtgcgttcgtgatttctcgaattttcaaatgcgtcactgcgaatttcacaatttgccaataaatcttggcgaaaatcaacacgcaagttttatttatagatttgtttgcgttttgatgccaattgattgggaaaacaagatgcgtggctgccaatttcttattttgtaattacgtagagcgttgaataaaaaaaaaatggccgaacaaagaccttgaaatgcagtttttcttgaaattactcaacgtcttgttgctcttattactaattggtaacagcgagttaaaaacttacgtttcttgtgactttcgagaatgttcttttaattgtactttaatcaccaacaattaagtataaatttttcgctgattgcgctttactttctgcttgtacttgctgctgcaaatgtcaattggttttgaaggcgaccgttcgcgaacgctgtttatataccttcggtgtccgttgaaaatcactaaaaaataccgtagtgttcgtaacactttagtacagagaaaaaaaattgtgccgaaatgtttttgatacgtacgaataccttgtattaaaattttttatgatttctgtgtatcactttttttttgtgtttttcgtttaaactcaccacagtacaaaacaataaaatatttttaagacaatttcaaattgagacctttctcgtactgacttgaccggctgaatgaggatttctacctagacgacctacttcttaccatgacattgaatgcaatgccacctttgatctaaacttacaaaagtccaaggcttgttaggattggtgtttatttagtttgcttttgaaatagcactgtcttctctaccggctataattttgaaactcgcagcttgactggaaatttaaaaagtaattctgtgtaggtaaagggtgttttaaaagtgtgatgtgttgagcgttgcggcaacgactgctatttatgtatatattttcaaaacttattgtttttgaagtgttttaaatggagctatctggcaacgctgcgcataatcttacacaagcttttcttaatccatttttaagtgaaatttgtttttactctttcggcaaataattgttaaatcgctttaagtgggcttacatctggataagtaatgaaaacctgcatattataatattaaaacatataatccactgtgctttccccgtgtgtggccatatacctaaaaaagtttattttcgcagagccccgcacggtcacactacggttcggcgattttcgattttggacagtactgattgcaagcgcaccgaaagcaaaatggagctggagattttgaacgcgaagaacagcaagccgtacggcaaggtgaaggtgccctccggcgccacgcccatcggcgatctgcgcgccctaattcacaagaccctgaagcagaccccacacgcgaatcgccagtcgcttcgtctggaactgaagggcaaaagcctgaaagatacggacacattggaatctctgtcgctgcgttccggcgacaagatcggggtaccatgcggccgctcatttaaatctggccggcctggccgatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgctttactcgacctaaactttaaacacgtcatagaatcttcgtttgacaaaaaccacattgtggccaagctgtgtgacgcgacgcgcgctaaagaatggcaaaccaagtcgcgcgagcgtcgacctgcaggcatgcaagcttgcatgcctgcaggtcgaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttgtttaaaatataacaaaattgtgatcccacaaaatgaagtggggcaaaatcaaataattaatagtgtccgtaaacttgttggtcttcaactttttgaggaacacgttggacggcaaatccgtgactataacacaagttgatttaataattttagccaacacgtcgggctgcgtgttttttgccgacgcgtctgtgtacacgttgattaactggtcgattaaactgttgaaataatttaatttttggttcttctttaaatctgtgatgaaattttttaaaataactttaaattcttcattggtaaaaaatgccacgttttgcaacttgtgagggtctaatatgaggtcaaactcagtaggagttttatccaaaaaagaaaacatgattacgtctgtacacgaacgcgtattaacgcagagtgcaaagtataagagggttaaaaaatatattttacgcaccatatacgcatcgggttgatatcgttaatatggatcaatttgaacagttgattaacgtgtctctgctcaagtctttgatcaaaacgcaaatcgacgaaaatgtgtcggacaatatcaagtcgatgagcgaaaaactaaaaaggctagaatacgacaatctcacagacagcgttgagatatacggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttggaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttgcggccagcccgggctatgg

SEQ ID NO: 54 - DNA, 15848 bp, artificial
  Sequence of pLA3166-Cctra intron-Ubiquitin- reaperKR construct.
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctggaaatgggatagatattggtgttattgttcatgtggcatataaaggacaagcaacaaaaaacgaacataacatgagagatggttctgaatcagaacttctgaatattatcctcccaaaagggttaaagtttttattaagcatattacgttttataccacttccttatgtaaaattttcttcgtagtttaatatcatgtgaaatcatatataatttctatcgaacgtttgttcaaattgaatgatgtcattttttgaataattggttataattttataacatctcccgacttcgacatgtggttggtactaatgattgcgaaatcgccctccgagaatgagaacaaccgaggtccaccgtctggtcgagattaaaacacttgaggagtgctttggtgactcgatcaataggtacagggctcgttgccaacaatctggccagctggacatccgggacctcgttcccccctggggtatcaaaatttttgtagtgtaaatagtagtacactcttaaaaataatgaaaattactgcggacgtaattcacattatgattgaatgacactatcattgacatttcccgaatcagacaccatcgtatttaaaatgtgacacaaattcacctcatttggctcgcttcttttatgtgcatccaaaagacgtaaaatcgcatgattttttcggagtgtgtagtaagattgtcaaattttaattttaaataaccagagcccataaagcaaagcaacactaggaaaaaacccacaaactcaacctgtccaaaaaaaaatataacaatcaaagttgagggaatcggggtcaaacgtcatgtaaaaatattttttgtaaaaaccaaaccaggaataaatatgaatttaatcggaaaaaattgcaaaatcgcataatttaatcctccaactgtactttatccagcctgttgcagaaatgatgtttaaaggttctaatctgtaattgttattagccttcaatactgatgtagtatttatttcttattgaaacattgagagctttattttccaaagttgtcattttctcattcgtatatcgtaatatgtatattcgtaaatggcaagcacaatgatacttagggtagtcaaggatatttcaattacgaaaagatcctgaaacgaccgggaatcgaacccttcagcatggttttgctttgtagctgctgaatctaaccactaggctgatgaagatcccattttagggttgcaagttctcaaagagcaagaatgccaaaatagtgtcaaaagaagccctatttgacgatataccttttagtctctacgttaatttgctatgataatttatcatcaattaattggcaaagcctgatgcacgaaaagatcttcttctaaaatttcagttgttcttttcaacacattatgtaatcataaaatttaattaataaaccttttttttttgtaactatccacagttgatcaggcataattttcttggaaagtaaagtccatatttaggttgatgttgaataaaaaaactttcaattcactcttctgtttcacttcagaacttacgtaatacgacattatgcatggtgcacacggaacaggataagacgttcacaagggatcaacatcacatcggatcgtaatcactggatctggaacacatatgacgccacaagacagcacattttacacgatcaccagacgtgaacaaggaactggatccacaagacgtcacaggaagacggcacatttccaacggcttcgatggaacttttctcgagtctttttccaccaatcataaacaccgacctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtggtttctaaaggtgttataaatcaaattagttttgttttttcttgaaaactttgcgtttcctttgatcaacttaccgccagggtaccgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggacgaggatggttcagagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccaacggcatcagccacttcgcccaggagtgcttccccgagggcctgagcatcgaccgcaccgtgcgcttcgagaacgacggcaccatgaccagccaccacacctacgagctggacggcacctgcgtggtcagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcaagatccccggcccccacttcgtgaccatcatcaccaagcagatgagggacaccagcgacaagcgcgaccacgtgtgccagcgcgaggtgacctacgcccacagcgtgccccgcatcaccagcgccatcggtagcgacgaggattccggactcagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaatccattgctgggcgagctgcgccaatcgatgccaacgccaccctgcatggcgagcggcaggccggcggctaccatgggcgtcaccatgccctgaccgcccccggagggcagtgaaaaatgtgtggggggtggtgggggctgcgcaggaactgattgtgattatggttgtgcccatggccatgttgtccaagtccatggacgtgggcatgcttgttgtagcccaaatcggcgtttccgtttccaccaggaaacatctctgcttgtagttcgaatatgctctttaaatcccagctgtattcctcagttatcgaggttttcttcacgagtgaaacgaattttcgtcgccttctacgccattttcttgctcagcccgttttgtcattcgcagcgaagcggtaacagcgggtcgctcatatgacggtattttttaatacacttcagctatactgttatttcaaaaacatatttcttttgttactttttatgcagttcatttgccaccaaaaagtagtcttttggattgatttatttcaaaaaatggtgtaattcaagaaattcagagggccaagtaatatacttaatgaccgttatttaaaacacactcaaggagatttatttaaacggctacaatggttttccaaataacttatttactgttgacttctataaaacataggtgtatatattattatttccttattgagtttgagataattttaatttccacaatattttttcttgtgattaacagagaaagtcaaactacataacatttatcgggtaaaagtctctatgaaggtagcggttaacagtgaagtcgcaaaagtggtggccgtacgccaatcgagcgtagtacccctaacctgcaatatttttagttggttttttccgcaatagccccagttttctcaaagagtgcaacaagtgattctgtttatgttttcaacaacttctctctgcggaacttaacgtgagcggacgtatgcggacgcgtttaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaccggtgttactggctcggacggcggtaacggccgctgcggcggccggtgcgcgggtggcagctggtgtactggcgcagggtctccagcaccacggtggccaggaagcgccactggctctcgcgcaggcgcaggatctgctgctcgcgctgctcggcctcgcgcagcagggtggcctgatccgggatgtagaaggccacggcgccaccacggagacgaaggaccaagtgaagggtggactccttctggatgttgtaatcggacagggtgcgtccgtcttccagttgcttacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccagcgaagatcagacgctgctgatctggggggattccctccttatcctgaatcttggcctttacattctcaatggtgtccgatggctctacctcgagggtgatggtctttccggtcaaagtcttcacaaagatctgcattttggattgctagcgcagattgtttagcttgttcagctgcgcttgtttatttgcttagctttcgcttagcgacgtgttcactttgcttgtttgaattgaattgtcgctccgtagacgaagcgcctctatttatactccggcgctcggtccgcatagtcgacatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggccc

SEQ ID NO: 55 - DNA, 17802 bp, artificial
  Sequence of pLA3376-Bztra intron-reaperKR and Bztra-intron-tTAV3.
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagacaccggtgttagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccatggtggctagcgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctgcgcgccgtttaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgctctagacaccggtgctacccgccatactcatcgatgcccagcgcgtcggtgaacatttgctcgaactcgaagtcggccatgtccagggcgccgtacggggcgctatcgtggggcgtgaagcccggtcccgggctatctccatcgcccagcatatccaggtcgaaatcgtccagggcgtcggcgtgggccattgccacatcctctccatccaggtgcagctcgtcgcccaggctcacatcggtcggcggggcggtgctcaggcggcgcgtgtgtccggcgggcaggaagctcaggcggggggcggccaggccggcttcctccggggcatcgtcatccggcaggtccagcagtccctcgatggtgctgccatagttgttcttggtacgggcgcggctgtaggcgctgccgctctcgcacttcagctgcttttccaggccgcagatgatcagctccaggccgaacaggaaggccggctcggcgccctggtgatcgaacagctcgatggcctggcgcagcagcggcggcatgctatcggtggtcggggtctcgcgctcctccttggccacctggtgctcctgatcctccagcacacagcccagggtgaagtggcccacggcgctcagggcgtacagggcgttctccaggctgaagccctgctggcacaggaaggccagctggttctccagggtctcgtactgcttctcggtcgggcgggtgcccaggtgcaccttggcgccatcgcggtgcgacagcagggcgcagcggaagctcttggcgttgttgcgcaggaaatcctgccagctctcgccctccagcgggcagaagtgggtgtggtggcgatccagcatttcgatggccagggcgtccagcagggcgcgcttgttcttcacgtgccagtacagggtcggctgttccacgcccagcttctgggccagcttgcgggtggtcaggccctcgataccaacttcgttcagcagctccagggcgctgttgatcaccttgctcttgtccaggcggctgacctgtgaatacggttaatgtcactattagtgatttataaaaataaatttgatttatatatcaacaatttttcatcgcagccttcagctttttgttgaataattataatgatattttttacgattcaaatcatttaattgttactcaacgaaataagtttaattcaaattttaaaacaagattatatattaagattagaataagaaagaactttgttagattatttaattaaaaagattaaaatttaagtctccagtcactatttaaagatcatctttcaaacgttaaagtgaattcaaacgagacgttcaaatttcgattaaacagtaattaactctaaatttctatcacgaattaagttattgaatatgaaggtttatatttatttacatcatctaataggtttgagttgattgttgtaatccgcatgtgccagaagatatcaatttccaaattgtccgagttcatggaatgttgattgttgtttgtgttgctttgtaattgttgcagggagtatttatggtttgttgattgtagtataaggctgtttctaaaggctagaaaataattttatttatttgaaaataagtaaatatacataatattactaacaataggtcgtcctattttttgatattctgcacaaatttttaaaacacaaagattgcaatacttttagacactaatactgcacactctgaaaaattattaaattatttttaaaaacttaccttaatactttagagaaaaatattataccgcacctttctactttatactcactttattataccagttgcatgttgattgtagttctttgacaagaaaatattccatattgctccaaattatcttggtaagttgattggtgcgtcatttgagcaagctaacaccttgtctcatttaagttcgcctcaagatctcatagcatttttaaatatcactatatttagtaagtaattagaattaccatggtggtttgctagccgttctatcagatgtgctccgggaaacagaaatgttcaactaagttctggcggacgacgcaacacctttatatactttgccaagcgcacaggtagaaaggacctattttggggattaaaaaacatctgcctgttttattgccatacccgcgaaaattcgcgaaatccgctactttacctactggggttcctggaaaatgggcgaagaacggcaaagaactggtactttccgtcaataattgtttagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttatcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccaggaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgcccggccgcgccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatccgtctacctgagcgatatataaactaatgcctgttgcaattgttcagtcagtcacgagtttgttaccactgcgacaagctagcaaccaccatggcggtaattctaattacttactaaatatagtgatatttaaaaatgctatgagatcttgaggcgaacttaaatgagacaaggtgttagcttgctcaaatgacgcaccaatcaacttaccaagataatttggagcaatatggaatattttcttgtcaaagaactacaatcaacatgcaactggtataataaagtgagtataaagtagaaaggtgcggtataatatttttctctaaagtattaaggtaagtttttaaaaataatttaataatttttcagagtgtgcagtattagtgtctaaaagtattgcaatctttgtgttttaaaaatttgtgcagaatatcaaaaaataggacgacctattgttagtaatattatgtatatttacttattttcaaataaataaaattattttctagcctttagaaacagccttatactacaatcaacaaaccataaatactccctgcaacaattacaaagcaacacaaacaacaatcaacattccatgaactcggacaatttggaaattgatatcttctggcacatgcggattacaacaatcaactcaaacctattagatgatgtaaataaatataaaccttcatattcaataacttaattcgtgatagaaatttagagttaattactgtttaatcgaaatttgaacgtctcgtttgaattcactttaacgtttgaaagatgatctttaaatagtgactggagacttaaattttaatctttttaattaaataatctaacaaagttctttcttattctaatcttaatatataatcttgttttaaaatttgaattaaacttatttcgttgagtaacaattaaatgatttgaatcgtaaaaaatatcattataattattcaacaaaaagctgaaggctgcgatgaaaaattgttgatatataaatcaaatttatttttataaatcactaatagtgacattaaccgtattcacaggtggccttctacatcccggatcaggccaccctgctgcgcgaggccgagcagcgcgagcagcagatcctgcgcctgcgcgagagccagtggcgcttcctggccaccgtggtgctggagaccctgcgccagtacaccagctgccacccgcgcaccggccgccgcagcggccgttaccgccgtccgagccagtaacaccggtgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagtttaggcgcg

SEQ ID NO: 56 - DNA, 15134 bp, artificial
  Sequence of pLA3242-Crtra intron-reaperKR construct.
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccatggtggctagcgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgcctaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaccggtgttactggctcggacggcggtaacggccgctgcggcggccggtgcgcgggtggcagctggtgtactggcgcagggtctccagcaccacggtggccaggaagcgccactggctctcgcgcaggcgcaggatctgctgctcgcgctgctcggcctcgcgcagcagggtggcctgatccgggatgtagaaggccacctaaagataccatggatgtatgaattagtatcatatacatataaatgcttttttttttggcatattaatgttaaaaatatcaacaatttccgtgatagtttttaccatttttgttgaatgtttactttgaaaacttaaatattttttaactaattttaccagtcatgtgttattaaaagtatttatgaataaaactgcaagtaaagcgtttcaaaaatttatcaagtaaaactttactttttttaaatcttaactgtcaattcattattaatttattaatttaaatttgcaatgtctatttactttaagacaaattaaagcaagaaactaaatattcgaatcaattcttttttaaatgaaattttacttcatcatcatgtatgtgtgtatcaatttttatttacattgtataataagtttcgagttgattgttgtaatccgcaggtgtcccgaagtattaaattccgaattcccaagtttattgaatgttgattgtagtttcagttgttttgttattgcaacaatggcttgttgattggagatattttccttttccttggtttacttactacatagactgaaaaagatgtttgacttttttgatactattgtaaaatttctattagtgattactaaccaatcgctataagtttaatagaaaacaaataaactctttgcatccagatatacctagcttcttaacccttatctattaactccattgcttgtaacaaatctagatattaatagatgtctaattacttagcaaaacttctttttgattaagcagccacagctgtcgattttggtcatatttaaaggaaataaatgcgtttaaaataataattaatataagttttgaaactttttactaacacttggcagcaggaagtaggtgtttttcacagctttctgaaccaccggcaccttccccggtctccgttgtcggagttcagcaggatttccggcccccaattaaccccgaaacaaaacatgtcttattaataaggtgtattcaaaatagtgggaatgtcatgactgctgaccttatttttattcctattgtaagtgttccggctataattttactcacttgtccattatcccatagaatgttatgttgattgtagttgtttgcttttccaggtgagagttgatcaagtcgcaaaagttagcgtgtgttgattgtagatttgaaggtaaaataattttgtacacattcatcaggcaaaacgttctccatcgaataaacttccatcgataattgatagcttatgaatttcaaaaaaaaatatgcttttaaaattaccgccatggtggttgctagcttgtcgcagtggtaacaaactcgtgactgactgaacaattgcaacaggcattagtttatatatcgctcaggtagacggatcgatctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggagtatgttctctctcttctcttctctctctctttctcgaatgttctctctcttctcttctctctctctttctcgatggccggcctggcttaattaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctgagtccggaatcctcgtcgctaccgatggcgctggtgatgcggggcacgctgtgggcgtaggtcacctcgcgctggcacacgtggtcgcgcttgtcgctggtgtccctcatctgcttggtgatgatggtcacgaagtgggggccggggatcttgatggcgcggctgccgttgaaggtcatcttgctgtcgaagtggcccatcatcaggccgccgtcggcggtggtgaagccgatgaaggccagctggcgcacggcgttggggccgtgggggaacatgtgggtctcgttgggcaggatgtccaccagctggtcgcgcatgatggggccgtcgggctggaagccgtcgcagttcacggtgatgcggctgaccacgcaggtgccgtccagctcgtaggtgtggtggctggtcatggtgccgtcgttctcgaagcgcacggtgcggtcgatgctcaggccctcggggaagcactcctgggcgaagtggctgatgccgttggggtagcgggcgaagaagggctcgccgtactggatcaggtggcagatgggcttccagctcatgggcagcttgccggtctcgcacacggcgtgcacgttgaagtcgccgtgggggaacttgctgctgccgtcggccacgatggtgaacttctggccgttcacctcgccgtcgatgaagattttgaaggtcatgtcgctctggaacagggcggggccgccctctgaaccatcctcgtccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacatcatccaaattaataaatgacaagcaatgggtaccatgcggccgcaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccaggaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgccc

SEQ ID NO: 57 - DNA, 1403 bp, artificial
  SEQ ID NO. 57 Partial sequence of a male transcript generated in Drosophila melanogaster from LA3077 transformants that differs tothe sequence generated in Medfly LA3077 lines.  T
ggccagatctgttgttattaaacgtagatttggtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaagcaaccaactacaatcaacattctatgggataatcgacaagtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataagtatcaaaaaagtaaacctctttttcagtctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaatgtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctataggtgaaggctcaaagcctctggctagc

SEQ ID NO: 58 - DNA, 972 bp, Bactrocera zonata
cggtaattctaattacttactaaatatagtgatatttaaaaatgctatgagatcttgaggcgaacttaaatgagacaaggtgttagcttgctcaaatgacgcaccaatcaacttaccaagataatttggagcaatatggaatattttcttgtcaaagaactacaatcaacatgcaactggtataataaagtgagtataaagtagaaaggtgcggtataatatttttctctaaagtattaaggtaagtttttaaaaataatttaataatttttcagagtgtgcagtattagtgtctaaaagtattgcaatctttgtgttttaaaaatttgtgcagaatatcaaaaaataggacgacctattgttagtaatattatgtatatttacttattttcaaataaataaaattattttctagcctttagaaacagccttatactacaatcaacaaaccataaatactccctgcaacaattacaaagcaacacaaacaacaatcaacattccatgaactcggacaatttggaaattgatatcttctggcacatgcggattacaacaatcaactcaaacctattagatgatgtaaataaatataaaccttcatattcaataacttaattcgtgatagaaatttagagttaattactgtttaatcgaaatttgaacgtctcgtttgaattcactttaacgtttgaaagatgatctttaaatagtgactggagacttaaattttaatctttttaattaaataatctaacaaagttctttcttattctaatcttaatatataatcttgttttaaaatttgaattaaacttatttcgttgagtaacaattaaatgatttgaatcgtaaaaaatatcattataattattcaacaaaaagctgaaggctgcgatgaaaaattgttgatatataaatcaaatttatttttataaatcactaatagtgacattaaccgtattcacaggt

SEQ ID NO: 59 - DNA, 1312 bp, Ceratitis rosa
tggtaattttaaaagcatatttttttttgaaattcataagctatcaattatcgatggaagtttattcgatggagaacgttttgcctgatgaatgtgtacaaaattattttaccttcaaatctacaatcaacacacgctaacttttgcgacttgatcaactctcacctggaaaagcaaacaactacaatcaacataacattctatgggataatggacaagtgagtaaaattatagccggaacacttacaataggaataaaaataaggtcagcagtcatgacattcccactattttgaatacaccttattaataagacatgttttgtttcggggttaattgggggccggaaatcctgctgaactccgacaacggagaccggggaaggtgccggtggttcagaaagctgtgaaaaacacctacttcctgctgccaagtgttagtaaaaagtttcaaaacttatattaattattattttaaacgcatttatttcctttaaatatgaccaaaatcgacagctgtggctgcttaatcaaaaagaagttttgctaagtaattagacatctattaatatctagatttgttacaagcaatggagttaatagataagggttaagaagctaggtatatctggatgcaaagagtttatttgttttctattaaacttatagcgattggttagtaatcactaatagaaattttacaatagtatcaaaaaagtcaaacatctttttcagtctatgtagtaagtaaaccaaggaaaaggaaaatatctccaatcaacaagccattgttgcaataacaaaacaactgaaactacaatcaacattcaataaacttgggaattcggaatttaatacttcgggacacctgcggattacaacaatcaactcgaaacttattatacaatgtaaataaaaattgatacacacatacatgatgatgaagtaaaatttcatttaaaaaagaattgattcgaatatttagtttcttgctttaatttgtcttaaagtaaatagacattgcaaatttaaattaataaattaataatgaattgacagttaagatttaaaaaaagtaaagttttacttgataaatttttgaaacgctttacttgcagttttattcataaatacttttaataacacatgactggtaaaattagttaaaaaatatttaagttttcaaagtaaacattcaacaaaaatggtaaaaactatcacggaaattgttgatatttttaacattaatatgccaaaaaaaaaagcatttatatgtatatgatactaattcatacatccatggtatctttagg

SEQ ID NO: 60 - DNA, 21 bp, artificial
  spl-agdsx-e3 primer
cgagcccaatggctgttggag

SEQ ID NO: 61 - DNA, 22 bp, artificial
  spl-agdsx-m primer
gtcaaggttcagggcccgatcg

SEQ ID NO: 62 - DNA, 21 bp, artificial
  primer spl-agdsx-e3
cgagcccaatggctgttggag

SEQ ID NO: 63 - DNA, 22 bp, artificial
  spl-agdsx-m primer
gtcaaggttcagggcccgatcg

SEQ ID NO: 64 - DNA, 20 bp, artificial
  aedesxF1 primer
tcaatggctcctggagaagc

SEQ ID NO: 65 - DNA, 25 bp, artificial
  aedesxR5 primer
accattcttgcagaagtcttgggac

SEQ ID NO: 66 - DNA, 19 bp, artificial
  aedesxR2 primer
aacattctccgcgcacagg

SEQ ID NO: 67 - DNA, 23 bp, artificial
  Agexon1 primer
gacgctcgctctggtacagttcg

SEQ ID NO: 68 - DNA, 20 bp, artificial
  Tra (tTAV) seq+ primer
cctgccaggactcgccttcc

SEQ ID NO: 69 - DNA, 23 bp, artificial
  Agexon1 primer
gacgctcgctctggtacagttcg

SEQ ID NO: 70 - DNA, 26 bp, artificial
  Exon 3 primer
gttgtcgctttgactggcaatgtcgc

SEQ ID NO: 71 - DNA, 632 bp, Pectinophora gossypiella
gaactgccacaaactgctggaaaagttccactactcctgggaaatgatgcccctggtgctggtcattctaaactacgccggctccgacctcgacgaggcttctagaaaaattgatgaagggaagatgatcatcaacgagtacgcgagggagcacaatctgaacatcttcgatggccacgagctgaggaactcgactcgccagaaaatgctgagcgaaattaataatataagtggtgtactatcgtcgtccatgaagttattttgcgaatgatactttgttttgtatgtgctgtgtgttgtgtggacttttgctgtgcgttgctgtttgcgatggaaggactattgtgtcgtcgccacgctggactattcgcacattgggtggtccaccagtggcggatgtacgagcggtcgctgtgctcgctcctggagctgcaagcgcgcaaagggacgtactcggtgtgctgctcaccccgctacgtcatcgcgcccgagtacgcgtcacacctgttgcctctgccgcttaccacgcagagatcatccccgccgcccgcgcacttgtagcgatgcgaacctgcgccgcgggaagcggcgcaagaacccgccgatgccccggcgtcgtcgtcgggtgccac

SEQ ID NO: 72 - DNA, 222 bp, Drosophila melanogaster
atgcagatctttgtgaagactttgaccggaaagaccatcaccctcgaggtagagccatcggacaccattgagaatgtaaaggccaagattcaggataaggagggaatccccccagatcagcagcgtctgatcttcgctggcaagcaactggaagacggacgcaccctgtccgattacaacatccagaaggagtccacccttcacttggtccttcgtctccgt

SEQ ID NO: 73 - protein, 74 aa, Drosophila melanogaster
MQIFVKTLTGKTITLEVEPSDTIENVKAKIQDKEGIPPDQQRLIFAGKQLEDGRTLSDYNIQKESTLHLVLRLR

SEQ ID NO: 74 - DNA, 34 bp, artificial
  primer
caagcaaagtgaacacgtcgctaagcgaaagcta

SEQ ID NO: 75 - DNA, 22 bp, artificial
  primer
gcgggtggcagctggtgtactg

SEQ ID NO: 76 - DNA, 34 bp, artificial; identical to SEQ ID NO: 74
  primer

SEQ ID NO: 77 - DNA, 24 bp, artificial
  primer
gcggaacgacttggcgttattgcg

SEQ ID NO: 78 - DNA, 28 bp, artificial
  primer
ggaagggtccttacgctatagagcgcag

SEQ ID NO: 79 - DNA, 31 bp, artificial
  primer
ccaggcgaagttgttattaagcgtagatttg

SEQ ID NO: 80 - DNA, 33 bp, artificial
  primer
cgtcgctttgaaacagaggctttgagccttctc

SEQ ID NO: 81 - DNA, 48 bp, artificial
  primer
gctagcaaccaccatggcggtaattctaattacttactaaatatagtg

SEQ ID NO: 82 - DNA, 41 bp, artificial
  primer
ccgggatgtagaaggccacctgtgaatacggttaatgtcac

SEQ ID NO: 83 - DNA, 31 bp, artificial
  primer
cagtcagtcacgagtttgttaccactgcgac

SEQ ID NO: 84 - DNA, 22 bp, artificial
  primer
gcgggtggcagctggtgtactg

SEQ ID NO: 85 - DNA, 21 bp, artificial
  primer
cggagcacatctgatagaacg

SEQ ID NO: 86 - DNA, 23 bp, artificial
  primer
cgcggctgtaggcgctgccgctc

SEQ ID NO: 87 - DNA, 31 bp, artificial; identical to SEQ ID NO: 79
  primer

SEQ ID NO: 88 - DNA, 33 bp, artificial; identical to SEQ ID NO: 80
  primer

SEQ ID NO: 89 - DNA, 52 bp, artificial
  primer
gctagcaaccaccatggcggtaattttaaaagcatatttttttttgaaattc

SEQ ID NO: 90 - DNA, 41 bp, artificial
  primer
ccgggatgtagaaggccacctaaagataccatggatgtatg

SEQ ID NO: 91 - DNA, 31 bp, artificial; identical to SEQ ID NO: 83
  primer

SEQ ID NO: 92 - DNA, 22 bp, artificial
  primer
gcgggtggcagctggtgtactg

SEQ ID NO: 93 - DNA, 21 bp, artificial
  primer
gttgcaagttgacactggcgg

SEQ ID NO: 94 - DNA, 23 bp, artificial
  primer
aggtgtgggaggttttttaaagc

SEQ ID NO: 95 - DNA, 52 bp, artificial
  primer
cctgtaatacgactcactatagggcgtttttttttttttttttttttttttt

SEQ ID NO: 96 - DNA, 33 bp, artificial
  primer
gcaaacggcaatcagacgggcccaggctcagga

SEQ ID NO: 97 - DNA, 28 bp, artificial
  primer
cctgtaatacgactcactatagggcgtt

SEQ ID NO: 98 - DNA, 37 bp, artificial
  primer
gggatcgagctagatcggcctgagccgccagtggtga

SEQ ID NO: 99 - DNA, 28 bp, artificial
  primer
cctgtaatacgactcactatagggcgtt

SEQ ID NO: 100 - DNA, 32 bp, artificial
  primer
cgctccatgggatcggcgagctgcgactccgt

SEQ ID NO: 101 - DNA, 27 bp, artificial
  primer
gcaacaaccagcggtgtcccttgaaac

SEQ ID NO: 102 - DNA, 28 bp, artificial
  primer
cctgtaatacgactcactatagggcgtt

SEQ ID NO: 103 - DNA, 28 bp, artificial
  primer
gctagtggagaactgccacaaactgctg

SEQ ID NO: 104 - DNA, 34 bp, artificial; identical to SEQ ID NO: 74
  primer

SEQ ID NO: 105 - DNA, 25 bp, artificial
  primer
gccctcgatggtagacccgtaattg

SEQ ID NO: 106 - DNA, 14874 bp, artificial
  LA1172 nucleotide sequence, including plasmid backbone
gggctggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggccaggccggccagatttaaatgagcggccgcatggtaccatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttacagcgacacaacatgagccggtccaacaacgccaacgcgcccacgccatccaaccgccgccgcaacctgtctctggtggatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaatccattgctgggcgagctgcgccaatcgatgccaacgccaccctgcatggcgagcggcaggccggcggctaccatgggcgtcaccatgccctgaccgcccccggagggcagtgaaaaatgtgtggggggtggtgggggctgcgcaggaactgattgtgattatggttgtgcccatggccatgttgtccaagtccatggacgtgggcatgcttgttgtagcccaaatcggcgtttccgtttccaccaggaaacatctctgcttgtagttcgaatatgctctttaaatcccagctgtattcctcagttatcgaggttttcttcacgagtgaaacgaattttcgtcgccttctacgccattttcttgctcagcccgttttgtcattcgcagcgaagcggtaacagcgggtcgctcatatgacggtattttttaatacacttcagctatactgttatttcaaaaacatatttcttttgttactttttatgcagttcatttgccaccaaaaagtagtcttttggattgatttatttcaaaaaatggtgtaattcaagaaattcagagggccaagtaatatacttaatgaccgttatttaaaacacactcaaggagatttatttaaacggctacaatggttttccaaataacttatttactgttgacttctataaaacataggtgtatatattattatttccttattgagtttgagataattttaatttccacaatattttttcttgtgattaacagagaaagtcaaactacataacatttatcgggtaaaagtctctatgaaggtagcggttaacagtgaagtcgcaaaagtggtggccgtacgccaatcgagcgtagtacccctaacctgcaatatttttagttggttttttccgcaatagccccagttttctcaaagagtgcaacaagtgattctgtttatgttttcaacaacttctctctgcggaacttaacgtgagcggacgtatgcggacgcgccatggtttaaactcgctagcactgggaagttgacgttgatatagagccgaattgaacttcaccgctgcttggtaattactctacaagttcatttaggagaaccggattcgaaagatgattttccagcgtttagctttcagatggccgcatacattttgcaccaccaaaccgaaactcactagcgtatccaatcgttcgttttttggtgccggtgtgttacgaactttagctatcaagctaaagcaatttgctctggtcttccgtgctaaaaagaaaaaaaaactgttttttttttggttttgatatttgcgctatttttacttgggccttaattgaacaaacttttgaaagtttccacagcgaaatcgttttcgacgatgccatttttggtaacatttgcattttcttgctcaaattgcttgcaaaacccgtgaaagacattaatattcgatagtgtcatccaaaatcacgaaaatgattgttgcaaaacgttgaacaatttacacatgtaaaaaacaaccatcgattaatgtttattcaaactttttacaagaagggttattctgatcaatgtcaccccgctgatgaatgttaccccggattacacttctcgaaaagtggttcaaaatgctacttgagaatttttatctgtcaaaggaagcaaattcgagtcgaattaaatggtatagtcctgaattaggtttccatttacttacaggtattccactaaatagctggaagatttattttacacaataatgataattcgtaccccaaagagtgtagccctacttttttctctcttttttttttgtaaattttcatcgctgcgtgccagcttaccgacatgtcgcgacagcataaagagcctgtcaagagatgaagaaaaatgacaaggagtcagtggtcaggtctctgtatcaatatttgacgtcctgactttccaatatacctttccttaaagagtagagatcatgcgatacgtgaataaatatcgtttggacttcgaaatagaacataatttaaggtagctgatcagtagttgaacatcttcagacttctgggacaagaagtgtttttttgtttgtagaaaaggtttttgttaaattatatttgtaagataattcaatgaatatatctctgattcagtaatcaatccgtaccacgcaccgtttaagaaacaccctgtaggtttgcatcacgtctcagacaaaagtgtatcgatgtgcgaacactgcataccggcgctttgcaaataatgccaaatttagatatgcattacattgtcacttcgcaaaacacacactcccaaatgcgtcggaaacctcacccgaacgcacgatcgtaacgcgatcgatcgccgattgattgatcggaattaactatctcaatcgatccttctatggactgatgcatgggccggcacttccgagtataaaaccccggtaaacccaaggaatcactcacaatcggattttgacgctcgctctggtacagttcgatacggtctagtgaaaccgaggataacgacgaaggtttttccccattgatccaggtcggtgtttatgattggtggaaaaagactcgagaaaagttccatcgaagccgttggaaatgtgccgtcttcctgtgacgtcttgtggatccagttccttgttcacgtctggtgatcgtgtaaaatgtgctgtcttgtggcgtcatatgtgttccagatccagtgattacgatccgatgtgatgttgatcccttgtgaacgtcttatcctgttccgtgtgcaccatgcataatgtcgtattacgtaagttctgaagtgaaacagaagagtgaattgaaagtttttttattcaacatcaacctaaatatggactttactttccaagaaaattatgcctgatcaactgtggatagttacaaaaaaaaaaggtttattaattaaattttatgattacataatgtgttgaaaagaacaactgaaattttagaagaagatcttttcgtgcatcaggctttgccaattaattgatgataaattatcatagcaaattaacgtagagactaaaaggtatatcgtcaaatagggcttcttttgacactattttggcattcttgctctttgagaacttgcaaccctaaaatgggatcttcatcagcctagtggttagattcagcagctacaaagcaaaaccatgctgaagggttcgattcccggtcgtttcaggatcttttcgtaattgaaatatccttgactaccctaagtatcattgtgcttgccatttacgaatatacatattacgatatacgaatgagaaaatgacaactttggaaaataaagctctcaatgtttcaataagaaataaatactacatcagtattgaaggctaataacaattacagattagaacctttaaacatcatttctgcaacaggctggataaagtacagttggaggattaaattatgcgattttgcaattttttccgattaaattcatatttattcctggtttggtttttacaaaaaatatttttacatgacgtttgaccccgattccctcaactttgattgttatatttttttttggacaggttgagtttgtgggttttttcctagtgttgctttgctttatgggctctggttatttaaaattaaaatttgacaatcttactacacactccgaaaaaatcatgcgattttacgtcttttggatgcacataaaagaagcgagccaaatgaggtgaatttgtgtcacattttaaatacgatggtgtctgattcgggaaatgtcaatgatagtgtcattcaatcataatgtgaattacgtccgcagtaattttcattatttttaagagtgtactactatttacactacaaaaattttgataccccaggggggaacgaggtcccggatgtccagctggccagattgttggcaacgagccctgtacctattgatcgagtcaccaaagcactcctcaagtgttttaatctcgaccagacggtggacctcggttgttctcattctcggagggcgatttcgcaatcattagtaccaaccacatgtcgaagtcgggagatgttataaaattataaccaattattcaaaaaatgacatcattcaatttgaacaaacgttcgatagaaattatatatgatttcacatgatattaaactacgaagaaaattttacataaggaagtggtataaaacgtaatatgcttaataaaaactttaacccttttgggaggataatattcagaagttctgattcagaaccatctctcatgttatgttcgttttttgttgcttgtcctttatatgccacatgaacaataacaccaatatctatcccatttccaggacctaacggaccttgaagcggcgccaaaacgtgtgacgatgatgctggtaccctggcggtaagttgatcaaaggaaacgcaaagttttcaagaaaaaacaaaactaatttgatttataacacctttagaaaccaccatgggcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagttctagaattgtccaccgcaagtgcttctaagccgatcccgattgtactgattaccataagcgacattgccagtgaaagcgacaacagcagcatcaaagtacatttgtcatactgattcggctactaccaccatccggaatcagcttgcatcgaacatcaaatcacgttattcaatgtatctgtcatccagctcagacaagtcggagcttttccagtcgcgaaaatctgcgactccagcggaaagcaccgaaccacagagaggactcgtatgaaagccagggaagaaaccatcattcaccttgcagcaaataggaaaaaaaacggacatcttcaacaaacaaaagcccatgcgctaacttggtttaggagtttagtgtgacaccatgaccccgctgatgatctttacttagcacaccataaccacctttatgcgttcgttcatccaaaatctacaggatatcactgcagccgcgagaagaactcgtgaaccatcctgttttcttttttattatattcttacttttaacttcaaattattttcagtaataaaacgtctcaaaataataagttcataatgagtttaattttacggaataagaacaaccatttaagttattaaatccttagatttaatggaattagattgattatatggaacccagacttggtaaaaaataaactccacgttaaatttctttctgagacttaaaattctttcgggaaagctgggagcaattctcgcaccggtgctagggccgcatagtcgacatttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatccccgctcgaccaccatgggcgctctcctgggcctgcccgaaagccaaacggagcttgataatcttacagaatacaacacggcccacaatcggcgcatctcaatgctgggcatcgatgatgataccaatatgcgaaagcaaaacgccttgaaacagggacggcgcactcgaaatgtcacatttaacgatgaggagattgtcatcaatcctgaggatgtggatcctaatgtgggacgcttcaggaacttggtacaaaccactgtggtgcccgccaagagggctcgctgcgacgtcaaccattagtgataacgcgtctagctagagctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatagcggccctagcggcgcgccatagccc

SEQ ID NO: 107 - DNA, 13 bp, Drosophila sp.
tcaataatcgtca

SEQ ID NO: 108 - DNA, 13 bp, Drosophila sp.
tcatcaaacgtca

SEQ ID NO: 109 - DNA, 13 bp, Drosophila sp.
ttatcgttaaaca

SEQ ID NO: 110 - DNA, 13 bp, Drosophila sp.
taaacagtcaata

SEQ ID NO: 111 - DNA, 13 bp, Drosophila sp.
tacacgatcagca

SEQ ID NO: 112 - DNA, 13 bp, Drosophila sp.
aatacaaacaaca

SEQ ID NO: 113 - DNA, 13 bp, Drosophila sp.
tcatcaacaagca

SEQ ID NO: 114 - DNA, 13 bp, Drosophila sp.
tctacaaaccaga

SEQ ID NO: 115 - DNA, 13 bp, Drosophila sp.
acatcgattcaca

SEQ ID NO: 116 - DNA, 13 bp, Drosophila sp.
cgctcaatcaaca

SEQ ID NO: 117 - DNA, 13 bp, Drosophila sp.
tctaccataaaaa

SEQ ID NO: 118 - DNA, 13 bp, Drosophila sp.
aaatgaatcaaca

SEQ ID NO: 119 - DNA, 13 bp, Drosophila sp.
acatcgttcaacg

SEQ ID NO: 120 - DNA, 13 bp, Drosophila sp.
tcttgattcacca

SEQ ID NO: 121 - DNA, 13 bp, Drosophila sp.
tctgcagacaaca

SEQ ID NO: 122 - DNA, 13 bp, Drosophila sp.
tcttcggtaatca

SEQ ID NO: 123 - DNA, 13 bp, Drosophila sp.
tctataaacaata

SEQ ID NO: 124 - DNA, 13 bp, Drosophila sp.
taaacaataaata

SEQ ID NO: 125 - DNA, 13 bp, Drosophila sp.
taaacaagcaaaa

SEQ ID NO: 126 - DNA, 13 bp, Drosophila sp.
tcaacgatcggcg

SEQ ID NO: 127 - DNA, 13 bp, Drosophila sp.
tgatccatcatca

SEQ ID NO: 128 - DNA, 13 bp, Drosophila sp.
tcaacatgcaaga

SEQ ID NO: 129 - DNA, 13 bp, Drosophila sp.
tcttaaataaaga

SEQ ID NO: 130 - DNA, 13 bp, Drosophila sp.
tcaaagatctata

SEQ ID NO: 131 - DNA, 13 bp, Drosophila sp.
taatgaattaaca

SEQ ID NO: 132 - DNA, 13 bp, Drosophila sp.
tttaccatcaact

SEQ ID NO: 133 - DNA, 13 bp, Drosophila sp.
taatgaaacaaca

SEQ ID NO: 134 - DNA, 13 bp, Drosophila sp.
gtttcaattaaaa

SEQ ID NO: 135 - DNA, 13 bp, Drosophila sp.
tattcaattataa

SEQ ID NO: 136 - DNA, 13 bp, Drosophila sp.
tcttcaatcgttt

SEQ ID NO: 137 - DNA, 13 bp, Drosophila sp.
tcaacgatccttt

SEQ ID NO: 138 - DNA, 13 bp, artificial
  Table 2 consensus sequence
tcwwcratcaaca

SEQ ID NO: 139 - DNA, 34 bp, artificial; identical to SEQ ID NO: 74
  primer HSP

SEQ ID NO: 140 - DNA, 25 bp, artificial
  VP16 primer
gccctcgatggtagacccgtaattg

SEQ ID NO: 141 - DNA, 24 bp, artificial
  primer Agexon1F
ggaaaccgaggataacgacgaagg

SEQ ID NO: 142 - DNA, 24 bp, artificial
  primer TETRR1
gcggaacgacttggcgttattgcg

SEQ ID NO: 143 - DNA, 6243 bp, artificial
  LA3576 plasmid sequence
cctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctggcgatcgcctaaaggtgttataaatcaaattagttttgttttttcttgaaaactttgcgtttcctttgatcaacttaccgccagggtacctgcagattgtttagcttgttcagctgcgcttgtttatttgcttagctttcgcttagcgacgtgttcactttgcttgtttgaattgaattgtcgctccgtagacgaagcgcctctatttatactccggcgctgtttaaacatccaccatgcgcccgcatcgatctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggagtatgttctctctcttctcttctctctctctttctcgaatgttctctctcttctcttctctctctctttctcgatggccggggcgcgccaggtttcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacggtatcgataagcttgggccccccctcgaggttcccacaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttatcagcgctttaaatttgcgcatg

SEQ ID NO: 144 - DNA, 5746 bp, artificial
  LA3582 plasmid sequence
cgcctaaaggtgttataaatcaaattagttttgttttttcttgaaaactttgcgtttcctttgatcaacttaccgccagggtacctgcagattgtttagcttgttcagctgcgcttgtttatttgcttagctttcgcttagcgacgtgttcactttgcttgtttgaattgaattgtcgctccgtagacgaagcgcctctatttatactccggcgctgtttaaacatccaccatgcgcccgcatcgatctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggagtatgttctctctcttctcttctctctctctttctcgaatgttctctctcttctcttctctctctctttctcgatggccggggcgcgccaggtttcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacggtatcgataagcttgggccccccctcgaggttcccacaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttatcagcgctttaaatttgcgcatgctagtttaatacacctttcgcagcaagtagtatagcttgttgcacagcagacatcgggaacgggttgggttattttcttgagcgtgacggaacagaatctcatgaaaggcctgcaccagatggtagcggttgtggtgaaggctgacttgcgtcatcgtcggagtcagtggaggagttggtggaattgactccgttggacttgttggcgacggtggtggcgaactgaattggttctgattttgctgttgttgcattaaaatctgctgctgctgttgcatcatttgcaactgatactgcttctcgatttcatcatcgatggcgggaatgtagaatgcgattgccatggtgggcgat

SEQ ID NO: 145 - DNA, 15121 bp, artificial
  LA3596 plasmid sequence
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagacaccggtgctacccgccatactcatcgatgcccagcgcgtcggtgaacatttgctcgaactcgaagtcggccatgtccagggcgccgtacggggcgctatcgtggggcgtgaagcccggtcccgggctatctccatcgcccagcatatccaggtcgaaatcgtccagggcgtcggcgtgggccattgccacatcctctccatccaggtgcagctcgtcgcccaggctcacatcggtcggcggggcggtgctcaggcggcgcgtgtgtccggcgggcaggaagctcaggcggggggcggccaggccggcttcctccggggcatcgtcatccggcaggtccagcagtccctcgatggtgctgccatagttgttcttggtacgggcgcggctgtaggcgctgccgctctcgcacttcagctgcttttccaggccgcagatgatcagctccaggccgaacaggaaggccggctcggcgccctggtgatcgaacagctcgatggcctggcgcagcagcggcggcatgctatcggtggtcggggtctcgcgctcctccttggccacctggtgctcctgatcctccagcacacagcccagggtgaagtggcccacggcgctcagggcgtacagggcgttctccaggctgaagccctgctggcacaggaaggccagctggttctccagggtctcgtactgcttctcggtcgggcgggtgcccaggtgcaccttggcgccatcgcggtgcgacagcagggcgcagcggaagctcttggcgttgttgcgcaggaaatcctgccagctctcgccctccagcgggcagaagtgggtgtggtggcgatccagcatttcgatggccagggcgtccagcagggcgcgcttgttcttcacgtgccagtacagggtcggctgttccacgcccagcttctgggccagcttgcgggtggtcaggccctcgataccaacttcgttcagcagctccagggcgctgttgatcaccttgctcttgtccaggcggctgacctgtgaatacggttaatgtcactattagtgatttataaaaataaatttgatttatatatcaacaatttttcatcgcagccttcagctttttgttgaataattataatgatattttttacgattcaaatcatttaattgttactcaacgaaataagtttaattcaaattttaaaacaagattatatattaagattagaataagaaagaactttgttagattatttaattaaaaagattaaaatttaagtctccagtcactatttaaagatcatctttcaaacgttaaagtgaattcaaacgagacgttcaaatttcgattaaacagtaattaactctaaatttctatcacgaattaagttattgaatatgaaggtttatatttatttacatcatctaataggtttgagttgattgttgtaatccgcatgtgccagaagatatcaatttccaaattgtccgagttcatggaatgttgattgttgtttgtgttgctttgtaattgttgcagggagtatttatggtttgttgattgtagtataaggctgtttctaaaggctagaaaataattttatttatttgaaaataagtaaatatacataatattactaacaataggtcgtcctattttttgatattctgcacaaatttttaaaacacaaagattgcaatacttttagacactaatactgcacactctgaaaaattattaaattatttttaaaaacttaccttaatactttagagaaaaatattataccgcacctttctactttatactcactttattataccagttgcatgttgattgtagttctttgacaagaaaatattccatattgctccaaattatcttggtaagttgattggtgcgtcatttgagcaagctaacaccttgtctcatttaagttcgcctcaagatctcatagcatttttaaatatcactatatttagtaagtaattagaattaccatggtggtttgctagcggtacctgcagattgtttagcttgttcagctgcgcttgtttatttgcttagctttcgcttagcgacgtgttcactttgcttgtttgaattgaattgtcgctccgtagacgaagcgcctctatttatactccggcgctcggtccgactctctatcactgatagggagtattgtcctctctatcactgatagggaatgctatgttctctatcactgatagggaagttgatagtctctatcactgatagggagtggtaatttctctatcactgatagggattagtgatgtctctatcactgatagggattggaatattctctatcactgatagggagtggtaatatctctatcactgatagggactggagttttctctatcactgatagggacacgctgactctctatcactgatagggataagcttactctctatcactgatagggagtattgtcctctctatcactgatagggaatgctatgttctctatcactgatagggaagttgatagtctctatcactgatagggagtggtaatttctctatcactgatagggattagtgatgtctctatcactgatagggattggaatattctctatcactgatagggagtggtaatatctctatcactgatagggactggagttttctctatcactgatagggacacgctgactctctatcactgatagggataagcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagatctcgacccaagaaaaagcggaaggtggaggacccgtctggaggcggtggatccggcggtggaggcatgcagatctttgtgaagactttgaccggaaagaccatcaccctcgaggtagagccatcggacaccattgagaatgtaaaggccaagattcaggataaggagggaatccccccagatcagcagcgtctgatcttcgctggtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaagcaaccaactacaatcaacattctatgggataatcgacaagtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataagtatcaaaaaagtaaacctctttttcagtctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaatgtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctataggtcgccaactggaagacggacgcaccctgtccgattacaacatccagaaggagtccacccttcacttggtccttcgtctccgcggtggcatgcagatcggggatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccaggaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgccc

SEQ ID NO: 146 - DNA, 533 bp, artificial
  146 PBW dsx fragment (Fig 6)
gtccaatcgatcacaatgtatcacaacgttgcgaattcagtttcacaatcacacgcaacaaacrcgrcacgttacaaattagttactttgaatcgatcgattatgatgcggccgactcarcggcccccggcagcactaaccagtagtgatttccactttgcagtgaccggaccaaaacttcgaaattcgaattgtaaagtgacagttcatttcccgccaagtgttgtgccagtgtcatgtcgatatttattttattttcttttttgtaggaaaatgctgagcgaaattaataatataagtggtgtactatcgtcgtccatgaagttattttgcgaatgatactttgttttgtatgtgctgtgtgttgtgtggacttttgctgtgcgttgctgtttgcgatggaaggactattgtgtcgtcgccacgctggactattcggtgagtggtagaataatattttatctatttcatcgcggtacaattgactttttattactactcactgctatggaggaatctcaggaacatcgtaa

SEQ ID NO: 147 - DNA, 611 bp, artificial
  Bombyx-dsx fragment (Fig 6)
gcgattatttaattctatatatttttcaaattcagtttctattccactaacaatgtacactacacgtacacatacacacaacaaaatagtgaatcgataaattagtgtgtcataacacattaacaacattgttacacacccacacatacaaatttgctaagttgatagtcgaataatcggaatggttcgcatcacactactaaccagtcgtgatttccactttacagtgaccggacgaaggtggagaaattcgaaatttaaatataaaagtgacaattcgaatttccacgcgcgcgctctagtgatgtgccagtgtgtgaatatcaatattattttttattttcttttttgtaggaaaatgctggaaattaataatataagtggtgtactgtcttcgtcaatgaagttattttgcgaatgatacttagttttacaagtgccgtggtgtgtgttgacacttgctgtgcgatgctgtgcgaatttcaacggaaatatttgttgtcgtaacattggatctatgggtaagtttagtataataactttactctgttcacattagtgaaacatacatttgtaaaatttgtgttttactaatgtgaaatttatttttg

SEQ ID NO: 148 - DNA, 570 bp, artificial
  codling-dsx fragment (Fig 6)
ttacaaacaatgtacggagctacaacgttgcaagttcggtccccacacaacacaatgtgtcataacacattaacaacattgttacacacccacacatacaaatttgctaagttgataaaagagtggtgtgtccgacgaatcagaacatcactaacccagtcgtgatttcatttccacagtgaccggacgaaggtggagaagttcgaaatttaaaaaaagtgaccacattttatttaatagtgatgtgcaagtgatactatttttattttgtttttcttttgtaggaaaatgctgagcgaaataaataattttagtggtgtgctatcgtcatcgatgaagttgttttgcgaatgatactatgttcttcaagtgctgtgttttgtggactgtggggtgactgttcctgtaaataagcttcgttggacattgtgtctcacacatcggatctcatggtaagtgctagtgctagcatyrmaacttaactctctgagcgaattcctttgactctaaagtcacacgracagccatacaatcaaagctacgctctaattttaagatgacawtctgtaa

SEQ ID NO: 149 - DNA, 4389 bp, artificial
  DSX Minigene1 rom construct LA3491
acgacgaacttgtcaaacgatctcaatggctcctggagaagctgcgatacccctgggagatgatgcccctgatgtacgtgatactgaaaggcgccgacggagacgtcaataaagcgcgccaacggattgacgaaggtatgggggttcttaccggttgggactgtttccgaggtatcgatcgggtgtcactcacttcctgggtgctcccattttgtaactgctaacgcttattattgagtttcaggacatctgggatcttcggtcgacggagtctattcccaacagtgccctggatcaaacactgccatcatgcagtttccgtagcctgttgggctacgctccccgacttgacatcccccattcttatcaaacaacaactcaaggcctgagacaacgagtggtggaatttgcgcacgaagtcattggtttgtcctggtaaaagttaaaagggttaactggagggttaattgacacggtttcaactgatggccttattgacacacggatgaaagacttgcacgcttgaccttctgtctgtactaataaaagttacgttggctgggttttggggtcataatggccccaaaatcgaatcgtcataacttcttgaaatacaactcacgtttaagaccattcaagagtattagatcatcgtctataatagcagatttgaaatttacttcacatttcggtattgcagtgccccttgcttccacaatggaattaggtcggtggtgcgtcgatcgtcgcaagtttatcgttaaacagtcaataaaatgagcattttatatcgtgatacatatgagaagatagaggtttcaattaaaacaaatccacatggtgtcgctaataaaattgtgcattttaagcgagttatatcctctgatcaagataaaatagaaaattcgatttttgaatattcaattataagagcctgaataactacaacatgtagtgaatcgaaactgatttatgacggtttgtgaaggttacacgtcctaagcatttggattcaagaaaagcaagagatatgacgaatgtaaactttatcgtatcaatgaagtaactagcgtccagaacagtacaaaccaacatcgtaccgtcgtattccactccggtcgttgcaatatctctaggtccaccgaaaaacactcatgaccaagatcgtgtcgtcgatcttggtccaccgaaacaccgatgtccatatcgtttcgtcgaacttggaccaacgattcatgcaactgatgacaacgcggcccccgggtcgtaccaatatccgaaaaatccaactgttcttctctgcctcgcaggtcaagccgtggtcaatgaatactcacgattgcacaatctgaacatgttcgacggtgtagagttgcgcagtacgacgcgccagtccggatgatagactttttacacgatcagcacgacccactgcgctgcggcaaaggtcgaaccgaaacaagaataaaccacgaagatcagatcgattcgacggaagaagcaatcgaatgcaaagaagaatcggaacgaagaaaactctaaagcatcgcatatttacaaagcataacggaaaacccgcaagttcaaactagtgattagtgtaagatgaagcaaagcagaaatgtagtatctagatttttcgacgttagtttacaaagataaaaaatgaggttggacatacaatcgtgggtattcgtctgagttcgtcacaactgcaccggaaactgtgaaacagaatagagccaacctgtgcgcggagaatgttgaggtcattataagcttccttagcatccacgggtgaaagtcgatcgacggaagcctgcaagactctgtcgatgggctttcgtcctagaagaataagattaaacctgaaatgtattctcccgtggaatggtttcatttgagtaattctgtatcttctccttcccaattccacgaacgcgacgaactctaatacaaacaacataatgaccacagtgcaaatgctgtttaacgataatagcgacatgcagccattctggggctaccacgtgtagctctacttgtgagacagcgttcctaaagagtgtgaaagtgcaaacaagtgatgaaaccaatagtgcaaagcaagtttagagggaaaatttaaaaaatgcaaaacagcagtagtacttaacttttaagattgtgtttcgaaagccgaagtgaggctgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtgcccaaaatgatctgataaacacttatctctctgtagcttgcatgccattgcgagcgtattttggtagctggccgttgccaaacggctccgacaggtactgctattggaggttgtgcacgaccacgttgagtttgccttttgagttggagagtgtgtcttttcgtcatatattcggccttttcaagggtgattttcaggctacgtaatgattgtatagtttaaccagctaaaacatattgatgacaagttctatttcagcaccacaaacaagcctgttaatgtctctcaccgcaaccattgttctgcgcgcgttataatcagcatagaagtttattttctttgggatgattcaaatattacgtgacgcaaagtttgccaattttagaacccctccctcctccacgtaacggcttttgtgtgaaaaatttaaattttgtgtatagaccgtagcatttcggaagaccccctcccttactctgttgagttacgtaaaatttcaacgatccttttgtagttctgaattttatatcagcgtgcagtgttatgaagatatccacagtataaaatattattttattttaaattctatgctgattatcaatgtgttactagtggcttttcatactcatgttgcgagctcgatttggcgcacggtacttatcaaggcatgtatgtatgttgtttgaagcaactgtataactgtttgaaactatctaattggtgagctcgtttcatttagtatataataatgataattgctatggagacgttatttactagcaagtgatttgacgacctgaaatcggaacaaatagacaacgtttttataaatacaataaatcagaactttccattattgggtacaaagagttgcgctatttcgatactgtcagatcagattttccagcacaacgataccttgatatgcgataacttagaattagaccttcaaatccatctctccagctatgaacagtcatatagataaagccaatggcgttatgaggtagcggaaagcgtcatctttccaatgctatctaagtacataatttgctatagctttctattaatcgtagtttgagagatgcaaagtcagttatctcgtatcaaggtttgattgttttggaaattagctaaacagttgacattatcacccgtctttaggggataagcgcatacaaatgtgtatttagttgttcattgaagtaacgtaagataggcaagtatggaaacgagctcaccaaacgtcgaaatacgtctaataaatttgtgttcagcaggatggttcaaaatttatttgcatcacctcaaaattacagtacctagtgctgtttgtgacaaacatcaaaaggtaaaatcaaactcgtggcgtcgtgcaatctccatagaatgaacaatttctaaccgtatttgatggaaagacattgagtctactatcctcttaacagcattgcacttgtctataaacaataaataatttgttcttttttacattttctttccccactttcgccccccccccccccaaaaatcaatccctcaaacaggatacgacatttgttgcatctactttccgaagcgttccagcagacacagacactggccggacgaggagaacatctccgtcacccgcactccgtctgcgtcacggtcgccatgtgccgattttcgtacccggtcacagtccagctcgccggataacaacggtggcgcgctcaatctggacacgaaatctaccaaagcgacgaccgccaccaccgacgacgaagaggttatgtacgagaaacgcagcccgaagtccattgaatctaccgagttgcggtgccgtctggaggaagccttacacagtggcgctgctgctgctgcggctgctgaagaacctctggcgggcggaagcggttcccactggaagagagaaagtttcggctctacggaggagattcccactcgacccgctcacagtgaaccggaagataatggatttgaaaacggattggaagcgcaccagtcccatattctgcacagcatacatcggaa

SEQ ID NO: 150 - DNA, 2572 bp, artificial
  DSX Minigene2 from construct LA3534
ctcgatttcccctcgtttccaatttcagacgacgaacttgtcaaacgatctcaatggctcctggagaagctgcgatacccctgggagatgatgcccctgatgtacgtgatactgaaaggcgccgacggagacgtcaataaagcgcgccaacggattgacgaaggtatgggggttcttaccggttgggactgtttccgaggtatcgatcgggtgtcactcacttcctgggtgctcccattttgtaactgctaacgcttattattgagtttcaggacatctgggatcttcggtcgacggagtctattcccaacagtgccctggatcaaacactgccatcatgcagtttccgtagcctgttgggctacgctccccgacttgacatcccccattcttatcaaacaacaactcaaggcctgagacaacgagtggtggaatttgcgcacgaagtcattggtttgtcctggtaaaagttaaaagggttaactggagggttaattgacacggtttcaactgatggccttattgacacacggatgaaagacttgcacgcttgaccttctgtctgtactaataaaagttacgttggctgggttttggggtcataatggccccaaaatcgaatcgtcataacttcttgaaatacaactcacgtttaagaccattcaagagtattagatcatcgtctataatagcagatttgaaatttacttcacatttcggtattgcagtgccccttgcttccacaatggaattagttaaagtttcgagagcattgtcaatatcaagtgttgttagcaaacaaatgctaacatcaagattactatcgatgtttgattcacatgtattccaatcagctcgtaaaaaatggaaagtggagctgatagggttgaggtctcacgtgctccaaatcatcacctccaagttagttctaatacactccgttatatgaaatatggtggtgcgtcgatcgtcgcaagtttatcgttaaacagtcaataaaatgagcattttatatcgtgatacatatgagaagatagaggtttcaattaaaacaaatccacatggtgtcgctaataaaattgtgcattttaagcgagttatatcctctgatcaagataaaatagaaaattcgatttttgaatattcaattataagagcctgaataactacaacatgtagtgaatcgaaactgatttatgacggtttgtgaaggttacacgtcctaagcatttggattcaagaaaagcaagagatatgacgaatgtaaactttatcgtatcaatgaagtaactagcgtccagaacagtacaaaccaacatcgtaccgtcgtattccactccggtcgttgcaatatctctaggtccaccgaaaaacactcatgaccaagatcgtgtcgtcgatcttggtccaccgaaacaccgatgtccatatcgtttcgtcgaacttggaccaacgattcatgcaactgatgacaacgcggcccccgggtcgtaccaatatccgaaaaatccaactgttcttctctgcctcgcaggtcaagccgtggtcaatgaatactcacgattgcacaatctgaacatgttcgacggtgtagagttgcgcagtacgacgcgccagtccggatgatagactttttacacgatcagcacgacccactgcgctgcggcaaaggtcgaaccgaaacaagaataaaccacgaagatcagatcgattcgacggaagaagcaatcgaatgcaaagaagaatcggaatgaagaaaactctaaagcatcgcatatttacaaagcataacggaaaacccgcaagttcaaactagtgattagtgtaagatgaagcaaagcagaaatgtagtatctagatttttcgacgttagtttacaaagataagaaatgaggttggacatacaatcgtgggtattcgtctgagttcgtcacaactgcaccggaaactgtgaaacagaatagagccaacctgtgcgcggagaatgttgaggtcattataagcttccttagcatccacgggtgaaagtcgatcgacggaagcctgcaagactctgtcgatgggctttcgtcctagaagaataagattaaacctgaaatgtattctcccgtggaatggtttcatttgagtaattctgtatcttctccttcccaattccacgaacgcgacgaactctaatacaaacaacataatgaccacagtgcaaatgctgtttaacgataatagcgacatgcagccattctggggctaccacgtgtagctctacttgtgagacagcgttcctaaagagtgtgaaagtgcaaacaagtgatgaaaccaatagtgcaaagcaagtttagagggaaaatttaaaaaatgcaaaacagcagtagtacttaacttttaagattgtgtttcgaaagccgaagtgtgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtg

SEQ ID NO: 151 - DNA, 18790 bp, artificial
  LA3619 whole plasmid sequence
cgcgcctaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgttgcacattccgatgtatgctgtgcagaatatgggactggtgcgcttccaatccgttttcaaatccattatcttccggttcactgtgagcgggtcgagtgggaatctcctccgtagagccgaaactttctctcttccagtgggaaccgcttccgcccgccagaggttcttcagcagccgcagcagcagcagcgccactgtgtaaggcttcctccagacggcaccgcaactcggtagattcaatggacttcgggctgcgtttctcgtacataacctcttcgtcgtcggtggtggcggtcgtcgctttggtagatttcgtgtccagattgagcgcgccaccgttgttatccggcgagctggactgtgaccgggtacgaaaatcggcacatggcgaccgtgacgcagacggagtgcgggtgacggagatgttctcctcgtccggccagtgtctgtgtctgctggaacgcttcggaaagtagatgcaacaaatgtcgtatcctgtttgagggattgatttttggggggggggggggcgaaagtggggaaagaaaatgtaaaaaagaacaaattatttattgtttatagacaagtgcaatgctgttaagaggatagtagactcaatgtctttccatcaaatacggttagaaattgttcattctatggagattgcacgacgccacgagtttgattttaccttttgatgtttgtcacaaacagcactaggtactgtaattttgaggtgatgcaaataaattttgaaccatcctgctgaacacaaatttattagacgtatttcgacgtttggtgagctcgtttccatacttgcctatcttacgttacttcaatgaacaactaaatacacatttgtatgcgcttatcccctaaagacgggtgataatgtcaactgtttagctaatttccaaaacaatcaaaccttgatacgagataactgactttgcatctctcaaactacgattaatagaaagctatagcaaattatgtacttagatagcattggaaagatgacgctttccgctacctcataacgccattggctttatctatatgactgttcatagctggagagatggatttgaaggtctaattctaagttatcgcatatcaaggtatcgttgtgctggaaaatctgatctgacagtatcgaaatagcgcaactctttgtacccaataatggaaagttctgatttattgtatttataaaaacgttgtctatttgttccgatttcaggtcgtcaaatcacttgctagtaaataacgtctccatagcaattatcattattatatactaaatgaaacgagctcaccaattagatagtttcaaacagttatacagttgcttcaaacaacatacatacatgccttgataagtaccgtgcgccaaatcgagctcgcaacatgagtatgaaaagccactagtaacacattgataatcagcatagaatttaaaataaaataatattttatactgtggatatcttcataacactgcacgctgatataaaattcagaactacaaaaggatcgttgaaattttacgtaactcaacagagtaagggagggggtcttccgaaatgctacggtctatacacaaaatttaaatttttcacacaaaagccgttacgtggaggagggaggggttctaaaattggcaaactttgcgtcacgtaatatttgaatcatcccaaagaaaataaacttctatgctgattataacgcgcgcagaacaatggttgcggtgagagacattaacaggcttgtttgtggtgctgaaatagaacttgtcatcaatatgttttagctggttaaactatacaatcattacgtagcctgaaaatcacccttgaaaaggccgaatatatgacgaaaagacacactctccaactcaaaaggcaaactcaacgtggtcgtgcacaacctccaatagcagtacctgtcggagccgtttggcaacggccagctaccaaaatacgctcgcaatggcatgcaagctacagagagataagtgtttatcagatcattttgggcaccgaaaccgaccgatgtcggaaacgattgaagagatataatctctggtttgtagattgtaggatggttggttgaagatccggtttcccggattttttcggatggatgttgcttgttgatgattctgctgtcgtcgtttttttccggtggcagatggaacagcctcacttcggctttcgaaacacaatcttaaaagttaagtactactgctgttttgcattttttaaattttccctctaaacttgctttgcactattggtttcatcacttgtttgcactttcacactctttaggaacgctgtctcacaagtagagcttgcggtggacaatcaccggtgttagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtggttttccagtggcgccgcttcacgtggtagccccagaatggctgcatgtcgctattatcgttaaacagcatttgcactgtggtcattatgttgtttgtattagagttcgtcgcgttcgtggaattgggaaggagaagatacagaattactcaaatgaaaccattccacgggagaatacatttcaggtttaatcttattcttctaggacgaaagcccatcgacagagtcttgcaggcttccgtcgatcgactttcacccgtggatgctaaggaagcttataatgacctcaacattctccgcgcacaggttggctctattctgtttcacagtttccggtgcagttgtgacgaactcagacgaatacccacgattgtatgtccaacctcattttttatctttgtaaactaacgtcgaaaaatctagatactacatttctgctttgcttcatcttacactaatcactagtttgaacttgcgggttttccgttatgctttgtaaatatgcgatgctttagagttttcttcgttccgattcttctttgcattcgattgcttcttccgtcgaatcgatctgatcttcgtggtttattcttgtttcggttcgacctttgccgcagcgcagtgggtcgtgctgatcgtgtaaaaagtctatcatccggactggcgcgtcgtactgcgcaactctacaccgtcgaacatgttcagattgtgcaatcgtgagtattcattgaccacggcttgacctgcgaggcagagaagaacagttggatttttcggatattggtacgacccgggggccgcgttgtcatcagttgcatgaatcgttggtccaagttcgacgaaacgatatggacatcggtgtttcggtggaccaagatcgacgacacgatcttggtcatgagtgtttttcggtggacctagagatattgcaacgaccggagtggaatacgacggtacgatgttggtttgtactgttctggacgctagttacttcattgatacgataaagtttacattcgtcatatctcttgcttttcttgaatccaaatgcttaggacgtgtaaccttcacaaaccgtcataaatcagtttcgattcactacatgttgtagttattcaggctcttataattgaatattcaaaaatcgaattttctattttatcttgatcagaggatataactcgcttaaaatgcacaattttattagcgacaccatgtggatttgttttaattgaaacctctatcttctcatatgtatcacgatataaaatgctcattttattgactgtttaacgataaacttgcgacgatcgacgcaccaccgacctaattccattgtggaagcaaggggcactgcaataccgaaatgtgaagtaaatttcaaatctgctattatagacgatgatctaatactcttgaatggtcttaaacgtgagttgtatttcaagaagttatgacgattcgattttggggccattatgaccccaaaacccagccaacgtaacttttattagtacagacagaaggtcaagcgtgcaagtctttcatccgtgtgtcaataaggccatcagttgaaaccgtgtcaattaaccctccagttaacccttttaacttttaccaggacaaaccaatgacttcgtgcgcaaattccaccactcgttgtctcaggccttgagttgttgtttgataagaatgggggatgtcaagtcggggagcgtagcccaacaggctacggaaactgcatgatggcagtgtttgatccagggcactgttgggaatagactccgtcgaccgaagatcccagatgtcctgaaactcaataataagcgttagcagttacaaaatgggagcacccaggaagtgagtgacacccgatcgatacctcggaaacagtcccaaccggtaagaacccccataccttcgtcaatccgttggcgcgctttattgacgtctccgtcggcgcctttcagtatcacgtacatcaggggcaccacctcctagggcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcaggtaccctggcggtaagttgatcaaaggaaacgcaaagttttcaagaaaaaacaaaactaatttgatttataacacctttagaaagcggggctagccaccatgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtaggtttaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctactagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacatcatccaaattaataaatgacaagcaatgggtaccatgcggcctggcctcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacctcgaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattacggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccgctcgcccggggaactagttcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctccggactcagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaccgcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgcccgggcgg

SEQ ID NO: 152 - DNA, 19053 bp, artificial
  LA3612 whole plasmid sequence
gggcatctcggactcctgctcaaataaaatgctgatgagcgaaatattcccaaagatttcgttaataaagtgcttacattcattttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttggaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcggtaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgagtccggagaagggcaccacggaggtgatgtgggccacggcgtgctcggtcagctgcacgctgttgccgcccttgtccaggtcggttctggcgatgcggtgctccaccacgtggttggggggcatggtcacgggcttcttggtcttgtaggaggtgtggaactggcatctgtagttgccgccgccctgcagcatcaggaaggcggtcacgtcgcccttcaagatgccgtcgcacacggtcatcttctcgaaggaggggtcccagccggtggtcttcttggccatcacggggccgtcggcggggaagttcacgccgtggaaggtggacttgtgctcgaagcagttgcccttcaggctgatctcccagctggcggtggccacgccgccgtcctcgtaggtgaaggttctctcgtaggacatgccgtcggggaaggcctgcttgaagtagtcgggcatgctggtggggtaggcggtgaagcagcggttgccgtacatgaacacggtggacaggatgtcgaaggagaaggccagggggccgccgttggccatggtcaccttgaaggtggaggtctgggtgccctcgtagggcttgccgctgccctcgcccttcacggtgaagtagtggccgttcacgcagccgtccatgtggtaggtcatcttcatgtcgtcgccgatgaacttgttggacagggccatggtggcgaccggtttgcgcttcttcttgggtggggtgggatcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcgaaatcgataagcttggatcctaattgaattagctctaattgaattagtctctaattgaactagttccccgggcgagcgggccgatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgctttactcgacctaaactttaaacacgtcatagaatcttcgtttgacaaaaaccacattgtggccaagctgtgtgacgcgacgcgcgctaaagaatggcaaaccaagtcgcgcgagcgtcgacctgcaggcatgcaagcttgcatgcctgcaggtcgaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttgtttaaaatataacaaaattgtgatcccacaaaatgaagtggggcaaaatcaaataattaatagtgtccgtaaacttgttggtcttcaactttttgaggaacacgttggacggcaaatccgtgactataacacaagttgatttaataattttagccaacacgtcgggctgcgtgttttttgccgacgcgtctgtgtacacgttgattaactggtcgattaaactgttgaaataatttaatttttggttcttctttaaatctgtgatgaaattttttaaaataactttaaattcttcattggtaaaaaatgccacgttttgcaacttgtgagggtctaatatgaggtcaaactcagtaggagttttatccaaaaaagaaaacatgattacgtctgtacacgaacgcgtattaacgcagagtgcaaagtataagagggttaaaaaatatattttacgcaccatatacgcatcgggttgatatcgttaatatggatcaatttgaacagttgattaacgtgtctctgctcaagtctttgatcaaaacgcaaatcgacgaaaatgtgtcggacaatatcaagtcgatgagcgaaaaactaaaaaggctagaatacgacaatctcacagacagcgttgagatatacggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttggaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttgcggccggccagatttaaatgagcggccgtaattaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatcacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtaccggtttgcgcttcttcttgggtggggtgggatcgactctagcggtaccccgattgtttagcttgttcagctgcgcttgtttatttgcttagctttcgcttagcgacgtgttcactttgcttgtttgaattgaattgtcgctccgtagacgaagcgcctctatttatactccggcggtcgagggttcgaaatcgataagcttggatcctcgaggtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaatatacttcaaggccaccgttatggtgtcattagggacgaggtccaaaacgtcaaaatattttgtaatgatgccgtgaattacgatttcggtgttaattaagtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgaggccaggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagtagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagtttaaacctacccgccatactcatcgatgcccagcgcgtcggtgaacatttgctcgaactcgaagtcggccatgtccagggcgccgtacggggcgctatcgtggggcgtgaagcccggtcccgggctatctccatcgcccagcatatccaggtcgaaatcgtccagggcgtcggcgtgggccattgccacatcctctccatccaggtgcagctcgtcgcccaggctcacatcggtcggcggggcggtgctcaggcggcgcgtgtgtccggcgggcaggaagctcaggcggggggcggccaggccggcttcctccggggcatcgtcatccggcaggtccagcagtccctcgatggtgctgccatagttgttcttggtacgggcgcggctgtaggcgctgcccatggtggctagccccgctttctaaaggtgttataaatcaaattagttttgttttttcttgaaaactttgcgtttcctttgatcaacttaccgccagggtacctgcagattgtttagcttgttcagctgcgcttgtttatttgcttagctttcgcttagcgacgtgttcactttgcttgtttgaattgaattgtcgctccgtagacgaagcgcctctatttatactccggcgctcgcggccgcatcgatctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggagtatgttctctctcttctcttctctctctctttctcgaatgttctctctcttctcttctctctctctttctcgatggccggggcgcgccaggtttcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgaaaacgagcgccggagtataaatagaggcgcttcgtctacggagcgacaattcaattcaaacaagcaaagtgaacacgtcgctaagcgaaagctaagcaaataaacaagcgcagctgaacaagctaaacaatctgccctaggatctcagtggctcctggagaagctgcgatacccctgggagatgatgcccctgatgtacgtgatactgaaaggcgccgacggagacgtcaataaagcgcgccaacggattgacgaaggtatgggggttcttaccggttgggactgtttccgaggtatcgatcgggtgtcactcacttcctgggtgctcccattttgtaactgctaacgcttattattgagtttcaggacatctgggatcttcggtcgacggagtctattcccaacagtgccctggatcaaacactgccatcatgcagtttccgtagcctgttgggctacgctccccgacttgacatcccccattcttatcaaacaacaactcaaggcctgagacaacgagtggtggaatttgcgcacgaagtcattggtttgtcctggtaaaagttaaaagggttaactggagggttaattgacacggtttcaactgatggccttattgacacacggatgaaagacttgcacgcttgaccttctgtctgtactaataaaagttacgttggctgggttttggggtcataatggccccaaaatcgaatcgtcataacttcttgaaatacaactcacgtttaagaccattcaagagtattagatcatcgtctataatagcagatttgaaatttacttcacatttcggtattgcagtgccccttgcttccacaatggaattaggtcggtggtgcgtcgatcgtcgcaagtttatcgttaaacagtcaataaaatgagcattttatatcgtgatacatatgagaagatagaggtttcaattaaaacaaatccacatggtgtcgctaataaaattgtgcattttaagcgagttatatcctctgatcaagataaaatagaaaattcgatttttgaatattcaattataagagcctgaataactacaacatgtagtgaatcgaaactgatttatgacggtttgtgaaggttacacgtcctaagcatttggattcaagaaaagcaagagatatgacgaatgtaaactttatcgtatcaatgaagtaactagcgtccagaacagtacaaaccaacatcgtaccgtcgtattccactccggtcgttgcaatatctctaggtccaccgaaaaacactcatgaccaagatcgtgtcgtcgatcttggtccaccgaaacaccgatgtccatatcgtttcgtcgaacttggaccaacgattcatgcaactgatgacaacgcggcccccgggtcgtaccaatatccgaaaaatccaactgttcttctctgcctcgcaggtcaagccgtggtcaatgaatactcacgattgcacaatctgaacatgttcgacggtgtagagttgcgcagtacgacgcgccagtccggatgatagactttttacacgatcagcacgacccactgcgctgcggcaaaggtcgaaccgaaacaagaataaaccacgaagatcagatcgattcgacggaagaagcaatcgaatgcaaagaagaatcggaacgaagaaaactctaaagcatcgcatatttacaaagcataacggaaaacccgcaagttcaaactagtgattagtgtaagatgaagcaaagcagaaatgtagtatctagatttttcgacgttagtttacaaagataaaaaatgaggttggacatacaatcgtgggtattcgtctgagttcgtcacaactgcaccggaaactgtgaaacagaatagagccaacctgtgcgcggagaatgttgaggtcattataagcttccttagcatccacgggtgaaagtcgatcgacggaagcctgcaagactctgtcgatgggctttcgtcctagaagaataagattaaacctgaaatgtattctcccgtggaatggtttcatttgagtaattctgtatcttctccttcccaattccacgaacgcgacgaactctaatacaaacaacataatgaccacagtgcaaatgctgtttaacgataatagcgacatgcagccattctggggctaccacgtgtggctctacttgcgatccaaaatgcagatcttcgtcaagaccctgaccggcaagaccatcaccctggaggtggagccgagcgataccatcgagaacgtgaaggccaagatccaggacaaggagggcatcccgccggatcagcagcgcctgatcttcgccggacgccagctggaggatggccgcaccctgagcgactacaacatccagaaggagagcaccctgcacctggtgctgcgcctgcgcggtggtatggtcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggctaacaccggtgattgtccaccgcaagctctacttgtgagacagcgttcctaaagagtgtgaaagtgcaaacaagtgatgaaaccaatagtgcaaagcaagtttagagggaaaatttaaaaaatgcaaaacagcagtagtacttaacttttaagattgtgtttcgaaagccgaagtgaggctgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtgcccaaaatgatctgataaacacttatctctctgtagcttgcatgccattgcgagcgtattttggtagctggccgttgccaaacggctccgacaggtactgctattggaggttgtgcacgaccacgttgagtttgccttttgagttggagagtgtgtcttttcgtcatatattcggccttttcaagggtgattttcaggctacgtaatgattgtatagtttaaccagctaaaacatattgatgacaagttctatttcagcaccacaaacaagcctgttaatgtctctcaccgcaaccattgttctgcgcgcgttataatcagcatagaagtttattttctttgggatgattcaaatattacgtgacgcaaagtttgccaattttagaacccctccctcctccacgtaacggcttttgtgtgaaaaatttaaattttgtgtatagaccgtagcatttcggaagaccccctcccttactctgttgagttacgtaaaatttcaacgatccttttgtagttctgaattttatatcagcgtgcagtgttatgaagatatccacagtataaaatattattttattttaaattctatgctgattatcaatgtgttactagtggcttttcatactcatgttgcgagctcgatttggcgcacggtacttatcaaggcatgtatgtatgttgtttgaagcaactgtataactgtttgaaactatctaattggtgagctcgtttcatttagtatataataatgataattgctatggagacgttatttactagcaagtgatttgacgacctgaaatcggaacaaatagacaacgtttttataaatacaataaatcagaactttccattattgggtacaaagagttgcgctatttcgatactgtcagatcagattttccagcacaacgataccttgatatgcgataacttagaattagaccttcaaatccatctctccagctatgaacagtcatatagataaagccaatggcgttatgaggtagcggaaagcgtcatctttccaatgctatctaagtacataatttgctatagctttctattaatcgtagtttgagagatgcaaagtcagttatctcgtatcaaggtttgattgttttggaaattagctaaacagttgacattatcacccgtctttaggggataagcgcatacaaatgtgtatttagttgttcattgaagtaacgtaagataggcaagtatggaaacgagctcaccaaacgtcgaaatacgtctaataaatttgtgttcagcaggatggttcaaaatttatttgcatcacctcaaaattacagtacctagtgctgtttgtgacaaacatcaaaaggtaaaatcaaactcgtggcgtcgtgcaatctccatagaatgaacaatttctaaccgtatttgatggaaagacattgagtctactatcctcttaacagcattgcacttgtctataaacaataaataatttgttcttttttacattttctttccccactttcgccccccccccccccaaaaatcaatccctcaaacaggatacgacatttgttgcatctactttccgaagcgttccagcagacacagacactggccggacgaggagaacatctccgtcacccgcactccgtctgcgtcacggtcgccatgtgccgattttcgtacccggtcacagtccagctcgccggataacaacggtggcgcgctcaatctggacacgaaatctaccaaagcgacgaccgccaccaccgacgacgaagaggttatgtacgagaaacgcagcccgaagtccattgaatctaccgagttgcggtgccgtctggaggaagccttacacagtggcgctgctgctgctgcggctgctgaagaacctctggcgggcggaagcggttcccactggaagagagaaagtttcggctctacggaggagattcccactcgacccgctcacagtgaaccggaagataatggatttgaaaacggattggaagcgcaccagtcccatattctgcacagcatacatcggaatgtgcaacgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaggcgcgccgccc

SEQ ID NO: 153 - DNA, 10540 bp, artificial
  LA3491 plasmid sequence
ctaggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagcttcacgacgaacttgtcaaacgatctcaatggctcctggagaagctgcgatacccctgggagatgatgcccctgatgtacgtgatactgaaaggcgccgacggagacgtcaataaagcgcgccaacggattgacgaaggtatgggggttcttaccggttgggactgtttccgaggtatcgatcgggtgtcactcacttcctgggtgctcccattttgtaactgctaacgcttattattgagtttcaggacatctgggatcttcggtcgacggagtctattcccaacagtgccctggatcaaacactgccatcatgcagtttccgtagcctgttgggctacgctccccgacttgacatcccccattcttatcaaacaacaactcaaggcctgagacaacgagtggtggaatttgcgcacgaagtcattggtttgtcctggtaaaagttaaaagggttaactggagggttaattgacacggtttcaactgatggccttattgacacacggatgaaagacttgcacgcttgaccttctgtctgtactaataaaagttacgttggctgggttttggggtcataatggccccaaaatcgaatcgtcataacttcttgaaatacaactcacgtttaagaccattcaagagtattagatcatcgtctataatagcagatttgaaatttacttcacatttcggtattgcagtgccccttgcttccacaatggaattaggtcggtggtgcgtcgatcgtcgcaagtttatcgttaaacagtcaataaaatgagcattttatatcgtgatacatatgagaagatagaggtttcaattaaaacaaatccacatggtgtcgctaataaaattgtgcattttaagcgagttatatcctctgatcaagataaaatagaaaattcgatttttgaatattcaattataagagcctgaataactacaacatgtagtgaatcgaaactgatttatgacggtttgtgaaggttacacgtcctaagcatttggattcaagaaaagcaagagatatgacgaatgtaaactttatcgtatcaatgaagtaactagcgtccagaacagtacaaaccaacatcgtaccgtcgtattccactccggtcgttgcaatatctctaggtccaccgaaaaacactcatgaccaagatcgtgtcgtcgatcttggtccaccgaaacaccgatgtccatatcgtttcgtcgaacttggaccaacgattcatgcaactgatgacaacgcggcccccgggtcgtaccaatatccgaaaaatccaactgttcttctctgcctcgcaggtcaagccgtggtcaatgaatactcacgattgcacaatctgaacatgttcgacggtgtagagttgcgcagtacgacgcgccagtccggatgatagactttttacacgatcagcacgacccactgcgctgcggcaaaggtcgaaccgaaacaagaataaaccacgaagatcagatcgattcgacggaagaagcaatcgaatgcaaagaagaatcggaacgaagaaaactctaaagcatcgcatatttacaaagcataacggaaaacccgcaagttcaaactagtgattagtgtaagatgaagcaaagcagaaatgtagtatctagatttttcgacgttagtttacaaagataaaaaatgaggttggacatacaatcgtgggtattcgtctgagttcgtcacaactgcaccggaaactgtgaaacagaatagagccaacctgtgcgcggagaatgttgaggtcattataagcttccttagcatccacgggtgaaagtcgatcgacggaagcctgcaagactctgtcgatgggctttcgtcctagaagaataagattaaacctgaaatgtattctcccgtggaatggtttcatttgagtaattctgtatcttctccttcccaattccacgaacgcgacgaactctaatacaaacaacataatgaccacagtgcaaatgctgtttaacgataatagcgacatgcagccattctggggctaccacgtgtagctctacttgtgagacagcgttcctaaagagtgtgaaagtgcaaacaagtgatgaaaccaatagtgcaaagcaagtttagagggaaaatttaaaaaatgcaaaacagcagtagtacttaacttttaagattgtgtttcgaaagccgaagtgaggctgttccatctgccaccggaaaaaaacgacgacagcagaatcatcaacaagcaacatccatccgaaaaaatccgggaaaccggatcttcaaccaaccatcctacaatctacaaaccagagattatatctcttcaatcgtttccgacatcggtcggtttcggtgcccaaaatgatctgataaacacttatctctctgtagcttgcatgccattgcgagcgtattttggtagctggccgttgccaaacggctccgacaggtactgctattggaggttgtgcacgaccacgttgagtttgccttttgagttggagagtgtgtcttttcgtcatatattcggccttttcaagggtgattttcaggctacgtaatgattgtatagtttaaccagctaaaacatattgatgacaagttctatttcagcaccacaaacaagcctgttaatgtctctcaccgcaaccattgttctgcgcgcgttataatcagcatagaagtttattttctttgggatgattcaaatattacgtgacgcaaagtttgccaattttagaacccctccctcctccacgtaacggcttttgtgtgaaaaatttaaattttgtgtatagaccgtagcatttcggaagaccccctcccttactctgttgagttacgtaaaatttcaacgatccttttgtagttctgaattttatatcagcgtgcagtgttatgaagatatccacagtataaaatattattttattttaaattctatgctgattatcaatgtgttactagtggcttttcatactcatgttgcgagctcgatttggcgcacggtacttatcaaggcatgtatgtatgttgtttgaagcaactgtataactgtttgaaactatctaattggtgagctcgtttcatttagtatataataatgataattgctatggagacgttatttactagcaagtgatttgacgacctgaaatcggaacaaatagacaacgtttttataaatacaataaatcagaactttccattattgggtacaaagagttgcgctatttcgatactgtcagatcagattttccagcacaacgataccttgatatgcgataacttagaattagaccttcaaatccatctctccagctatgaacagtcatatagataaagccaatggcgttatgaggtagcggaaagcgtcatctttccaatgctatctaagtacataatttgctatagctttctattaatcgtagtttgagagatgcaaagtcagttatctcgtatcaaggtttgattgttttggaaattagctaaacagttgacattatcacccgtctttaggggataagcgcatacaaatgtgtatttagttgttcattgaagtaacgtaagataggcaagtatggaaacgagctcaccaaacgtcgaaatacgtctaataaatttgtgttcagcaggatggttcaaaatttatttgcatcacctcaaaattacagtacctagtgctgtttgtgacaaacatcaaaaggtaaaatcaaactcgtggcgtcgtgcaatctccatagaatgaacaatttctaaccgtatttgatggaaagacattgagtctactatcctcttaacagcattgcacttgtctataaacaataaataatttgttcttttttacattttctttccccactttcgccccccccccccccaaaaatcaatccctcaaacaggatacgacatttgttgcatctactttccgaagcgttccagcagacacagacactggccggacgaggagaacatctccgtcacccgcactccgtctgcgtcacggtcgccatgtgccgattttcgtacccggtcacagtccagctcgccggataacaacggtggcgcgctcaatctggacacgaaatctaccaaagcgacgaccgccaccaccgacgacgaagaggttatgtacgagaaacgcagcccgaagtccattgaatctaccgagttgcggtgccgtctggaggaagccttacacagtggcgctgctgctgctgcggctgctgaagaacctctggcgggcggaagcggttcccactggaagagagaaagtttcggctctacggaggagattcccactcgacccgctcacagtgaaccggaagataatggatttgaaaacggattggaagcgcaccagtcccatattctgcacagcatacatcggaatgtgcaacgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggcccaagcttatcgataccgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacctcgagggggggccccccctcgaggttcccacaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccgtttgacggtatcgataagcttgatggggatccggaacccttaattaccgttcgtataatgtatgctatacgaagttattaggtccctcgacctgcagcccgggggatcca

SEQ ID NO: 154 - DNA, 4446 bp, artificial
  LA3515 plasmid sequence
ggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacggtatcgataagcttgggccccccctcgaggttcccacaatggttaattcgagctcgcccggggatctaattcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttaaatctggcc

SEQ ID NO: 155 - DNA, 12991 bp, artificial
  LA3545 Plasmid sequence
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagcactgggaagttgacgttgatatagagccgaattgaacttcaccgctgcttggtaattactctacaagttcatttaggagaaccggattcgaaagatgattttccagcgtttagctttcagatggccgcatacattttgcaccaccaaaccgaaactcactagcgtatccaatcgttcgttttttggtgccggtgtgttacgaactttagctatcaagctaaagcaatttgctctggtcttccgtgctaaaaagaaaaaaaaactgttttttttttggttttgatatttgcgctatttttacttgggccttaattgaacaaacttttgaaagtttccacagcgaaatcgttttcgacgatgccatttttggtaacatttgcattttcttgctcaaattgcttgcaaaacccgtgaaagacattaatattcgatagtgtcatccaaaatcacgaaaatgattgttgcaaaacgttgaacaatttacacatgtaaaaaacaaccatcgattaatgtttattcaaactttttacaagaagggttattctgatcaatgtcaccccgctgatgaatgttaccccggattacacttctcgaaaagtggttcaaaatgctacttgagaatttttatctgtcaaaggaagcaaattcgagtcgaattaaatggtatagtcctgaattaggtttccatttacttacaggtattccactaaatagctggaagatttattttacacaataatgataattcgtaccccaaagagtgtagccctacttttttctctcttttttttttgtaaattttcatcgctgcgtgccagcttaccgacatgtcgcgacagcataaagagcctgtcaagagatgaagaaaaatgacaaggagtcagtggtcaggtctctgtatcaatatttgacgtcctgactttccaatatacctttccttaaagagtagagatcatgcgatacgtgaataaatatcgtttggacttcgaaatagaacataatttaaggtagctgatcagtagttgaacatcttcagacttctgggacaagaagtgtttttttgtttgtagaaaaggtttttgttaaattatatttgtaagataattcaatgaatatatctctgattcagtaatcaatccgtaccacgcaccgtttaagaaacaccctgtaggtttgcatcacgtctcagacaaaagtgtatcgatgtgcgaacactgcataccggcgctttgcaaataatgccaaatttagatatgcattacattgtcacttcgcaaaacacacactcccaaatgcgtcggaaacctcacccgaacgcacgatcgtaacgcgatcgatcgccgattgattgatcggaattaactatctcaatcgatccttctatggactgatgcatgggccggcacttccgagtataaaaccccggtaaacccaaggaatcactcacaatcggattttgacgctcgctctggtacagttcgatacggtctagtgaaaccgaggataacgacgaaggtttttccccattgatccaggtcggtgtttatgattggtggaaaaagactcgagaaaagttccatcgaagccgttggaaatgtgccgtcttcctgtgacgtcttgtggatccagttccttgttcacgtctggtgatcgtgtaaaatgtgctgtcttgtggcgtcatatgtgttccagatccagtgattacgatccgatgtgatgttgatcccttgtgaacgtcttatcctgttccgtgtgcaccatgcataatgtcgtattacgtaagttctgaagtgaaacagaagagtgaattgaaagtttttttattcaacatcaacctaaatatggactttactttccaagaaaattatgcctgatcaactgtggatagttacaaaaaaaaaaggtttattaattaaattttatgattacataatgtgttgaaaagaacaactgaaattttagaagaagatcttttcgtgcatcaggctttgccaattaattgatgataaattatcatagcaaattaacgtagagactaaaaggtatatcgtcaaatagggcttcttttgacactattttggcattcttgctctttgagaacttgcaaccctaaaatgggatcttcatcagcctagtggttagattcagcagctacaaagcaaaaccatgctgaagggttcgattcccggtcgtttcaggatcttttcgtaattgaaatatccttgactaccctaagtatcattgtgcttgccatttacgaatatacatattacgatatacgaatgagaaaatgacaactttggaaaataaagctctcaatgtttcaataagaaataaatactacatcagtattgaaggctaataacaattacagattagaacctttaaacatcatttctgcaacaggctggataaagtacagttggaggattaaattatgcgattttgcaattttttccgattaaattcatatttattcctggtttggtttttacaaaaaatatttttacatgacgtttgaccccgattccctcaactttgattgttatatttttttttggacaggttgagtttgtgggttttttcctagtgttgctttgctttatgggctctggttatttaaaattaaaatttgacaatcttactacacactccgaaaaaatcatgcgattttacgtcttttggatgcacataaaagaagcgagccaaatgaggtgaatttgtgtcacattttaaatacgatggtgtctgattcgggaaatgtcaatgatagtgtcattcaatcataatgtgaattacgtccgcagtaattttcattatttttaagagtgtactactatttacactacaaaaattttgataccccaggggggaacgaggtcccggatgtccagctggccagattgttggcaacgagccctgtacctattgatcgagtcaccaaagcactcctcaagtgttttaatctcgaccagacggtggacctcggttgttctcattctcggagggcgatttcgcaatcattagtaccaaccacatgtcgaagtcgggagatgttataaaattataaccaattattcaaaaaatgacatcattcaatttgaacaaacgttcgatagaaattatatatgatttcacatgatattaaactacgaagaaaattttacataaggaagtggtataaaacgtaatatgcttaataaaaactttaacccttttgggaggataatattcagaagttctgattcagaaccatctctcatgttatgttcgttttttgttgcttgtcctttatatgccacatgaacaataacaccaatatctatcccatttccaggacctaacggaccttgaagcggcgccactagtaaaccaccatgggcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggctaacaccggaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacatcatccaaattaataaatgacaagcaatgggtaccatgcggccgctcatttaaatctggccggcctggccgatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgctttactcgacctaaactttaaacacgtcatagaatcttcgtttgacaaaaaccacattgtggccaagctgtgtgacgcgacgcgcgctaaagaatggcaaaccaagtcgcgcgagcgtcgacctgcaggcatgcaagcttgcatgcctgcaggtcgaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttgtttaaaatataacaaaattgtgatcccacaaaatgaagtggggcaaaatcaaataattaatagtgtccgtaaacttgttggtcttcaactttttgaggaacacgttggacggcaaatccgtgactataacacaagttgatttaataattttagccaacacgtcgggctgcgtgttttttgccgacgcgtctgtgtacacgttgattaactggtcgattaaactgttgaaataatttaatttttggttcttctttaaatctgtgatgaaattttttaaaataactttaaattcttcattggtaaaaaatgccacgttttgcaacttgtgagggtctaatatgaggtcaaactcagtaggagttttatccaaaaaagaaaacatgattacgtctgtacacgaacgcgtattaacgcagagtgcaaagtataagagggttaaaaaatatattttacgcaccatatacgcatcgggttgatatcgttaatatggatcaatttgaacagttgattaacgtgtctctgctcaagtctttgatcaaaacgcaaatcgacgaaaatgtgtcggacaatatcaagtcgatgagcgaaaaactaaaaaggctagaatacgacaatctcacagacagcgttgagatatacggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttggaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttgcggccagccc

SEQ ID NO: 156 - DNA, 18411 bp, artificial
  LA3604 Plasmid sequence
ttaaaatgaatgtaagcactttattaacgaaatctttgggaatatttcgctcatcagcattttatttgagcaggagtccgagatgcccgggcggcgcgaaactcccctgcaggataacttcgtatagcatacattatacgaagttatcctagggaagttcctatactttctagagaataggaacttcggaataggaacttcttcgaacggccaaaaaggccggccggggcacgggcgccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagacaccggtgttagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgaccataccaccgcgcaggcgcagcaccaggtgcagggtgctctccttctggatgttgtagtcgctcagggtgcggccatcctccagctggcgtccggcgaagatcaggcgctgctgatccggcgggatgccctccttgtcctggatcttggccttcacgttctcgatggtatcgctcggctccacctccagggtgatggtcttgccggtcagggtcttgacgaagatctgcatcgagctagccgtcacacgttttggcgccgcttcaaggtccgttaggtcctggaaatgggatagatattggtgttattgttcatgtggcatataaaggacaagcaacaaaaaacgaacataacatgagagatggttctgaatcagaacttctgaatattatcctcccaaaagggttaaagtttttattaagcatattacgttttataccacttccttatgtaaaattttcttcgtagtttaatatcatgtgaaatcatatataatttctatcgaacgtttgttcaaattgaatgatgtcattttttgaataattggttataattttataacatctcccgacttcgacatgtggttggtactaatgattgcgaaatcgccctccgagaatgagaacaaccgaggtccaccgtctggtcgagattaaaacacttgaggagtgctttggtgactcgatcaataggtacagggctcgttgccaacaatctggccagctggacatccgggacctcgttcccccctggggtatcaaaatttttgtagtgtaaatagtagtacactcttaaaaataatgaaaattactgcggacgtaattcacattatgattgaatgacactatcattgacatttcccgaatcagacaccatcgtatttaaaatgtgacacaaattcacctcatttggctcgcttcttttatgtgcatccaaaagacgtaaaatcgcatgattttttcggagtgtgtagtaagattgtcaaattttaattttaaataaccagagcccataaagcaaagcaacactaggaaaaaacccacaaactcaacctgtccaaaaaaaaatataacaatcaaagttgagggaatcggggtcaaacgtcatgtaaaaatattttttgtaaaaaccaaaccaggaataaatatgaatttaatcggaaaaaattgcaaaatcgcataatttaatcctccaactgtactttatccagcctgttgcagaaatgatgtttaaaggttctaatctgtaattgttattagccttcaatactgatgtagtatttatttcttattgaaacattgagagctttattttccaaagttgtcattttctcattcgtatatcgtaatatgtatattcgtaaatggcaagcacaatgatactcagggcagtcaaggatatttcaattacgaaaagatcctgaaacgaccgggaatcgaacccttcagcatggctttgctttgtagctgctgaatctaaccactaggctgatgaagatcccattttagggttgcaagttctcaaagagcaagaatgccaaaatagtgtcaaaagaagccctatttgacgatataccttttagtctctacgttaatttgctatgataatttatcatcaattaattggcaaagcctgatgcacgaaaagatcttcttctaaaatttcagttgttcttttcaacacattatgtaatcataaaatttataataaaccttttttttttgtaactatccacagttgatcaggcataattttcttggaaagtaaagtccatatttaggttgatgttgaataaaaaaactttcaattcactcttctgtttcacttcagaacttacgtaatacgacattatgcatggtgcacacggaacaggataagacgttcacaagggatcaacatcacatcggatcgtaatcactggatctggaacacatatgacgccacaagacagcacattttacacgatcaccagacgtgaacaaggaactggatccacaagacgtcacaggaagacggcacatttccaacggcttcgatggaacttttctcgagtctttttccaccaatcataaacaccgacctggatcaatggggaaaaaccttcgtcgttatcctcggtttccatggtggcggtccgtatcgaactgtaccagagcgagcgtcaaaatccgattgtgagtgattccttgggtttaccggggttttatactcggaagtgccggcccatgcatcagtccatagaaggatcgattgagatagttaattccgatcaatcaatcggcgatcgatcgcgttacgatcgtgcgttcgggtgaggtttccgacgcatttgggagtgtgtgttttgcgaagtgacaatgtaatgcatatctaaatttggcattatttgcaaagcgccggtatgcagtgttcgcacatcgatacacttttgtctgagacgtgatgcaaacctacagggtgtttcttaaacggtgcgtggtacggattgattactgaatcagagatatattcattgaattatcttacaaatataatttaacaaaaaccttttctacaaacaaaaaaacacttcttgtcccagaagtctgaagatgttcaactactgatcagctaccttaaattatgttctatttcgaagtccaaacgatatttattcacgtatcgcatgatctctactctttaaggaaaggtatattggaaagtcaggacgtcaaatattgatacagagacctgaccactgactccttgtcatttttcttcatctcttgacaggctctttatgctgtcgcgacatgtcggtaagctggcacgcagcgatgaaaatttacaaaaaaaaaagagagaaaaaagtagggctacactctttggggtacgaattatcattattgtgtaaaataaatcttccagctatttagtggaatacctgtaagtaaatggaaacctaattcaggactataccatttaattcgactcgaatttgcttcctttgacagataaaaattctcaagtagcattttgaaccacttttcgagaagtgtaatccggggtaacattcatcagcggggtgacattgatcagaataacccttcttgtaaaaagtttgaataaacattaatcgatggttgttttttacatgtgtaaattgttcaacgttttgcaacaatcattttcgtgattttggatgacactatcgaatattaatgtctttcacgggttttgcaagcaatttgagcaagaaaatgcaaatgttaccaaaaatggcatcgtcgaaaacgatttcgctgtggaaactttcaaaagtttgttcaattaaggcccaagtaaaaatagcgcaaatatcaaaaccaaaaaaaaaacagtttttttttctttttagcacggaagaccagagcaaattgctttagcttgatagctaaagttcgtaacacaccggcaccaaaaaacgaacgattggatacgctagcgagtttcggtttggtggtgcaaaatgtatgcggccatctgaaagctaaacgctggaaaatcatctttcgaatccggttctcctaaatgaacttgtagagtaattaccaagcagcggtgaagttcaattcggctctatatcaacgtcaacttcccagtgcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcaggtaccctggcggtaagttgatcaaaggaaacgcaaagttttcaagaaaaaacaaaactaatttgatttataacacctttagaaagcggggctagccaccatgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtaggtttaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctactagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacatcatccaaattaataaatgacaagcaatgggtaccatgcggcctggcctcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacctcgaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattacggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccgctcgcccggggaactagttcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctccggactcagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaccgcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 157 - DNA, 18073 bp, artificial
  LA3646 Plasmid sequence
ctaggtaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcggtaacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacgcggccgcattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacctcgaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattacggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccgctcgcccggggaactagttcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctccggactcagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaccgcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgcccgggcggcgcgccgaattcttaattaacgccctagccgcgatcgcatccgccgcggtggcggccctaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgttgcacattccgatgtatgctgtgcagaatatgggactggtgcgcttccaatccgttttcaaatccattatcttccggttcactgtgagcgggtcgagtgggaatctcctccgtagagccgaaactttctctcttccagtgggaaccgcttccgcccgccagaggttcttcagcagccgcagcagcagcagcgccactgtgtaaggcttcctccagacggcaccgcaactcggtagattcaatggacttcgggctgcgtttctcgtacataacctcttcgtcgtcggtggtggcggtcgtcgctttggtagatttcgtgtccagattgagcgcgccaccgttgttatccggcgagctggactgtgaccgggtacgaaaatcggcacatggcgaccgtgacgcagacggagtgcgggtgacggagatgttctcctcgtccggccagtgtctgtgtctgctggaacgcttcggaaagtagatgcaacaaatgtcgtatcctgtttgagggattgatttttggggggggggggggcgaaagtggggaaagaaaatgtaaaaaagaacaaattatttattgtttatagacaagtgcaatgctgttaagaggatagtagactcaatgtctttccatcaaatacggttagaaattgttcattctatggagattgcacgacgccacgagtttgattttaccttttgatgtttgtcacaaacagcactaggtactgtaattttgaggtgatgcaaataaattttgaaccatcctgctgaacacaaatttattagacgtatttcgacgtttggtgagctcgtttccatacttgcctatcttacgttacttcaatgaacaactaaatacacatttgtatgcgcttatcccctaaagacgggtgataatgtcaactgtttagctaatttccaaaacaatcaaaccttgatacgagataactgactttgcatctctcaaactacgattaatagaaagctatagcaaattatgtacttagatagcattggaaagatgacgctttccgctacctcataacgccattggctttatctatatgactgttcatagctggagagatggatttgaaggtctaattctaagttatcgcatatcaaggtatcgttgtgctggaaaatctgatctgacagtatcgaaatagcgcaactctttgtacccaataatggaaagttctgatttattgtatttataaaaacgttgtctatttgttccgatttcaggtcgtcaaatcacttgctagtaaataacgtctccatagcaattatcattattatatactaaatgaaacgagctcaccaattagatagtttcaaacagttatacagttgcttcaaacaacatacatacatgccttgataagtaccgtgcgccaaatcgagctcgcaacatgagtatgaaaagccactagtaacacattgataatcagcatagaatttaaaataaaataatattttatactgtggatatcttcataacactgcacgctgatataaaattcagaactacaaaaggatcgttgaaattttacgtaactcaacagagtaagggagggggtcttccgaaatgctacggtctatacacaaaatttaaatttttcacacaaaagccgttacgtggaggagggaggggttctaaaattggcaaactttgcgtcacgtaatatttgaatcatcccaaagaaaataaacttctatgctgattataacgcgcgcagaacaatggttgcggtgagagacattaacaggcttgtttgtggtgctgaaatagaacttgtcatcaatatgttttagctggttaaactatacaatcattacgtagcctgaaaatcacccttgaaaaggccgaatatatgacgaaaagacacactctccaactcaaaaggcaaactcaacgtggtcgtgcacaacctccaatagcagtacctgtcggagccgtttggcaacggccagctaccaaaatacgctcgcaatggcatgcaagctacagagagataagtgtttatcagatcattttgggcaccgaaaccgaccgatgtcggaaacgattgaagagatataatctctggtttgtagattgtaggatggttggttgaagatccggtttcccggattttttcggatggatgttgcttgttgatgattctgctgtcgtcgtttttttccggtggcagatggaacagcctcacttcggctttcgaaacacaatcttaaaagttaagtactactgctgttttgcattttttaaattttccctctaaacttgctttgcactattggtttcatagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtcacttgtttgcactttcacactctttaggaacgctgtctcacaagtagagctacacgtggtagccccagaatggctgtatgtcgctattatcgttaaacagtatttgcactgtggtcattatgttgtttgtattagagttcgtcgcgttcgtggaattgggaaggagaagatacagaattactcaaatgaaaccattccacgggagaatacatttcaggtttaatcttattcttctaggacgaaagcccatcgacagagtcttgcaggcttccgtcgatcgactttcacccgtggatgctaaggaagcttataatgacctcaacattctccgcgcacaggttggctctattctgtttcacagtttccggtgcagttgtgacgaactcagacgaatacccacgattgtatgtccaacctcattttttatctttgtaaactaacgtcgaaaaatctagatactacatttctgctttgcttcatcttacactaatcactagtttgaacttgcgggttttccgttatgctttgtaaatatgcgatgctttagagttttcttcgttccgattcttctttgcattcgattgcttcttccgtcgaatcgatctgatcttcgtggtttattcttgtttcggttcgacctttgccgcagcgcagtgggtcgtgctgatcgtgtaaaaagtctatcatccggactggcgcgtcgtactgcgcaactctacaccgtcgaacatgttcagattgtgcaatcgtgagtattcattgaccacggcttgacctgcgaggcagagaagaacagttggatttttcggatattggtacgacccgggggccgcgttgtcatcagttgcatgaatcgttggtccaagttcgacgaaacgatatggacatcggtgtttcggtggaccaagatcgacgacacgatcttggtcatgagtgtttttcggtggacctagagatattgcaacgaccggagtggaatacgacggtacgatgttggtttgtactgttctggacgctagttacttcattgatacgataaagtttacattcgtcatatctcttgcttttcttgaatccaaatgcttaggacgtgtaaccttcacaaaccgtcataaatcagtttcgattcactacatgttgtagttattcaggctcttataattgaatattcaaaaatcgaattttctattttatcttgatcagaggatataactcgcttaaaatgcacaattttattagcgacaccatgtggatttgttttaattgaaacctctatcttctcatatgtatcacgatataaaatgctcattttattgactgtttaacgataaacttgcgacgatcgacgcaccaccgacctaattccattgtggaagcaaggggcactgcaataccgaaatgtgaagtaaatttcaaatctgctattatagacgatgatctaatactcttgaatggtcttaaacgtgagttgtatttcaagaagttatgacgattcgattttggggccattatgaccccaaaacccagccaacgtaacttttattagtacagacagaaggtcaagcgtgcaagtctttcatccgtgtgtcaataaggccatcagttgaaaccgtgtcaattaaccctccagttaacccttttaacttttaccaggacaaaccaatgacttcgtgcgcaaattccaccactcgttgtctcaggccttgagttgttgtttgataagaatgggggatgtcaagtcggggagcgtagcccaacaggctacggaaactgcatgatggcagtgtttgatccagggcactgttgggaatagactccgtcgaccgaagatcccagatgtcctgaaactcaataataagcgttagcagttacaaaatgggagcacccaggaagtgagtgacacccgatcgatacctcggaaacagtcccaaccggtaagaacccccataccttcgtcaatccgttggcgcgctttattgacgtctccgtcggcgcctttcagtatcacgtacgtcaggggcgtcgtctcccaggggtatcgcagcttctccaggagccgttgagatcgtttgacaagttcgtcgtggtacctggcctgaatctcaacttgcacctgaaggtagtgcagcaaggatgagcaaaagggaagaacccagaaaagaacgggaaaacttaccccaattagaattggctagcgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcggcggatgcgatcgcgg

SEQ ID NO: 158 - DNA, 13293 bp, artificial
  LA3054 plasmid sequence
gggcgccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgacgcgcctcttactagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgccaccacggagacgaaggaccaagtgaagggtggactccttctggatgttgtaatcggacagggtgcgtccatcctcaagctgcttgccggcgaagatcagacgctgctgatctggggggattccctccttatcctgaatcttggccttcacattctcaatggtgtccgaaggctctacctcgagggtgatggtctttccggtcaaagtcttcacgaaaatctgcatcgagctagccagaggctttgagccttcacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccaaatctacgtttaataacaacagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatccaccagagacaggttgcggcggcggttggatggcgtgggcgcgttggcgttgttggaccggctcatgttgtgtcgctgtaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtatggtaccgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggccc

SEQ ID NO: 159 - DNA, 13515 bp, artificial
  LA3056 plasmid sequence
gggcgccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgacgcgcctcttactagaactacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgccaccacggagacgaaggaccaagtgaagggtggactccttctggatgttgtaatcggacagggtgcgtccatcctcaagctgcttgccggcgaagatcagacgctgctgatctggggggattccctccttatcctgaatcttggccttcacattctcaatggtgtccgaaggctctacctcgagggtgatggtctttccggtcaaagtcttcacgaaaatctgcatcgagctagcaaatcgttctgggctgctggaatccttttaaaaaaaatgattttttttttgctataaagctatgaagtagttcacttactgtcgatttgtgacgctctttgcgccattgatttcaacctcctctttactgttgttactccgatctttaggctgtgtttcaaaatgagcacccacattacttacaacattatcagggtttacaacgatgtcgtcgcgttgaaacagaggctttgagccttcacctatagataccatagatgtatggattagtatcatatacatacaaaggctatttttgggacatattaatattaacaatttccgtgatagttttcaccatttttgttgaatgttacgttgaaaatttaaatttgttttaaattaattttaccagtcatgtgttcttaaaagtttttatgattgaaacggcataaagtggttcaaaaatttatcaagaaaggctttccttttttaaatcttatctttttctcttaaaaatcactagtcaattcattattaatttgttaacttgaatttggaatgtctatttactttcagataaattaaagcaagaaacttaatattcgaaaaaaattgattctaaatggaatttcacttgatcttcatgtatgcatatcaatttttatttacattgtataataagtttcgagttgattgttgtaatccacaggtgtcccagagaattaaattccaaattacccaagtttattgaatgttgattgtagtttcagttgctttgttgctgcaacaatggcttgttgattgtagatattttccctttccttggtttacttattacatagactgaaaaagaggtttacttttttgatacttatgaaaaatttctattagtgattactaaccaatcgctatatgtttactagaaaacaaataaactctttacattaacattcaataatgtttgctctgtaaccgacaattgaaggcgttacagcaacagtaatataactagcttcttaaccctcatctattaaccccatcgtttaaaacactatgttaaatggtctaacaaatctagatactaatagatgtcttattacttagcagccacagctgcaacatccaagacaatttttgaaacttcttattgagctcttggcagcagaaatgttggtatttttcacagctttctgaaagaccggcaccttcctccggttcccgtttctgaattcaagaggatttccgacccccaattaatcccgaaacaaataaggtatattcaaaatgatggaaaagtcatggctgctgaccttatttttattcctattgatagaatattattccccttttaaatacactgtactaagaggtccggctataattttactcacttgtcgattatcccatagaatgttgattgtagttggttgcttttccaggtgagagttgatcaagtcacaaaagttagcgtgtgttgattgtagatttgaaggtaaaataatttttgcacccattcatcgggtaaaacgttctccatagaatacatttccatcgataattgataacttatgaatttcaaagaaaaaaatatgcttttaaaattaccaaatctacgtttaataacaacagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatccaccagagacaggttgcggcggcggttggatggcgtgggcgcgttggcgttgttggaccggctcatgttgtgtcgctgtaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtatggtaccgcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcatggtacccattgcttgtcatttattaatttggatgatgtcatttgtttttaaaattgaactggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaaggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggccc

SEQ ID NO: 160 - DNA, 9423 bp, artificial
  LA3488 plasmid sequence
cggcgcgccggctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacgctcgaggttgcacaacactattatcgatttgcagttcgggacataaatgtttaaatatatcgatgtctttgtgatgcgcgcgacatttttgtaggttattgataaaatgaacggatacgttgcccgacattatcattaaatccttggcgtagaatttgtcgggtccattgtccgtgtgcgctagcatgcccgtaacggacctcgtacttttggcttcaaaggttttgcgcacagacaaaatgtgccacacttgcagctctgcatgtgtgcgcgttaccacaaatcccaacggcgcagtgtacttgttgtatgcaaataaatctcgataaaggcgcggcgcgcgaatgcagctgatcacgtacgctcctcgtgttccgttcaaggacggtgttatcgacctcagattaatgtttatcggccgactgttttcgtatccgctcaccaaacgcgtttttgcattaacattgtatgtcggcggatgttctatatctaatttgaataaataaacgataaccgcgttggttttagagggcataataaaagaaatattgttatcgtgttcgccattagggcagtataaattgacgttcatgttggatattgtttcagttgcaagttgacactggcggcgacaagcaattctaattggggtaagttttcccgttcttttctgggttcttcccttttgctcatccttgctgcactaccttcaggtgcaagttgagattcaggccaccatgggagatcccaccccacccaagaagaagcgcaaaccggtcgccaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaaagatctcgacccaagaaaaagcggaaggtggaggacccgtctggaggcggtggatccggcggtggaggcatgcagatctttgtgaagactttgaccggaaagaccatcaccctcgaggtagagccatcggacaccattgagaatgtaaaggccaagattcaggataaggagggaatccccccagatcagcagcgtctgatcttcgctggtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaagcaaccaactacaatcaacattctatgggataatcgacaagtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataagtatcaaaaaagtaaacctctttttcagtctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaatgtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctataggtaagcaactggaagacggacgcaccctgtccgattacaacatccagaaggagtccacccttcacttggtccttcgtctccgcggtggcatgcagatcggggatcccaccccacccaagaagaagcgcaaaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgacccaagaaaaagcggaaggtggaggacccgtaagatccaccgggtctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattaagaggcgcggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacgctcgcgcgacttggtttgccattctttagcgcgcgtcgcgtcacacagcttggccacaatgtggatgtcgacttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatctaccggtcatactcggtggcctccccaccaccaacttttttgcactgcaaaaaaacacgcttttgcacgcgggcccggcgcgccatctgccggccgcatggtac

SEQ ID NO: 161 - DNA, 17781 bp, artificial
  LA3641plasmid sequence
ttaaaatgaatgtaagcactttattaacgaaatctttgggaatatttcgctcatcagcattttatttgagcaggagtccgagatgcccccttcccttaagtcaatattacaaacaatgtggttttccgccaaccacagtgtggttaaattttatcaccgatgatatgaaatttctagctgcaacatgtccacaagaaataccataattctcatggttgcttaacaactgttaattatacatcaggcaaagtattcactggttttcttaatatatctgggaataattacttcaaggaccgggttaacaagataaggtaaccgctctccaacttaatgtccgtgataatatacaaatatgcgtgttgtaacacgtatagcacatataaaactaggtaaagtccggaatagcccactcgggtcctctcgggtcgggctcgggtcggcctcgggcctactcgggtttggccgaagtagatggctcagtgggctcgtgcgccgtctgtcgcggcgcggtgtggggctacaagtgcgctggcggcggcgagcgcatgagcggcacgggcaggtgcggcgcgtactcgcgcgacagcacatagcgcggcgagcagcaaaacgactctttgcgcgcttgcagctccagcagcgagcacagtgagcgctcgtacagccgccactggtgcaccacccaggaggctggaattaacaagacaggtttaaataaaaactacacaaaaaacaaataccctgcctacatgcccaccaagcacttccacgtgacctgggaaaactagaaagccaatttgaatgtacattttgatatctaaattatgtaattttgttattttgtattaaatatgcataacacattacaaattataacaaaatgaccttgttgatgtcacaacgtaagaattggccgtcgtatcgcgttatcacgtttttcgatttaaatcgaggctttaacgtagrcttaggtacgaaagaagcactctaagcgaattaccttgacgctgacgcttgcagcgttgagcgtaaaaactataactgagtcacacgaactcgccgcgaaaaagctggcctaatacaagaaaacagagtgagtagaagttttgtagtcactctaatttctttgtattacaatttgtagcaacgaattgtattatctatatccagatatagatatatgttacatgaatattcctgtcaaattgttacagawtgtcatcttaaaattagagcgtagctttgattgtatggctgtycgtgtgactttagagtcaaaggaattcgctcagagagttaagttkyratgctagcactagcacttaccatgagatccgatgtgtgagacacaatgtccaacgaagcttatttacaggaacagtcaccccacagtccacaaaacacagcacttgaagaacatagtatcattcgcaaaacaacttcatcgatgacgatagcacaccactaaaattatttatttcgctcagcattttcctacaaaagaaaaacaaaataaaaatagtatcacttgcacatcactattaaataaaatgtggtcactttttttaaatttcgaacttctccaccttcgtccggtcactgtggaaatgaaatcacgactgggttagtgatgttctgattcgtcggacacaccactcttttatcaacttagcaaatttgtatgtgtgggtgtgtaacaatgttgttaatgtgttatgacacattgtgttgtgtggggaccgaacttgcaacgttgtagctccgtacattgtttgtaaacggcaggctacgttactatacgtagtacgtaagcgacgtaagcgtgactcaacttcttatcgattacagcgtctttataaatgtaagttatttataatacagtggaacctcgataaggcgaaaataaaacgctgtctcgctccgctcacaccagtgagagtgagagtgagagaagaacctgaactcgcggcgccttaacggtccccagcaagctcggttgtaagtaaacgatggatggattgtgtgaaagaggatatgagaaagaaaggagtgagaagagaggaacatgttgtgccgaccccacataacgtgggataagagcaggaggaagaagagcaagctaacgtagttacgctctcattttaaaacgactagctaaattgctctgaaactttgtactaacaataggattaggcatatcggagtcgcctttaagagcttattacccctccgtcgaaataccacggccaatagtcatatgtattgtttggactgacgtttaactgacatatttgctcctcccccgtaaaatcgttgtacagagaattacagacaaggtgtttccagttgttaaatcctccaagtctaaggctgtaattagtttatgtagcctcagataccaagtataaactaatttcagccctagacatacctcatgtcattgtatgtgcaaagttccattacaatccaacacgcagttttataatgagaacgaaactccgtttgtatgtgaaattcagccgagcttaccattgctagttttaggaataaggggttaaaatttgcaaattcggtctaagtgtgtgtaaaaaacaaaggtcggtttccgaacagaattttggtttctttttgagtgtttctaacggttttgagatgatttaaatggaaccttactttgagacttgcttaggttgcggtgggcgtttttcatcgccatccgaaatggagttagccgccgtattcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgaccattttgcctggggacaacggaaatcgcacagtttgaacgttcgcttggcggcgcggagactgcattttggagaacacgtacatgtatcgggcgataaaaaaaacyttgtcattgtttcattatgaccatgacaaattaaggtgggttattttttgctacttgaatttaattgtcgaamagtaaaaaaaaacgatgcaacttttttatattgaaattgactgattacaaaatgcagccttgctttataatagacacaacatacacggaggaatggactaggaacatctattttatgtaaccttgtacataactaagacctaggttaaaataacgatgtgttaatataatatatagagaacaatataaagcattttgtaccatttggcgttgaaacttttttgcagcaacgaagcgtttggtatacgtactcgtaaatggtgaccgaaaagctggcggcttcctcgcacaagtaattccgccagcatccttagtacaatgcctgaagggtatattttagatttagctaatttattaatttagtttagtatttgtacagttactgcaatacctctgtaccggaactccagctgtgaccttgacttgtttcatgtgtcaactcgccacagtcccaacttgcttaccttcatcaatcttccgtaacaaaaaagtctcggcgaatccctgggctttgctccaatctaagtactctgcgttttcgtaatcgtttcgtgtccgcgagtttacccatattaatactccgtacgacataagtgaatggaagtgagcgtagtatacggatcttaaagtatcactatcagaagacggcggcatcaacggcggtggcagaataacagcgtccgttgctagaattctttagcgcactctacaaaattatatcctggtggattagccgagtcacattccctttcaattcgtctgtaagcattgttatgtacttaaatttaaacttaccttcgtcaatcttccgcgaggcctcctccaggtcggagccggcgtagttgaggatgaccagcacgagcggcaccacctcccaactgttctagggcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcaggtaccctggcggtaagttgatcaaaggaaacgcaaagttttcaagaaaaaacaaaactaatttgatttataacacctttagaaagcggggctagccaccatgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtaggtttaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctactagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacatcatccaaattaataaatgacaagcaatgggtaccatgcggcctggcctcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacctcgaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgagttaattacggccgctcatttaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccgctcgcccggggaactagttcaattagagactaattcaattagagctaattcaattaggatccaagcttatcgatttcgaaccctcgaccgccggagtataaatagaggcgcttcgtctacggagcgacaattcaattcaaacaagcaaagtgaacacgtcgctaagcgaaagctaagcaaataaacaagcgcagctgaacaagctaaacaatcggggtaccgctagagtcgatcccaccccacccaagaagaagcgcaaaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctccggactcagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaccgcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctaggg

SEQ ID NO: 162 - DNA, 15482 bp, artificial
  LA3570 plasmid sequence
gggcggccgtttttcttgaaatattgctctctctttctaaatagcgcgaatccgtcgctgtgcatttaggacatctcagtcgccgcttggagctcccaaacgcgccagtggtagtacacagtactgtgggtgttcagtttgaaatcctcttgcttctccattgtctcggttacctttggtcaaatccatgggttctattgcctatatactcttgcgattaccagtgattgcgctattagctattagatggattgttggccaaacttgtcgcttaagtggctgggaattgtaaccgtaggcccgagtgtaatgatcccccataaaaagttttcgcaatgcctttattttttgttgcaaatctctctttattctgcggtattcttcattattgcggggatggggaaagtgtttatatagaagcaacttacgattgaacccaaatgcacctgacaagcaaggtcaaagggccagatttttaaatatattatttagtcttaggactctctatttgcaattaaattactttgctacctgagggttaaatcttccccattgataataataattccactatatgttcaattgggtttcaccgcgcttagttacatgacgagccctaatgagccgtcggtggtctataaactgtgccttacaaatacttgcaactcttctcgttttgaagtcagcagagttattgctaattgctaattgctaattgcttttaactgatttcttcgaaattggtgctatgtttatggcgctattaacaagtatgaatgtcaggtttaaccaggggatgcttaattgtgttctcaacttcaaaggcagaaatgtttactcttgaccatgggtttaggtataatgttatcaagctcctcgagttaacgttacgttaacgttaacgttcgaggtcgactctagggcctctctagatttacaggtctattttgagctctttgtcagacactgtttgcttgaaattcaagtctgtcagcaccttaaaaccaaaaataaaaagaataataaatgaaatagtacttacttcccgcggcgcaggttcgcatcgctacaagtgcgcgggcggcggggatgatctctgcgtggtaagcggcagaggcaacaggtgtggcgcgtactcgggcgcgatgacgtagcggggtgagcagcacaccgagtacgtccctttgcgcgcttgcagctccaggagcgagcacagcgaccgctcgtacatccgccactggtggaccacccaatgtgcacccaatgtgctgcaaggaaggcggggttaagtcgtcgagaagtgatacaagaaatcggtctttaaagtcgtaaggtccattacctttaaaaatcgaaaacccttaaactactgtgtctagaaatctggaccttacgaggttaagtcgttagagaattgaaagaaagcataaagaaactagaccatatcatcgccttgtagcgaaaccacgtaagcgtttttttgaaaatcaaattaaaaacattctgatacgattttcttcaacaaaatttcattacaggtaaaaattaagaccacraattattgcctgggttgaattgaaacaagcttgtctattgtgtggttttattaacaaaaatcacatccgaaggcgcttrtgtgggtttcattataaagccacgatatacagtctatacatttagctgttcaagttacaggtgaatcgcaacctccaaggttacaagcggtataaaattwatattgttaataatgtcaaatgtaccaactatagttttacattggtcaaatgagcaatgtacggccgtaaaatggccagtcgcagtgccagtaatgtagttttttaaatccgtaaaaattaagtgccatacyttttttanctaccttaaaatacaaaaatattgggaacmcacgaacaccccaataatagtgtttaaacagtcgttgtcataaaacgatatcaataatctttgatgttataaaaatatatgtttttctttattttaattgcccggtagtcatgttgtatacgagtattgtataaagcaatcgttctacaaatgactcgttacgatgttcctgagattcctccatagcagtgagtagtaataaaaagtcaattgtaccgcgatgaaatagataaaatattattctaccactcaccgaatagtccagcgtggcgacgacacaatagtccttccatcgcaaacagcaacgcacagcaaaagtccacacaacacacagcacatacaaaacaaagtatcattcgcaaaataacttcatggacgacgatagtacaccacttatattattaatttcgctcagcattttccaccggtgttagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtggtttcggtccgttagcgagtcgagttcctcagctcgtggccatcgaagatgttcagattgtgcttcctcgcgtactcgttgatgatcatcttccctggaaacatatgacgctagctttacattcgcacagcggggtatgaggaactgcatttattacaatttattatactattattataattcccgtcgtcataattgtcgtcggtcatgtcgtatcaggaggtgaaggatttggtaggaagaagagaggaatggcgattactccaccgacaagagcgcagctcttaaaaaaaaagagagataattcccgtgaccttaatataagcatcatggcttcataacctcgtgagaaaacgcacataatttcccgagaaatgcgtttcggaggtgacctaaccagcccaatacctgtgttgtttgccttcgggttggaaggtcagataggcattcaattctgtaatgaaccggacctgtcaaatcttcaggctaagtacagaaattataccatcaaataaggtaacataattttgatcagatttctttattatttatttatctttagaagacagagagatgaggaggaagggtgcagacaacattgcatcctacgtgcactcaagaacaagtagaatgtctacttgtatttactacctaaaatacattttattggacctcctagatttaattacagttttgaaatctctaacatctaaaataatagccccgggccttcaattattgtaaaaggggaatgaatcttatgttactataggtagtttcgcctcgagaggcattcgcaacttgaccgaacaaacggtttcttcctttagcgaatgtattataattatccaacacacaagactgcacgcagtacaagtaggtaataatgcaatagattgacataaacggcaattaacgaacgacagacgtacctaccgcggtgtagagttgtagacctatgattattcttcacggagttttttattacaaactgtggtaaaacctttataaaccaccgtaatatacaagaataaagaacgaaactaattatgtataaacaacttatataaataccactgctggacgcagacgtcccctcaatcaactggacagggaagatcgtactccaccacgctgcttcgttacgggttggtagagaattaaataaatgaattgtatgaaaaaaaaaacgtaagtaaacatataaaaaatgtaagttttctatcaaaaacttcacctcgtattcaaagaacgcaaagaacttgtaatcaatcagtaattatcgtaccttcatcaatttttccagaagcctcgtcgaggcctagggcagattgtttagcttgttcagctgcgcttgtttatttgcttagctttcgcttagcgacgtgttcactttgcttgtttgaattgaattgtcgctccgtagacgaagcgcctctatttatactccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatgcggccgcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcaggtaccctggcggtaagttgatcaaaggaaacgcaaagttttcaagaaaaaacaaaactaatttgatttataacacctttagaaagcggggctagccaccatgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtaggtttaaactcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcttacgggtcctccaccttccgctttttcttgggtcgagatctcaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccggtttgcgcttcttcttgggtggggtgggatctcccatggtggcctgaatctcaacttgcacctgaaggtagtgcagcaaggatgagcaaaagggaagaacccagaaaagaacgggaaaacttaccccaattagaattgcttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcgataacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctactagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaacctcgagcgtccgtttgatttaacgtatagcttgcaaatgaattatttaattatcaatcatgttttacgcgtagaattctacccgtaaagcgagtttagttatgagccatgtgcaaaacatgacatcagcttttatttttataacaaatgacatcatttcttgattgtgttttacacgtagaattctactcgtaaagcgagttcagttttgaaaaacaaatgacatcatctttttgattgtgctttacaagtagaattctacccgtaaatcaagttcggttttgaaaaacaaatgagtcatattgtatgatatcatattgcaaaacaaatgactcatcaatcgatcgtgcgttacacgtagaattctactcgtaaagcgagtttatgagccgtgtgcaaaacatgacatcatctcgatttgaaaaacaaatgacatcatccactgatcgtgcattacaagtagaattctactcgtaaagccagttcggttatgagccgtgtacaaaacatgacatcagattatgactcatacttgattgtgttttacgcgtagaattctactcgtaaagccagttcaattttaaaaacaaatgacatcatccaaattaataaatgacaagcaatgggtaccatgcggcctggcctcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacttaattaacaccgaaatcgtaattcacggcatcattacaaaatattttgacgttttggacctcgtccctaatgacaccataacggtggccttgaagtatatttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccaggaaatctggccggccgcaaccattgtgggaaccgtgcgatcaaacaaacgcgagataccggaagtactgaaaaacagtcgctccaggccagtgggaacatcgatgttttgttttgacggaccccttactctcgtctcatataaaccgaagccagctaagatggtatacttattatcatcttgtgatgaggatgcttctatcaacgaaagtaccggtaaaccgcaaatggttatgtattataatcaaactaaaggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgtatatctcaacgctgtctgtgagattgtcgtattctagcctttttagtttttcgctcatcgacttgatattgtccgacacattttcgtcgatttgcgttttgatcaaagacttgagcagagacacgttaatcaactgttcaaattgatccatattaacgatatcaacccgatgcgtatatggtgcgtaaaatatattttttaaccctcttatactttgcactctgcgttaatacgcgttcgtgtacagacgtaatcatgttttcttttttggataaaactcctactgagtttgacctcatattagaccctcacaagttgcaaaacgtggcattttttaccaatgaagaatttaaagttattttaaaaaatttcatcacagatttaaagaagaaccaaaaattaaattatttcaacagtttaatcgaccagttaatcaacgtgtacacagacgcgtcggcaaaaaacacgcagcccgacgtgttggctaaaattattaaatcaacttgtgttatagtcacggatttgccgtccaacgtgttcctcaaaaagttgaagaccaacaagtttacggacactattaattatttgattttgccccacttcattttgtgggatcacaattttgttatattttaaacaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttcgacctgcaggcatgcaagcttgcatgcctgcaggtcgacgctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggtgaggattctgacagtgaaatatcagatcacgtaagtgaagatgacgtccagagcgatacagaagaagcgtttatagatgaggtacatgaagtgcagccaacgtcaagcggtagtgaaatattagacgaacaaaatgttattgaacaaccaggttcttcattggcttctaacagaatcttgaccttgccacagaggactattagaggtaagaataaacattgttggtcaacttcaaagtccacgaggcgtagccgagtctctgcactgaacattgtcagatcggcccggcggagtggacacgctagaccaaatgtgttctgtgatgacctgcagtaggaagacgaataggtggcctatggcattattgtacggaatgataaacattgcctgcataaattcttttattatatacagccataatgtcagtagcaagggagaaaaggtccaaagtcgcaaaaaatttatgagaaacctttacatgagcctgacgtcatcgtttatgcgtaagcgtttagaagctcctactttgaagagatatttgcgcgataatatctctaatattttgccaaatgaagtgcctggtacatcagatgacagtactgaagagccagtaatgaaaaaacgtacttactgtacttactgcccctctaaaataaggcgaaaggcaaatgcatcgtgcaaaaaatgcaaaaaagttatttgtcgagagcataatattgatatgtgccaaagttgtttctgactgactaataagtataatttgtttctattatgtataagttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaaatgaatgtaagcactttattaacgaaatctttgggaatatttcgctcatcagcattttatttgagcaggagtccgagatgccc